(19) United States
(12) Patent Application Publication
(10) Pub. No.: US 2024/0335557 A1
(43) Pub. Date: Oct. 10, 2024

(54) BIS-BENZIMIDAZOLE STING AGONIST IMMUNOCONJUGATES, AND USES THEREOF

(71) Applicant: BOLT BIOTHERAPEUTICS, INC., Redwood City, CA (US)

(72) Inventors: Gary BRANDT, Redwood City, CA (US); Romas KUDIRKA, Redwood City, CA (US); Brian SAFINA, Redwood City, CA (US); Matthew ZHOU, Redwood City, CA (US)

(73) Assignee: BOLT BIOTHERAPEUTICS, INC., Redwood City, CA (US)

(21) Appl. No.: 18/573,094

(22) PCT Filed: Jun. 24, 2022
(86) PCT No.: PCT/US2022/034865

§ 371 Date: Dec. 21, 2023

Related U.S. Application Data
(60) Provisional application No. 63/215,100, filed on Jun. 25, 2021.

(51) Int. Cl. A61K 47/68; A61K 47/60

(52) U.S. Cl. CPC A61K 47/6853; A61K 47/60; A61K 47/6803; A61K 47/6851; A61K 47/6889

(57) ABSTRACT

STING pathway agonism has emerged as a potential therapeutic mechanism to stimulate an innate anti-tumor immune response. While in principle systemic administration of a STING agonist would have many therapeutic benefits, including the delivery of STING to all tumor lesions, such an approach may be limited by toxicity. Antibody-drug conjugates (ADCs) constitute a proven therapeutic modality that is ideally suited to enable systemic administration without associated toxicity concerns via a targeted delivery strategy. Herein, we demonstrate that systemically administered STING agonist ADCs have greater anti-tumor activity as well as greatly improved tolerability compared to an intravenously (IV) administered, unconjugated (free) agonist. We generated novel STING agonist ADCs by leveraging our Immunosynthen platform, in which the chemical scaffold for bioconjugation is optimized for the STING agonist, resulting in an ADC that has desirable physicochemical and drug-like properties. We have studied the in vitro activity and mechanism of action of STING agonist ADCs in monoculture and co-culture systems. STING agonist ADCs were at least 100-fold more potent in inducing interferon and cytokines as well as tumor cell-killing relative to free agonist. STING agonist ADCs against several targets (antigens) have been evaluated for anti-tumor activity and pharmacodynamic and pharmacokinetic properties in multiple xenograft and syngeneic models. A single administration of STING agonist ADC resulted in target-dependent, durable, and complete regressions. Importantly, the STING agonist ADC led to an increase in tumor-localized inflammatory cytokines and significant immune cell infiltration, while levels of systemic cytokines remained low. In contrast, IV administered free agonist induced up to 100-fold higher levels of systemic cytokines with concomitant body weight loss but only modest tumor growth delay. In summary, Immunosynthen represents a novel STING agonist ADC platform. We have demonstrated target-dependent anti-tumor immune responses in vitro and in vivo for multiple targets, tumor models, and mouse strains. In each case the STING agonist ADC was more active and better tolerated than the IV administered free agonist.

### CROSS REFERENCE TO RELATED APPLICATIONS

This non-provisional application claims the benefit of priority to U.S. Provisional Application No. 63/215,100, filed 25 Jun. 2021, which is incorporated by reference in its entirety.

### FIELD OF THE INVENTION

The invention relates generally to an immunoconjugate comprising an antibody conjugated to one or more bis-benzimidazole molecules.

### BACKGROUND OF THE INVENTION

STING (Stimulator of Interferon Genes), also known as transmembrane protein 173 (TMEM173) and MPYS/MITA/ERIS, is a protein encoded in humans by the STING1 gene. STING is broadly expressed, particularly in immune cells, lung, and ovary. STING plays a role in innate immunity by inducing type I interferon production when cells are infected with intracellular pathogens, such as viruses, mycobacteria, and intracellular parasites. Type I interferon, mediated by STING, protects infected cells and nearby cells from local infection by binding to the same cell that secretes it by autocrine signaling and nearby cells by paracrine signaling. STING works as both a direct cytosolic DNA sensor (CDS) and an adaptor protein in Type I interferon signaling through different molecular mechanisms. STING has been shown to activate downstream transcription factors STAT6 and IRF3 through TBK1, which are responsible for antiviral response and innate immune response against intracellular pathogens. Compounds that bind to STING and act as an agonist have been shown to induce secretion of proinflammatory cytokines including type 1 interferons on incubation with human PBMCs (WO 2017/175147). STING modulators may be useful in the treatment of various disorders, for example, allergic diseases, neurodegenerative diseases, pre-cancerous syndromes, and cancer, and may also be useful in immunogenic compositions or vaccine adjuvants.

New compositions and methods for the delivery of antibodies and immune adjuvants are needed in order to reach inaccessible tumors and/or to expand treatment options for cancer patients and other subjects.

### SUMMARY OF THE INVENTION

The invention is generally directed to immunoconjugates comprising an antibody covalently attached to one or more STING agonist moieties by a linker, and having Formula I:

Ab-[L-D]ₚ I
or a pharmaceutically acceptable salt thereof,
wherein:
Ab is the antibody;
p is an integer from 1 to 8;
D is the STING agonist moiety having the formula:

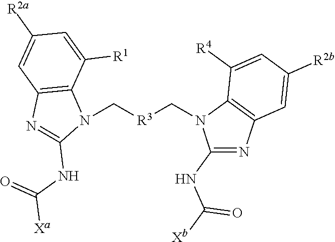

where the substituents are defined herein.

The invention is further directed to use of such an immunoconjugates in the treatment of an illness, in particular cancer.

Another aspect of the invention is a bis-benzimidazole-linker compound.

Another aspect of the invention is a method for treating cancer comprising administering a therapeutically effective amount of an immunoconjugate comprising an antibody linked by conjugation to one or more bis-benzimidazole moieties.

Another aspect of the invention is a use of an immunoconjugate comprising an antibody linked by conjugation to one or more bis-benzimidazole moieties for treating cancer.

Another aspect of the invention is a method of preparing an immunoconjugate by conjugation of one or more bis-benzimidazole moieties with an antibody.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the invention as defined by the claims.

One skilled in the art will recognize many methods and materials similar or equivalent to those described herein, which could be used in the practice of the present invention. The invention is in no way limited to the methods and materials described.

### Definitions

The term “immunoconjugate” refers to an antibody construct that is covalently bonded to an adjuvant moiety via a linker, the term “adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

The terms “immunostimulant” and “immunostimulatory” are used equivalently and refer to a moiety, substance or adjuvant capable of eliciting an immune response in a subject exposed to the immunostimulatory moiety or the immunostimulatory compound after in vivo cleavage of the linker. The terms “adjuvant moiety” or “immunostimulatory moiety” refer to an adjuvant, alternatively referred to as a “payload”, that is covalently bonded to a cell-binding agent, such as an antibody construct, through an elastase-substrate, peptide linker, as described herein. The adjuvant moiety can elicit the immune response while bonded to the antibody construct or after cleavage (e.g., enzymatic cleavage) from the antibody construct following administration of an immunoconjugate to the subject. Immunoconjugates allow targeted delivery of an active adjuvant moiety while the target antigen is bound.

The term “pattern-recognition receptor” (PRR) refers to germline-encoded host sensors which detect molecules typical for pathogens and modulate function of the innate immune system (Mahla, R S et al (2013) Frontiers in Immunology 4:248; Kumar, H et al (2011) Intl. Rev. of Immun. 30:16-34; Schroder K et al (2010) Cell 140(6):821-832). PRRs are proteins expressed mainly by cells of the innate immune system such as dendritic cells, macrophages, monocytes, neutrophils and epithelial cells, to identify pathogen-associated molecular patterns (PAMPs) associated with microbial pathogens, and damage-associated molecular patterns (DAMPs) associated with components of host cells released during cell damage or death. PRRs are also called primitive pattern recognition receptors because they evolved before other parts of the immune system, particularly before adaptive immunity. PRRs also mediate the initiation of antigen-specific adaptive immune response and release of inflammatory cytokines. PRRs include but are not limited to: Toll-like receptors (TLRs), STING-like receptors, RIG-1-like receptors (RLRs), NOD-like receptors (NLRs), C-type lectin-like receptors (CLRs), and DNA sensors.

“Adjuvant” refers to a substance capable of eliciting an immune response in a subject exposed to the adjuvant. The phrase “adjuvant moiety” refers to an adjuvant that is covalently bonded to an antibody construct, e.g., through a linker, as described herein. The adjuvant moiety can elicit the immune response while bonded to the antibody construct or after cleavage (e.g., enzymatic cleavage) from the antibody construct following administration of an immunoconjugate to the subject.

The term “antibody” is used in the broadest sense and specifically encompasses monoclonal antibodies (including full length monoclonal antibodies), polyclonal antibodies, multispecific antibodies (e.g., bispecific antibodies), and antibody fragments so long as they exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

“Antibody” refers to a polypeptide comprising an antigen binding region (including the complementarity determining region (CDRs)) from an immunoglobulin gene or fragments thereof. The term “antibody” specifically encompasses monoclonal antibodies (including full length monoclonal antibodies), polyclonal antibodies, multispecific antibodies (e.g., bispecific antibodies), and antibody fragments that exhibit the desired biological activity. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light is chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding domain. There are four IgG subclasses (IgG1, IgG2, IgG3, and IgG4) in humans, named in order of their abundance in serum (i.e., IgG1 is the most abundant). Typically, the antigen binding domain of an antibody will be most critical in specificity and affinity of binding to cancer cells.

An antibody that targets a particular antigen includes a bispecific or multispecific antibody with at least one antigen binding region that targets the particular antigen. In some embodiments, the targeted monoclonal antibody is a bispecific antibody with at least one antigen binding region that targets tumor cells. Such antigens include but are not limited to: mesothelin, prostate specific membrane antigen (PSMA), HER2, TROP2, CEA, EGFR, 5T4, Nectin4, CD19, CD20, CD22, CD30, CD70, B7H3, B7H4 (also known as 08E), protein tyrosine kinase 7 (PTK7), glypican-3, RG1, fucosyl-GM1, CTLA-4, and CD44 (WO 2017/196598).

“Antibody construct” refers to an antibody or a fusion protein comprising (i) an antigen binding domain and (ii) an Fc domain.

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a T-cell receptor, so as to provide a T-cell receptor like construct that binds TROP2. In yet another embodiment, the antibody fragment is part of a bispecific T-cell engager (BiTEs) comprising a CD1 or CD3 binding domain and linker.

“Epitope” means any antigenic determinant or epitopic determinant of an antigen to which an antigen binding domain binds (i.e., at the paratope of the antigen binding domain). Antigenic determinants usually consist of chemically active surface groupings of molecules, such as amino acids or sugar side chains, and usually have specific three dimensional structural characteristics, as well as specific charge characteristics.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fcγ receptors differ in their affinity for IgG and also have different affinities for the IgG subclasses (e.g., IgG1, IgG2, IgG3, and IgG4).

As used herein, the phrase “immune checkpoint inhibitor” refers to any modulator that inhibits the activity of the immune checkpoint molecule. Immune checkpoint inhibitors can include, but are not limited to, immune checkpoint molecule binding proteins, small molecule inhibitors, antibodies (including bispecific and multispecific antibodies with at least one antigen binding region that targets an immune checkpoint protein, e.g., bispecific or multispecific antibodies that do not exclusively target immune checkpoint proteins, as well as antibodies that are dual immunomodulators (simultaneous targeting two immunomodulating targets), which result in blockade of inhibitory targets, depletion of suppressive cells, and/or activation of effector cells; tumor-targeted immunomodulators (directs potent costimulation to the tumor-infiltrating immune cells by targeting a tumor antigen and costimulatory molecules such as CD40 or 4-1BB); NK-cell redirectors (redirects NK cells to malignant cells by targeting a tumor antigen and CD16A); or T-cell redirectors (redirects T cells to malignant cells by targeting a tumor antigen and CD3)), antibody-derivatives (including Fc fusions, Fab fragments, and scFvs), antibody-drug conjugates, antisense oligonucleotides, siRNA, aptamers, peptides and peptide mimetics.

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The binding agent comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The binding agent can be any of a variety of types of binding agents known in the art that comprise Ig heavy and light chains. For instance, the binding agent can be an antibody, an antigen-binding antibody “fragment,” or a T-cell receptor.

“Biosimilar” refers to an antibody construct that has active properties similar to, for example, sacituzumab, a Trop2-targeting antibody construct previously approved in sacituzumab govitecan (TRODELVY®, Immunomedics, IMMU-132).

“Biobetter” refers to an antibody construct that is an improvement of a previously approved antibody construct, such as sacituzumab or sacituzumab govitecan. The biobetter can have one or more modifications (e.g., an altered glycan profile, or a unique epitope) over the previously approved antibody construct.

“Amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypication) or deglycosylated. Amino acids may be referred to herein by either the commonly known three letter symbols or by the one-letter symbols recommended by the IUPAC-IUB Biochemical Nomenclature Commission.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Naturally-occurring amino acids include those formed in proteins by post-translational modification, such as citrulline (Cit).

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

“Linker” refers to a functional group that covalently bonds two or more moieties in a compound or material. For example, the linking moiety can serve to covalently bond an adjuvant moiety to an antibody construct in an immunoconjugate.

“Linking moiety” refers to a functional group that covalently bonds two or more moieties in a compound or material. For example, the linking moiety can serve to covalently bond an adjuvant moiety to an antibody in an immunoconjugate. Useful bonds for connecting linking moieties to proteins and other materials include, but are not limited to, amides, amines, esters, carbamates, ureas, thioethers, thiocarbamates, thiocarbonates, and thioureas.

“Divalent” refers to a chemical moiety that contains two points of attachment for linking two functional groups, polyvalent linking moieties can have additional points of attachment for linking further functional groups. Divalent radicals may be denoted with the suffix “diyl”. For example, divalent linking moieties include divalent polymer moieties such as divalent poly(ethylene glycol), divalent cycloalkyl, divalent heterocycloalkyl, divalent aryl, and divalent heteroaryl group. A “divalent cycloalkyl, heterocycloalkyl, aryl, or heteroaryl group” refers to a cycloalkyl, heterocycloalkyl, aryl, or heteroaryl group having two points of attachment for covalently linking two moieties in a molecule or material. Cycloalkyl, heterocycloalkyl, aryl, or heteroaryl groups can be substituted or unsubstituted. Cycloalkyl, heterocycloalkyl, aryl, or heteroaryl groups can be substituted with one or more groups selected from halo, hydroxy, amino, alkylamino, amido, acyl, nitro, cyano, and alkoxy.

A wavy line (“ [symbol] ”) represents a point of attachment of the specified chemical moiety. If the specified chemical moiety has two wavy lines (“ [symbol] ”) present, it will be understood that the chemical moiety can be used bilaterally, i.e., as read from left to right or from right to left. In some embodiments, a specified moiety having two wavy lines (“ [symbol] ”) present is considered to be used as read from left to right.

“Alkyl” refers to a straight (linear) or branched, saturated, aliphatic radical having the number of carbon atoms indicated. Alkyl can include any number of carbons, for example from one to twelve. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, and the like. Alkyl groups can be substituted or unsubstituted. Substituted alkyl groups can be substituted with one or more groups selected from halo, hydroxy, amino, oxo (═O), alkylamino, amido, acyl, nitro, cyano, and alkoxy. Substituted alkyl groups can be geminally substituted where a carbon atom of the alkyl forms a spiro, cycloalkyl group such as cyclopropyl, cyclobutyl, cyclopentyl, or cyclohexyl.

The term “alkyldiyl” refers to a divalent alkyl radical. Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group. Alkyldiyl groups can be substituted or unsubstituted. Substituted alkyldiyl groups can be substituted with one or more groups selected from halo, hydroxy, amino, oxo (═O), alkylamino, amido, acyl, nitro, cyano, and alkoxy. Substituted alkyldiyl groups can be geminally substituted where a carbon atom of the alkyl forms a spiro, cycloalkyl group such as cyclopropyl, cyclobutyl, cyclopentyl, or cyclohexyl.

“Alkenyl” refers to a straight (linear) or branched, unsaturated, aliphatic radical having the number of carbon atoms indicated and at least one carbon-carbon double bond, sp2. Alkenyl can include from two to about 12 or more carbons atoms. Alkenyl groups are radicals having “cis” and “trans” orientations, or alternatively, “E” and “Z” orientations. Examples include, but are not limited to, ethylenyl or vinyl (—CH═CH₂), allyl (—CH₂CH═CH₂). butenyl, pentenyl, and isomers thereof. Alkenyl groups can be substituted or unsubstituted. “Substituted alkenyl” groups can be substituted with one or more groups selected from halo, hydroxy, amino, oxo (═O), alkylamino, amido, acyl, nitro, cyano, and alkoxy.

The terms “alkenylene” or “alkenyldiyl” refer to a linear or branched-chain divalent hydrocarbon radical. Examples include, but are not limited to, ethylenylene or vinylene (—CH═CH—), allyl (—CH₂CH═CH—), and the like.

“Alkynyl” refers to a straight (linear) or branched, unsaturated, aliphatic radical having the number of carbon atoms indicated and at least one carbon-carbon triple bond, sp. Alkynyl can include from two to about 12 or more carbons atoms. For example, C₂-C₆ alkynyl includes, but is not limited to ethynyl (—C≡CH), propynyl (propargyl, —CH₂C≡CH), butynyl, pentynyl, hexynyl, and isomers thereof Alkynyl groups can be substituted or unsubstituted. “Substituted alkynyl” groups can be substituted with one or more groups selected from halo, hydroxy, amino, oxo (═O), alkylamino, amido, acyl, nitro, cyano, and alkoxy.

The term “alkynylene” or “alkynyldiyl” refer to a divalent alkynyl radical.

The terms “carbocycle”, “carbocyclyl”, “carbocyclic ring” and “cycloalkyl” refer to a saturated or partially unsaturated, monocyclic, fused bicyclic, spiro, or bridged polycyclic ring assembly containing from 3 to 12 ring atoms, or the number of atoms indicated. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene and adamantane. Carbocyclic groups can also be partially unsaturated, having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

The term “cycloalkyldiyl” refers to a divalent cycloalkyl radical.

“Aryl” refers to a monovalent aromatic hydrocarbon radical of 6-20 carbon atoms (C₆-C₂₀) derived by the removal of one hydrogen atom from a single carbon atom of a parent aromatic ring system. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl.

The terms “arylene” or “aryldiyl” mean a divalent aromatic hydrocarbon radical of 6-20 carbon atoms (C₆-C₂₀) derived by the removal of two hydrogen atom from a two carbon atoms of a parent aromatic ring system. Some aryldiyl groups are represented in the exemplary structures as “Ar”. Aryldiyl includes bicyclic radicals comprising an aromatic ring fused to a saturated, partially unsaturated ring, or aromatic carbocyclic ring. Typical aryldiyl groups include, but are not limited to, radicals derived from benzene (phenyldiyl), substituted benzenes, naphthalene, anthracene, biphenylene, indenylene, indanylene, 1,2-dihydronaphthalene, 1,2,3,4-tetrahydronaphthyl, and the like. Aryldiyl groups are also referred to as “arylene”, and are optionally substituted with one or more substituents described herein.

The terms “heterocycle”, “heterocyclyl”, and “heterocyclic ring” are used interchangeably herein and refer to a saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents described below. A heterocycle may be a monocycle having 3 to 7 ring members (2 to 6 carbon atoms and 1 to 4 heteroatoms selected from N, O, P, and S) or a bicycle having 7 to 10 ring members (4 to 9 carbon atoms and 1 to 6 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. “Heterocyclyl” also includes radicals where heterocycle radicals are fused with a saturated, partially unsaturated ring, or aromatic carbocyclic or heterocyclic ring. Examples of heterocyclic rings include, but are not limited to, morpholin-4-yl, piperidin-1-yl, piperazinyl, piperazin-4-yl-2-one, piperazin-4-yl-3-one, pyrrolidin-1-yl, thiomorpholin-4-yl, S-dioxothiomorpholin-4-yl, azocan-1-yl, azetidin-1-yl, octahydropyrido[1,2-a]pyrazin-2-yl, [1,4]diazepan-1-yl, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyclo[3.1.0]hexanyl, 3-azabicyclo[4.1.0]heptanyl, azabicyclo[2.2.2]hexanyl, 3H-indolyl quinolizinyl and N-pyridyl ureas. Spiro heterocyclyl moieties are also included within the scope of this definition. Examples of spiro heterocyclyl moieties include azaspiro[2.5]octanyl and azaspiro[2.4]heptanyl. Examples of a heterocyclic group wherein 2 ring atoms are substituted with oxo (═O) moieties are pyrimidinonyl and 1,1-dioxo-thiomorpholinyl. The heterocycle groups herein are optionally substituted independently with one or more substituents described herein.

The term “heterocyclyldiyl” refers to a divalent, saturated or a partially unsaturated (i.e., having one or more double and/or triple bonds within the ring) carbocyclic radical of 3 to about 20 ring atoms in which at least one ring atom is a heteroatom selected from nitrogen, oxygen, phosphorus and sulfur, the remaining ring atoms being C, where one or more ring atoms is optionally substituted independently with one or more substituents as described. Examples of 5-membered and 6-membered heterocyclyldiyls include morpholinyldiyl, piperidinyldiyl, piperazinyldiyl, pyrrolidinyldiyl, dioxanyldiyl, thiomorpholinyldiyl, and S-dioxothiomorpholinyldiyl.

The term “heteroaryl” refers to a monovalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Heteroaryl groups are optionally substituted independently with one or more substituents described herein.

The term “heteroaryldiyl” refers to a divalent aromatic radical of 5-, 6-, or 7-membered rings, and includes fused ring systems (at least one of which is aromatic) of 5-20 atoms, containing one or more heteroatoms independently selected from nitrogen, oxygen, and sulfur. Examples of 5-membered and 6-membered heteroaryldiyls include pyridyldiyl, imidazolyldiyl, pyrimidinyldiyl, pyrazolyldiyl, triazolyldiyl, pyrazinyldiyl, tetrazolyldiyl, furyldiyl, thienyldiyl, isoxazolyldiyldiyl, thiazolyldiyl, oxadiazolyldiyl, oxazolyldiyl, isothiazolyldiyl, and pyrrolyldiyl.

The heterocycle or heteroaryl groups may be carbon (carbon-linked), or nitrogen (nitrogen-linked) bonded where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline.

The terms “halo” and “halogen,” by themselves or as part of another substituent, refer to a fluorine, chlorine, bromine, or iodine atom.

The term “carbonyl,” by itself or as part of another substituent, refers to C(═O) or —C(═O)—, i.e., a carbon atom double-bonded to oxygen and bound to two other groups in the moiety having the carbonyl.

As used herein, the phrase “quaternary ammonium salt” refers to a tertiary amine that has been quaternized with an alkyl substituent (e.g., a C₁-C₄ alkyl such as methyl, ethyl, propyl, or butyl).

The terms “treat,” “treatment,” and “treating” refer to any indicia of success in the treatment or amelioration of an injury, pathology, condition (e.g., cancer), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

The phrases “effective amount” and “therapeutically effective amount” refer to a dose or amount of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)). In the case of cancer, the therapeutically effective amount of the immunoconjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the immunoconjugate may prevent growth and/or kill existing cancer cells, it may be cytostatic and/or cytotoxic. For cancer therapy, efficacy can, for example, be measured by assessing the time to disease progression (TTP) and/or determining the response rate (RR)

“Recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of the claimed adjuvant and antibody construct. These synergistic combinations upon administration elicit a greater effect on immunity, e.g., relative to when the antibody construct or adjuvant is administered in the absence of the other moiety. Further, a decreased amount of the immunoconjugate may be administered (as measured by the total number of antibody constructs or the total number of adjuvants administered as part of the immunoconjugate) compared to when either the antibody construct or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

### Antibodies

The immunoconjugate of the invention comprises an antibody. Included in the scope of the embodiments of the invention are functional variants of the antibody constructs or antigen binding domain described herein. The term “functional variant” as used herein refers to an antibody construct having an antigen binding domain with substantial or significant sequence identity or similarity to a parent antibody construct or antigen binding domain, which functional variant retains the biological activity of the antibody construct or antigen binding domain of which it is a variant. Functional variants encompass, for example, those variants of the antibody constructs or antigen binding domain described herein (the parent antibody construct or antigen binding domain) that retain the ability to recognize target cells expressing Trop2 to a similar extent, the same extent, or to a higher extent, as the parent antibody construct or antigen binding domain.

In reference to the antibody construct or antigen binding domain, the functional variant can, for instance, be at least about 30%, about 50%, about 75%, about 80%, about 85%, about 90%, about 91%, about 92%, about 93%, about 94%, about 95%, about 96%, about 97%, about 98%, about 99% or more identical in amino acid sequence to the antibody construct or antigen binding domain.

A functional variant can, for example, comprise the amino acid sequence of the parent antibody construct or antigen binding domain with at least one conservative amino acid substitution. Alternatively, or additionally, the functional variants can comprise the amino acid sequence of the parent antibody construct or antigen binding domain with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent antibody construct or antigen binding domain.

The antibodies comprising the immunoconjugates of the invention include Fc engineered variants. In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, US 2016/0145350, U.S. Pat. Nos. 7,416,726 and 5,624,821, which are hereby incorporated by reference in their entireties herein.

The antibodies comprising the immunoconjugates of the invention include glycan variants, such as afucosylation. In some embodiments, the Fc region of the binding agents are modified to have an altered glycosylation pattern of the Fc region compared to the native non-modified Fc region.

Amino acid substitutions of the inventive antibody constructs or antigen binding domains are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The antibody construct or antigen binding domain can consist essentially of the specified amino acid sequence or sequences described herein, such that other components, e.g., other amino acids, do not materially change the biological activity of the antibody construct or antigen binding domain functional variant.

In some embodiments, the antibodies in the immunoconjugates contain a modified Fc region, wherein the modification modulates the binding of the Fc region to one or more Fc receptors.

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (Jefferis et al., (2009) mAbs, 1(4):332-338).

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while afucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726, US 2007/0014795 and US 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

In some embodiments, the antibodies in the immunoconjugates contain a modified Fc region, wherein the modification modulates the binding of the Fc region to one or more Fc receptors.

In some embodiments, the Fc region is modified by inclusion of a transforming growth factor beta 1 (TGFβ1) receptor, or a fragment thereof, that is capable of binding TGFβ1. For example, the receptor can be TGFβ receptor II (TGFβRII). In some embodiments, the TGFβ receptor is a human TGFβ receptor. In some embodiments, the IgG has a C-terminal fusion to a TGFβRII extracellular domain (ECD) as described in U.S. Pat. No. 9,676,863, incorporated herein. An “Fc linker” may be used to attach the IgG to the TGFβRII extracellular domain. The Fc linker may be a short, flexible peptide that allows for the proper three-dimensional folding of the molecule while maintaining the binding-specificity to the targets. In some embodiments, the N-terminus of the TGFβ receptor is fused to the Fc of the antibody construct (with or without an Fc linker). In some embodiments, the C-terminus of the antibody construct heavy chain is fused to the TGFβ receptor (with or without an Fc linker). In some embodiments, the C-terminal lysine residue of the antibody construct heavy chain is mutated to alanine.

In some embodiments, the antibodies in the immunoconjugates are glycosylated.

In some embodiments, the antibodies in the immunoconjugates are a cysteine-engineered antibody which provides for site-specific conjugation of an adjuvant, label, or drug moiety to the antibody through cysteine substitutions at sites where the engineered cysteines are available for conjugation but do not perturb immunoglobulin folding and assembly or alter antigen binding and effector functions (Junutula, et al., (2008) Nature Biotech., 26(8):925-932; Doman et al. (2009) Blood 114(13):2721-2729; U.S. Pat. Nos. 7,521,541; 7,723,485; US 2012/0121615; WO 2009/052249). A “cysteine engineered antibody” or “cysteine engineered antibody variant” is an antibody in which one or more residues of an antibody are substituted with cysteine residues. Cysteine-engineered antibodies can be conjugated to the thienoazepine adjuvant moiety as a thienoazepine-linker compound with uniform stoichiometry (e.g., up to two thienoazepine moieties per antibody in an antibody that has a single engineered cysteine site).

In some embodiments, cysteine-engineered antibodies are used to prepare immunoconjugates. Immunoconjugates may have a reactive cysteine thiol residue introduced at a site on the light chain, such as the 149-lysine site (LC K149C), or on the heavy chain such as the 122-serine site (HC S122C), as numbered by Kabat numbering. In other embodiments, the cysteine-engineered antibodies have a cysteine residue introduced at the 118-alanine site (EU numbering) of the heavy chain (HC A118C). This site is alternatively numbered 121 by Sequential numbering or 114 by Kabat numbering. In other embodiments, the cysteine-engineered antibodies have a cysteine residue introduced in: (i) the light chain at G64C, R142C, K188C, L201C, T129C, S114C, or E105C according to Kabat numbering; (ii) the heavy chain at D101C, V184C, T205C, or S122C according to Kabat numbering; or (iii) other cysteine-mutant antibodies, and as described in Bhakta, S. et al, (2013) “Engineering THIOMABs for Site-Specific Conjugation of Thiol-Reactive Linkers”, Laurent Ducry (ed.), Antibody-Drug Conjugates, Methods in Molecular Biology, vol. 1045, pages 189-203; WO 2011/156328; U.S. Pat. No. 9,000,130.

### Immune Checkpoint Inhibitors

In some embodiments, the antibody of an immunoconjugate is an immune checkpoint inhibitor. In some embodiments, the immune checkpoint inhibitor reduces the expression or activity of one or more immune checkpoint proteins. In another embodiment, the immune checkpoint inhibitor reduces the interaction between one or more immune checkpoint proteins and their ligands. Inhibitory nucleic acids that decrease the expression and/or activity of immune checkpoint molecules can also be used in the methods disclosed herein.

Immune checkpoint inhibitors nivolumab and atezolizumab can be modified to include an IgG1 Fc, and subsequently converted into an immunoconjugate of the invention.

Most checkpoint antibodies are designed not to have effector function to kill cells, but rather to block the signalling. Immunoconjugates of the present invention can add back the “effector functionality” needed to activate myeloid immunity.

In some embodiments, the immune checkpoint inhibitor is cytotoxic T-lymphocyte antigen 4 (CTLA4, also known as CD152), T cell immunoreceptor with Ig and ITIM domains (TIGIT), glucocorticoid-induced TNFR-related protein (GITR, also known as TNFRSF18), inducible T cell costimulatory (ICOS, also known as CD278), CD96, poliovirus receptor-related 2 (PVRL2, also known as CD112R, programmed cell death protein 1 (PD-1, also known as CD279), programmed cell death 1 ligand 1 (PD-L1, also known as B7-H3 and CD274), programmed cell death ligand 2 (PD-L2, also known as B7-DC and CD273), lymphocyte activation gene-3 (LAG-3, also known as CD223), B7-H4, killer immunoglobulin receptor (KIR), Tumor Necrosis Factor Receptor superfamily member 4 (TNFRST4, also known as OX40 and CD134) and its ligand OX40L (CD252), indoleamine 2,3-dioxygenase 1 (IDO-1), indoleamine 2,3-dioxygenase 2 (IDO-2), carcinoembryonic antigen-related cell adhesion molecule 1 (CEACAM1), B and T lymphocyte attenuator (BTLA, also known as CD272), T-cell membrane protein 3 (TIM3), the adenosine A2A receptor (A2Ar), and V-domain Ig suppressor of T cell activation (VISTA protein). In some embodiments, the immune checkpoint inhibitor is an inhibitor of CTLA4, PD-1, or PD-L1.

In some embodiments, the antibody is selected from: ipilimumab (also known as YERVOY®) pembrolizumab (also known as KEYTRUDA®), nivolumab (also known as OPDIVO®), atezolizumab (also known as TECENTRIQ®), avelumab (also known as BAVENCIO®), and durvalumab (also known as IMFINZI®).

In some embodiments, the immune checkpoint inhibitor is an inhibitor of CTLA4. In some embodiments, the immune checkpoint inhibitor is an antibody against CTLA4. In some embodiments, the immune checkpoint inhibitor is a monoclonal antibody against CTLA4. In some embodiments, the immune checkpoint inhibitor is a human or humanized antibody against CTLA4. In some embodiments, the immune checkpoint inhibitor reduces the expression or activity of one or more immune checkpoint proteins, such as CTLA4.

In some embodiments, the immune checkpoint inhibitor is an inhibitor of PD-1. In some embodiments, the immune checkpoint inhibitor is an antibody against PD-1. In some embodiments, the immune checkpoint inhibitor is a monoclonal antibody against PD-1. In some embodiments, the immune checkpoint inhibitor is a human or humanized antibody against PD-1. In some embodiments, the immune checkpoint inhibitor reduces the expression or activity of one or more immune checkpoint proteins, such as PD-1.

In some embodiments, the immune checkpoint inhibitor is an inhibitor of PD-L1. In some embodiments, the immune checkpoint inhibitor is an antibody against PD-L1. In some embodiments, the immune checkpoint inhibitor is a monoclonal antibody against PD-L1. In some embodiments, the immune checkpoint inhibitor is a human or humanized antibody against PD-L1. In some embodiments, the immune checkpoint inhibitor reduces the expression or activity of one or more immune checkpoint proteins, such as PD-L1. In some embodiments, the immune checkpoint inhibitor reduces the interaction between PD-1 and PD-L1.

In some embodiments, the immune checkpoint inhibitor is an inhibitor of PD-L2. In some embodiments, the immune checkpoint inhibitor is an antibody against PD-L2. In some embodiments, the immune checkpoint inhibitor is a monoclonal antibody against PD-L2. In some embodiments, the immune checkpoint inhibitor is a human or humanized antibody against PD-L2. In some embodiments, the immune checkpoint inhibitor reduces the expression or activity of one or more immune checkpoint proteins, such as PD-L2. In some embodiments, the immune checkpoint inhibitor reduces the interaction between PD-1 and PD-L2.

In some embodiments, the immune checkpoint inhibitor is an inhibitor of LAG-3. In some embodiments, the immune checkpoint inhibitor is an antibody against LAG-3. In some embodiments, the immune checkpoint inhibitor is a monoclonal antibody against LAG-3. In some embodiments, the immune checkpoint inhibitor is a human or humanized antibody against LAG-3. In some embodiments, the immune checkpoint inhibitor reduces the expression or activity of one or more immune checkpoint proteins, such as LAG-3.

In some embodiments, the immune checkpoint inhibitor is an inhibitor of B7-H4. In some embodiments, the immune checkpoint inhibitor is an antibody against B7-H4. In some embodiments, the immune checkpoint inhibitor is a monoclonal antibody against B7-H4. In some embodiments, the immune checkpoint inhibitor is a human or humanized antibody against B7-H4. In some embodiments, the immune checkpoint inhibitor reduces the expression or activity of one or more immune checkpoint proteins, such as B7-H4.

In some embodiments, the immune checkpoint inhibitor is an inhibitor of KIR. In some embodiments, the immune checkpoint inhibitor is an antibody against KIR. In some embodiments, the immune checkpoint inhibitor is a monoclonal antibody against KIR. In some embodiments, the immune checkpoint inhibitor is a human or humanized antibody against KIR. In some embodiments, the immune checkpoint inhibitor reduces the expression or activity of one or more immune checkpoint proteins, such as KIR.

In some embodiments, the immune checkpoint inhibitor is an inhibitor of TNFRSF4. In some embodiments, the immune checkpoint inhibitor is an antibody against TNFRSF4. In some embodiments, the immune checkpoint inhibitor is a monoclonal antibody against TNFRSF4. In some embodiments, the immune checkpoint inhibitor is a human or humanized antibody against TNFRSF4. In some embodiments, the immune checkpoint inhibitor reduces the expression or activity of one or more immune checkpoint proteins, such as TNFRSF4.

In some embodiments, the immune checkpoint inhibitor is an inhibitor of OX40L. In some embodiments, the immune checkpoint inhibitor is an antibody against OX40L. In some embodiments, the immune checkpoint inhibitor is a monoclonal antibody against OX40L. In some embodiments, the immune checkpoint inhibitor is a human or humanized antibody against OX40L. In some embodiments, the immune checkpoint inhibitor reduces the expression or activity of one or more immune checkpoint proteins, such as OX40L. In some embodiments, the immune checkpoint inhibitor reduces the interaction between TNFRSF4 and OX40L. In some embodiments, the immune checkpoint inhibitor is an inhibitor of IDO-1. In some embodiments, the immune checkpoint inhibitor is an antibody against IDO-1. In some embodiments, the immune checkpoint inhibitor is a monoclonal antibody against IDO-1, in some embodiments, the immune checkpoint inhibitor is a human or humanized antibody against IDO-1. In some embodiments, the immune checkpoint inhibitor reduces the expression or activity of one or more immune checkpoint proteins, such as IDO-1.

In some embodiments, the immune checkpoint inhibitor is an inhibitor of IDO-2. In some embodiments, the immune checkpoint inhibitor is an antibody against IDO-2. In some embodiments, the immune checkpoint inhibitor is a monoclonal antibody against IDO-2. In some embodiments, the immune checkpoint inhibitor is a human or humanized antibody against IDO-2. In some embodiments, the immune checkpoint inhibitor reduces the expression or activity of one or more immune checkpoint proteins, such as IDO-2.

In some embodiments, the immune checkpoint inhibitor is an inhibitor of CEACAM1. In some embodiments, the immune checkpoint inhibitor is an antibody against CEACAM1. In some embodiments, the immune checkpoint inhibitor is a monoclonal antibody against CEACAM1. In some embodiments, the immune checkpoint inhibitor is a human or humanized antibody against CEACAM1. In some embodiments, the immune checkpoint inhibitor reduces the expression or activity of one or more immune checkpoint proteins, such as CEACAM1.

In some embodiments, the immune checkpoint inhibitor is an inhibitor of BTLA. In some embodiments, the immune checkpoint inhibitor is an antibody against BTLA. In some embodiments, the immune checkpoint inhibitor is a monoclonal antibody against BTLA. In some embodiments, the immune checkpoint inhibitor is a human or humanized antibody against BMA. In some embodiments, the immune checkpoint inhibitor reduces the expression or activity of one or more immune checkpoint proteins, such as BTLA.

In some embodiments, the immune checkpoint inhibitor is an inhibitor of TIM3. In some embodiments, the immune checkpoint inhibitor is an antibody against TIM3. In some embodiments, the immune checkpoint inhibitor is a monoclonal antibody against TIM3. In some embodiments, the immune checkpoint inhibitor is a human or humanized antibody against TIM3. In some embodiments, the immune checkpoint inhibitor reduces the expression or activity of one or more immune checkpoint proteins, such as TIM3.

In some embodiments, the immune checkpoint inhibitor is an inhibitor of A2Ar. In some embodiments, the immune checkpoint inhibitor is an antibody against A2Ar. In some embodiments, the immune checkpoint inhibitor is a monoclonal antibody against A2Ar. In some embodiments, the immune checkpoint inhibitor is a human or humanized antibody against A2Ar. In some embodiments, the immune checkpoint inhibitor reduces the expression or activity of one or more immune checkpoint proteins, such as A2Ar.

In some embodiments, the immune checkpoint inhibitor is an inhibitor of VISTA protein. In some embodiments, the immune checkpoint inhibitor is an antibody against VISTA protein. In some embodiments, the immune checkpoint inhibitor is a monoclonal antibody against VISTA protein. In some embodiments, the immune checkpoint inhibitor is a human or humanized antibody against VISTA protein. In some embodiments, the immune checkpoint inhibitor reduces the expression or activity of one or more immune checkpoint proteins, such as VISTA protein.

### Antibody Targets

In some embodiments, the antibody of an immunoconjugate is capable of binding one or more targets selected from (e.g., specifically binds to a target selected from) 5T4, ABL, ABCF1, ACVR1, ACVR1B, ACVR2, ACVR2B, ACVRL1, ADORA2A, Aggrecan, AGR2, AICDA, AIF1, AIGI, AKAP1, AKAP2, AMH, AMHR2, ANGPT1, ANGPT2, ANGPTL3, ANGPTL4, ANPEP, APC, APOC1, AR, aromatase, ATX, AX1, AZGP1 (zinc-a-glycoprotein), B7.1, B7.2, B7-H1, BAD, BAFF, BAG1, BAI1, BCR, BCL2, BCL6, BDNF, BLNK, BLR1 (MDR15), BIyS, BMP1, BMP2, BMP3B (GDFIO), BMP4, BMP6, BMP8, BMPRTA, BMPR1B, BMPR2, BPAG1 (plectin), BRCA1, C19orflO (IL27w), C3, C4A, C5, C5R1, CANT1, CAPRIN-1, CASP1, CASP4, CAV1, CCBP2 (D6/JAB61), CCLI (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22 (MDC/STC-1), CCL23 (MPIF-I), CCL24 (MPIF-2/eotaxin-2), CCL25 (TECK), CCL26 (eotaxin-3), CCL27 (CTACK/ILC), CCL28, CCL3 (MIP-Ia), CCL4 (MIPIb), CCL5 (RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR5 (CMKBR5/ChemR13), CCR6 (CMKBR6/CKR-L3/STRL22/DRY6), CCR7 (CKR7/EBI1), CCR8 (CMKBR8/TERI/CKR-L1), CCR9 (GPR-9-6), CCRL1 (VSHK1), CCRL2 (L-CCR), CD164, CD19, CDIC, CD2, CD20, CD21, CD200, CD-22, CD24, CD27, CD28, CD3, CD33, CD35, CD37, CD38, CD3E, CD3G, CD3Z, CD4, CD38, CD40, CD40L, CD44, CD45RB, CD47, CD52, CD69, CD72, CD74, CD79A, CD79B, CD8, CD80, CD81, CD83, CD86, CD137, CD152, CD274, CDH1 (Ecadherin), CDH1O, CDH12, CDH13, CDH18, CDH19, CDH2O, CDH5, CDH7, CDH8, CDH9, CDK2, CDK3, CDK4, CDK5, CDK6, CDK7, CDK9, CDKN1A (p21Wap1/Cip1), CDKN1B (p27Kip1), CDKN1C, CDKN2A (p16INK4a), CDKN2B, CDKN2C, CDKN3, CEBPB, CERI, CHGA, CHGB, Chitinase, CHST1O, CKLFSF2, CKLFSF3, CKLFSF4, CKLFSF5, CKLFSF6, CKLFSF7, CKLFSF8, CLDN3, CLDN7 (claudin-7), CLDN18.2 (claudin 18.2), CLN3, CLU (clusterin), CMKLR1, CMKOR1 (RDC1), CNR1, COL18A1, COLIA1, COL4A3, COL6A1, CR2, Cripto, CRP, CSF1 (M-CSF), CSF2 (GM-CSF), CSF3 (GCSF), CTL8, CTNNB1 (b-catenin), CTSB (cathepsin B), CX3CL1 (SCYD1), CX3CR1 (V28), CXCL1 (GRO1), CXCL1O (IP-IO), CXCLI1 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp451J0118, DNCL1, DPP4, E2F1, Engel, Edge, Fennel, EFNA3, EFNB2, EGF, EGFR, ELAC2, ENG, Enola, ENO2, ENO3, EPHA1, EPHA2, EPHA3, EPHA4, EPHA5, EPHA6, EPHA7, EPHA8, EPHA9, EPRA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (Her-2), EREG, ERK8, Estrogen receptor, Earl, ESR2, F3 (TF), FADD, famesyltransferase, FasL, FASNf, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF). FGF20, FGF21, FGF22, FGF23, FGF3 (int-2), FGF4 (HST), FGF5, FGF6 (HST-2), FGF7 (KGF), FGF8, FGF9, FGFR3, FIGF (VEGFD), FILI (EPSILON), FBL1 (ZETA), FLJ12584, FLJ25530, FLRT1 (fibronectin), FLT1, FLT-3, FOS, FOSL1 (FRA-1), FY (DARC), GABRP (GABAa), GAGEB1, GAGEC1, GALNAC4S-6ST, GATA3, GD2, GDF5, GFI1, GGT1, GM-CSF, GNAS1, GNRH1, GPR2 (CCR10), GPR31, GPR44, GPR81 (FKSG80), GRCC1O (C1O), GRP, GSN (Gelsolin), GSTP1, HAVCR2, HDAC, HDAC4, HDAC5, HDAC7A, HDAC9, Hedgehog, HGF, HIF1A, HIP1, histamine and histamine receptors, HLA-A, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFNgamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2 (CD121b), IL-IRA, IL-2, IL2RA (CD25), IL2RB (CD122), IL2RG (CD132), IL-4, IL-4R (CD123), IL-5, IL5RA (CD125), IL3RB (CD131), IL-6, IL6RA, (CD126), IR6RB (CD130), IL-7, IL7RA (CD127), IL-8, CXCR1 (IL8RA), CXCR2, (IL8RB/CD128), IL-9, IL9R (CD129), IL-10, IL10RA (CD210), IL10RB (CDW210B), IL-11, IL11RA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, IL18RAP, IL19, ILIA, ILIB, ILIF10, ILIF5, IL1F6, ILIF7, IL1F8, DL1F9, ILIHYI, ILIR1, ILIR2, ILIRAP, ILIRAPL1, ILIRAPL2, ILIRL1, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4, IL6ST (glycoprotein 130), ILK, INHA, INHBA, INSL3, INSL4, IRAK1, IRAK2, ITGA1, ITGA2, ITGA3, ITGA6 (.alpha.6 integrin), ITGAV, ITGB3, ITGB4 (.beta.4 integrin), JAG1, JAK1, JAK3, JTB, JUN, K6HF, KAI1, KDR, KITLG, KLF5 (GC Box BP), KLF6, KLK10, KLK12, KLK13, KLK14, KLK15, KLK3, KLK4, KLK5, KLK6, KLK9, KRT1, KRT19 (Keratin 19), KRT2A, KRTHB6 (hair-specific type II keratin), LAMA5, LEP (leptin), Lingo-p75, Lingo-Troy, LPS, LRRC15, LTA (TNF-b)), LTB, LTB4R (GPR16), LTB4R2, LTBR, MACMARCKS, MAG or OMgp, MAP2K7 (c-Jun), MCP-1, MDK, MIB1, midkine, MIF, MISRII, MJP-2, MK, MKI67 (Ki-67), MMP2, MMP9, MS4A1, MSMB, MT3 (metallothionectin-UI), mTOR, MTSS1, MUC1 (mucin), MYC, MYD88, NCK2, neurocan, Nectin-4, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOX5, NPPB, NROB1, NROB2, NRID1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, ODZI, OPRDI, P2RX7, PAP, PART1, PATE, PAWR, PCA3, PCDGF, PCNA, PDGFA, PDGFB, PDGFRA, PDGFRB, PECAMI, peg-asparaginase, PF4 (CXCL4), PGF, PGR, phosphacan, PIAS2, PI3 Kinase, PIK3CG, PLAU (uPA), PLG, PLXDCI, PKC, PKC-beta, PPBP (CXCL7), PPID, PR1, PRKCQ, PRKD1, PRL, PROC, PROK2, PSAP, PSCA, PTAFR, PTEN, PTGS2 (COX-2), PIN, RAC2 (P21Rac2), RANK, RANK ligand, RARB, RGS1, RGS13, RGS3, RNFI1O (ZNF144), Ron, ROBO2, RXR, S100A2, SCGB 1D2 (lipophilin B), SCGB2A1 (mammaglobin 2), SCGB2A2 (mammaglobin 1), SCYE1 (endothelial Monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLC2A2, SLC33A1, SLC43A1, SLIT2, SPP1, SPRR1B (Spr1), ST6GAL1, STAB1, STATE, STEAP, STEAP2, TB4R2, TBX21, TCP1O, TDGF1, TEK, TGFA, TGFB1, TGFBI1I1, TGFB2, TGFB3, TGFBI, TGEBR1, TGFBR2, TGFBR3, THIL, THBS1 (thrombospondin-1), THBS2, THBS4, THPO, TIE (Tie-1), TIMP3, tissue factor, TLR1, TLR2, TLR3, TLR4, TLR5, TLR6, TLR7, TLR8, TLR9, TLR10, TLR11, TNF, TNF-a, TNFAIP2 (B94), TNFAIP3, TNFRSF11A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFSF11 (TRANCE), TNFSF12 (APO3L), TNFSF13 (April), TNFSF13B, TNSF14 (HVEM-L), TNFRSF14 (HVEM), TNFSF15 (VEGI), TNFSF18, TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TOLLIP, Toll-like receptors, TOP2A (topoisomerase 1ia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, Tyrosinase, uPAR, VEGF, VEGFB, VEGFC, versican, VHL C5, VLA-4, Wnt-1, XCL1 (tymphotactin), XCL2 (SCM-Ib), XCRI (GPR5/CCXCR1), YYI, ZFPM2, CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), CLEC7A (Dectin-1), PDGFRa, SLAMF7, GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, TARM1, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC14, SIGLEC15 (CD33L3), SIGLEC16, SIRPB1 (CD172B), TREM1 (CD354), TREM2, and KLRF1 (NKp80).

In some embodiments, the antibody binds to an FcR.gamma-coupled receptor. In some embodiments, the FcR.gamma-coupled receptor is selected from the group consisting of GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), NCR1 (CD335, LY94, NKp46), NCR3 (CD335, LY94, NKp46), NCR3 (CD337, NKp30), OSCAR, and TARM1.

In some embodiments, the antibody binds to a DAP12-coupled receptor. In some embodiments, the DAP12-coupled receptor is selected from the group consisting of CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC14, SIGLEC15 (CD33L3), SIGLEC16, SIRPB1 (CD172B), TREM1 (CD354), and TREM2.

In some embodiments, the antibody binds to a hemITAM-bearing receptor. In some embodiments, the hemITAM-bearing receptor is KLRF1 (NKp80).

In some embodiments, the antibody is capable of binding one or more targets selected from CLEC4C (BDCA-2, DLEC, CD303, CLECSF7), CLEC4D (MCL, CLECSF8), CLEC4E (Mincle), CLEC6A (Dectin-2), CLEC5A (MDL-1, CLECSF5), CLEC1B (CLEC-2), CLEC9A (DNGR-1), and CLEC7A (Dectin-1). In some embodiments, the antibody is capable of binding CLEC6A (Dectin-2) or CLEC5A. In some embodiments, the antibody is capable of binding CLEC6A (Dectin-2).

In some embodiments, the antibody is capable of binding one or more targets selected from (e.g., specifically binds to a target selected from): ATP5I (Q06185), OAT (P29758), AIFM1 (Q9Z0X1), AOFA (Q64133), MTDC (P18155), CMC1 (Q8BH59), PREP (Q8K411), YMEL1 (O88967), LPPRC (Q6PB66), LONM (Q8CGK3), ACON (Q99KI0), ODO1 (Q60597), IDHP (P54071), ALDH2 (P47738), ATPB (P56480), AATM (P05202), TMM93 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (O35887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (O55143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (O55222), GPNMB (Q99P91), ENV1 (P10404), ERO1A (Q8R180), CLH, (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433). In the preceding list, accession numbers are shown in parentheses.

In some embodiments, the antibody binds to an antigen selected from CDH1, CD19, CD20, CD29, CD30, CD38, CD40, CD47, EpCAM, MUC1, MUC16, EGFR, Her2, SLAMF7, and gp75. In some embodiments, the antigen is selected from CD19, CD20, CD47, EpCAM, MUC1, MUC16, EGFR, and Her2. In some embodiments, the antibody binds to an antigen selected from the Tn antigen and the Thomsen-Friedenreich antigen.

In some embodiments, the antibody or Fc fusion protein is selected from: abagovomab, abatacept (also known as ORENCIA®), abciximab (also known as REOPRO®), c7E3 Fab), adalimumab (also known as HUMIRA®), adecatumumab, alemtuzumab (also known as CAMPATH®), MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT®), bavituximab, bectumomab (also known as LYMPHOSCAN®), belimumab (also known as LYMPHO-STAT-B®), bertilimumab, besilesomab, bevacizumab (also known as AVASTIN®), biciromab brallobarbital, bivatuzumab mertansine, campath, canakinumab (also known as ACZ885), cantuzumab mertansine, capromab (also known as PROSTASCNT®), catumaxomab (also known as REMOVAB®), cedelizumab (also known as CIMZIA®), certolizumab pegol, cetuximab (also known as ERBITUX®), clenoliximab, dacetuzumab, dacliximab, daclizumab (also known as ZENAPAX®), denosumab (also known as AMG 162), detumomab, dorlimomab aritox, dorlixizumab, duntumumab, durimulumab, durmulumab, ecromeximab, eculizumab (also known as SOLIRIS®), edobacomab, edrecolomab (also known as Mab17-1A, PANOREX®), efalizumab (also known as RAPTIVA®), efungumab (also known as MYCOGRAB®), elsilimomab, enlimomab pegol, epitumomab cituxetan, efalizumab, epitumomab, epratuzumab, erlizumab, ertumaxomab (also known as REXOMUN®), etanercept (also known as ENBREL®), etaracizumab (also known as etaratuzumab, VITAXIN®, ABEGRIN®), exbivirumab, fanolesomab (also known as NEUTROSPEC®), faralimomab, felvizumab, fontolizumab (also known as HUZAF®), galiximab, gantenerumab, gavilimomab (also known as ABXCBL®), gemtuzumab ozogamicin (also known as MYLOTARG®), golimumab (also known as CNTO 148), gomiliximab, ibalizumab (also known as TNX-355), ibritumomab tiuxetan (also known as ZEVALIN®), igovomab, imciromab, infliximab (also known as REMICADE®), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA®), metelimumab, milatuzumab, minretumomab, mitumomab, morolimumab, motavizwnab (also known as NUMAX®), muromonab (also known as OKT3), nacolomab tafenatox, naptumomab estafenatox, natalizumab (also known as TYSABRI®, ANTEGREN®), nebacumab, nerelimomab, nimotuzumab (also known as THERACIM hR3®, THERA-CIM-hR3®, THERALOC®), nofetumomab merpentan (also known as VERLUMA®), ocrelizumab, odulimomab, ofatumumab, omalizumab (also known as XOLAIR®), oregovomab (also known as OVAREX®), otelixizumab, pagibaximab, palivizumab (also known as SYNAGIS®), panitumumab (also known as ABX-EGF, VECTIBIX®), pascolizumab, pemiumomab (also known as THERAGYN®), pertuzumab (also known as 2C4, OMNITARG®), pexelizumab, pintumomab, priliximab, pritumumab, ranibizumab (also known as LUCENTIS®), raxibacumab, regavirumab, reslizumab, rituximab (also known as RITUXAN®, MabTHERA®), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN®), tacatuzumab tetraxetan, tadocizumab, talizumab, taplitumomab paptox, tefibazumab (also known as AUREXIS®), telimomab aritox, teneliximab, teplizumab, ticilimumab, tocilizumab (also known as ACTEMRA®), toralizumab, tositumomab, trastuzumab (also known as HERCEPTIN®), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION®), volociximab (also known as M200), votumumab (also known as HUMASPECT®), zalutumumab, zanolimumab (also known as HuMAX-CD4), ziralimumab, zolimomab aritox, daratumumab, elotuxumab, obintunzumab, olaratumab, brentuximab vedotin, afibercept, abatacept, belatacept, afibercept, etanercept, romiplostim, SBT-040 (sequences listed in US 2017/0158772. In some embodiments, the antibody is rituximab.

In an exemplary embodiment, the immunoconjugates of the invention comprise an antibody construct that comprises an antigen binding domain that specifically recognizes and binds PD-L1.

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers, or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1-binding agents, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

In an exemplary embodiment, the immunoconjugates of the invention comprise an antibody construct that comprises an antigen binding domain that specifically recognizes and binds HER2.

In certain embodiments, immunoconjugates of the invention comprise anti-HER2 antibodies. In one embodiment of the invention, an anti-HER2 antibody of an immunoconjugate of the invention comprises a humanized anti-HER2 antibody, e.g., huMAb4D5-1, huMAb4D5-2, huMAb4D5-3, huMAb4D5-4, huMAb4D5-5, huMAb4D5-6, huMAb4D5-7 and huMAb4D5-8, as described in Table 3 of U.S. Pat. No. 5,821,337, which is specifically incorporated by reference herein. Those antibodies contain human framework regions with the complementarity-determining regions of a murine antibody (4D5) that binds to HER2. The humanized antibody huMAb4D5-8 is also referred to as trastuzumab, commercially available under the tradename HERCEPTIN™ (Genentech, Inc.).

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Eng. J. Med. 344:783-792).

In an embodiment of the invention, the antibody construct or antigen binding domain comprises the CDR regions of trastuzumab. In an embodiment of the invention, the anti-HER2 antibody further comprises the framework regions of the trastuzumab. In an embodiment of the invention, the anti-HER2 antibody further comprises one or both variable regions of trastuzumab.

In another embodiment of the invention, an anti-HER2 antibody of an immunoconjugate of the invention comprises a humanized anti-HER2 antibody, e.g., humanized 2C4, as described in U.S. Pat. No. 7,862,817. An exemplary humanized 2C4 antibody is pertuzumab (CAS Reg. No. 380610-27-5), PERJETA™ (Genentech, Inc.). Pertuzumab is a HER dimerization inhibitor (HDI) and functions to inhibit the ability of HER2 to form active heterodimers or homodimers with other HER receptors (such as EGFR/HER1, HER2, HER3 and HER4). See, for example, Harari and Yarden, Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003). PERJETA™ is approved for the treatment of breast cancer.

In an embodiment of the invention, the antibody construct or antigen binding domain comprises the CDR regions of pertuzumab. In an embodiment of the invention, the anti-HER2 antibody further comprises the framework regions of the pertuzumab. In an embodiment of the invention, the anti-HER2 antibody further comprises one or both variable regions of pertuzumab.

In an exemplary embodiment, the immunoconjugates of the invention comprise an antibody construct that comprises an antigen binding domain that specifically recognizes and binds CEA. Carcinoembryonic antigen-related cell adhesion molecule 5 (CEACAM5) also known as CD66e (Cluster of Differentiation 66e), is a member of the carcinoembryonic antigen (CEA) gene family.

In an exemplary embodiment, the immunoconjugates of the invention comprise an antibody construct that comprises an antigen binding domain that specifically recognizes and binds CEA.

Elevated expression of carcinoembryonic antigen (CEA, CD66e, CEACAM5) has been implicated in various biological aspects of neoplasia, especially tumor cell adhesion, metastasis, the blocking of cellular immune mechanisms, and having antiapoptosis functions. CEA is also used as a blood marker for many carcinomas. Labetuzumab (CEA-CIDE™, Immunomedics, CAS Reg. No. 219649-07-7), also known as MN-14 and hMN14, is a humanized IgG1 monoclonal antibody and has been studied for the treatment of colorectal cancer (Blumenthal, R. et al (2005) Cancer Immunology Immunotherapy 54(4):315-327). Labetuzumab conjugated to a camptothecin analog (labetuzumab govitecan, IMMU-130) targets carcinoembryonic antigen-related cell adhesion mol. 5 (CEACAM5) and is being studied in patients with relapsed or refractory metastatic colorectal cancer (Sharkey, R. et al, (2018), Molecular Cancer Therapeutics 17(1):196-203; Cardillo, T. et al (2018) Molecular Cancer Therapeutics 17(1):150-160).

In an exemplary embodiment, the immunoconjugates of the invention comprise an antibody construct that comprises an antigen binding domain that specifically recognizes and binds Trop2. Tumor-associated calcium signal transducer 2 (TROP-2) is a transmembrane glycoprotein encoded by the TACSTD2 gene (Linnenbach A J, et al (1993) Mol Cell Biol. 13(3): 1507-15; Calabrese G, et al (2001) Cytogenet Cell Genet. 92(1-2): 164-5). Trop2 is an intracellular calcium signal transducer that is differentially expressed in many cancers and signals cells for self-renewal, proliferation, invasion, and survival. Trop2 is considered a stem cell marker and is expressed in many normal tissues, though in contrast, it is overexpressed in many cancers (Ohmachi T, et al., (2006) Clin. Cancer Res., 12(10), 3057-3063; Muhlmann G, et al., (2009) J. Clin. Pathol., 62(2), 152-158; Fong D, et al., (2008) Br. J. Cancer, 99(8), 1290-1295; Fong D, et al., (2008) Mod. Pathol., 21(2), 186-191; Ning S, et al., (2013) Neurol. Sci., 34(10), 1745-1750). Overexpression of Trop2 is of prognostic significance. Several ligands have been proposed that interact with Trop2. Trop2 signals the cells via different pathways and it is transcriptionally regulated by a complex network of several transcription factors.

Human Trop2 (TACSTD2: tumor-associated calcium signal transducer 2, GA733-1, EGP-1, M1S1; hereinafter, referred to as hTrop2) is a single-pass transmembrane type 1 cell membrane protein consisting of 323 amino acid residues. While the presence of a cell membrane protein involved in immune resistance, which is common to human trophoblasts and cancer cells (Faulk W P, et al., Proc. Natl. Acad. Sci. 75(4):1947-1951 (1978)), has previously been suggested, an antigen molecule recognized by a monoclonal antibody against a cell membrane protein in a human choriocarcinoma cell line was identified and designated as Trop2 as one of the molecules expressed in human trophoblasts (Lipinski M, et al., Proc. Natl. Acad. Sci. 78(8), 5147-5150 (1981)). This molecule was also designated as tumor antigen GA733-1 recognized by a mouse monoclonal antibody GA733 (Linnenbach A J, et al., Proc. Natl. Acad. Sci. 86(1), 27-31 (1989)) obtained by immunization with a gastric cancer cell line or an epithelial glycoprotein (EGP-1; Basu A, et al., Int. J. Cancer, 62 (4), 472-479 (1995)) recognized by a mouse monoclonal antibody RS7-3G11 obtained by immunization with non-small cell lung cancer cells. In 1995, however, the Trop2 gene was cloned, and all of these molecules were confirmed to be identical molecules (Fornaro M, et al., Int. J. Cancer, 62(5), 610-618 (1995)). The DNA sequence and amino acid sequence of hTrop2 are available on a public database and can be referred to, for example, under Accession Nos. NM_002353 and NP_002344 (NCBI).

In response to such information suggesting the association with cancer, a plurality of anti-hTrop2 antibodies have been established so far and studied for their antitumor effects. Among these antibodies, there is disclosed, for example, an unconjugated antibody that exhibits in itself antitumor activity in nude mouse xenograft models (WO 2008/144891; WO 2011/145744; WO 2011/155579; WO 2013/077458) as well as an antibody that exhibits antitumor activity as ADC with a cytotoxic drug (WO 2003/074566; WO 2011/068845; WO 2013/068946; U.S. Pat. No. 7,999,083). However, the strength or coverage of their activity is still insufficient, and there are unsatisfied medical needs for hTrop2 as a therapeutic target.

TROP2 expression in cancer cells has been correlated with drug resistance. Several strategies target TROP2 on cancer cells that include antibodies, antibody fusion proteins, chemical inhibitors, nanoparticles, etc. The in vitro studies and pre-clinical studies, using these various therapeutic treatments, have resulted in significant inhibition of tumor cell growth both in vitro and in vivo in mice. Clinical studies have explored the potential application of Trop2 as both a prognostic biomarker and as a therapeutic target to reverse resistance.

Sacituzumab govitecan (TRODELVY®, Immunomedics, IMMU-132), an antibody-drug conjugate comprising a Trop2-directed antibody linked to a topoisomerase inhibitor drug, is indicated for the treatment of metastatic triple-negative breast cancer (mTNBC) in adult patients that have received at least two prior therapies. The Trop2 antibody in sacituzumab govitecan is conjugated to SN-38, the active metabolite of irinotecan (US 2016/0297890; WO 2015/098099).

In an exemplary embodiment, the immunoconjugates of the invention comprise an antibody construct that comprises an antigen binding domain that specifically recognizes and binds Caprin-1 (Ellis J A, Luzio J P (1995) J Biol Chem. 270(35):20717-23; Wang B, et al (2005) J Immunol. 175 (7):4274-82; Solomon S, et al (2007) Mol Cell Biol. 27(6):2324-42). Caprin-1 is also known as GPIAP1, GPIP137, GRIP137, M11S1, RNG105, p137GPI, and cell cycle associated protein 1.

Cytoplasmic activation/proliferation-associated protein-1 (caprin-1) is an RNA-binding protein that participates in the regulation of cell cycle control-associated genes. Caprin-1 selectively binds to c-Myc and cyclin D2 mRNAs, which accelerates cell progression through the G₁ phase into the S phase, enhances cell viability and promotes cell growth, indicating that it may serve an important role in tumorigenesis (Wang B, et al (2005) J Immunol. 175:4274-4282). Caprin-1 acts alone or in combination with other RNA-binding proteins, such as RasGAP SH3-domain-binding protein 1 and fragile X mental retardation protein. In the tumorigenesis process, caprin-1 primarily functions by activating cell proliferation and upregulating the expression of immune checkpoint proteins. Through the formation of stress granules, caprin-1 is also involved in the process by which tumor cells adapt to adverse conditions, which contributes to radiation and chemotherapy resistance. Given its role in various clinical malignancies, caprin-1 holds the potential to be used as a biomarker and a target for the development of novel therapeutics (Yang, Z-S, et al (2019) Oncology Letters 18:15-21).

Antibodies that target caprin-1 for treatment and detection have been described (WO 2011/096519; WO 2013/125654; WO 2013/125636; WO 2013/125640; WO 2013/125630; WO 2013/018889; WO 2013/018891; WO 2013/018883; WO 2013/018892; WO 2014/014082; WO 2014/014086; WO 2015/020212; WO 2018/079740).

In an exemplary embodiment, the immunoconjugates of the invention comprise an antibody construct that comprises an antigen binding domain that specifically recognizes and binds Claudin-1.

Claudin-1 is a member of the transmembrane protein family claudins located in cell-cell tight junctions and it acts as a co-receptor for HCV entry into hepatic cells (Kniesel U, et al (2000). Cell. Mol. Neurobiol. 20(1):57-76; Furuse M, et al (1998). J. Cell Biol. 141(7):1539-50; Swisshelm K, et al (2005) Adv. Drug Deliv. Rev. 57(6):919-28). Claudin 1 is also known as Senescence-associated epithelial membrane protein, senescence-associated epithelial membrane protein 1, CLDN1, CLD1, ILVASC, SEMP1.

Claudins are abundant in luminal epithelial sheets where they maintain epithelial cell polarity. Claudin-1 is expressed in most tissues such as bladder, fallopian tube, liver, pancreas, prostate, and skin.

In an exemplary embodiment, the immunoconjugates of the invention comprise an antibody construct that comprises an antigen binding domain that specifically recognizes and binds Nectin-4.

The nectins are a protein family of cell adhesion molecules involved in calcium-dependent cell adhesion (Takai Y. et al (2003) Cancer Science 94(8):655-67; Fuchs, A. et al (2006) Seminars in Cancer Biology 16(5):359-366; Miyoshi J. et al (2007) American journal of nephrology 27(6):590-604). Nectins play an important role in the bonding between cells in many different tissues, including the intermediate junction of epithelial cells or the chemical synapse of nerve cells.

In an exemplary embodiment, the immunoconjugates of the invention comprise an antibody construct that comprises an antigen binding domain that specifically recognizes and binds LRRC15 (Leucine rich repeat containing 15).

LRRC15 is a cell membrane-expressed protein that in humans is encoded by the LRRC15 gene. LRRC15 is expressed on stromal fibroblasts in many solid tumors (e.g., breast, head and neck, lung, pancreatic) as well as directly on a subset of cancer cells of mesenchymal origin (e.g., sarcoma, melanoma, glioblastoma). LRRC15 may have utility as a therapeutic target for the treatment of cancers with LRRC15-positive stromal desmoplasia or cancers of mesenchymal origin (Purcell, J W et al (2018) Cancer Res., 78(14):4059-4072).

### Bis-Benzimidazole Adjuvant Compounds

The immunoconjugate of the invention comprises a bis-benzimidazole adjuvant moiety. The adjuvant moiety described herein is a compound that elicits an immune response (i.e., an immunostimulatory agent). Generally, the adjuvant moiety described herein is a STING agonist.

Certain amido benzimidazole compounds are demonstrated STING receptor agonists with systemic activity (Ramanjulu, J. M. et al (2018) Nature 564.439-443; Barber, G. N. (2015) Nature Rev Immunol 15:760-770; US 2019/0300513).

STING is a dimeric structure with a large and symmetrical binding pocket. The bis-benzimidazole compounds of Table 1 when conjugated to a targeting antibody were designed to target and bind to the open conformation of the binding pocket of STING. Binding to a small molecule agonist usually induces a closed conformation of the STING protein. This introduces the risk that a linker, particularly if “noncleavable”, will interfere with binding and activation. The bis-benzimidazoles are reported to bind and activate through an open conformation, which we predicted would be more amenable to attachment of a linker (Ramanjulu, J. M. et al (2018) Nature 564:439-443; Barber, G. N. (2015) Nature Rev Immunol 15:760-770).

### Bis-Benzimidazole Linker Compounds

The immunoconjugates of the invention are prepared by conjugation of an antibody with a bis-benzimidazole-linker compound. The bis-benzimidazole-linker compounds comprise a bis-benzimidazole (BBI) moiety covalently attached to a linker unit (L). The linker units comprise functional groups and subunits which affect stability, permeability, solubility, and other pharmacokinetic, safety, and efficacy properties of the immunoconjugates. The linker unit includes a reactive functional group which reacts, i.e. conjugates, with a reactive functional group of the antibody. For example, a nucleophilic group such as a lysine side chain amino of the antibody reacts with an electrophilic reactive functional group of the BBI-linker compound to form the immunoconjugate. Also, for example, a cysteine thiol of the antibody reacts with a maleimide or bromoacetamide group of the BBI-linker compound to form the immunoconjugate.

Considerations for the design of the immunoconjugates of the invention include: (1) preventing the premature release of the bis-benzimidazole (BBI) moiety during in vivo circulation and (2) ensuring that a biologically active form of the BBI moiety is released at the desired site of action at an adequate rate. The complex structure of the immunoconjugate together with its functional properties requires careful design and selection of every component of the molecule including antibody, conjugation site, linker structure, and the bis-benzimidazole compound. The linker determines the mechanism and rate of adjuvant release.

Generally, the linker unit (L) may be cleavable or non-cleavable. Cleavable linker units may include a peptide sequence which is a substrate for certain proteases such as Cathepsins which recognize and cleave the peptide linker unit, separating the STING agonist from the antibody (Caculitan N G, et al (2017) Cancer Res. 77(24):7027-7037).

Cleavable linker units may include labile functionality such as an acid-sensitive disulfide group (Kellogg, B A et al (2011) Bioconjugate Chem. 22, 717-727; Ricart, A. D. et al (2011) Clin. Cancer Res. 17, 6417-6427; Pillow, T., et al (2017) Chem. Sci. 8, 366-370; Zhang), et al (2016) ACS Med Chem Lett. 7(11):988-993).

In some embodiments, the linker is non-cleavable under physiological conditions. As used herein, the term “physiological conditions” refers to a temperature range of 20-40 degrees Celsius, atmospheric pressure (i.e., 1 atm), a pH of about 6 to about 8, and the one or more physiological enzymes, proteases, acids, and bases. One advantage of a non-cleavable linker between the antibody and STING agonist in an immunoconjugate is minimizing premature payload release and corresponding toxicity. STING is a broadly expressed receptor, therefore a particularly relevant consideration.

In one embodiment, the invention includes a peptide linking unit, i.e. L or linker, between the cell-binding agent and the immunostimulatory moiety, comprising a peptide radical based on a linear sequence of specific amino acid residues which can be selectively cleaved by a protease such as a cathepsin, a tumor-associated elastase enzyme or an enzyme with protease-like or elastase-like activity. The peptide radical may be about two to about twelve amino acids. Enzymatic cleavage of a bond within the peptide linker releases an active form of the immunostimulatory moiety. This leads to an increase in the tissue specificity of the conjugates according to the invention and thus to an additional decrease of toxicity of the conjugates according to the invention in other tissue types.

The linker provides sufficient stability of the immunoconjugate in biological media, e.g. culture medium or serum and, at the same time, the desired intracellular action within tumor tissue as a result of its specific enzymatic or hydrolytic cleavability with release of the immunostimulatory moiety, i.e. “payload”.

The enzymatic activity of a protease, cathepsin, or elastase can catalyze cleavage of a covalent bond of the immunoconjugate under physiological conditions. The enzymatic activity being the expression product of cells associated with tumor tissue. The enzymatic activity on the cleavage site of the targeting peptide converts the immunoconjugate to an active immunostimulatory drug free of targeting peptide and linking group. The cleavage site may be specifically recognized by the enzyme. Cathepsin or elastase may catalyze the cleavage of a specific peptidic bond between the C-terminal amino acid residue of the specific peptide and the immunostimulatory moiety of the immunoconjugate.

In one embodiment, the invention includes a linking unit, i.e. L or linker, between the cell-binding agent and the immunostimulatory moiety, comprising a substrate for glucuronidase (Jeffrey S C, et al (2006) Bioconjug Chem. 17(3):831-40), or sulfatase (Bargh J D, et al (2020) Chem Sci. 11(9):2375-2380) cleavage. In particular, L include a Gluc unit and comprise a formula selected from:

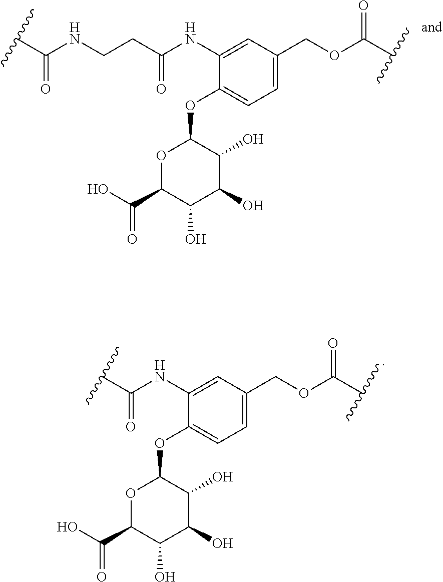 [SMILES: *C(=O)NCCC(=O)Nc1cc(COC(*)=O)ccc1O[C@@H]1O[C@H](C(=O)O)[C@@H](O)[C@H](O)[C@H]1O ; *C(=O)Nc1cc(COC(*)=O)ccc1O[C@@H]1O[C@H](C(=O)O)[C@@H](O)[C@H](O)[C@H]1O]

Specific cleavage of the immunoconjugates of the invention takes advantage of the presence of tumor infiltrating cells of the immune system and leukocyte-secreted enzymes, to promote the activation of an anticancer drug at the tumor site.

Reactive electrophilic reactive functional groups (Q in Formula II) suitable for the BBI-linker compounds include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (thiol reactive); halogenated acetamides such as N-iodoacetamides (thiol reactive); aryl azides (primary amine reactive); fluorinated aryl azides (reactive via carbon-hydrogen (C—H) insertion); pentafluorophenyl (PFP) esters (amine reactive); tetrafluorophenyl (TFP) and sulfotetrafluorophenyl (STP) esters (amine reactive); imidoesters (amine reactive); isocyanates (hydroxyl reactive); vinyl sulfones (thiol, amine, and hydroxyl reactive); pyridyl disulfides (thiol reactive); and benzophenone derivatives (reactive via C—H bond insertion). Further reagents include, but are not limited, to those described in Hermanson, Bioconjugate Techniques 2^{nd} Edition, Academic Press, 2008.

The invention provides solutions to the limitations and challenges to the design, preparation and use of immunoconjugates. Some linkers such as those comprising peptide units and substrates for protease may be labile in the blood stream, thereby releasing unacceptable amounts of the adjuvant/drug prior to internalization in a target cell (Khot, A. et al (2015) Bioanalysis 7(13):1633-1648). Other linkers may provide stability in the bloodstream, but intracellular release effectiveness may be negatively impacted. Linkers that provide for desired intracellular release typically have poor stability in the bloodstream. Alternatively stated, bloodstream stability and intracellular release are typically inversely related. In addition, in standard conjugation processes, the amount of adjuvant/drug moiety loaded on the antibody, i.e. drug loading, the amount of aggregate that is formed in the conjugation reaction, and the yield of final purified conjugate that can be obtained are interrelated. For example, aggregate formation is generally positively correlated to the number of equivalents of adjuvant/drug moiety and derivatives thereof conjugated to the antibody. Under high drug loading, formed aggregates must be removed for therapeutic applications. As a result, drug loading-mediated aggregate formation decreases immunoconjugate yield and can render process scale-up difficult.

Exemplary embodiments include a bis-benzimidazole-linker compound of Formula II:

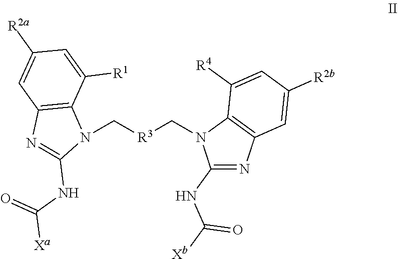

wherein
X^{a} and X^{b} are independently selected from a five-membered heteroaryl, optionally substituted with R⁵;
R¹ and R⁴ are independently selected from the group consisting of H, F, Cl, Br, I, —CN, —OH, —O—(C₁-C₆ alkyl), and R⁵;
R^{2a} and R^{2b} are independently selected from H, —C(═O)N(R⁶)₂, and R⁵;
where one of X^{a}, X^{b}, R¹, R⁴, R^{2a} and R^{2b} is substituted with R⁵;
R³ is selected from C₁-C₆ alkyldiyl, —(C₁-C₃ alkyldiyl)-O—(C₁-C₃ alkyldiyl)-, C₂-C₆ alkenyldiyl and C₂-C₆ alkynyldiyl, optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂;
R⁵ is selected from the group consisting of:
   (C₁-C₁₂ alkyldiyl)-L;
   (C₁-C₁₂ alkyldiyl)-N(R⁶)-L;
   (C₁-C₁₂ alkyldiyl)-O-L;
   (C₁-C₁₂ alkyldiyl)-(C₂-C₂₀ heterocyclyldiyl)-L;
   O—(C₁-C₁₂ alkyldiyl)-L;
   O—(C₁-C₁₂ alkyldiyl)-N(R⁶)-L;
   O(C₁-C₁₂ alkyldiyl)-O-L;
   O—(C₁-C₁₂ alkyldiyl)-(C₂-C₂₀ heterocyclyldiyl)-L;
   O—(C₁-C₁₂ alkyldiyl)-(C₂-C₂₀ heterocyclyldiyl)-N(R⁶)-L;
   OC(═O)N(R⁶)-L;
   OC(═O)N(R⁶)—(C₁-C₁₂ alkyldiyl)-N(R⁶)-L;
   N(R⁶)-L;
   N(R⁶)—(C₁-C₁₂ alkyldiyl)-L;
   N(R⁶)—(C₁-C₁₂ alkyldiyl)-N(R⁶)-L;
   N(R⁶)—(C₁-C₁₂ alkyldiyl)-O-L;
   N(R⁶)—(C₁-C₁₂ alkyldiyl)-(C₂-C₂₀ heterocyclyldiyl)-L;
   C(═O)N(R⁶)-L;
   C(═O)N(R⁶)—(C₁-C₁₂ alkyldiyl)-L;
   C(═O)N(R⁶)—(C₁-C₁₂ alkyldiyl)-N(R⁶)-L;
   C(═O)N(R⁶)—(C₁-C₁₂ alkyldiyl)-O-L;
   (C₂-C₂₀ heterocyclyldiyl)-L;
   S(═O)₂—(C₂-C₂₀ heterocyclyldiyl)-L; and
   S(═O)₂—(C₂-C₂₀ heterocyclyldiyl)-(C₁-C₁₂ alkyldiyl)-N(R⁶)-L;
R⁶ is independently H or C₁-C₆ alkyl;
L is a linker selected from the group consisting of:
   Q-C(═O)-PEG-;
   Q-C(═O)-PEG-C(═O)N(R⁶)—(C₁-C₁₂ alkyldiyl)-C(═O)-Gluc-;
   Q-C(═O)-PEG-O—;
   Q-C(═O)-PEG-O—C(═O)—;
   Q-C(═O)-PEG-C(═O)—;
   Q-C(═O)-PEG-C(═O)-PEP-;
   Q-C(═O)-PEG-N(R⁶)—;
   Q-C(═O)-PEG-N(R⁶)—C(═O)—;
   Q-C(═O)-PEG-N(R⁶)-PEG-C(═O)-PEP-;
   Q-C(═O)-PEG-N⁺(R⁶)₂-PEG-C(═O)-PEP-;
   Q-C(═O)-PEG-C(═O)-PEP-N(R⁶)—(C₁-C₁₂ alkyldiyl)-;
   Q-C(═O)-PEG-C(═O)-PEP-N(R⁶)—(C₁-C₁₂ alkyldiyl)N(R⁶)C(═O)—(C₂-C₅ monoheterocyclyldiyl)-;
   Q-C(═O)-PEG-C(═O)N(R⁶)—(C₁-C₁₂ alkyldiyl)-C(═O)-PEP-;
   Q-C(═O)-PEG-SS-(C₁-C₁₂ alkyldiyl)-OC(═O)—;
   Q-C(═O)-PEG-SS-(C₁-C₁₂ alkyldiyl)-C(═O)—;
   Q-C(═O)—(C₁-C₁₂ alkyldiyl)-C(═O)-PEP-;
   Q-C(═O)—(C₁-C₁₂ alkyldiyl)-C(═O)-PEP-N(R⁶)—(C₁-C₁₂ alkyldiyl)-;
   Q-C(═O)—(C₁-C₁₂ alkyldiyl)-C(═O)-PEP-N(R⁶)—(C₁-C₁₂ alkyldiyl)-N(R⁵)—C(═O);
   Q-C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
   Q-(CH₂)ₘ—C(═O)N(R⁶)-PEG-;
   Q-(CH₂)ₘ—C(═O)N(R⁶)-PEG-C(═O)N(R⁶)—(C₁-C₁₂ alkyldiyl)-C(═O)-Gluc-;
   Q-(CH₂)ₘ—C(═O)N(R⁶)-PEG-O—;
   Q-(CH₂)ₘ—C(═O)N(R⁶)-PEG-O—C(═O)—;
   Q-(CH₂)ₘ—C(═O)N(R⁶)-PEG-C(═O)—;
   Q-(CH₂)ₘ—C(═O)N(R⁶)-PEG-N(R⁵)—;
   Q-(CH₂)ₘ—C(═O)N(R⁶)-PEG-N(R⁵)—C(═O)—;
   Q-(CH₂)ₘ—C(═O)N(R⁶)-PEG-C(═O)-PEP-;
   Q-(CH₂)ₘ—C(═O)N(R⁶)-PEG-SS-(C₁-C₁₂ alkyldiyl)-OC(═O)—;
   Q-(CH₂)ₘ—C(═O)-PEP-N(R⁶)—(C₁-C₁₂ alkyldiyl)-;
   Q-(CH₂)ₘ—C(═O)N(R⁶)-PEG-C(═O)N(R⁶)—(C₁-C₁₂ alkyldiyl)-C(═O)-PEP-;
   Q-(CH₂)ₘ—C(═O)-PEP-N(R⁶)—(C₁-C₁₂ alkyldiyl)N(R⁶)C(═O)—; and
   Q-(CH₂)ₘ—C(═O)-PEP-N(R⁶)—(C₁-C₁₂ alkyldiyl)N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
Gluc has the formula:

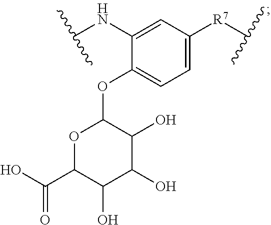

PEP has the formula:

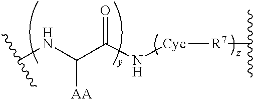

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

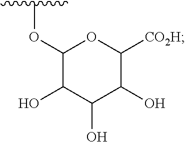 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1;
Q is selected from the group consisting of N-hydroxysuccinimidyl, N-hydroxysulfosuccinimidyl, maleimide, and phenoxy substituted with one or more groups independently selected from F, Cl, NO₂, and SO₃⁻; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

In an exemplary embodiment, L comprises PEP and PEP is a dipeptide and has the formula:

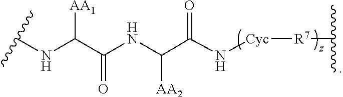

In an exemplary embodiment, L comprises PEP and PEP is a tripeptide and has the formula:

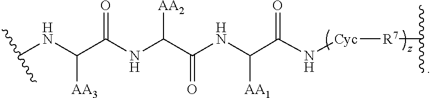

In an exemplary embodiment, PEP is a tripeptide wherein AA₁ is methyl, AA₂ forms proline, and AA₃ is isopropyl.

In an exemplary embodiment, L comprises PEP and PEP is a tetrapeptide and has the formula:

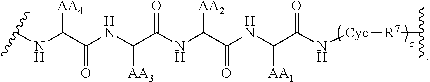

In an exemplary embodiment, PEP is a tetrapeptide wherein:
AA₁ is selected from the group consisting of Abu, Ala, and Val;
AA₂ is selected from the group consisting of Nle(O-Bzl), Oic and Pro;
AA₃ is selected from the group consisting of Ala and Met(O)₂; and
AA₄ is selected from the group consisting of Oic, Arg(NO₂), Bpa, and Nle(O-Bzl).

In an exemplary embodiment, PEP is comprised of amino acid residues of amino acids selected from the group consisting of:

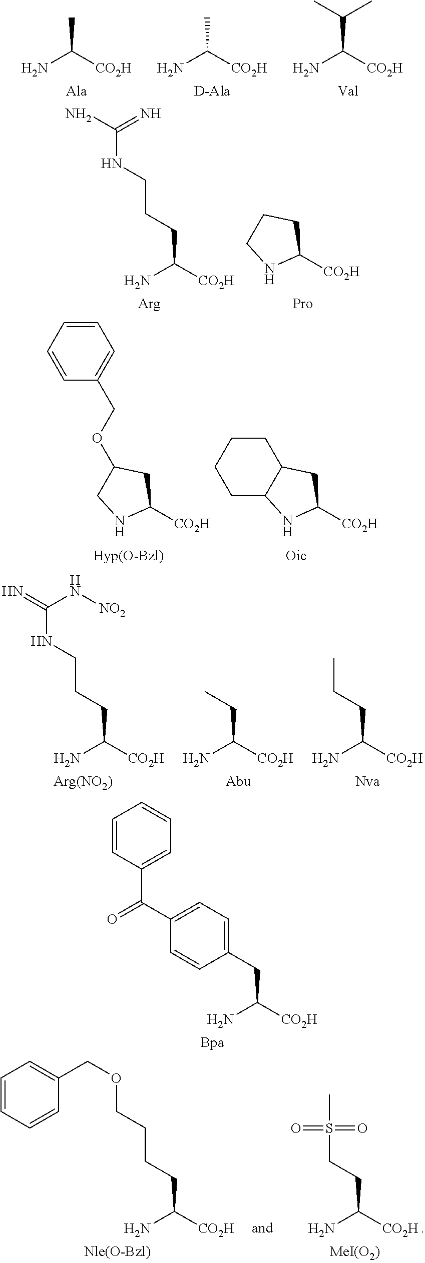

In an exemplary embodiment, PEP is selected from the group consisting of Ala-Pro-Val, Asn-Pro-Val, Ala-Ala-Val, and other peptide sequences as described in WO 2021/226440.

In an exemplary embodiment, PEP has the formula:

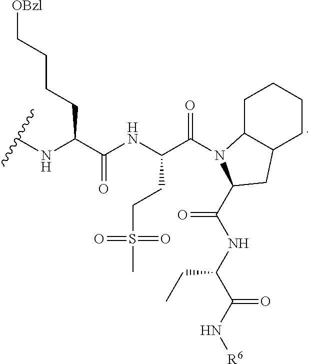

In an exemplary embodiment, PEP has the formula:

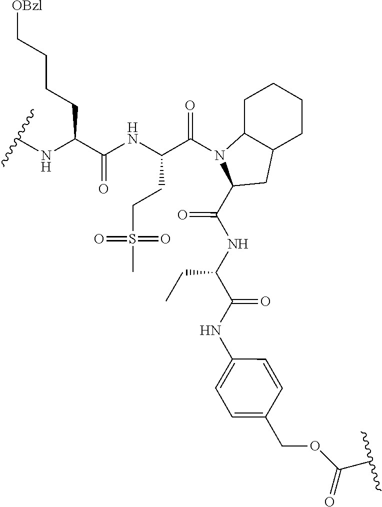

An exemplary embodiment of the STING agonist-linker intermediate compound includes wherein L comprises the formulas:

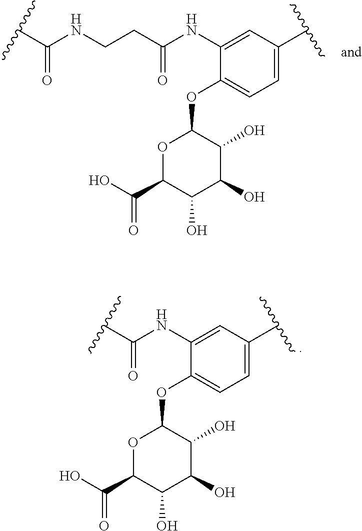 [SMILES: *C(=O)NCCC(=O)Nc1cc(*)ccc1O[C@@H]1O[C@H](C(=O)O)[C@@H](O)[C@H](O)[C@H]1O ; *C(=O)Nc1cc(*)ccc1O[C@@H]1O[C@H](C(=O)O)[C@@H](O)[C@H](O)[C@H]1O]

An exemplary embodiment of the STING agonist-linker intermediate compound includes wherein Q is selected from:

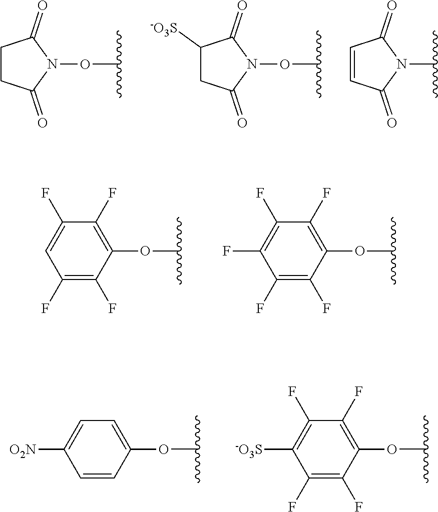

An exemplary embodiment of the STING agonist-linker intermediate compound includes wherein Q is phenoxy substituted with one or more groups independently selected from F, Cl, NO₂, and SO₃⁻.

An exemplary embodiment of the STING agonist-linker intermediate compound includes wherein Q is 2,3,5,6-tetrafluorophenoxy.

An exemplary embodiment of the STING agonist-linker intermediate compound includes wherein Q is 2,3,5,6-tetrafluoro-4-sulfonato-phenoxy.

An exemplary embodiment of the STING agonist-linker intermediate compound includes wherein Q is maleimide.

An exemplary embodiment of the STING agonist-linker intermediate compound includes wherein L is selected from the structures:

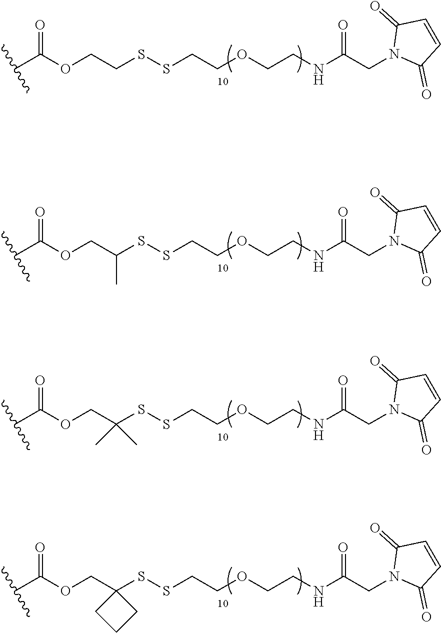

where the wavy line indicates the attachment to R⁵.

Exemplary embodiments of the Bis-benzimidazole, STING agonist-linker intermediate compound are shown in Tables 1a and 1b. Each STING agonist-linker intermediate compound was prepared and characterized by mass spectrometry and shown to have the mass indicated. The STING agonist-linker intermediate compounds of Tables 1a and 1b demonstrate the surprising and unexpected property of STING agonist selectivity which may predict useful therapeutic activity to treat cancer and other disorders when conjugated to an antibody.

TABLE 1a
Bis-benzimidazole-linker (BBI-L) Formula II compounds

| BBI-L No. | Structure | MW |
|---|---|---|
| BBI-L-1 | 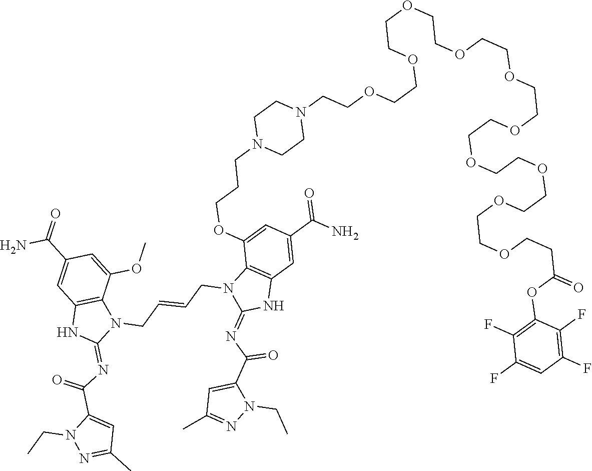 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(CCOCCOCCOCCOCCOCCOCCOCCOCCOCCOCCC(=O)Oc4c(F)c(F)cc(F)c4F)CC3)c21] | 1509.6 |
| BBI-L-2 | 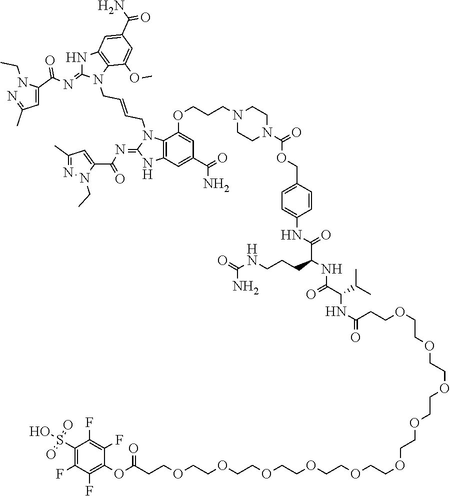 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21] | 2023.1 |
| BBI-L-3 | 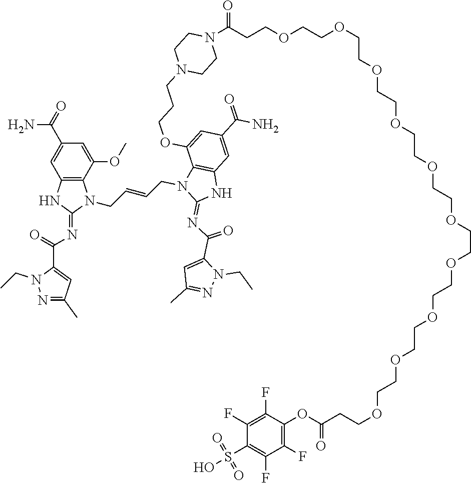 | 1617.7 |
| BBI-L-4 | 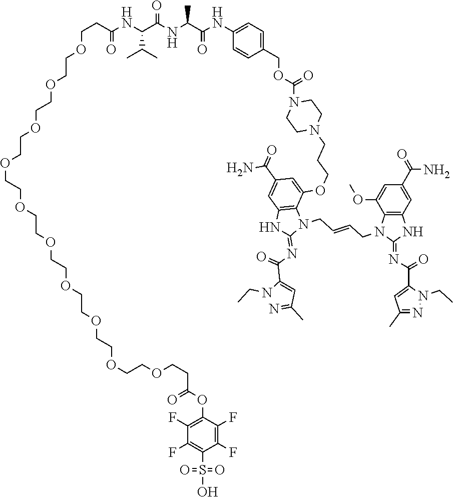 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21] | 1937.1 |
| BBI-L-5 | 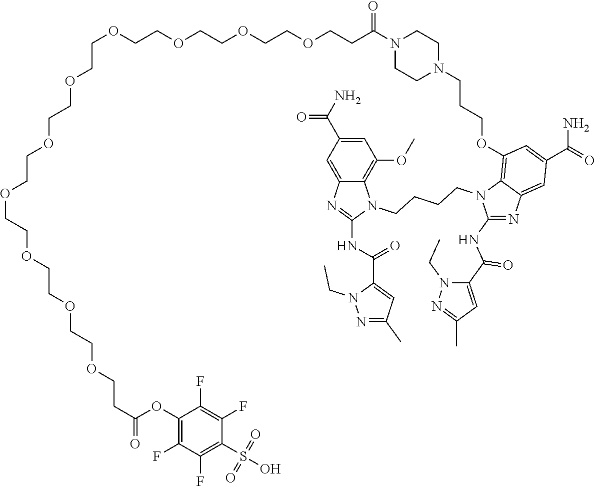 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(C(=O)CCOCCOCCOCCOCCOCCOCCOCCOCCOCCOCCC(=O)Oc4c(F)c(F)c(S(=O)(=O)O)c(F)c4F)CC3)c21] | 1619.7 |
| BBI-L-6 | 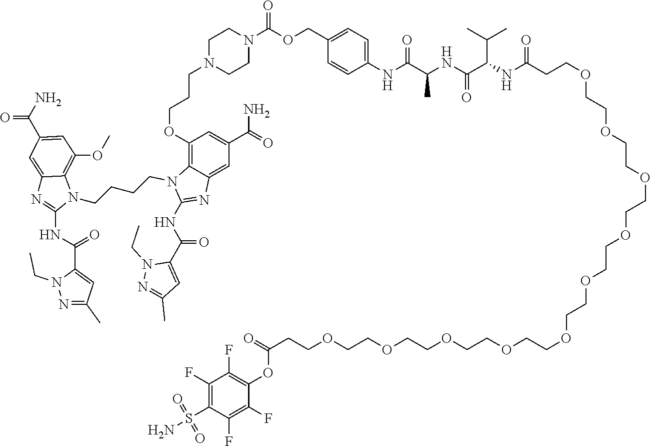 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(N)(=O)=O)c(F)c5F)C(C)C)cc4)CC3)c21] | 1939.1 |
| BBI-L-7 | 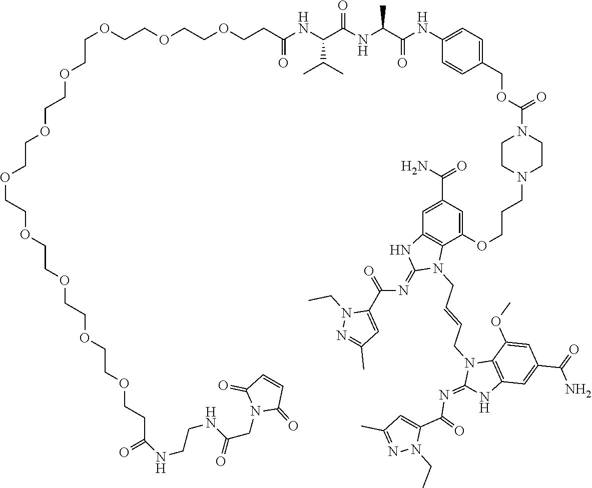 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCC(=O)NCCNC(=O)CN5C(=O)C=CC5=O)C(C)C)cc4)CC3)c21] | 1888.1 |
| BBI-L-8 | 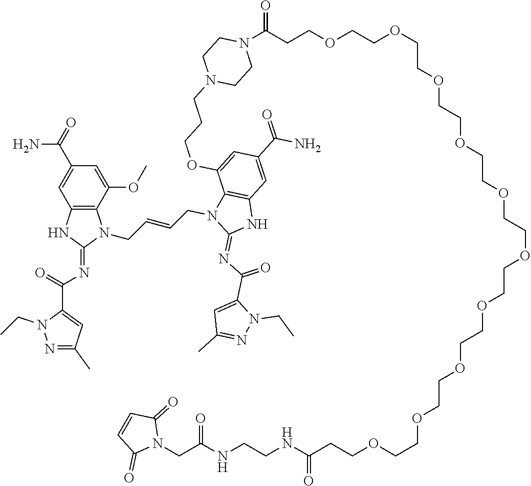 | 1568.7 |
| BBI-L-9 | 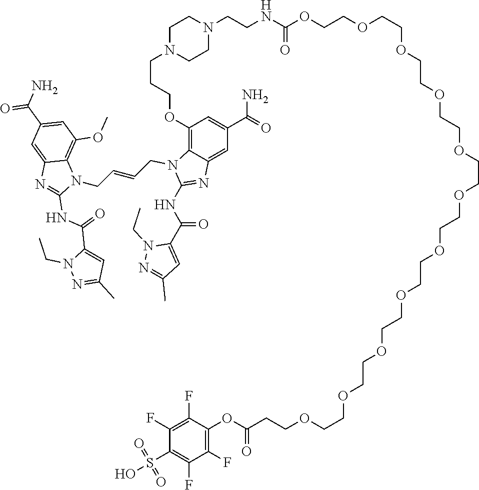 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCNC(=O)OCCOCCOCCOCCOCCOCCOCCOCCOCCOCCOCCC(=O)Oc4c(F)c(F)c(S(=O)(=O)O)c(F)c4F)CC3)c21] | 1676.7 |
| BBI-L-10 | 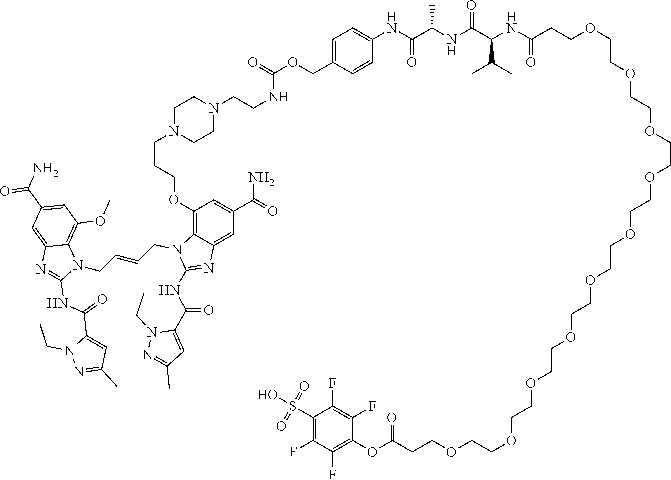 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCNC(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21] | 1980.1 |
| BBI-L-11 | 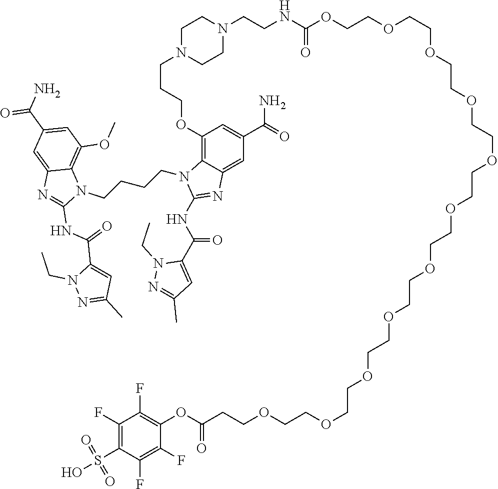 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(CCNC(=O)OCCOCCOCCOCCOCCOCCOCCOCCOCCOCCOCCC(=O)Oc4c(F)c(F)c(S(=O)(=O)O)c(F)c4F)CC3)c21] | 1678.8 |
| BBI-L-12 | 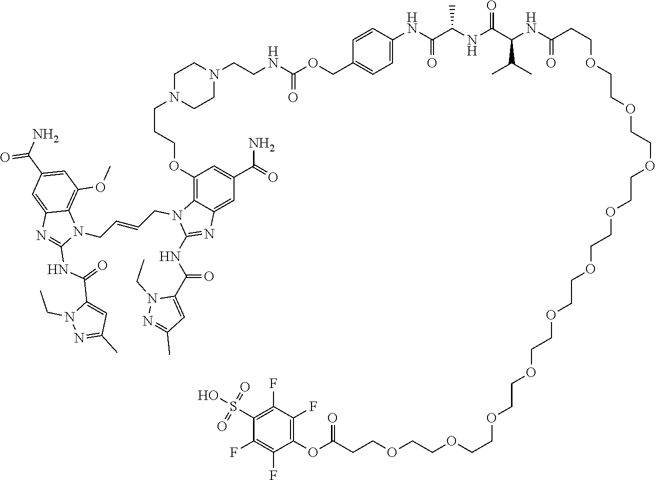 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCNC(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21] | 1982.1 |
| BBI-L-13 | 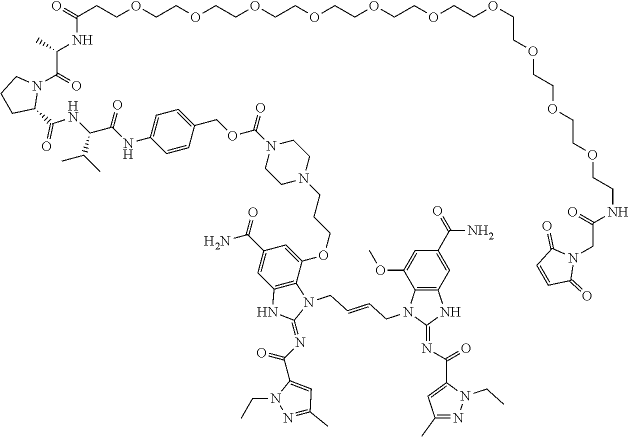 | 1914.1 |
| BBI-L-14 | 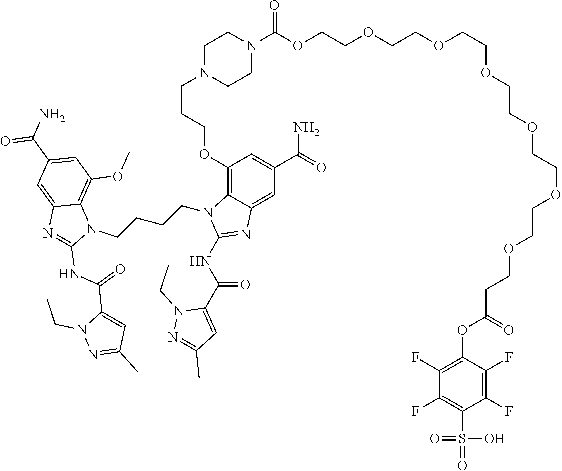 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(C(=O)OCCOCCOCCOCCOCCOCCOCCC(=O)Oc4c(F)c(F)c(S(=O)(=O)O)c(F)c4F)CC3)c21] | 1459.5 |
| BBI-L-15 | 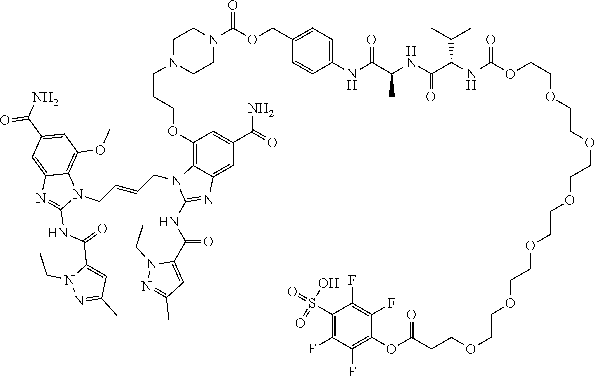 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(NC(=O)[C@H](C)NC(=O)[C@@H](NC(=O)OCCOCCOCCOCCOCCOCCOCCC(=O)Oc5c(F)c(F)c(S(=O)(=O)O)c(F)c5F)C(C)C)cc4)CC3)c21] | 1776.8 |
| BBI-L-16 | 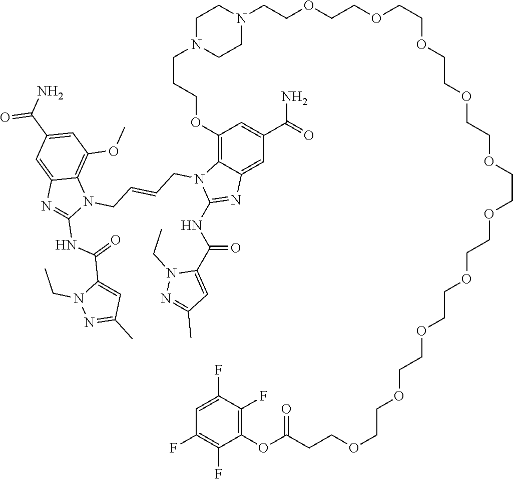 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCC(=O)Oc4c(F)c(F)cc(F)c4F)CC3)c21] | 1509.6 |
| BBI-L-17 | 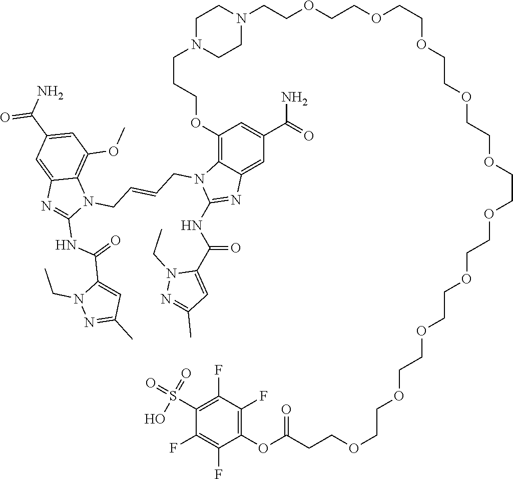 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCC(=O)Oc4c(F)c(F)c(S(=O)(=O)O)c(F)c4F)CC3)c21] | 1589.7 |
| BBI-L-18 | 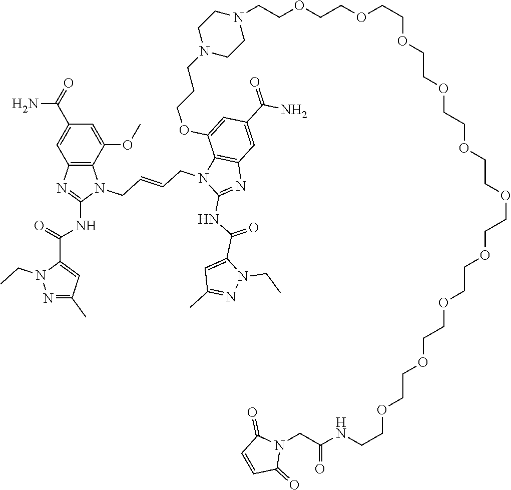 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21] | 1469.6 |

TABLE 1b
Bis-benzimidazole-linker (BBI-L) Formula II compounds

| BBI-L No. | Structure | MW |
|---|---|---|
| BBI-L-19 | 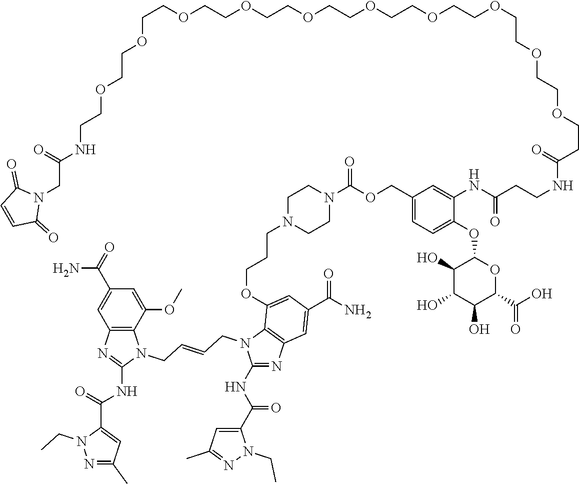 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(O[C@@H]5O[C@H](C(=O)O)[C@@H](O)[C@H](O)[C@H]5O)c(NC(=O)CCNC(=O)CCOCCOCCOCCOCCOCCOCCOCCOCCOCCOCCNC(=O)CN5C(=O)C=CC5=O)c4)CC3)c21] | 1910.0 |
| BBI-L-20 | 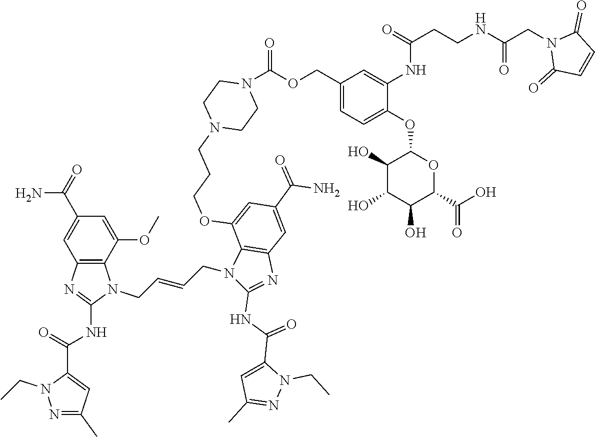 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(O[C@@H]5O[C@H](C(=O)O)[C@@H](O)[C@H](O)[C@H]5O)c(NC(=O)CCNC(=O)CN5C(=O)C=CC5=O)c4)CC3)c21] | 1398.4 |
| BBI-L-21 | 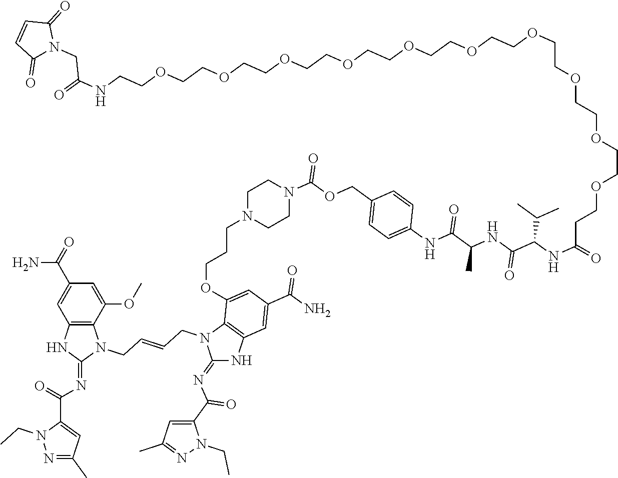 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCNC(=O)CN5C(=O)C=CC5=O)C(C)C)cc4)CC3)c21] | 1817.0 |
| BBI-L-22 | 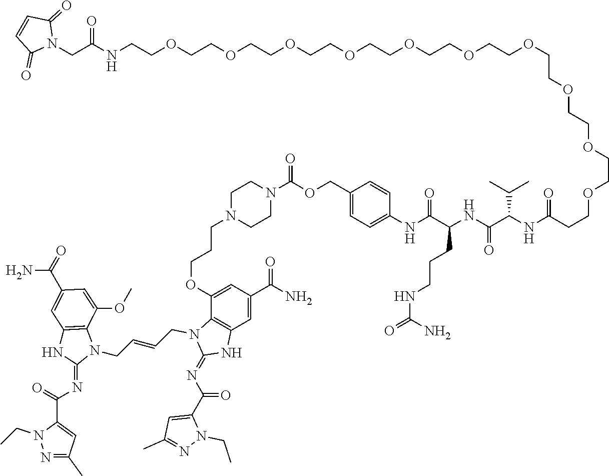 | 1903.1 |
| BBI-L-23 | 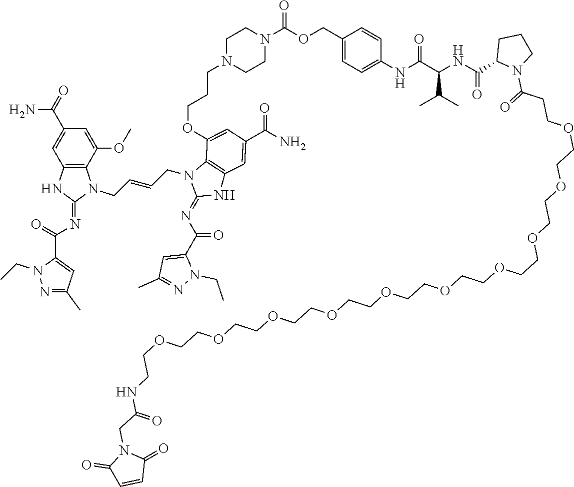 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@@H](NC(=O)[C@@H]5CCCN5C(=O)CCOCCOCCOCCOCCOCCOCCOCCOCCOCCOCCNC(=O)CN5C(=O)C=CC5=O)C(C)C)cc4)CC3)c21] | 1843.1 |
| BBI-L-24 | 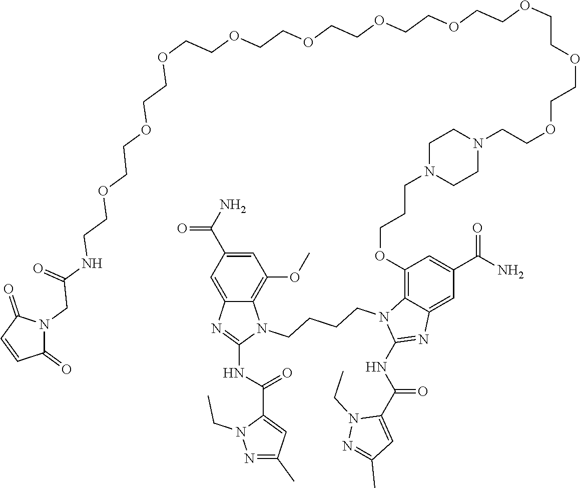 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21] | 1471.7 |
| BBI-L-25 | 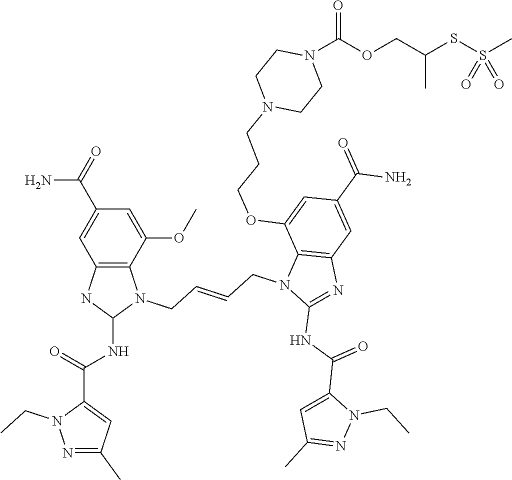 | 1045.2 |
| BBI-L-26 | 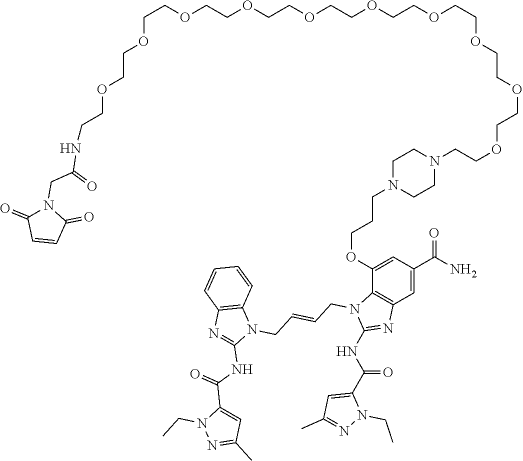 [SMILES: CCn1nc(C)cc1C(=O)Nc1nc2ccccc2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21] | 1396.6 |
| BBI-L-27 | 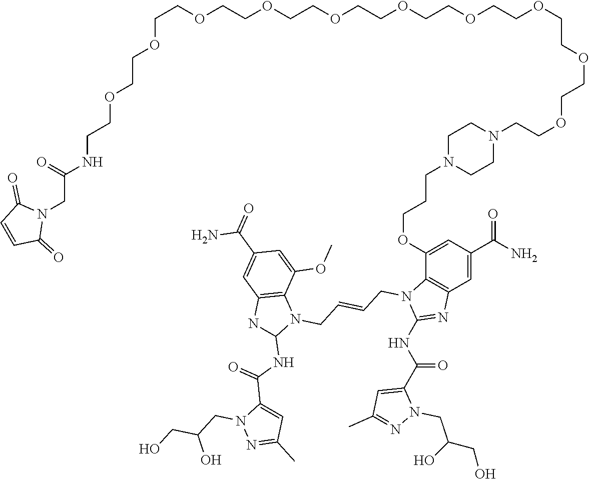 | 1561.7 |
| BBI-L-28 | 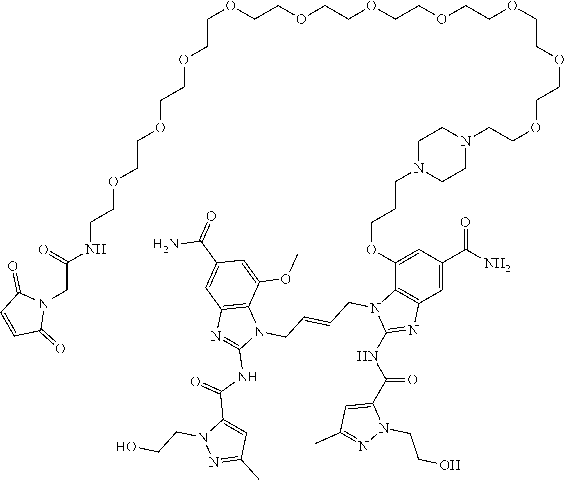 [SMILES: COc1cc(C(N)=O)cc2nc(NC(=O)c3cc(C)nn3CCO)n(C/C=C/Cn3c(NC(=O)c4cc(C)nn4CCO)nc4cc(C(N)=O)cc(OCCCN5CCN(CCOCCOCCOCCOCCOCCOCCOCCOCCOCCOCCNC(=O)CN6C(=O)C=CC6=O)CC5)c43)c12] | 1501.6 |
| BBI-L-29 | 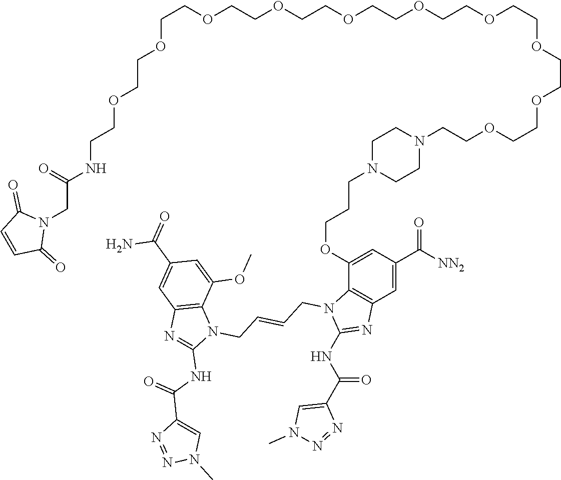 | 1415.5 |
| BBI-L-30 | 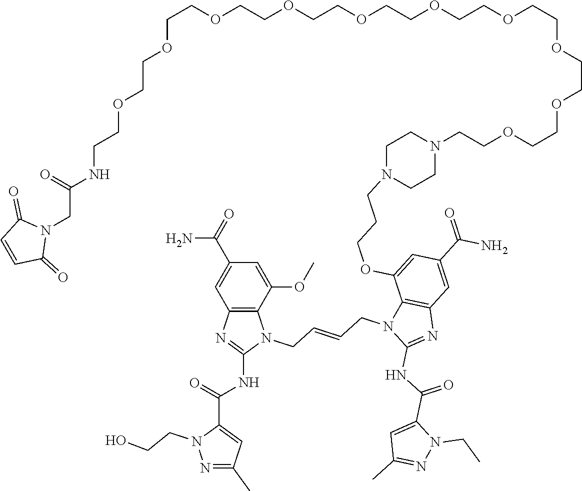 [SMILES: CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2CCO)nc2cc(C(N)=O)cc(OC)c21] | 1485.6 |
| BBI-L-31 | 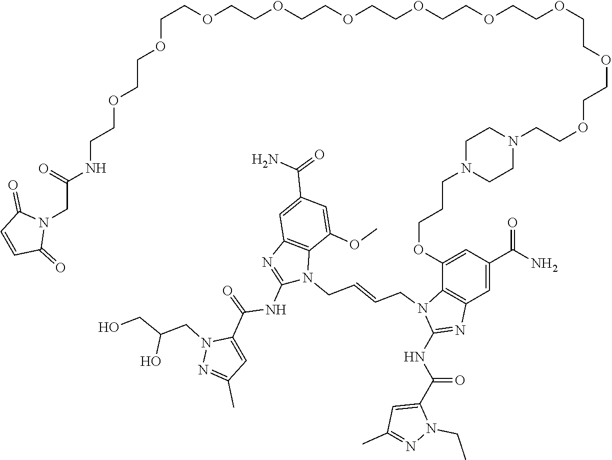 | 1515.7 |
| BBI-L-32 | 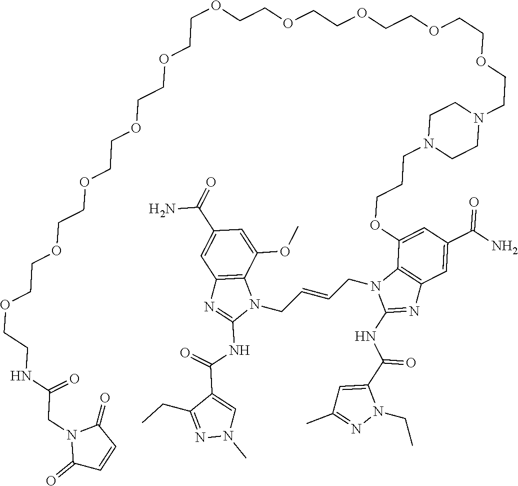 [SMILES: CCc1nn(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21] | 1469.9 |
| BBI-L-33 | 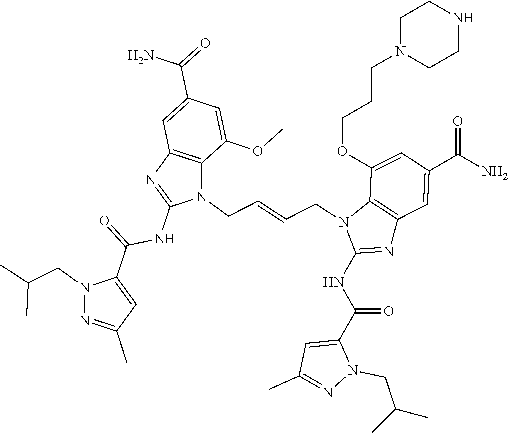 | 905.1 |
| BBI-L-34 | 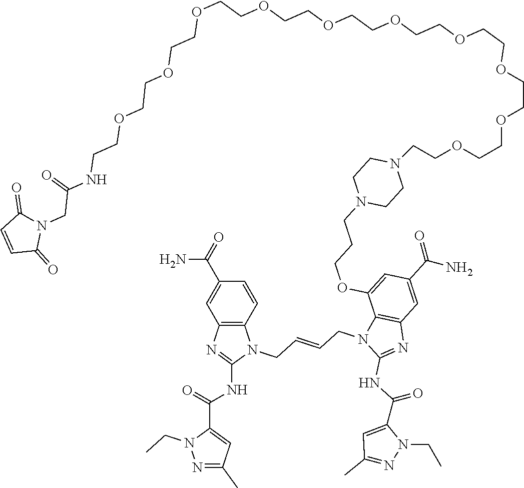 | 1439.6 |
| BBI-L-35 | 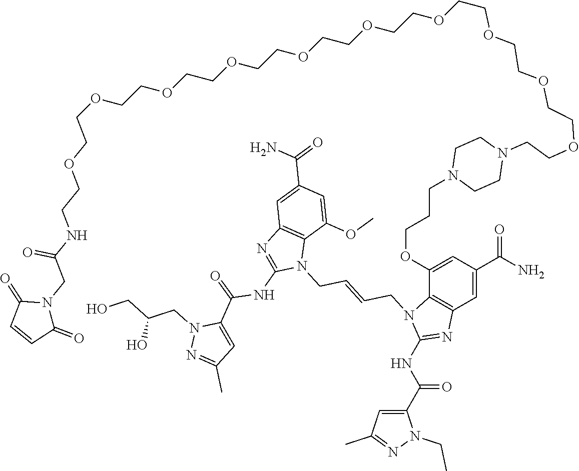 [SMILES: CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2C[C@H](O)CO)nc2cc(C(N)=O)cc(OC)c21] | 1515.7 |
| BBI-L-36 | 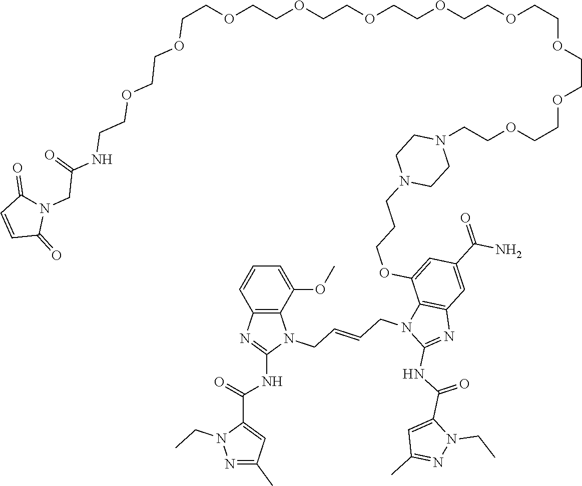 [SMILES: CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21] | 1426.6 |
| BBI-L-37 | 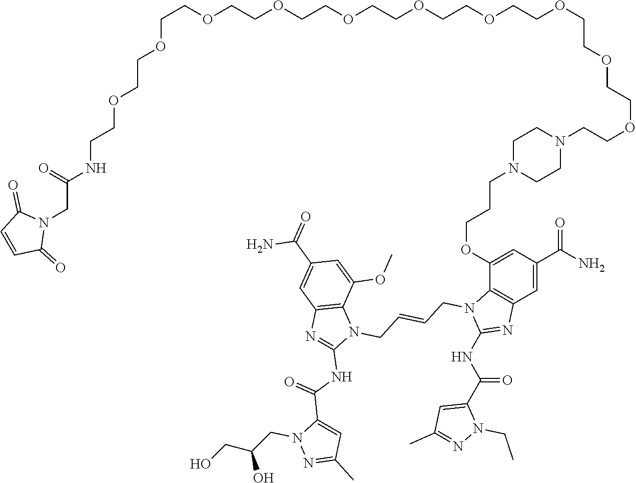 [SMILES: CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2C[C@@H](O)CO)nc2cc(C(N)=O)cc(OC)c21] | 1515.7 |
| BBI-L-38 | 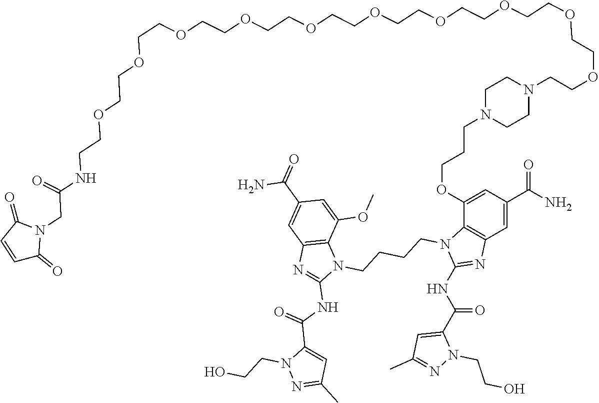 [SMILES: COc1cc(C(N)=O)cc2nc(NC(=O)c3cc(C)nn3CCO)n(CCCCn3c(NC(=O)c4cc(C)nn4CCO)nc4cc(C(N)=O)cc(OCCCN5CCN(CCOCCOCCOCCOCCOCCOCCOCCOCCOCCOCCNC(=O)CN6C(=O)C=CC6=O)CC5)c43)c12] | 1503.7 |

### Immunoconjugates

The immunoconjugates of the invention induce target-specific activation of immune effector cells such as myeloid cells as well as tumor cells expressing STING themselves. Tumor targeting brings specificity to minimize off-target STING activation, and the immunoconjugate enables phagocytosis to not only increase activation of the effector cells but also immune complex uptake and subsequent tumor antigen processing and presentation.

Exemplary embodiments of immunoconjugates comprise an antibody covalently attached to one or more STING agonist, bis-benzimidazole (BBI) moieties by a linker, and having Formula I:

Ab-[L-D]ₚ I
or a pharmaceutically acceptable salt thereof,
wherein:
Ab is the antibody;
p is an integer from 1 to 8;
D is the STING agonist moiety having the formula:

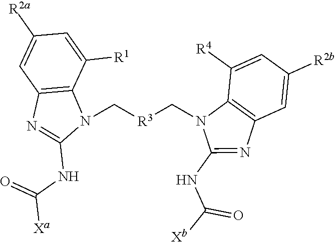

X^{a} and X^{b} are independently selected from a five-membered heteroaryl, optionally substituted with R⁵;
R¹ and R⁴ are independently selected from the group consisting of H, F, Cl, Br, I, —CN, —OH, —O—(C₁-C₆ alkyl), and R⁵;
R^{2a} and R^{2b} are independently selected from H, —C(═O)N(R⁶)₂, and R⁵;
where one of X^{a}, X^{b}, R¹, R⁴, R^{2a} and R^{2b} is substituted with R⁵;
R³ is selected from C₁-C₆ alkyldiyl, —(C₁-C₃ alkyldiyl)-O—(C₁-C₃ alkyldiyl)-, C₂-C₆ alkenyldiyl and C₂-C₆ alkynyldiyl, optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂;
R⁵ is selected from the group consisting of:
   (C₁-C₁₂ alkyldiyl)-*;
   (C₁-C₁₂ alkyldiyl)-N(R⁶)—*;
   (C₁-C₁₂ alkyldiyl)-O—*;
   (C₁-C₁₂ alkyldiyl)-(C₂-C₂₀ heterocyclyldiyl)-*;
   O—(C₁-C₁₂ alkyldiyl)-*;
   O—(C₁-C₁₂ alkyldiyl)-N(R⁶)—*;
   O—(C₁-C₁₂ alkyldiyl)-O—*;
   O—(C₁-C₁₂ alkyldiyl)-(C₂-C₂₀ heterocyclyldiyl)-*;
   O—(C₁-C₁₂ alkyldiyl)-(C₂-C₂₀ heterocyclyldiyl)-N(R⁶)—*;
   OC(═O)N(R⁶)—*;
   OC(═O)N(R⁶)—(C₁-C₁₂ alkyldiyl)-N(R⁶)—*;
   N(R⁶)—*;
   N(R⁶)—(C₁-C₁₂ alkyldiyl)-*;
   N(R⁶)—(C₁-C₁₂ alkyldiyl)-N(R⁶)—*;
   N(R⁶)—(C₁-C₁₂ alkyldiyl)-O—*;
   N(R⁶)—(C₁-C₁₂ alkyldiyl)-(C₂-C₂₀ heterocyclyldiyl)-*;
   C(═O)N(R⁶)—*;
   C(═O)N(R⁶)—(C₁-C₁₂ alkyldiyl)-*;
   C(═O)N(R⁶)—(C₁-C₁₂ alkyldiyl)-N(R⁶)—*;
   C(═O)N(R⁶)—(C₁-C₁₂ alkyldiyl)-O—*;
   (C₂-C₂₀ heterocyclyldiyl)-*;
   S(═O)₂—(C₂-C₂₀ heterocyclyldiyl)-*; and
   S(═O)₂—(C₂-C₂₀ heterocyclyldiyl)-(C₁-C₁₂ alkyldiyl)-N(R⁶)—*;
where the asterisk * indicates the attachment site of L;
R⁶ is independently H or C₁-C₆ alkyl;
L is the linker selected from the group consisting of:
   C(═O)-PEG-;
   C(═O)-PEG-C(═O)N(R⁶)—(C₁-C₁₂ alkyldiyl)-C(═O)-Gluc-;
   C(═O)-PEG-O—;
   C(═O)-PEG-O—C(═O)—;
   C(═O)-PEG-C(═O)—;
   C(═O)-PEG-C(═O)-PEP-;
   C(═O)-PEG-N(R⁶)—;
   C(═O)-PEG-N(R⁶)—C(═O)—;
   C(═O)-PEG-N(R⁶)-PEG-C(═O)-PEP-;
   C(═O)-PEG−N⁺(R⁶)₂-PEG-C(═O)-PEP-;
   C(═O)-PEG-C(═O)-PEP-N(R⁶)—(C₁-C₁₂alkyldiyl)-;
   C(═O)-PEG-C(═O)-PEP-N(R⁶)—(C₁-C₁₂ alkyldiyl)N(R⁶)C(═O)(C₂-C₅ monoheterocyclyldiyl)-;
   C(═O)-PEG-C(═O)N(R⁶)—(C₁-C₁₂ alkyldiyl)-C(═O)-PEP-;
   C(═O)-PEG-SS-(C₁-C₁₂ alkyldiyl)-OC(═O)—;
   C(═O)-PEG-SS-(C₁-C₁₂ alkyldiyl)-C(═O)—;
   C(═O)—(C₁-C₁₂ alkyldiyl)-C(═O)-PEP-;
   C(═O)—(C₁-C₁₂ alkyldiyl)-C(═O)-PEP-N(R⁶)—(C₁-C₁₂ alkyldiyl)-;
   C(═O)—(C₁-C₁₂ alkyldiyl)-C(═O)-PEP-N(R⁶)—(C₁-C₁₂ alkyldiyl)-N(R⁵)—C(═O);
   C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
   succinimidyl-(CH₂)ₘ—C(═O)N(R⁶)-PEG-;
   succinimidyl-(CH₂)ₘ—C(═O)N(R⁶)-PEG-C(═O)N(R⁶)—(C₁-C₁₂ alkyldiyl)-C(═O)-Gluc-;
   succinimidyl-(CH₂)ₘ—C(═O)N(R⁶)-PEG-O—;
   succinimidyl-(CH₂)ₘ—C(═O)N(R⁶)-PEG-O—C(═O)—;
   succinimidyl-(CH₂)ₘ—C(═O)N(R⁶)-PEG-C(═O)—;
   succinimidyl-(CH₂)ₘ—C(═O)N(R⁶)-PEG-N(R⁵)—;
   succinimidyl-(CH₂)ₘ—C(═O)N(R⁶)-PEG-N(R⁵)—C(═O)—;
   succinimidyl-(CH₂)ₘ—C(═O)N(R⁶)-PEG-C(═O)-PEP-;
   succinimidyl-(CH₂)ₘ—C(═O)N(R⁶)-PEG-SS-(C₁-C₁₂ alkyldiyl)-OC(═O)—;
   succinimidyl-(CH₂)ₘ—C(═O)-PEP-N(R⁶)—(C₁-C₁₂ alkyldiyl)-;
   succinimidyl-(CH₂)ₘ—C(═O)N(R⁶)-PEG-C(═O)N(R⁶)—(C₁-C₁₂ alkyldiyl)-C(═O)-PEP-;
   succinimidyl-(CH₂)ₘ—C(═O)-PEP-N(R⁶)—(C₁-C₁₂ alkyldiyl)N(R⁶)C(═O)—; and
   succinimidyl-(CH₂)ₘ—C(═O)-PEP-N(R⁶)—(C₁-C₁₂ alkyldiyl)N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
Gluc has the formula:

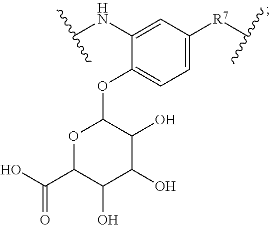

PEP has the formula:

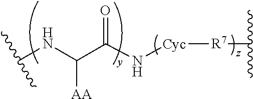

where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure:

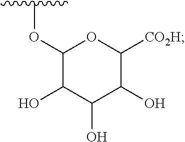 [SMILES: *OC1OC(C(=O)O)C(O)C(O)C1O]

R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring:
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

Antibodies target tumor-specific and/or immune-specific (e.g. PD-L1) antigens to bring specificity into the targeting of the immunoconjugate to enable safe and systemic delivery

An exemplary embodiment of the immunoconjugate of Formula I includes wherein the antibody is an antibody construct that has an antigen binding domain that binds PD-L1, such as atezolizumab, durvalumab, and avelumab, or a biosimilar or a biobetter thereof.

An exemplary embodiment of the immunoconjugate of Formula I includes wherein the antibody is an antibody construct that has an antigen binding domain that binds HER2, such as trastuzumab and pertuzumab, or a biosimilar or a biobetter thereof.

An exemplary embodiment of the immunoconjugate of Formula I includes wherein the antibody is an antibody construct that has an antigen binding domain that binds CEA, such as labetuzumab, or a biosimilar or a biobetter thereof.

An exemplary embodiment of the immunoconjugate of Formula I includes wherein the antibody is an antibody construct that has an antigen binding domain that binds Trop2, such as sacituzumab, or a biosimilar or a biobetter thereof.

An exemplary embodiment of the immunoconjugate of Formula I includes wherein X^{a} and X^{b} are independently selected from the group consisting of imidazolyl, pyrazolyl, triazolyl, tetrazolyl, furyl, thienyl, isoxazolyl, thiazolyl, oxadiazolyl, oxazolyl, isothiazolyl, pyrrolyl, oxadiazolyl, and thiadiazolyl.

An exemplary embodiment of the immunoconjugate of Formula I includes wherein X^{a} and X^{b} are each pyrazolyl, substituted with one or more groups selected from —CH₃, —CH₂CH₃, —CH═CH₂, —C≡CH, —C≡CCH₃, —CH₂CH₂CH₃, —CH(CH₃)₂, and —CH₂CH(CH₃)₂.

An exemplary embodiment of the immunoconjugate of Formula I includes wherein one of X^{a} and X^{b} is substituted with R⁵.

An exemplary embodiment of the immunoconjugate of Formula I includes wherein R¹ is selected from the group consisting of —OCH₃, —OCH₂CH₃, —OCH₂CH₂OCH₃, —OCH₂CH₂OH, and —OCH₂CH₂N(CH₃)₂.

An exemplary embodiment of the immunoconjugate of Formula I includes wherein R¹ is —OCH₃.

An exemplary embodiment of the immunoconjugate of Formula I includes wherein R¹ is F.

An exemplary embodiment of the immunoconjugate of Formula I includes wherein R^{2a} and R^{2b} are each —C(═O)NH₂.

An exemplary embodiment of the immunoconjugate of Formula I includes wherein one of R^{2a} and R^{2b} is substituted with R⁵.

An exemplary embodiment of the immunoconjugate of Formula I includes wherein R³ is selected from —CH₂CH₂—, —CH═CH—, and —C≡C—.

An exemplary embodiment of the immunoconjugate of Formula I includes wherein R³ is C₂-C₄ alkenyldiyl, substituted with one or more groups selected from F, —OH, and —OCH₃.

An exemplary embodiment of the immunoconjugate of Formula I includes wherein R⁴ is —O—(C₁-C₁₂ alkyldiyl)-(C₂-C₂₀ heterocyclyldiyl)-*.

An exemplary embodiment of the immunoconjugate of Formula I includes wherein C₁-C₁₂ alkyldiyl is propyldiyl and C₂-C₂₀ heterocyclyldiyl is piperidiyl.

An exemplary embodiment of the immunoconjugate of Formula I includes wherein one of R¹ and R⁴ is substituted with R⁵.

An exemplary embodiment of the immunoconjugate of Formula I includes wherein L is —C(═O)-PEG- or —C(═O)-PEG-C(═O)—.

An exemplary embodiment of the immunoconjugate of Formula I includes wherein L is attached to a cysteine thiol of the antibody.

An exemplary embodiment of the immunoconjugate of Formula I includes wherein for PEG, m is 1 or 2, and n is an integer from 2 to 10.

An exemplary embodiment of the immunoconjugate of Formula I includes wherein for PEG, n is 10.

An exemplary embodiment of the immunoconjugate of Formula I includes wherein L comprises PEP and PEP is a dipeptide and has the formula:

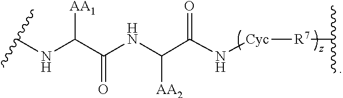

An exemplary embodiment of the immunoconjugate of Formula I includes wherein L comprises PEP and PEP is a tripeptide and has the formula:

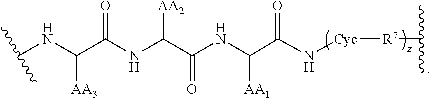

An exemplary embodiment of the immunoconjugate of Formula I includes wherein L comprises PEP and PEP is a tetrapeptide and has the formula:

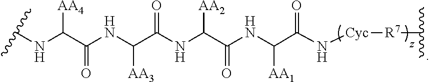

An exemplary embodiment of the immunoconjugate of Formula I includes wherein L comprises Gluc, selected from the formulas:

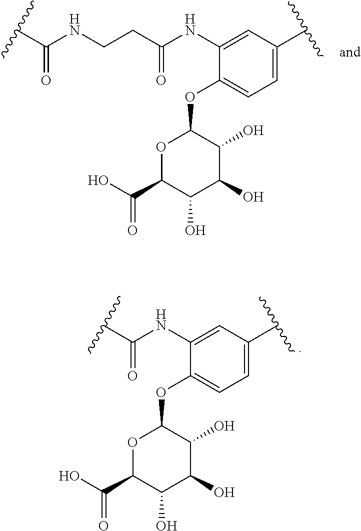 [SMILES: *C(=O)NCCC(=O)Nc1cc(*)ccc1O[C@@H]1O[C@H](C(=O)O)[C@@H](O)[C@H](O)[C@H]1O ; *C(=O)Nc1cc(*)ccc1O[C@@H]1O[C@H](C(=O)O)[C@@H](O)[C@H](O)[C@H]1O]

An exemplary embodiment of the immunoconjugate of Formula I includes wherein wherein L is selected from the structures:

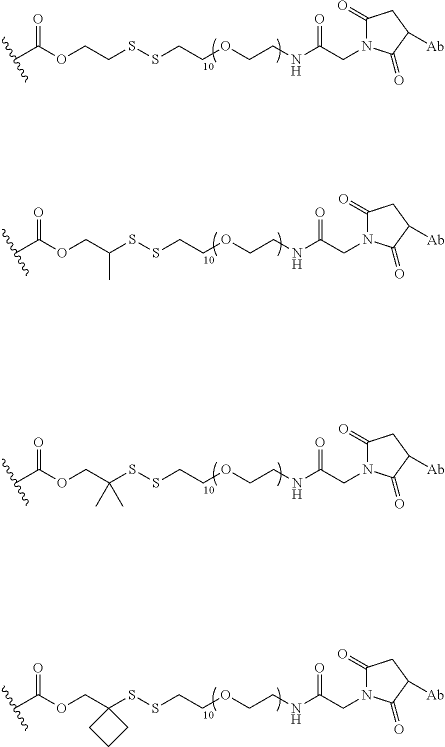

where the wavy line indicates the attachment to R⁵.

The invention includes all reasonable combinations, and permutations of the features, of the Formula I embodiments.

In certain embodiments, the immunoconjugate compounds of the invention include those with immunostimulatory activity. The antibody-drug conjugates of the invention selectively deliver an effective dose of a bis-benzimidazole drug to tumor tissue, whereby greater selectivity (i.e., a lower efficacious dose) may be achieved while increasing the therapeutic index (“therapeutic window”) relative to unconjugated bis-benzimidazole.

Drug loading is represented by p, the number of BBI moieties per antibody in an immunoconjugate of Formula I. Drug (BBI) loading may range from 1 to about 8 drug moieties (D) per antibody. Immunoconjugates of Formula I include mixtures or collections of antibodies conjugated with a range of drug moieties, from 1 to about 8. In some embodiments, the number of drug moieties that can be conjugated to an antibody is limited by the number of reactive or available amino acid side chain residues such as lysine and cysteine. In some embodiments, free cysteine residues are introduced into the antibody amino acid sequence by the methods described herein. In such aspects, p may be 1, 2, 3, 4, 5, 6, 7, or 8, and ranges thereof, such as from 1 to 8 or from 2 to 5. In any such aspect, p and n are equal (i.e., p=n=1, 2, 3, 4, 5, 6, 7, or 8, or some range there between). Exemplary immunoconjugates of Formula I include, but are not limited to, antibodies that have 1, 2, 3, or 4 engineered cysteine amino acids (Lyon, R. et al. (2012) Methods in Enzym. 502:123-138). In some embodiments, one or more free cysteine residues are already present in an antibody forming intra-chain and inter-chain disulfide bonds (native disulfide groups), without the use of engineering, in which case the existing free, reduced cysteine residues may be used to conjugate the antibody to a drug. In some embodiments, an antibody is exposed to reducing conditions prior to conjugation of the antibody in order to generate one or more free cysteine residues.

For some immunoconjugates, p may be limited by the number of attachment sites on the antibody. For example, where the attachment is a cysteine thiol, as in certain exemplary embodiments described herein, an antibody may have only one or a limited number of cysteine thiol groups, or may have only one or a limited number of sufficiently reactive thiol groups, to which the drug may be attached. In other embodiments, one or more lysine amino groups in the antibody may be available and reactive for conjugation with a BBI-linker compound of Formula II. In certain embodiments, higher drug loading, e.g. p>5, may cause aggregation, insolubility, toxicity, or loss of cellular permeability of certain antibody-drug conjugates. In certain embodiments, the average drug loading for an immunoconjugate ranges from 1 to about 8; from about 2 to about 6; or from about 3 to about 5. In certain embodiments, an antibody is subjected to denaturing conditions to reveal reactive nucleophilic groups such as lysine or cysteine.

The loading (drug/antibody ratio) of an immunoconjugate may be controlled in different ways, and for example, by: (i) limiting the molar excess of the BBI-linker intermediate compound relative to antibody, (ii) limiting the conjugation reaction time or temperature, and (iii) partial or limiting reductive denaturing conditions for optimized antibody reactivity.

It is to be understood that where more than one nucleophilic group of the antibody reacts with a drug, then the resulting product is a mixture of immunoconjugate compounds with a distribution of one or more drug moieties attached to an antibody. The average number of drugs per antibody may be calculated from the mixture by a dual ELISA antibody assay, which is specific for antibody and specific for the drug. Individual immunoconjugate molecules may be identified in the mixture by mass spectroscopy and separated by HPLC, e.g. hydrophobic interaction chromatography (see, e.g., McDonagh et al. (2006) Prot. Engr. Design & Selection 19(7):299-307; Hamblett et al. (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting. Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous immunoconjugate with a single loading value may be isolated from the conjugation mixture by electrophoresis or chromatography.

An exemplary embodiment of the immunoconjugate of Formula I is selected from the Tables 2a and 2b Immunoconjugates. Assessment of Immunoconjugate Activity In Vitro may be conducted according to the methods of Example 202.

STING activation is canonically associated with induction of type I/III IFNs (interferons) through IRF3 (interferon regulatory factor 3) signaling, but can also induce proinflammatory cytokines such as TNFα (tumor necrosis factor alpha) through the NF-κB (nuclear factor kappa-light-chain-enhancer of activated B cells) pathway. Certain immunoconjugates demonstrate the ability to elicit IFNλ1 (interferon lambda 1) as well as TNFα, consistent with STING activation. As a comparator, trastuzumab elicited essentially no TNFα or IFNλ1 in the PBMC tumor co-culture assay (Example 202).

TABLE 2a
BBI Immunoconjugates (IC)

| | | | | PBMC | PBMC |
|---|---|---|---|---|---|
| Immuno- | | | | Assay TNFα | Assay IFNλ1 |
| conjugate | BBI-L- | Antibody | | Secretion | Secretion |
| No. | Table 1a | Target | DAR | EC50 [nM] | EC50 [nM] |
| IC-1 | BBI-L-1 | trastuzumab | 2.2 | 1.0 nM | 9.6 nM |
| | | HER2 | | | |
| IC-2 | BBI-L-3 | trastuzumab | 2.4 | 0.6 nM | 0.3 nM |
| | | HER2 | | | |
| IC-3 | BBI-L-8 | trastuzumab | 3.4 | 1.1 nM | 2.0 nM |
| | | HER2 | | | |
| IC-4 | BBI-L-9 | trastuzumab | 2.1 | 1.5 nM | 4.4 nM |
| | | HER2 | | | |

TABLE 2b
BBI Immunoconjugates (IC)

| | | | | PBMC | PBMC |
|---|---|---|---|---|---|
| | BBI-L- | | | Assay TNFα | Assay IFNλ1 |
| Immunoconjugate | Tables 1a | | | Secretion | Secretion |
| No. | and 1b | Antibody | DAR | EC50 [nM] | EC50 [nM] |
| IC-5 | BBI-L-18 | trastuzumab | 4.0 | 0.7 | 1.8 |
| | | HER2 | 3.6 | | |
| IC-6 | BBI-L-18 | tras-G1f-N297A | 3.3 | | 9.6 |
| | | HER2 | | | |
| IC-7 | BBI-L-18 | avelumab | 3.5 | 0.3 | 1.5 (X-PDL1) |
| | | PD-L1 | | 0.3 (X-PDL1) | |
| IC-8 | BBI-L-18 | PDL1.110-G1f | 3.4 | 0.9 | 1.9 (X-PDL1) |
| | | PD-L1 | | 0.7 (X-PDL1) | |
| IC-9 | BBI-L-18 | bezlotox-G1f | 3.2 | | |
| IC-10 | BBI-L-7 | trastuzumab | 3.3 | | |
| | | HER2 | | | |
| IC-11 | BBI-L-18 | avelu-G1za- | 4.3 | | |
| | | N297AhL1 | | | |
| | | PD-L1 | | | |
| IC-12 | BBI-L-18 | CLDN.1-G1f | 3.1 | | |
| | | | 3.7 | | |
| IC-13 | BBI-L-24 | trastuzumab | 3.7 | 1.4 | 2.0 |
| | | HER2 | | | |
| IC-14 | BBI-L-17 | BSA monomer | 3.4 | | |
| IC-15 | BBI-L-19 | trastuzumab | 3.8 | | |
| | | HER2 | | | |
| IC-16 | BBI-L-20 | trastuzumab | 3.9 | | |
| | | HER2 | | | |
| IC-17 | BBI-L-23 | trastuzumab | 4.0 | | |
| | | HER2 | | | |
| IC-18 | BBI-L-13 | trastuzumab | 3.7 | | |
| | | HER2 | | | |
| IC-19 | BBI-L-22 | trastuzumab | 4.1 | | |
| | | HER2 | | | |
| IC-20 | BBI-L-21 | trastuzumab | 3.9 | | |
| | | HER2 | | | |
| IC-21 | BBI-L-25 | trastuzumab | 4.5 | | |
| | | HER2 | | | |
| IC-22 | BBI-L-26 | trastuzumab | 3.3 | 3.5 | 1.3 |
| | | HER2 | | | |
| IC-23 | BBI-L-27 | trastuzumab | 3.0 | | |
| | | HER2 | | | |
| IC-24 | BBI-L-28 | trastuzumab | 3.0 | 1.5 | 3.7 |
| | | HER2 | | | |
| IC-25 | BBI-L-29 | trastuzumab | 3.6 | | |
| | | HER2 | | | |
| IC-26 | BBI-L-24 | avelumab | 3.7 | | |
| | | PD-L1 | | | |
| IC-27 | BBI-L-30 | trastuzumab | 3.6 | | |
| | | HER2 | | | |
| IC-28 | BBI-L-31 | trastuzumab | 3.3 | | |
| | | HER2 | | | |
| IC-29 | BBI-L-32 | trastuzumab | 3.3 | | |
| | | HER2 | | | |
| IC-30 | BBI-L-34 | trastuzumab | 3.2 | | |
| | | HER2 | | | |
| IC-31 | BBI-L-18 | bezlotox-G1f- | 3.2 | | |
| | | G236A | | | |
| IC-32 | BBI-L-18 | M25-mG2a | 3.9 | | |
| | | LRRC15 | | | |
| IC-33 | BBI-L-18 | anti-Mouse | 4.0 | | |
| | | Thy 1.1 (19E12) | | | |
| | | LRRC15 | | | |
| IC-34 | BBI-L-35 | trastuzumab | 3.6 | | |
| | | HER2 | | | |
| IC-35 | BBI-L-37 | trastuzumab | 3.5 | | |
| | | HER2 | | | |
| IC-36 | BBI-L-36 | trastuzumab | 3.6 | | |
| | | HER2 | | | |
| IC-37 | BBI-L-38 | trastuzumab | 3.1 | | |
| | | HER2 | | | |

### Compositions of Immunoconjugates

The invention provides a composition, e.g., a pharmaceutically or pharmacologically acceptable composition or formulation, comprising a plurality of immunoconjugates as described herein and optionally a carrier therefor, e.g., a pharmaceutically or pharmacologically acceptable carrier. The immunoconjugates can be the same or different in the composition, i.e., the composition can comprise immunoconjugates that have the same number of BBI adjuvants linked to the same positions on the antibody construct and/or immunoconjugates that have the same number of BBI adjuvants linked to different positions on the antibody construct, that have different numbers of adjuvants linked to the same positions on the antibody construct, or that have different numbers of adjuvants linked to different positions on the antibody construct.

In an exemplary embodiment, a composition comprising the immunoconjugate compounds comprises a mixture of the immunoconjugate compounds, wherein the average drug (BBI) loading per antibody in the mixture of immunoconjugate compounds is about 2 to about 5.

A composition of immunoconjugates of the invention can have an average adjuvant to antibody construct ratio (DAR) of about 0.4 to about 10. A skilled artisan will recognize that the number of thienoazepine adjuvants conjugated to the antibody construct may vary from immunoconjugate to immunoconjugate in a composition comprising multiple immunoconjugates of the invention and thus the adjuvant to antibody construct (e.g., antibody) ratio can be measured as an average which may be referred to as the drug to antibody ratio (DAR). The adjuvant to antibody construct (e.g., antibody) ratio can be assessed by any suitable means, many of which are known in the art.

The average number of adjuvant moieties per antibody (DAR) in preparations of immunoconjugates from conjugation reactions may be characterized by conventional means such as mass spectrometry, ELISA assay, and HPLC. The quantitative distribution of immunoconjugates in a composition in terms of p may also be determined. In certain instances, separation, purification, and characterization of homogeneous immunoconjugates where p is a certain value from immunoconjugates with other drug loadings may be achieved by means such as reverse phase HPLC or electrophoresis.

In some embodiments, the composition further comprises one or more pharmaceutically or pharmacologically acceptable excipients. For example, the immunoconjugates of the invention can be formulated for parenteral administration, such as IV administration or administration into a body cavity or lumen of an organ. Alternatively, the immunoconjugates can be injected into the tumor (intra-tumorally). Compositions for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the immunoconjugate. The concentration of the immunoconjugate in the composition can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

Method of Treating Cancer with Immunoconjugates

The invention provides a method for treating cancer. The method includes administering a therapeutically effective amount of an immunoconjugate as described herein (e.g., as a composition as described herein) to a subject in need thereof. e.g., a subject that has cancer and is in need of treatment for the cancer. The method includes administering a therapeutically effective amount of an immunoconjugate (IC) selected from Table 2.

It is contemplated that the immunoconjugate of the present invention may be used to treat various hyperproliferative diseases or disorders, e.g. characterized by the overexpression of a tumor antigen. Exemplary hyperproliferative disorders include benign or malignant solid tumors and hematological disorders such as leukemia and lymphoid malignancies.

In another aspect, an immunoconjugate for use as a medicament is provided. In certain embodiments, the invention provides an immunoconjugate for use in a method of treating an individual comprising administering to the individual an effective amount of the immunoconjugate. In one such embodiment, the method further comprises administering to the individual an effective amount of at least one additional therapeutic agent, e.g., as described herein.

In a further aspect, the invention provides for the use of an immunoconjugate in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin.

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyxoid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, Askin's tumor; sarcoma botryoides; chondrosarcoma; Ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); Kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule on the face, head or neck Merkel cell carcinoma is also called neuroendocrine carcinoma of the skin. In some embodiments, methods for treating Merkel cell carcinoma include administering an immunoconjugate containing an antibody construct that is capable of binding Trop2 (e.g., sacituzumab, sacituzumab govitecan, biosimilars thereof, or biobetters thereof). In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Stemberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

Immunoconjugates of the invention can be used either alone or in combination with other agents in a therapy. For instance, an immunoconjugate may be co-administered with at least one additional therapeutic agent, such as a chemotherapeutic agent. Such combination therapies encompass combined administration (where two or more therapeutic agents are included in the same or separate formulations), and separate administration, in which case, administration of the immunoconjugate can occur prior to, simultaneously, and/or following, administration of the additional therapeutic agent and/or adjuvant. Immunoconjugates can also be used in combination with radiation therapy.

The immunoconjugates of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The immunoconjugate described herein can be used to treat the same types of cancers as sacituzumab, sacituzumab govitecan, biosimilars thereof, and biobetters thereof, particularly breast cancer, especially triple negative (test negative for estrogen receptors, progesterone receptors, and excess HER2 protein) breast cancer, bladder cancer, and Merkel cell carcinoma.

In some embodiments, the immunoconjugates described herein may be effective in the treatment of bladder cancer, salivary gland cancer, endometrial cancer, urinary tract cancer, urothelial carcinoma, lung cancer, non-small cell lung cancer, Merkel cell carcinoma, colon cancer, colorectal cancer, gastric cancer, and breast cancer.

The immunoconjugate is administered to a subject in need thereof in any therapeutically effective amount using any suitable dosing regimen, such as the dosing regimens utilized for sacituzumab, sacituzumab govitecan, biosimilars thereof, and biobetters thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer such as triple negative (test negative for estrogen receptors, progesterone receptors, and excess HER2 protein) breast cancer.

In some embodiments, the cancer is susceptible to a pro-inflammatory response induced by STING.

### EXAMPLES

### Preparation of Bis-Benzimidazole-Linker (BBI-L) Formula II Compounds and Intermediates

### Example 3 Synthesis of (2E)-1-[(E)-4-[(2E)-5-carbamoyl-2-(2-ethyl-5-methyl-pyrazole-3-carbonyl)imino-7-(3-piperazin-1-ylpropoxy)-3H-benzimidazol-1-yl]but-2-enyl]-2-(2-ethyl-5-methyl-pyrazole-3-carbonyl)imino-7-methoxy-3H-benzimidazole-5-carboxamide, 3

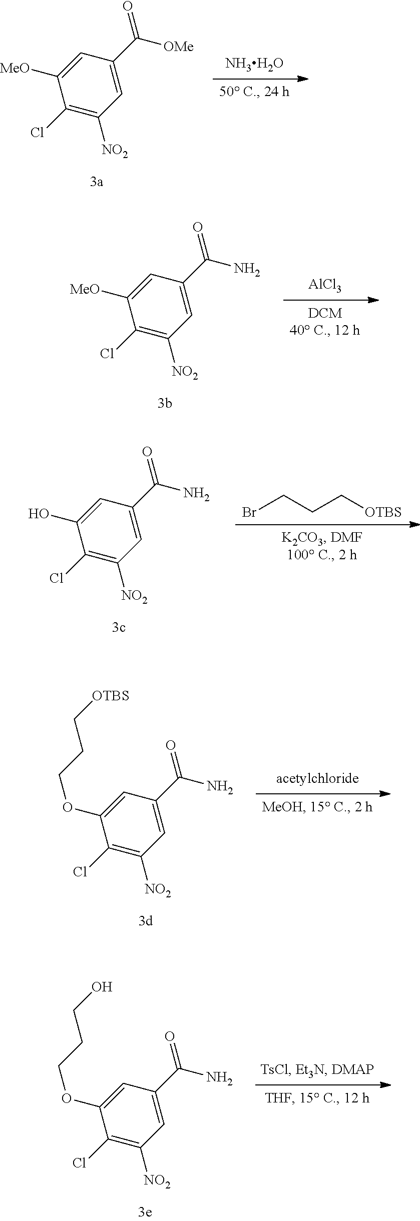

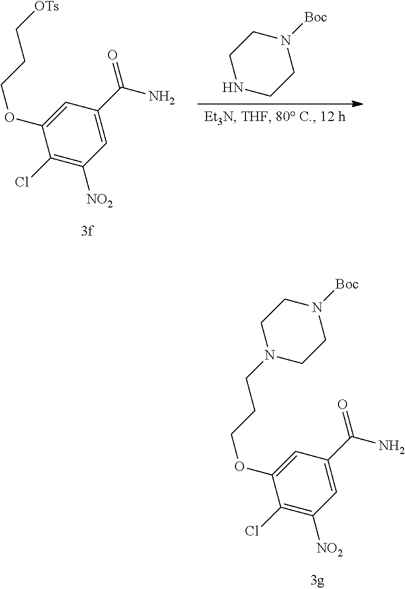

### Preparation of 4-chloro-3-methoxy-5-nitro-benzamide, 3b

To a solution of methyl 4-chloro-3-methoxy-5-nitro-benzoate, 3a (15 g, 61.0 mmol, 1 eq) in NH₃·H₂O (136.5 g, 973.7 mmol, 150 mL, 25% purity, 16.0 eq) and it was stirred at 50° C. for 24 h. The mixture was filtered, the cake was washed with water (300 mL). The cake was dried under reduced pressure to give 3b (11 g, 47.70 mmol, 78.11% yield) as off-white solid. ¹H NMR (DMSO-d₆, 400 MHz) δ8.29 (s, 1H), 8.05 (d, J=1.6 Hz, 1H), 7.88 (d, J=1.6 Hz, 1H), 7.78 (s, 1H), 4.02 (s, 3H).

### Preparation of 4-chloro-3-hydroxy-5-nitro-benzamide, 3c

To a solution of 3b (6 g, 26.0 mmol, 1 eq) in DCM (100 mL) was added AlCl₃ (21.0 g, 156 mmol, 8.50 mL, 6 eq) and it was stirred at 40° C. for 12 h. The reaction mixture was cooled to 0° C., and quenched by addition aq 2 M HCl 50 mL at 0° C., and then stirred at 15° C. for 30 min, and extracted with EtOAc (50 mL×3). The combined organic layers were washed with brine (50 mL), dried over Na₂SO₄, filtered and concentrated under reduced pressure to give 3c (4 g, crude) as light yellow solid. ¹H NMR (DMSO-d₆, 400 MHz) δ11.52 (s, 1H), 8.17 (s, 1H), 7.92 (d, J=2.0 Hz, 1H), 7.71 (d, J=2.0 Hz, 1H), 7.66 (s, 1H).

### Preparation of 3-[3-[tert-butyl(dimethyl)silyl]oxypropoxy]-4-chloro-5-nitro-benzamide, 3d

To a mixture of 3-bromopropan-1-ol (25 g, 180.0 mmol, 16.0 mL, 1 eq) in THF (250 mL) was added imidazole (25.0 g, 360 mmol, 2 eq) and TBSCl (32.6 g, 216 mmol, 26.50 mL, 1.2 eq) and it was stirred at 15° C. for 2 h. The mixture was diluted with water (300 mL) and extracted with EtOAc (100 mL×3). The organic layer was washed with brine (100 mL), dried over Na₂SO₄, filtered and concentrated. The residue was purified by flash silica gel chromatography (ISCO®; 70 g SepaFlash® Silica Flash Column, Eluent of 0˜10% Ethyl acetate/Petroleum ether gradient at 100 mL/min) to give 3-bromopropoxy-tert-butyl-dimethyl-silane (43 g, 170 mmol, 94.40% yield) as colorless oil. ¹H NMR (CDCl₃, 400 MHz) δ3.75 (t, J=5.6 Hz, 2H), 3.53 (t, J=6.4 Hz, 2H), 2.11-1.99 (m, 2H), 0.91 (s, 9H), 0.08 (s, 6H).

To a solution of 3-bromopropoxy-tert-butyl-dimethyl-silane (19.8 g, 78.0 mmol, 1.3 eq) in DMF (200 mL) was added 3c (13 g, 60.0 mmol, 1 eq) and K₂CO₃ (16.60 g, 120.0 mmol, 2 eq) at 15° C. and it was stirred at 100° C. for 2 h. The mixture was diluted with water (600 mL) and extracted with EtOAc (200 mL×3). The organic layer was washed with brine (200 mL×3), dried over Na₂SO₄, filtered and concentrated. The residue was purified by flash silica gel chromatography (ISCO®; 35 g SepaFlash® Silica Flash Column, Eluent of 0˜50% Ethyl acetate/Petroleum ether gradient at 65 mL/min) to give 3d (24 g, crude) as light yellow solid. ¹H NMR (DMSO-d₆, 400 MHz) δ8.28 (s, 1H), 8.04 (d, J=1.6 Hz, 1H), 7.88 (d, J=1.6 Hz, 1H), 7.76 (s, 1H), 4.29 (t, J=6.0 Hz, 2H), 3.79 (t, J=6.0 Hz, 2H), 2.01-1.93 (m, 2H), 0.83 (s, 9H), 0.01 (s, 6H).

### Preparation of 4-chloro-3-(3-hydroxypropoxy)-5-nitro-benzamide, 3e

To a solution of 3d (24 g, 61.7 mmol, 1 eq) in MeOH (300 mL) was added acetyl chloride (48.4 g, 617 mmol, 44.0 mL, 10 eq) and it was stirred at 15° C. for 2 h. The pH of the mixture was adjusted to around ˜8 by progressively adding solid NaHCO₃, and then filtered and concentrated to give 3e (15 g, 54.61 mmol, 88.50% yield) as light yellow solid. ¹H NMR (DMSO-d₆, 400 MHz) δ8.35 (br s, 1H), 8.04 (d, J=1.6 Hz, 1H), 7.91 (d, J=1.6 Hz, 1H), 7.76 (s, 1H), 4.64 (t, J=5.2 Hz, 1H), 4.30 (t, J=6.4 Hz, 2H), 3.64-3.52 (m, 2H), 1.97-1.89 (m, 2H).

### Preparation of 3-(5-carbamoyl-2-chloro-3-nitro-phenoxy)propyl 4-methylbenzenesulfonate, 3f

To a solution of 3e (15 g, 54.6 mmol, 1 eq) in THF (150 mL) was added Et₃N (27.6 g, 273 mmol, 38.0 mL, 5 eq), DMAP (667 mg, 5.5 mmol, 0.1 eq) and TsCl (11.60 g, 164 mmol, 3 eq) and it was stirred at 15° C. for 12 h. The mixture was diluted with water (300 mL) and extracted with EtOAc (100 mL×3). The organic layer was washed with brine (100 mL), dried over Na₂SO₄, filtered and concentrated. The residue was purified by flash silica gel chromatography (ISCO®; 40 g SepaFlash® Silica Flash Column, Eluent of 0˜80% Ethyl acetate/Petroleum ether gradient at 45 mL/min) to give 3f (20 g, 46.6 mmol, 85.39% yield) as light yellow solid. ¹H NMR (DMSO-d₆, 400 MHz) δ8.28 (s, 1H), 8.04 (d, J=1.6 Hz, 1H), 7.79 (s, 1H), 7.75-7.69 (m, 3H), 7.27 (d, J=8.0 Hz, 2H), 4.22 (t, J=5.6 Hz, 2H), 4.13 (t, J=5.6 Hz, 2H), 2.27 (s, 3H), 2.15-2.07 (m, 2H).

### Preparation of tert-butyl 4-[3-(5-carbamoyl-2-chloro-3-nitro-phenoxy)propyl]piperazine-1-carboxylate, 3g

To a solution of 3f (13.5 g, 31.5 mmol, 1 eq) in THF (150 mL) was added Et₃N (6.40 g, 63.0 mmol, 8.80 mL, 2 eq) and tert-butyl piperazine-1-carboxylate; hydrochloride (9.80 g, 44.0 mmol, 1.4 eq) and it was stirred at 80° C. for 12 h. The mixture was diluted with water (300 mL) and extracted with EtOAc (100 mL×3). The organic layer was washed with brine (100 mL), dried over Na₂SO₄, filtered and concentrated. The residue was purified by flash silica gel chromatography (ISCO®; 20 g SepaFlash® Silica Flash Column, Eluent of 0 to 80% Ethyl acetate/Petroleum ether gradient at 45 mL/min) to give 3g (10 g, 22.58 mmol, 71.72% yield) as light yellow solid. ¹H NMR (DMSO-d₆, 400 MHz) δ8.28 (br s, 1H), 8.04 (d, J=1.6 Hz, 1H), 7.87 (d, J=1.6 Hz, 1H), 7.77 (br s, 1H), 4.27 (t. J=6.4 Hz, 2H), 3.30 (br t, J=4.8 Hz, 4H), 2.48-2.44 (m, 2H), 2.32 (br t, J=4.8 Hz, 4H), 1.95 (br t, J=6.4 Hz, 2H), 1.39 (s, 9H).

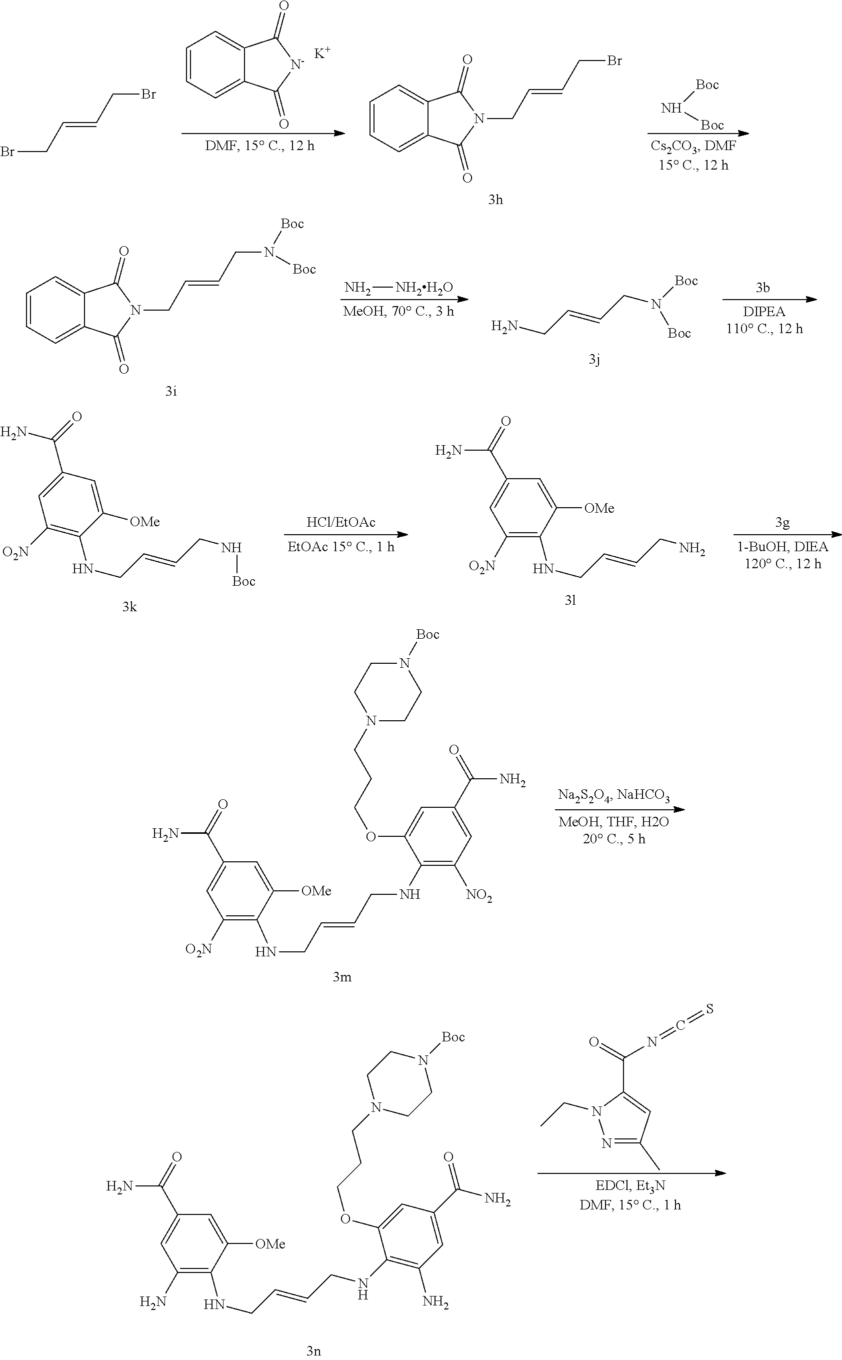

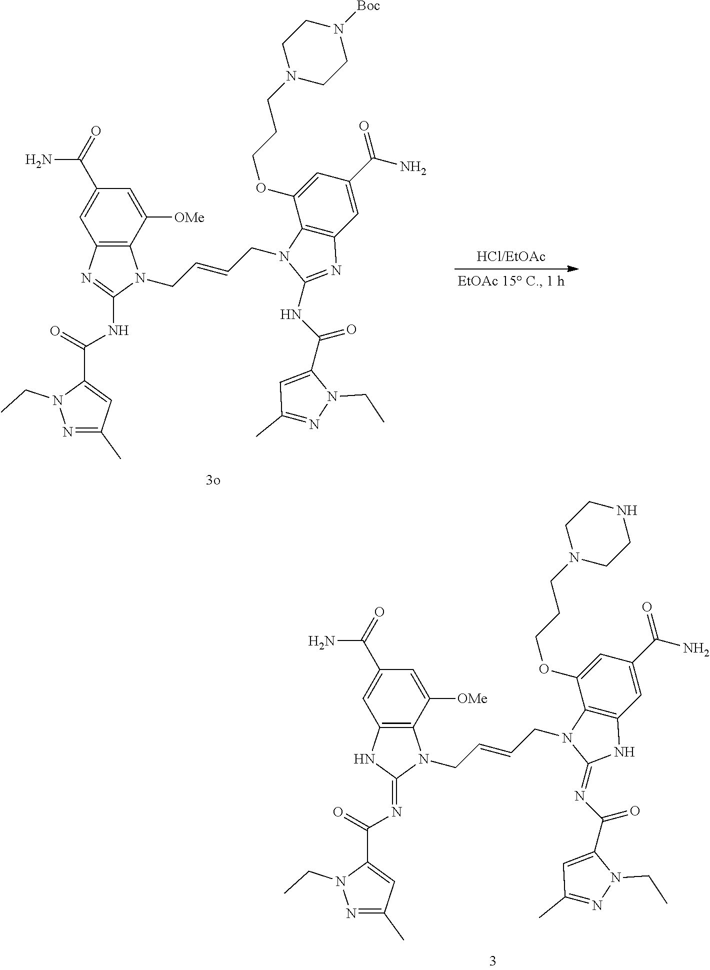 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OC(C)(C)C)CC3)c21 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

### Preparation of 2-[(E)-4-bromobut-2-enyl]isoindoline-1,3-dione, 3h

To a solution of (1,3-dioxoisoindolin-2-yl)potassium (33.6 g, 181 mmol, 0.8 eq) in DMF (300 mL) was added (E)-1,4-dibromobut-2-ene (48.5 g, 227 mmol, 1 eq) and it was stirred at 15° C. for 12 h. The mixture was diluted with water (1000 mL) and extracted with EtOAc (500 mL×3). The organic layer was washed with brine (300 mL×3), dried over Na₂SO₄, filtered and concentrated. The residue was purified by flash silica gel chromatography (ISCO®; 100 g SepaFlash® Silica Flash Column, Eluent of 0 to 60% Ethyl acetate/Petroleum ether gradient di) 65 mL/min) to give 2-[(E)-4-bromobut-2-enyl]isoindoline-1,3-dione (50 g, 178 mmol, 78.72% yield) as white solid. ¹H NMR (CDCl₃, 400 MHz) δ7.89-7.83 (m, 2H), 7.77-7.69 (m, 2H), 6.00-5.90 (m, 1H), 5.88-5.79 (m, 1H), 4.35-4.28 (m, 2H), 3.93-3.88 (m, 2H).

### Preparation of tert-butyl N-tert-butoxycarbonyl-N-[(E)-4-(1,3-dioxoisoindolin-2-yl)but-2-enyl]carbamate, 3i

To a solution of 3h (50 g, 178 mmol, 1 eq) in DMF (500 mL) was added Cs₂CO₃ (87.2 g, 268 mmol, 1.5 eq) and tert-butyl N-tert-butoxycarbonylcarbamate (50.0 g, 232 mmol, 1.3 eq) and it was stirred at 15° C. for 12 h. The mixture was diluted with water (2000 mL) and extracted with EtOAc (500 mL×3). The organic layer was washed with brine (500 mL×3), dried over Na₂SO₄, filtered and concentrated. The residue was purified by flash silica gel chromatography (ISCO®; 100 g SepaFlash® Silica Flash Column, Eluent of 0 to 70% Ethyl acetate/Petroleum ether gradient at 65 mL/min) to give 3i (46.5 g, 112 mmol, 62.55% yield) as white solid. ¹H NMR (CDCl₃, 400 MHz) δ7.87-7.82 (m, 2H), 7.75-7.69 (m, 2H), 5.82-5.63 (m, 2H), 4.27 (d, J=5.6 Hz, 2H), 4.14 (d, J=5.6 Hz, 2H), 1.46 (s, 18H).

### Preparation of tert-butyl N-[(E)-4-aminobut-2-enyl]-N-tert-butoxycarbonyl-carbamate, 3j

To a solution of 3i (46.5 g, 112 mmol, 1 eq) in MeOH (500 mL) was added hydrazine; hydrate (19.7 g, 335 mmol, 19.0 mL, 85% purity, 3 eq) at 20° C. and it was stirred at 70° C. for 3 h. The mixture was filtered and the filtrate was concentrated. The crude product was triturated with MTBE at 15° C. for 20 min and the filtrate was concentrated to give 3j (33 g, crude) as light yellow oil. ¹H NMR (CDCl₃, 400 MHz) δ5.79-5.69 (m, 1H), 5.66-5.55 (m, 1H), 4.15 (dd, J=6.0, 1.2 Hz, 2H), 3.29 (dd, J=5.6, 1.2, Hz, 2H), 1.50 (s, 18H)

### Preparation of tert-butyl N-[(E)-4-(4-carbamoyl-2-methoxy-6-nitro-anilino)but-2-enyl]carbamate, 3k

A mixture of 4-chloro-3-methoxy-5-nitro-benzamide, 3b (5 g, 21.7 mmol, 1.0 eq) and 3j (18.6 g, 65.1 mmol, 3.0 eq) in DIEA (11.21 g, 86.73 mmol, 15.11 mL, 4.0 eq) (neat reaction) was stirred at 110° C. for 5.5 hours. Then the reaction mixture was cooled to 25° C., ethyl acetate (100 mL) was added and stirred for 10 min, then it was poured into ice-water (100 mL) and stirred for 10 min, desired solid precipitated from the mixture, and then filtered to give 3k (10 g, crude) as orange solid.

### Preparation of 4-[[(E)-4-aminobut-2-enyl]amino]-3-methoxy-5-nitro-benzamide, 3l

To a mixture of 3k (10 g, 26.29 mmol, 1 eq) in EtOAc (80 mL) was added HCl/EtOAc (4 M, 197 mL, 30 eq) at 15° C., and then stirred at 15° C. for 1 hour. The mixture was concentrated to give 3l (8.5 g, 24.07 mmol, 91.54% yield, 2HCl) as red solid. ¹H NMR (MeOD, 400 MHz) δ8.21 (d, J=2.0 Hz, 1H), 8.05 (s, 4H), 7.60 (d, J=2.0 Hz, 1H), 7.33 (s, 1H), 5.91-5.84 (m, 1H), 5.66-5.61 (m, 1H), 4.17 (d, J=5.2 Hz, 2H), 3.89 (s, 3H), 3.39 (t, J=5.6 Hz, 2H).

### Preparation of tert-butyl 4-[3-[5-carbamoyl-2-[[(E)-4-(4-carbamoyl-2-methoxy-6-nitro-anilino)but-2-enyl]amino]-3-nitro-phenoxy]propyl]piperazine-1-carboxylate, 3m

To a solution of 3l (5.31 g, 15.0 mmol, 1.26 eq, 2HCl) in butan-1-ol (106.00 mL) was added NaHCO₃ (5.03 g, 59.8 mmol, 2.33 mL, 5 eq) and DIEA (7.73 g, 59.8 mmol, 10.4 mL, 5 eq). The mixture was stirred at 20° C. for 30 min, then tert-butyl 4-[3-(5-carbamoyl-2-chloro-3-nitro-phenoxy)propyl] piperazine-1-carboxylate, 3g (5.3 g, 11.97 mmol, 1 eq) was added at 20° C. under N₂, the mixture was stirred at 120° C. for 12 hrs. The reaction mixture was concentrated in vacuum to give a residue. The residue was diluted with EtOAc (200 mL) and ice water (200 mL), it was stirred for 10 min. The precipitate was filtered to give 3m (14 g, crude) as red solid. ¹H NMR (DMSO-d₆, 400 MHz) δ8.15 (dd, J=5.6, 1.6 Hz, 2H), 8.03 (s, 2H), 7.81-7.65 (m, 3H), 7.53-7.47 (m, 2H), 7.33 (s, 2H), 5.65-5.55 (m, 2H), 4.12-4.10 (m, 2H), 4.08-3.97 (m, 4H), 3.81 (s, 3H), 3.30-3.27 (m, 4H), 2.40 (t, J=7.2 Hz, 2H), 2.31-2.24 (m, 4H), 1.93-1.82 (m, 2H), 1.38 (s, 9H).

### Preparation of tert-butyl 4-[3-[3-amino-2-[[(E)-4-(2-amino-4-carbamoyl-6-methoxy-anilinobut-2-enyl]amino]-5-carbamoyl-phenoxy]propyl]piperazine-1-carboxylate, 3n

To a solution of 3m (10.5 g, 15.0 mmol, 1 eq) in MeOH (50 mL), THF (50 mL) and H₂O (10 mL) was added NaHCO₃ (6.5 g, 76.0 mmol, 3.0 mL, 5 eq) and disodium dithionite, Na₂S₂O₄ (40.0 g, 229 mmol, 15 eq) and it was stirred at 20° C. for 5 h. The mixture was diluted with water (200 mL) and extracted with DCM:i-PrOH=3:1 (100 mL×6). The organic layer was dried over Na₂SO₄, filtered and concentrated to give 3n (4.6 g, 7.34 mmol, 48.00% yield) as yellow solid. ¹H NMR (MeOD, 400 MHz) δ6.94-6.91 (m, 2H), 6.88-6.83 (m, 2H), 5.77-5.64 (m, 2H), 4.00 (t, J=6.0 Hz, 2H), 3.78 (s, 3H), 3.59 (br d, J=4.4 Hz, 2H), 3.56 (br d, J=4.4 Hz, 2H), 3.51 (br s, 4H), 2.89-2.64 (m, 6H), 2.11-2.02 (m, 2H), 1.46 (s, 9H)

### Preparation of 2-ethyl-5-methyl-pyrazole-3-carbonyl isothiocyanate

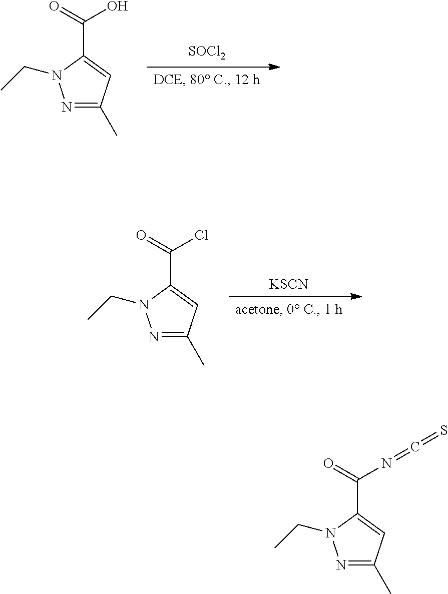

To a solution of 2-ethyl-5-methyl-pyrazole-3-carboxylic acid (6 g, 39.0 mmol, 1 eq) in DCE (60 mL) was added thionyl chloride, SOCl₂ (23.0 g, 195 mmol, 14.0 mL, 5 eq) at 0° C. under N₂ and it was stirred at 80° C. for 12 h. The mixture was concentrated to give 2-ethyl-5-methyl-pyrazole-3-carbonyl chloride (8 g, crude) as colorless oil. ¹H NMR (CDCl₃, 400 MHz) δ6.91 (s, 1H), 4.45 (q, J=7.2 Hz, 2H), 2.31 (s, 3H), 1.41 (t, J=7.2 Hz, 3H).

To a solution of thiocyanatopotassium (4.0 g, 41.7 mmol, 4.0 mL, 1.2 eq) in acetone (20 mL) was added 2-ethyl-5-methyl-pyrazole-3-carbonyl chloride (6 g, 34.8 mmol, 1 eq) under N₂ and it was stirred at 0° C. for 1 h. The mixture was filtered and concentrated. The residue was purified by flash silica gel chromatography (ISCO®; 10 g SepaFlash® Silica Flash Column, Eluent of 0˜40% Ethyl acetate/Petroleum ether gradient @ 45 mL/min) to give 2-ethyl-5-methyl-pyrazole-3-carbonyl isothiocyanate (5.5 g, 28.17 mmol, 81.04% yield) as light yellow oil. ¹H NMR (CDCl₃, 400 MHz) δ6.73 (s, 1H), 4.50 (q, J=7.2 Hz, 2H), 2.30 (s, 3H), 1.41 (t, J=7.2 Hz, 3H).

### Preparation of tert-butyl 4-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]piperazine-1-carboxylate, 3o

A solution of 3n (500 mg, 798 umol (micromoles), 1 eq) and 2-ethyl-5-methyl-pyrazole-3-carbonyl isothiocyanate (327 mg, 1.68 mmol, 2.1 eq) in DMF (5 mL) was stirred 30 min at 0° C., and then Et3N (565 mg, 5.60 mmol, 777 uL, 7 eq) and EDCI (765.0 mg, 4.0 mmol, 5 eq) was added, the mixture was stirred at 15° C. for another 12 h. The residue was poured into the saturation aqueous of NaHCO₃ (30 mL), filtered and the cake was washed with H₂O (10 mL×3), dried to give a crude product. The crude product was triturated with CH₃CN at 20° C. for 20 min to give 3o (500 mg, 527 umol, 66.04% yield) as light yellow solid. ¹H NMR (MeOD, 400 MHz) δ7.58 (d, J=14.0 Hz, 2H), 7.29-7.17 (m, 2H), 6.63-6.52 (m, 2H), 5.89-5.76 (m, 2H), 5.00 (br dd, J=4.4, 8.8 Hz, 4H), 4.64-4.55 (m, 4H), 3.89 (br t, J=5.2 Hz, 2H), 3.71 (s, 3H), 3.37-3.33 (m, 4H), 2.39-2.33 (m, 2H), 2.29-2.26 (m, 4H), 2.03 (s, 6H), 1.78-1.67 (m, 2H), 1.46 (s, 9H), 1.38-1.32 (m, 6H).

### Preparation of 3

To a solution of 3o (50 mg, 47.0 umol, 1 eq, TFA) in EtOAc (10 mL) was added HCl/EtOAc (4 M, 11.0 mL) and it was stirred at 15° C. for 1 h. The mixture was concentrated, and then freeze-drying to give 3 (39 mg, 42.3 umol, 89.95% yield, 2HCl) as light yellow solid. ¹H NMR (MeOD, 400 MHz) δ7.66 (d, J=2.8 Hz, 2H), 7.37 (d, J=6.4 Hz, 2H), 6.66 (d, J=11.2 Hz, 2H), 6.00 (br d, J=14.8 Hz, 1H), 5.88-5.78 (m, 1H), 5.13 (br d, J=6.4 Hz, 4H), 4.69-4.59 (m, 4H), 4.19-4.07 (m, 2H), 3.84 (s, 3H), 3.69 (br s, 8H), 3.43-3.37 (m, 2H), 2.23 (s, 6H), 2.22-2.15 (m, 2H), 1.44-1.35 (m, 6H). HPLC: 95.099% (220 nm). LCMS (ESI): mass calculated for C₄₂H₅₂N₄O₆ 848.42, m/z found 849.4 [M+H]⁺

### Example L-1 Synthesis of (2,3,5,6-tetrafluorophenyl) 3-[2-[2-[2-[2-[2-[2-[2-[2-[2-[2-[4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazin-1-yl]ethoxy]ethoxy]ethoxy]ethoxy]ethoxy]ethoxy]ethoxy]ethoxy]ethoxy]ethoxy]propanoate, BBI-L-1

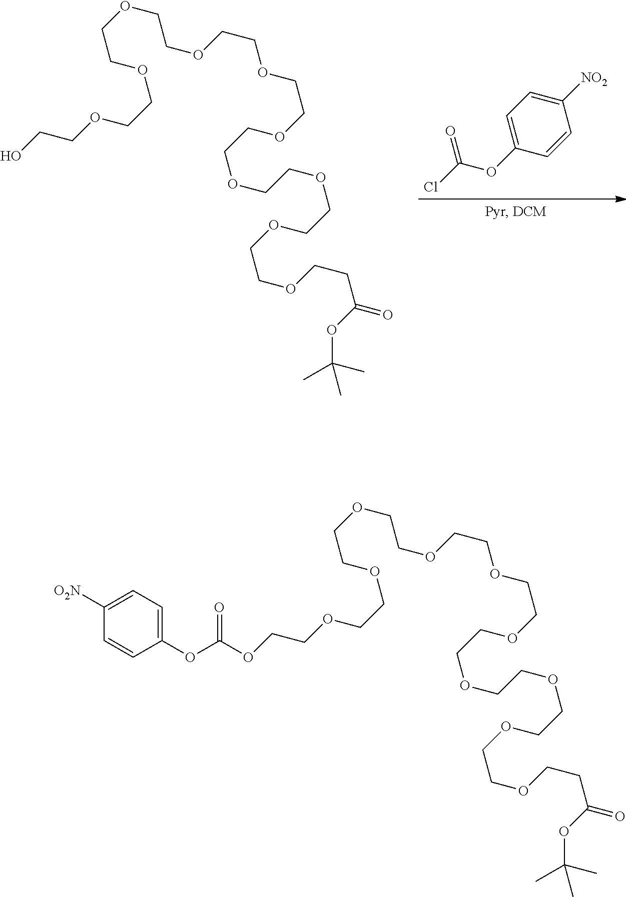 [SMILES: CC(C)(C)OC(=O)CCOCCOCCOCCOCCOCCOCCOCCOCCOCCOCCO ; O=C(Cl)Oc1ccc([N+](=O)[O-])cc1 ; "Pyr, DCM" ; CC(C)(C)OC(=O)CCOCCOCCOCCOCCOCCOCCOCCOCCOCCOCCOC(=O)Oc1ccc([N+](=O)[O-])cc1]

### Preparation of tert-butyl 3-[2-[2-[2-[2-[2-[2-[2-[2-[2-[2-(4-nitrophenoxy)carbonyloxyethoxy]ethoxy]ethoxy]ethoxy]ethoxy]ethoxy]ethoxy]ethoxy]ethoxy]ethoxy]propanoate

To mixture of tert-butyl 3-[2-[2-[2-[2-[2-[2-[2-[2-[2-(2-hydroxyethoxy)ethoxy]ethoxy] ethoxy]ethoxy]ethoxy]ethoxyethoxy]ethoxy]ethoxy]propanoate (5 g, 8.52 mmol, 1 eq) and (4-nitrophenyl) carbonochloridate (1.89 g, 9.37 mmol, 1.1 eq) in DCM (100 mL) was added pyridine (1.01 g, 12.8 mmol, 1.03 mL, 1.5 eq) at 0° C., and then stirred at 25° C. for 2 hrs. The mixture was poured into ice-water (w/w=1/1) (100 mL) and stirred for 10 min, and the pH of mixture was adjusted to about 4 with HCl (1M). The aqueous phase was extracted with DCM (50 mL×2), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=1/0, 0/1, Ethyl acetate/Methanol=1/0, 2/1) to afford tert-butyl 3-[2-[2-[2-[2-[2-[2-[2-[2-[2-[2-(4-nitrophenoxy)carbonyloxyethoxy] ethoxy]ethoxy]ethoxy]ethoxy]ethoxyethoxy]ethoxy]ethoxy]ethoxy]propanoate (3 g, 3.99 mmol, 46.82% yield) as colorless oil. ¹H NMR (400 MHz, MeOD) δ 8.36-8.28 (m, 2H), 7.54-7.46 (m, 2H), 4.46-4.40 (m, 2H), 3.83-3.77 (m, 2H), 3.72-3.57 (m, 40H), 1.45 (s, 9H)

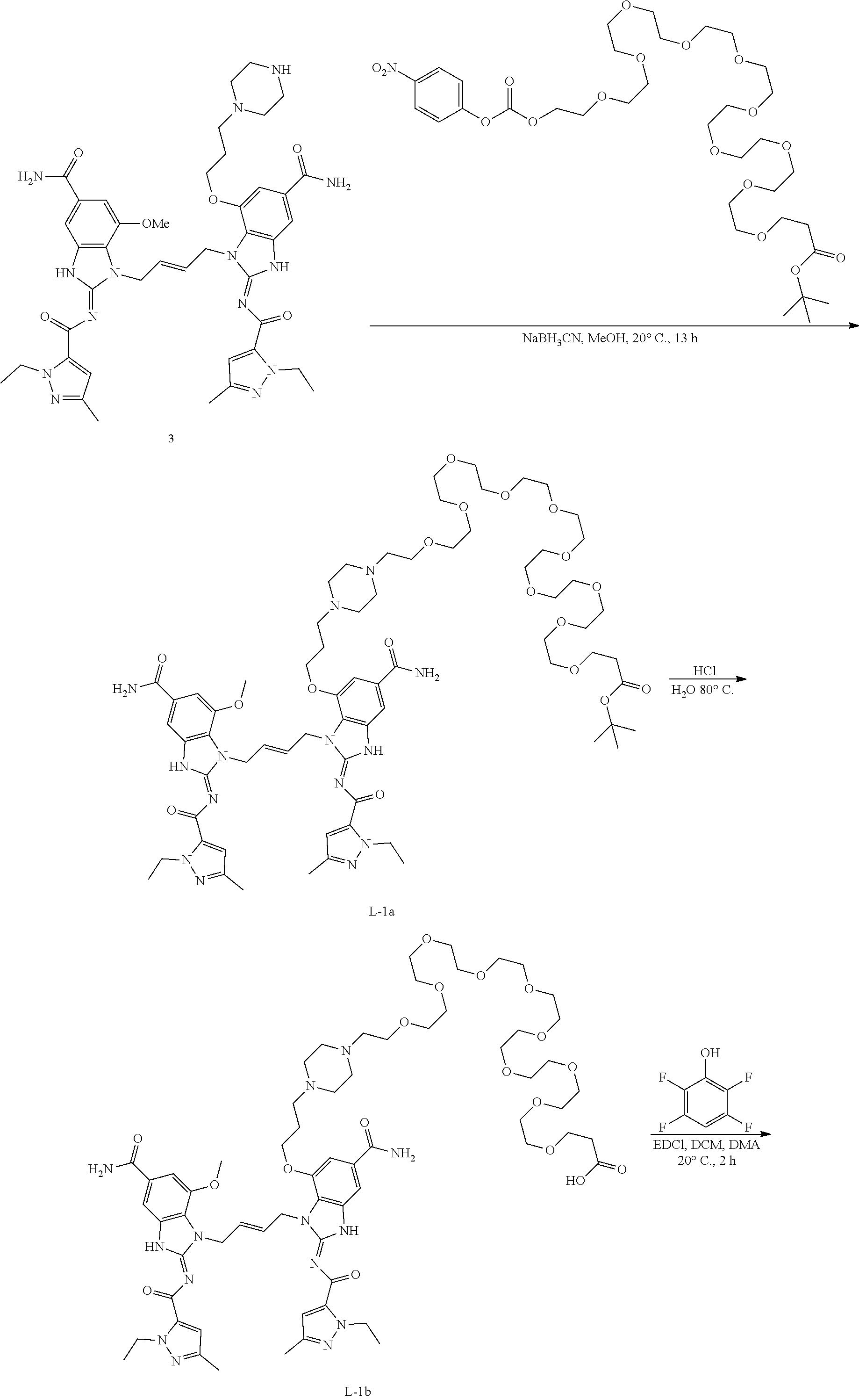

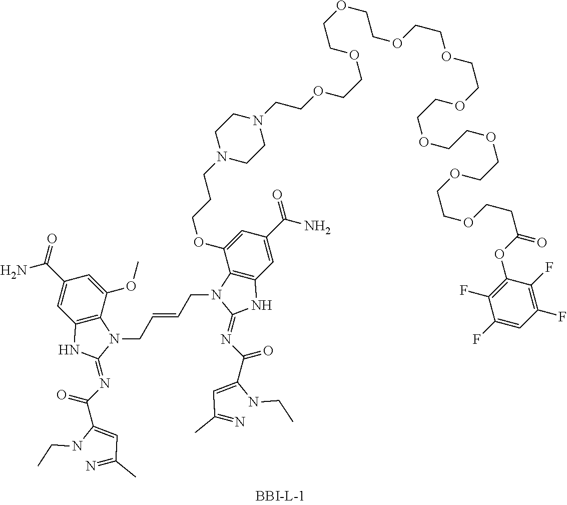 [SMILES: CCN/C(=C\C(C)=N)C(=O)/N=c1\[nH]c2cc(C(N)=O)cc(OC)c2n1C/C=C/Cn1/c(=N/C(=O)/C(=C/C(C)=N)NCC)[nH]c2cc(C(N)=O)cc(OCCCN3CCN(CCOCCOCCOCCOCCOCCOCCOCCOCCOCCOCCC(=O)Oc4c(F)c(F)cc(F)c4F)CC3)c21]

### Preparation of tert-butyl 3-[2-[2-[2-[2-[2-[2-[2-[2-[2-[2-[4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazin-1-yl]ethoxy]ethoxy]ethoxy]ethoxy]ethoxy]ethoxy]ethoxy]ethoxy]ethoxy]ethoxy]propanoate, L-1a

To a mixture of (2E)-1-[(E)-4-[(2E)-5-carbamoyl-2-(2-ethyl-5-methyl-pyrazole-3-carbonyl) imino-7-(3-piperazin-1-ylpropoxy)-3H-benzimidazol-1-yl]but-2-enyl]-2-(2-ethyl-5-methyl-pyrazole-3-carbonyl)imino-7-methoxy-3H-benzimidazole-5-carboxamide, 3 (70 mg, 79.1 umol, 1 eq, HCl) and tert-butyl 3-[2-[2-[2-[2-[2-[2-[2-[2-[2-[2-(4-nitrophenoxy)carbonyloxyethoxy]ethoxy]ethoxy]ethoxy]ethoxy]ethoxy]ethoxy]ethoxy]ethoxy]ethoxy]propanoate (55.5 mg, 94.8 umol, 1.2 eq) in MeOH (5 mL) was added NaBH₃CN (14.9 mg, 237 umol, 3 eq) in one portion at 15° C. The mixture was stirred at 15° C. for 16 hours. Then it was concentrated in reduced pressure at 35° C. The residue was purified by prep-HPLC (column: Phenomenex Synergi C18 150*25*10 um; mobile phase: [water (0.1% TFA)-ACN]; B %: 10%-40%, 10 min) to afford L-1a (80 mg, 56.43 umol, 71.38% yield) as yellow solid.

### Preparation of 3-[2-[2-[2-[2-[2-[2-[2-[2-[2-[2-[4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazin-1-yl]ethoxy]ethoxy]ethoxy]ethoxy]ethoxy]ethoxy]ethoxy]ethoxy]ethoxy]ethoxy]propanoic Acid, L-1b

To a mixture of L-1a (80 mg, 52.2 umol, 1 eq, TFA) in H₂O (2 mL) was added HCl (12 M, 65.3 uL, 15 eq) at 15° C. The mixture was stirred at 80° C. for 1 hour and then concentrated in reduced pressure at 50° C. to afford L-1b (80 mg, crude, HCl) as light yellow oil.

### Preparation of BBI-L-1

To a mixture of L-1b (68.1 mg, 50.07 umol, 1 eq, HCl) in DCM (0.3 mL) and DMA (0.03 mL) was added 2,3,5,6-tetrafluorophenol (66.5 mg, 400 umol, 8 eq) and EDCI (96.0 mg, 501 umol, 10 eq) in one portion at 15° C. The mixture was stirred at 15° C. for 30 min. Then it was concentrated in reduced pressure at 35° C. The residue was purified by prep-HPLC (column: Phenomenex Synergi C18 150*25*10 um (microns); mobile phase: [water (0.1% TFA)-ACN]; B %: 20%-50%, 8 min) to afford BBI-L-1 (17.4 mg, 10.72 umol, 21.40% yield, TFA) as light yellow oil. ¹H NMR (MeOD, 400 MHz) δ7.63 (s, 1H), 7.58 (s, 1H), 7.48-7.37 (m, 1H), 7.33 (s, 1H), 7.30 (s, 1H), 6.65 (s, 1H), 6.59 (s, 1H), 5.87 (s, 2H), 5.10-5.00 (m, 4H), 4.74-4.55 (m, 4H), 4.00 (t, J=6.4 Hz, 2H), 3.89-3.82 (m, 4H), 3.78 (s, 3H), 3.73-3.56 (m, 42H), 3.28-3.21 (m, 2H), 3.15 (2H), 2.97 (t, J=6.0 Hz, 2H), 2.79 (s, 2H), 2.63-2.55 (m, 2H), 2.24 (s, 3H), 2.22 (s, 3H), 1.79-1.77 (m 2H), 1.44-1.32 (m, 6H). HPLC: 96.08% (220 nm). LCMS (ESI): mass calcd. for C₇₁H₉₆F₄N₁₄O₁₆ 1508.70, m/z found 1509.5 [M+H]⁺

### Example L-3 Synthesis of 4-[3-[2-[2-[2-[2-[2-[2-[2-[2-[2-[3-[4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazin-1-yl]-3-oxo-propoxy]ethoxy]ethoxy]ethoxy]ethoxy]ethoxy]ethoxy] ethoxy]ethoxy]ethoxy]propanoyl oxy]-2,3,5,6-tetrafluoro-benzenesulfonic Acid, BBI-L-3

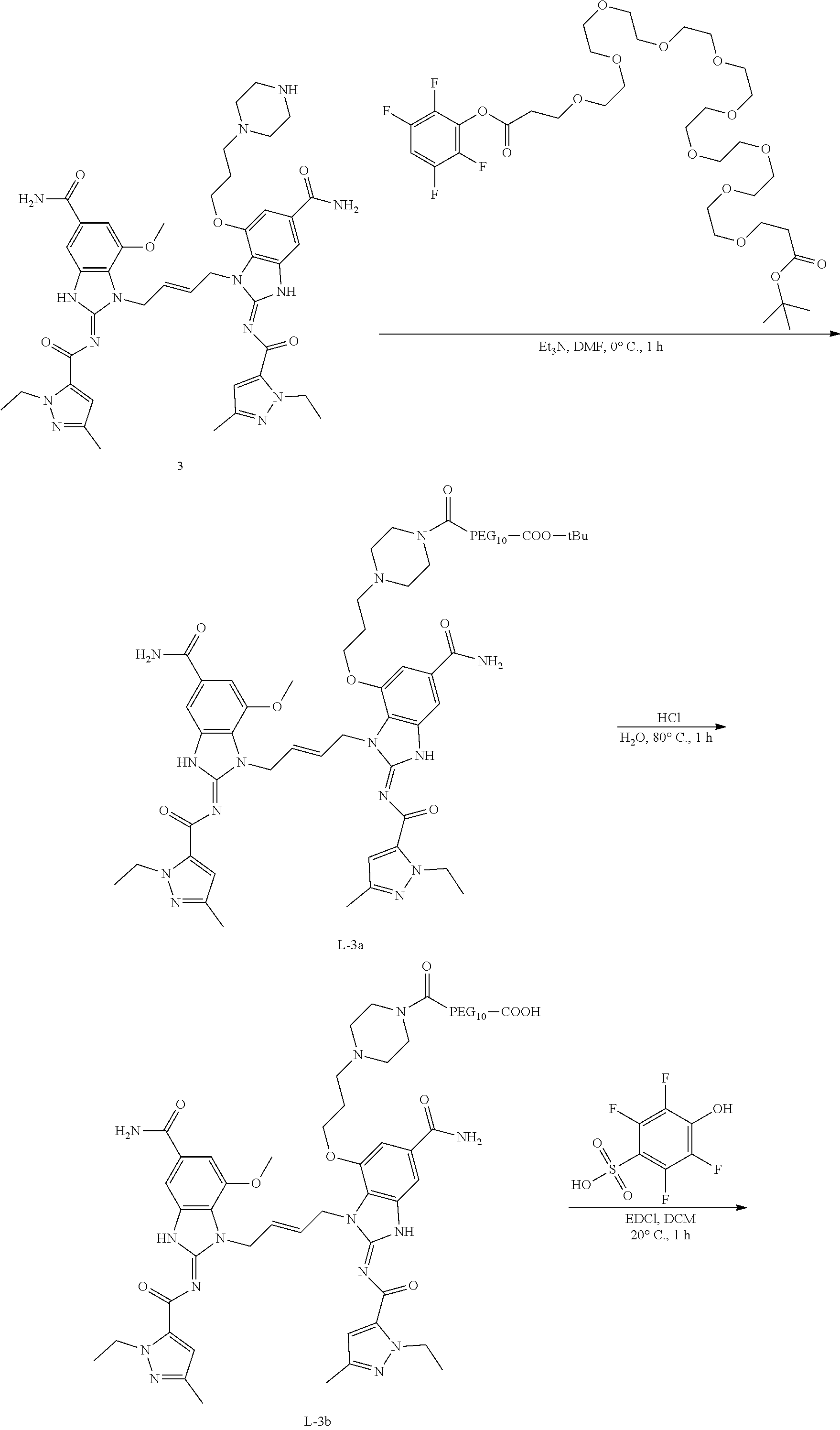

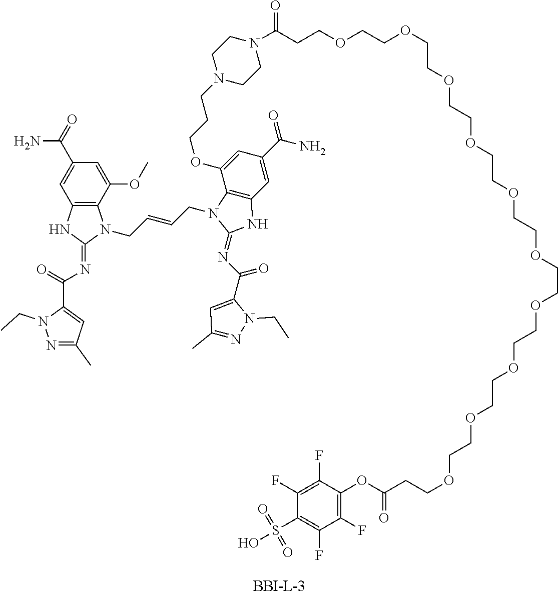 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCOCCOCCOCCOCCOCCOCCOCCOCCOCCOCCC(=O)Oc4c(F)c(F)c(S(=O)(=O)O)c(F)c4F)CC3)c21]

### Preparation of tert-butyl 3-[2-[2-[2-[2-[2-[2-[2-[2-[2-[3-[4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazin-1-yl]-3-oxo-propoxy]ethoxy]ethoxy]ethoxy] ethoxy]ethoxy]ethoxy]ethoxy]ethoxy]ethoxy]propanoate, L-3a

To a solution of (2E)-1-[(E)-4-[(2E)-5-carbamoyl-2-(2-ethyl-5-methyl-pyrazole-3-carbonyl)imino-7-(3-piperazin-1-ylpropoxy)-3H-benzimidazol-1-yl]but-2-enyl]-2-(2-ethyl-5-methyl-pyrazole-3-carbonyl)imino-7-methoxy-3H-benzimidazole-5-carboxamide, 3 (180 mg, 203 umol, 1 eq, HCl) in DMF (3.00 mL) was added Et₃N (82.0 mg, 813 umol, 113 uL, 4 eq) and (2,3,5,6-tetrafluorophenyl) 3-[2-[2-[2-[2-[2-[2-[2-[2-[2-(3-tert-butoxy-3-oxo-propoxy)ethoxy]ethoxy]ethoxy]ethoxy]ethoxy]ethoxy]ethoxy]ethoxy]ethoxy]propanoate, (171 mg, 223 umol, 1.1 eq), and then stirred at 0° C. for 1 h. The mixture was concentrated to give a residue. The residue was purified by prep-HPLC (column: Phenomenex Luna 80*30 mm*3 um; mobile phase: [water (0.1% TFA)-ACN]; B %: 20%-50%, 8 min) to give L-3a (280 mg, 194 umol, 95.3% yield) as light yellow solid.

### Preparation of 3-[2-[2-[2-[2-[2-[2-[2-[2-[2-[3-[4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazin-1-yl]-3-oxo-propoxy]ethoxy]ethoxy]ethoxy]ethoxy]ethoxy]ethoxy] ethoxy]ethoxy]ethoxy]propanoic Acid, L-3b

To a solution of L-3a (280 mg, 194 umol, 1 eq) in H₂O (3.00 mL) was added HCl (12 M, 0.20 mL, 12.4 eq), and then stirred at 80° C. for 1 h. The mixture was concentrated to give a residue. The residue was purified by prep-HPLC (column: Phenomenex Luna 80*30 mm*3 um; mobile phase: [water (0.1% TFA)-ACN]; B %: 10%-30%, 8 min) to give L-3b (100 mg, 66.51 umol, 34.34% yield, TFA) as light yellow solid.

### Preparation of BBI-L-3

To a solution of L-3b (100 mg, 71.9 umol, 1 eq) and (2,3,5,6-tetrafluoro-4-hydroxy-phenyl)sulfonyloxysodium (77.0 mg, 288 umol, 4 eq) in DCM (2.00 mL) and DMA (0.10 mL) was added EDCI (55.0 mg, 288 umol, 4 eq)., and then stirred at 20° C. for 1 h. The mixture was concentrated to give a residue. The residue was purified by prep-HPLC (column: Phenomenex Luna 80*30 mm*3 um; mobile phase: [water (0.1% TFA)-ACN]; B %: 15%-40%, 8 min) to give BBI-L-3 (28 mg, 16.17 umol, 22.47% yield, TFA) as light yellow solid. ¹H NMR (MeOD, 400 MHz) δ7.66-7.58 (m, 2H), 7.28 (br d, J=14.0 Hz, 2H), 6.60 (br d, J=12.0 Hz, 2H), 5.85-5.78 (m, 2H), 5.06-4.97 (m, 4H), 4.70-4.51 (m, 4H), 4.34-4.23 (m, 2H), 4.05-3.98 (m, 2H), 3.86-3.80 (m, 2H), 3.78-3.66 (m, 5H), 3.64-3.47 (m, 38H), 3.38-3.32 (m, 2H), 3.18-3.07 (m, 2H), 2.94 (br t, J=5.6 Hz, 6H), 2.57-2.45 (m, 2H), 2.28-2.14 (m, 6H), 2.10-2.03 (m, 2H), 1.43-1.30 (m, 6H). HPLC: 96.21% (220 nm), 97.95% (254 nm). LCMS (ESI): mass calcd. For C₇₂H₉₆F₄N₁₄O₂₂S 1616.65, m/z found 1617.5 [M+H]⁺.

### Example L-9 Synthesis of 4-[3-[2-[2-[2-[2-[2-[2-[2-[2-[2-[2-[2-[4-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]piperazin-1-yl]ethylcarbamoyloxy]ethoxy]ethoxy]ethoxy]ethoxy]ethoxy]ethoxy]ethoxy]ethoxy]ethoxy]ethoxy]propanoyloxy]-2,3,5,6-tetrafluoro-benzenesulfonic Acid, BBI-L-9

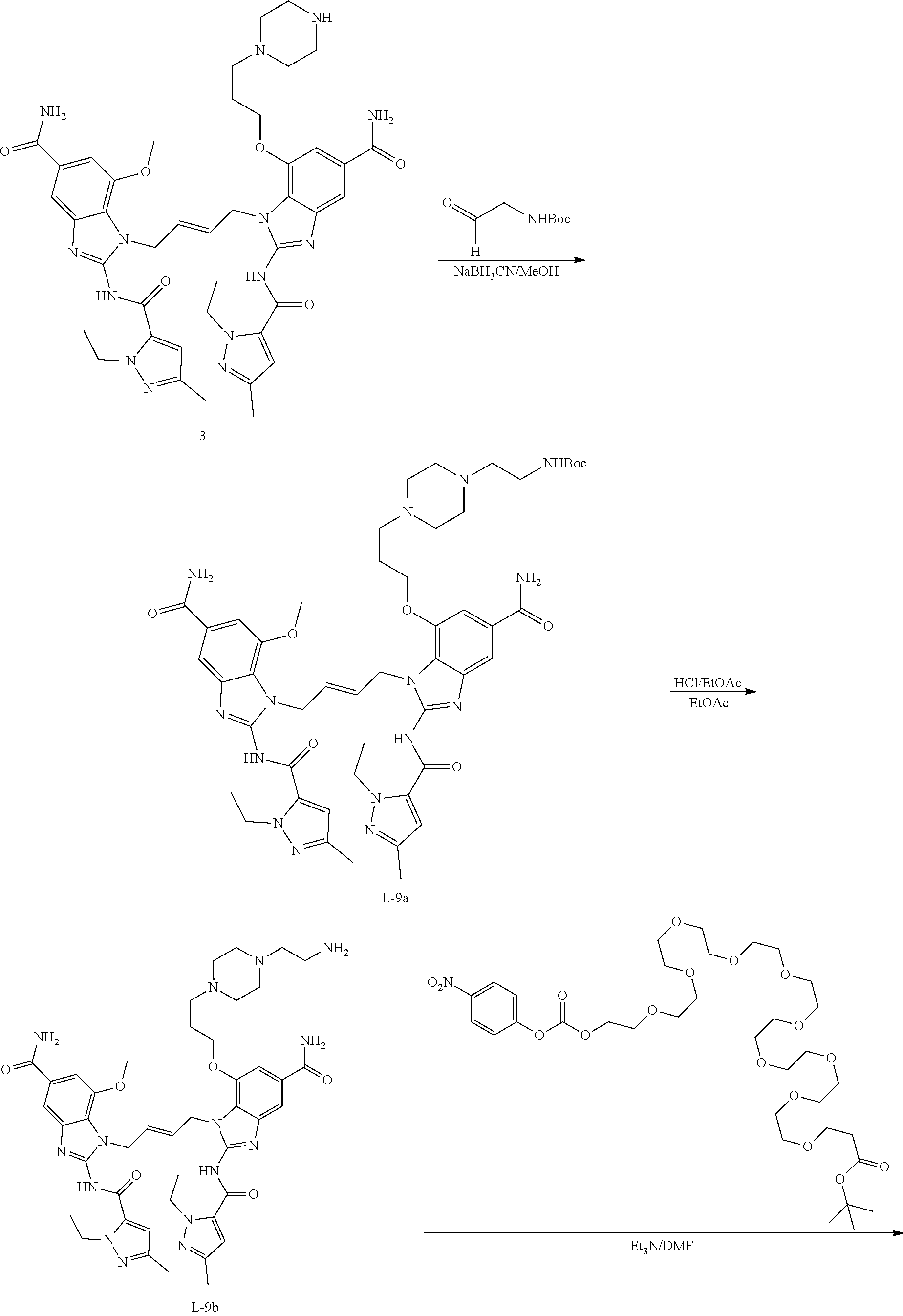

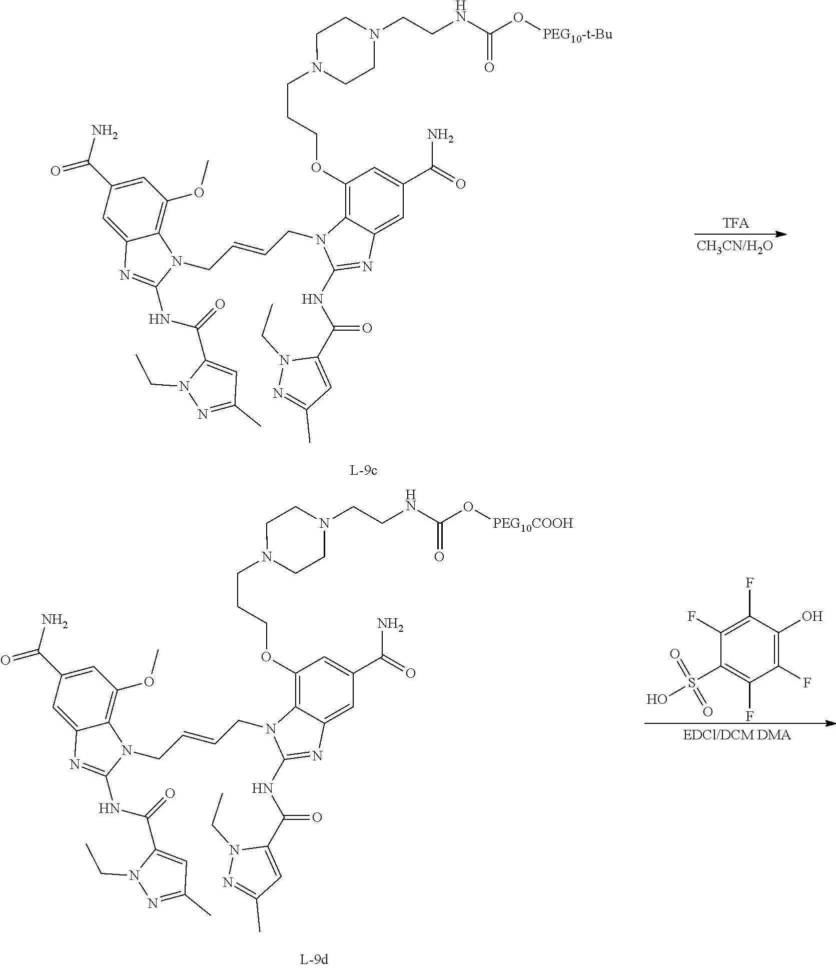

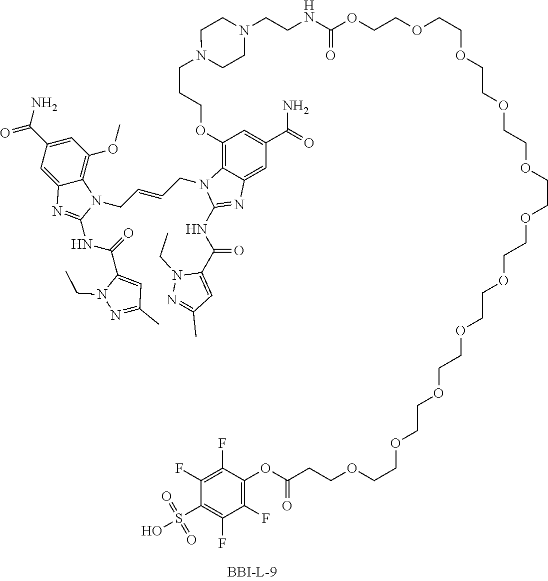 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCNC(=O)OCCOCCOCCOCCOCCOCCOCCOCCOCCOCCOCCC(=O)Oc4c(F)c(F)c(S(=O)(=O)O)c(F)c4F)CC3)c21]

### Preparation of tert-butyl N-[2-[4-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]piperazin-1-yl]ethyl]carbamate, L-9a

To a solution of 1-[(E)-4-[5-carbamoyl-2-[(2-ethyl-5-methyl-pyrazole-3-carbonyl) amino]-7-(3-piperazin-1-ylpropoxy)benzimidazol-1-yl]but-2-enyl]-2-[(2-ethyl-5-methyl-pyrazole-3-carbonyl)amino]-7-methoxy-benzimidazole-5-carboxamide, 3 (530 mg, 496 umol, 1.0 eq, 2HCl) and tert-butyl N-(2-oxoethyl)carbamate (395 mg, 2.48 mmol, 5.0 eq) in MeOH (20 mL) was added acetic acid, AcOH (2.98 mg, 49.6 umol, 2.84 uL, 0.1 eq), and it was stirred at 20° C. for 0.5 hr. Sodium cyanoborohydride, NaBH₃CN (62.4 mg, 993 umol, 2.0 eq) was added at 0° C., and the mixture was stirred at 20° C. for another 1 hr. The reaction mixture was quenched by addition H₂O (1 mL) at 0° C., and then the pH of mixture was adjusted to 8 with sat.NaHCO₃. The aqueous phase was extracted with ethyl acetate (10 mL×5). The combined organic phase was washed with brine (20 mL×1), dried with anhydrous Na₂SO₄, filtered and concentrated in vacuum. The crude product was triturated with MTBE at 20° C. for 10 min to afford L-9a (600 mg, 450 umol, 90.60% yield) as yellow solid.

### Preparation of 1-[(E)-4-[7-[3-[4-(2-aminoethyl)piperazin-1-yl]propoxyl-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]-7-methoxy-benzimidazole-5-carboxamide, L-9b

To a solution of L-9a (20 mg, 20.2 umol, 1.0 eq) in EtOAc (1 mL) was added HCl/EtOAc (4 M, 1 mL), and then stirred at 20° C. for 1 hr. The mixture was concentrated in vacuum. The residue was purified by prep-HPLC (column: Phenomenex Luna 80*30 mm*3 um; mobile phase: [water (0.1% TFA)-ACN]; B %: 5%-35%, 8 min) to afford L-9b (5 mg, 4.05 umol, 20.10% yield, 3TFA) as yellow solid. ¹H NMR (400 MHz, MeOD) δ 7.60 (d, J=8.4 Hz, 2H), 7.27 (d, J=8.4 Hz, 2H), 6.59 (d, J=16.8 Hz, 2H), 5.80 (s, 2H), 5.06-5.00 (m, 4H), 4.67-4.53 (m, 4H), 4.05-3.95 (m, 4H), 3.75 (s, 3H), 3.33-3.80 (m, 10H), 2.75-2.70 (m, 2H), 2.52-2.45 (m, 2H), 2.21 (d, J=3.6 Hz, 6H), 2.10-1.91 (m, 2H), 1.40-1.32 (m, 6H). HPLC: 100.00% (220 nm). LCMS (ESI): mass calcd. for C₄₄H₅₇N₁₅O₆ 891.46, m/z found 892.4 [M+H]⁺.

### Preparation of tert-butyl 3-[2-[2-[2-[2-[2-[2-[2-[2-[2-[2-[2-[4-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]piperazin-1-yl]ethylcarbamoyloxy]ethoxy]ethoxy]ethoxy]ethoxy]ethoxy]ethoxy]ethoxy]ethoxy]ethoxy]ethoxy]propanoate, L-9c

To a solution of L-9b (150 mg, 145 umol, 1 eq, 4HCl) in DMF (2 mL) was added Et₃N (58.5 mg, 578 umol, 80.5 uL, 4.0 eq) and tert-butyl 3-[2-[2-[2-[2-[2-[2-[2-[2-[2-[2-(4-nitrophenoxy)carbonyloxyethoxy]ethoxy]ethoxy]ethoxy]ethoxy]ethoxy]ethoxy]ethoxy]ethoxy]ethoxy]propanoate (120 mg, 159 umol, 1.1 eq). The mixture was stirred at 20° C. for 1 hr. The water (5 mL) was added to the mixture and stirred for 10 min. The aqueous phase was extracted with DCM/i-PrOH=3/1 (5 mL×3). The combined organic phase was dried with anhydrous Na₂SO₄, filtered and concentrated in vacuum, to afford L-9c (150 mg, 81.2 umol, 56.20% yield) as yellow oil.

### Preparation of 3-[2-[2-[2-[2-[2-[2-[2-[2-[2-[2-[2-[4-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]piperazin-1-yl]ethylcarbamoyloxy]ethoxy]ethoxy]ethoxy]ethoxy]ethoxy]ethoxy]ethoxy]ethoxy]ethoxy]ethoxy]propanoic Acid, L-9d

To a solution of L-9c (150 mg, 81.2 umol, 1.0 eq) in MeCN (0.5 mL) and H₂O (2 mL) was added TFA (92.6 mg, 812 umol, 60.1 uL, 10 eq), and then stirred at 80° C. for 1 hr. The mixture was filtered. The residue was purified by prep-HPLC (column: Phenomenex Luna 80*30 mm*3 um; mobile phase: [water (0.1% TFA)-ACN]; B %: 15%-40%, 8 min) to afford L-9d (100 mg, 55.8 umol, 68.7% yield, 3TFA) as yellow oil. ¹H NMR (400 MHz, MeOD) δ 7.59 (d, J=15.2 Hz, 2H), 7.29 (d, J=19.2 Hz, 2H), 6.65-6.56 (m, 2H), 5.83 (s, 2H), 5.02 (s, 4H), 4.69-4.49 (m, 4H), 4.21 (s, 2H), 4.01-3.91 (m, 2H), 3.75 (s, 3H), 3.73-3.67 (m, 6H), 3.64-3.57 (m, 36H), 3.43-3.35 (m, 2H), 2.98-2.92 (m, 4H), 2.78-2.70 (m, 4H), 2.53 (t, J=6.4 Hz, 2H), 2.26-2.16 (m, 6H), 1.83-1.79 (m, 2H), 1.39-1.32 (m, 6H).

### Preparation of BBI-L-9

To a solution of L-9d (100 mg, 55.8 umol, 1.0 eq, 3TFA) in DCM (2 mL) and DMA (1 mL) was added (2,3,5,6-tetrafluoro-4-hydroxy-phenyl)sulfonyloxysodium (59.9 mg, 223 umol, 4.0 eq) and EDCI (42.8 mg, 223 umol, 4.0 eq). The mixture was stirred at 20° C. for 1 hr. The mixture was concentrated in vacuum. The residue was purified by prep-HPLC (column: Phenomenex Luna 80*30 mm*3 um; mobile phase: [water (0.1% TFA)-ACN]; B %: 15%-35%, 8 min) to afford BBI-L-9 (43 mg, 22.6 umol, 40.42% yield, 2TFA) as yellow solid. ¹H NMR (400 MHz, MeOD) δ7.59 (d, J=18.8 Hz, 2H), 7.26 (d, J=22.8 Hz, 2H), 6.57 (d, J=22.4 Hz, 2H), 5.86-5.72 (m, 2H), 5.02-4.95 (m, 4H), 4.65-4.50 (m, 4H), 4.19 (s, 2H), 4.00-3.94 (m, 2H), 3.84 (t, J=5.6 Hz, 2H), 3.75 (s, 3H), 3.69-3.54 (m, 40H), 3.48-3.42 (m, 4H), 3.24-3.07 (m, 6H), 2.95 (t, J=5.6 Hz, 2H), 2.19 (d, J=10.4 Hz, 6H), 1.95-1.90 (m, 2H), 1.39-1.30 (m, 6H). HPLC: 100.00% (220 nm). LCMS (ESI): mass calcd. for C₇₄H₁₀₁F₄N₁₅O₂₃S 1675.69, m/z found 1676.6 [M+H]⁺.

### Example L-13 Synthesis of of [4-[[(2S)-2-[[(2S)-1-[(2S)-2-[3-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]propanoylamino]propanoyl]pyrrolidine-2-carbonyl]amino]-3-methyl-butanoyl]amino]phenyl]methyl 4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazine-1-carboxylate, BBI-L-13

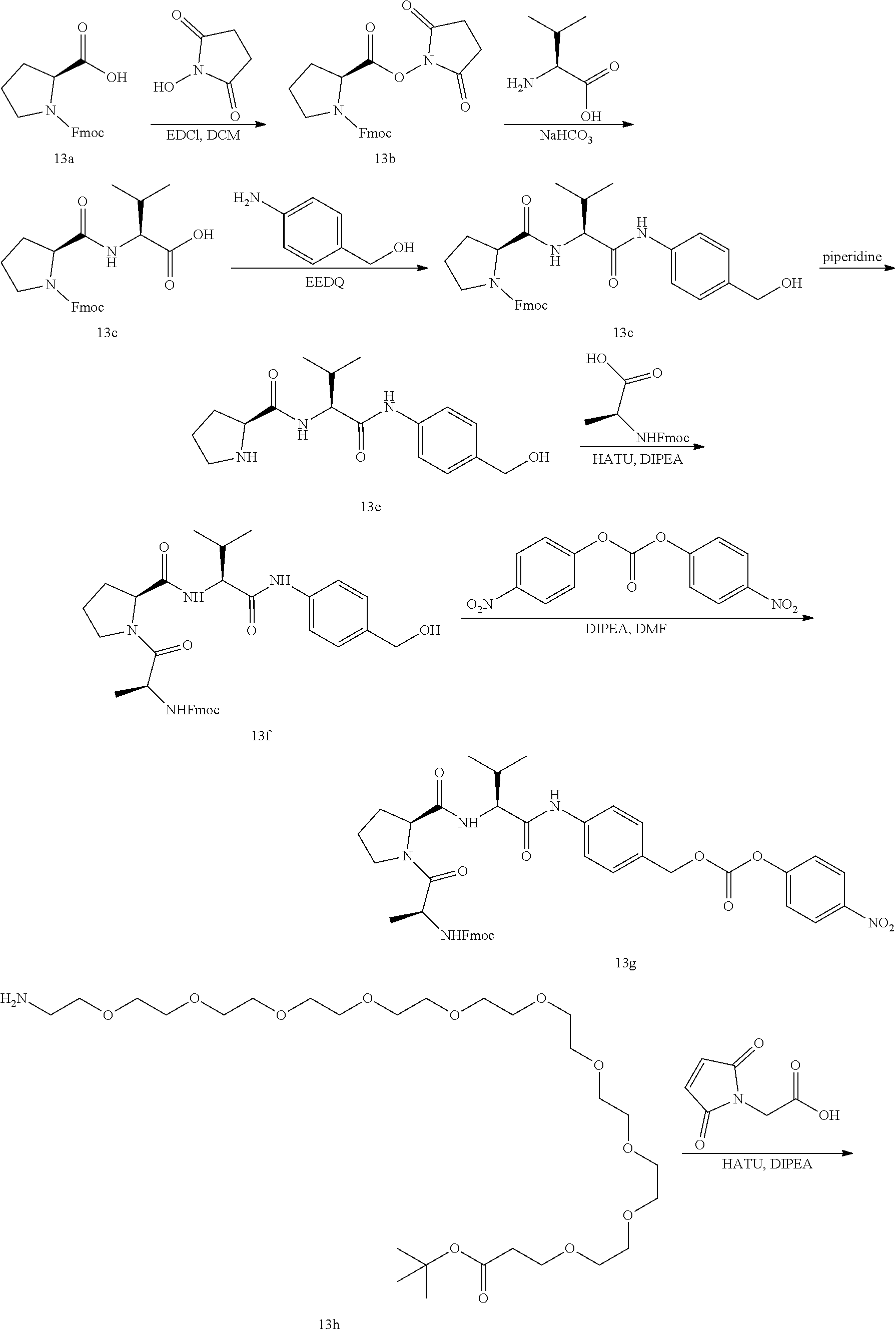

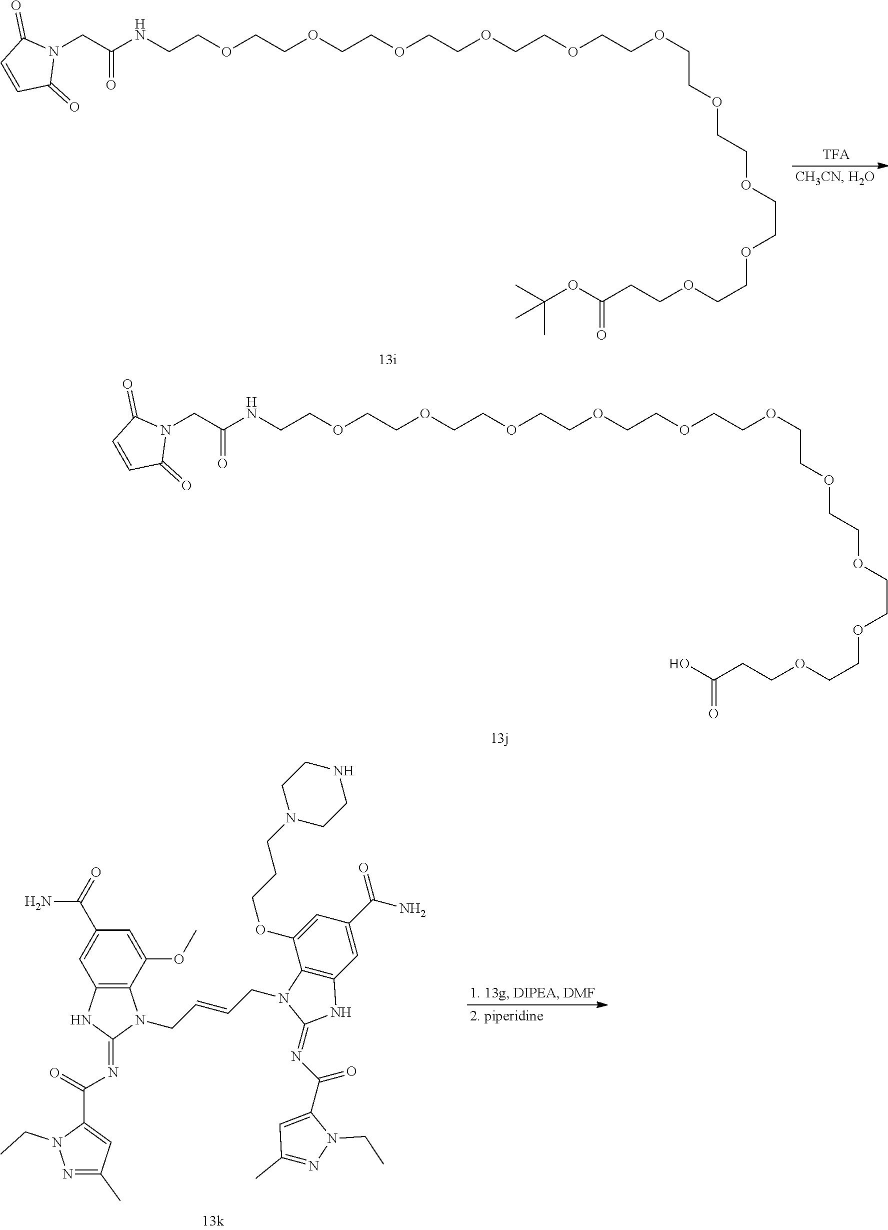 [SMILES: CC(C)(C)OC(=O)CCOCCOCCOCCOCCOCCOCCOCCOCCOCCOCCNC(=O)CN1C(=O)C=CC1=O ; O=C(O)CCOCCOCCOCCOCCOCCOCCOCCOCCOCCOCCNC(=O)CN1C(=O)C=CC1=O ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCNCC3)c21]

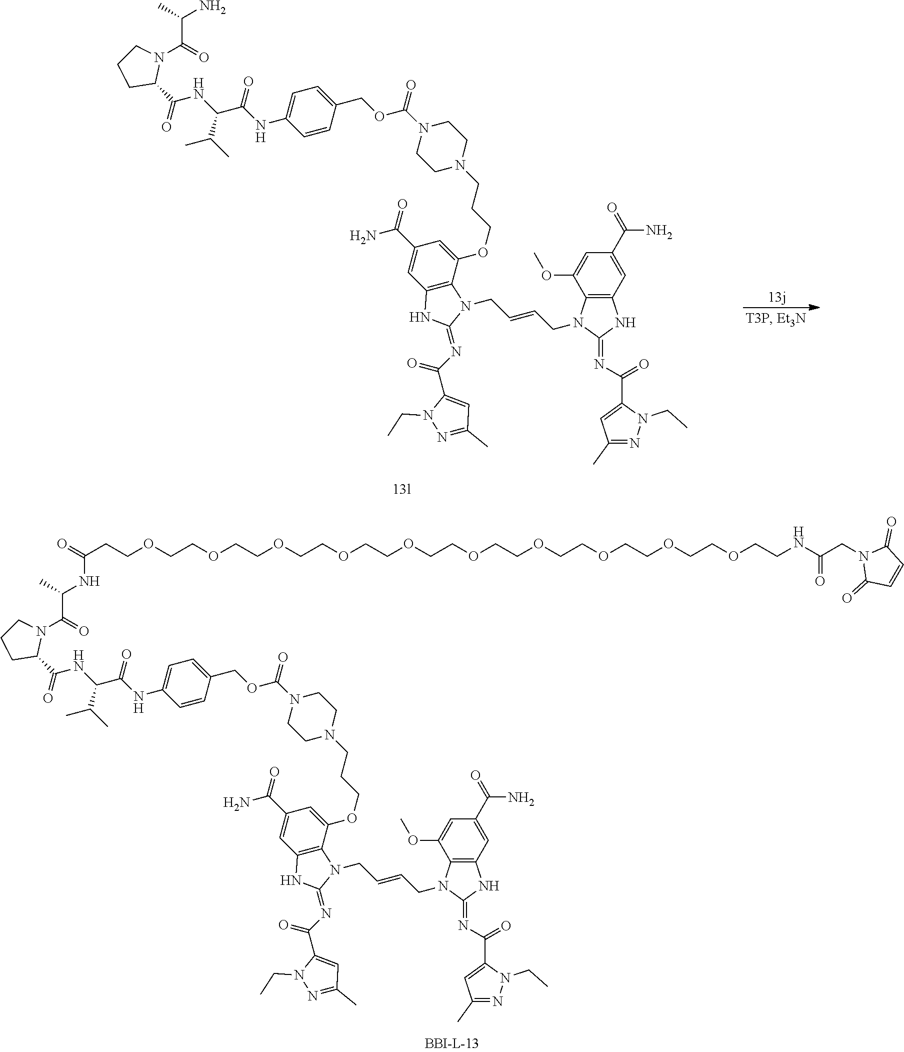 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@@H](NC(=O)[C@@H]5CCCN5C(=O)[C@H](C)N)C(C)C)cc4)CC3)c21 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@@H](NC(=O)[C@@H]5CCCN5C(=O)[C@H](C)NC(=O)CCOCCOCCOCCOCCOCCOCCOCCOCCOCCOCCNC(=O)CN5C(=O)C=CC5=O)C(C)C)cc4)CC3)c21]

### Preparation of (S)-1-((9H-fluoren-9-yl)methyl) 2-(2,5-dioxopyrrolidin-1-yl) pyrrolidine-1,2-dicarboxylate, 13b

To a solution of (2S)-1-(9H-fluoren-9-ylmethoxycarbonyl)pyrrolidine-2-carboxylic acid, 13a (15 g, 44.5 mmol, 1.0 eq) in DCM (200 mL) was added 1-hydroxypyrrolidine-2,5-dione (5.12 g, 44.5 mmol, 1.0 eq) and 1-ethyl-3-(3-dimethylaminopropyl)carbodiimide, EDCI, EDAC, EDC, CAS Reg. No. 25952-53-8 (10.2 g, 53.44 mmol, 1.2 eq), and it was stirred at 20° C. for 12 h. The mixture was washed by saturated aqueous solution of NaHCO₃ (70 mL×3). The organic layer was dried over Na₂SO₄ and concentrated to give 13b (17.5 g, 40.28 mmol, 90.60% yield) as a white solid.

### Preparation of (2S)-2-[[(2S)-1-(9H-fluoren-9-ylmethoxycarbonyl)pyrrolidine-2-carbonyl]amino]-3-methyl-butanoic Acid, 13c

To a solution of (2S)-2-amino-3-methyl-butanoic acid, L-valine (4.95 g, 42.3 mmol, 1.05 eq) in THF (200 mL) was added NaHCO₃ (3.55 g, 42.3 mmol, 1.64 mL, 1.05 eq) in H₂O (50 mL) and 13b (17.5 g, 40.28 mmol, 1.0 eq) and it was stirred at 20° C. for 12 h. The mixture was extracted with MTBE (2×100 mL) (discarded). The pH of aqueous layer was adjusted to 5-6 with HCl (6 M) and extracted with EtOAc (3×200 ml). The combined organic layer was dried over Na₂SO₄ and concentrated to give 13c (15 g, 34.36 mmol, 85.31% yield) as white solid. ¹H NMR (MeOD, 400 MHz) δ7.80 (d, J=7.2 Hz, 2H), 7.70-7.54 (m, 2H), 7.43-7.28 (m, 4H), 4.49-4.15 (m, 5H), 3.69-3.38 (m, 2H), 2.42-2.01 (m, 3H), 2.00-1.82 (m, 2H), 1.01-0.86 (m, 6H).

### Preparation of 9H-fluoren-9-ylmethyl(2S)-2-[[(1S)-1-[[4-(hydroxymethyl)phenyl] carbamoyl]-2-methyl-propyl]carbamoyl]pyrrolidine-1-carboxylate, 13d

To a solution 13c (10 g, 22.9 mmol, 1.0 eq) and (4-aminophenyl)methanol (4.23 g, 34.4 mmol, 1.5 eq) in MeOH (80 mL) and DCM (80 mL) was added 2-ethoxy-1-ethoxycarbonyl-1,2-dihydroquinoline, EEDQ, CAS Reg. No. 16357-59-8 (8.50 g, 34.36 mmol, 1.5 eq) and then stirred at 20° C. for 12 h. The mixture was concentrated in vacuum to give a residue and the residue was purified by flash silica gel chromatography (Silica Flash Column, Eluent of 0-40% Ethyl acetate/MeOH @ 65 mL/min) to give 13d (13 g, 24.0 mmol, 52.38% yield) as yellow solid. ¹H NMR (MeOD, 400 MHz) δ7.85-7.71 (m, 2H), 7.68-7.48 (m, 3H), 7.47-7.16 (m, 7H), 4.53 (d, J=15.2 Hz, 2H), 4.49-4.41 (m, 1H), 4.40-4.33 (m, 2H), 4.32-4.27 (m, 1H), 4.26-4.17 (m, 1H), 4.16-4.07 (m, 1H), 3.69-3.38 (m, 2H), 2.40-2.05 (m, 2H), 1.99-1.82 (m, 2H), 1.08-0.88 (m, 6H).

### Preparation of (2S)—N-[(1S)-1-[[4-(hydroxymethyl)phenyl]carbamoyl]-2-methyl-propyl]pyrrolidine-2-carboxamide, 13e

To a solution of 13d (13 g, 24.0 mmol, 1.0 eq) in DCM (130 mL) was added piperidine (10.22 g, 120 mmol, 11.85 mL, 5.0 eq) and then stirred at 20° C. for 2 h. The mixture was concentrated to give a residue and the residue was triturated with EtOAc at 20° C. for 20 min to give 7e (8 g, crude) as white solid.

### Preparation of 9H-fluoren-9-ylmethyl N-[(1S)-2-[(2S)-2-[[(1S)-1-[[4-(hydroxymethyl) phenyl]carbamoyl]-2-methyl-propyl]carbamoyl]pyrrolidin-1-yl]-1-methyl-2-oxoethyl]carbamate, 13f

To a solution of (2S)-2-(9H-fluoren-9-ylmethoxycarbonylamino)propanoic acid (3.95 g, 12.68 mmol, 1.5 eq) in DMF (30 mL) was added 1-Bis(dimethylamino)methylene]-1H-1,2,3-triazolo[4,5-b]pyridinium 3-oxide hexafluorophosphate. Hexafluorophosphate Azabenzotriazole Tetramethyl Uronium, HATU, CAS Reg. No. 148893-10-1 (4.82 g, 12.68 mmol, 1.5 eq) and DIPEA (3.28 g, 25.36 mmol, 4.42 mL, 3 eq) at 0° C. After addition, the mixture was stirred at this temperature for 5 minutes, and then 13e (2.7 g, 8.45 mmol, 1 eq) was added at 0° C. and then the resulting mixture was stirred at 0° C. for 25 min. The reaction mixture was quenched by addition of H₂O (150 mL) and then extracted with EtOAc (70 mL×3). The combined organic layers were washed with brine (50 mL×3), dried over Na₂SO₄, filtered and concentrated under reduced pressure. The residue was purified by column chromatography (SiO₂, Petroleum ether:Ethyl acetate=1:0 to 0:1) and then (SiO₂, EtOAc:MeOH=1:0 to 10:1) to give 13f (2.94 g, 4.80 mmol, 56.76% yield) as an off-white solid. ¹H NMR (MeOD-d₄, 400 MHz) δ7.79 (d, J=7.6 Hz, 2H), 7.66 (t, J=6.4 Hz, 2H), 7.54 (d, J=8.4 Hz, 2H), 7.39 (t, J=7.2 Hz, 2H), 7.35-7.26 (m, 4H), 4.59-4.51 (m, 3H), 4.50-4.40 (m, 1H), 4.39-4.30 (m, 2H), 4.29-4.18 (m, 2H), 3.83-3.71 (m, 1H), 3.68-3.63 (m, 1H), 2.31-2.09 (m, 2H), 2.07-1.91 (m, 3H), 1.36 (dd, J=6.4, 9.6 Hz, 4H), 1.03 (dd, J=4.0, 6.8 Hz, 6H).

### Preparation of [4-[[(2S)-2-[[(2S)-1-[(2S)-2-(9H-fluoren-9-ylmethoxycarbonylamino) propanoyl]pyrrolidine-2-carbonyl]amino]-3-methyl-butanoyl]amino]phenyl]methyl (4-nitrophenyl) carbonate, 13g

To a solution of 13f (2.4 g, 3.92 mmol, 1 eq) in DMF (20 mL) was added bis(4-nitrophenyl) carbonate (2.38 g, 7.83 mmol, 2 eq) and DIPEA (1.01 g, 7.83 mmol, 1.36 mL, 2 eq) and then stirred at 20° C. for 1 h. The reaction mixture was quenched by addition of H₂O (100 mL) at 0° C., and then extracted with EtOAc (80 mL×3). The combined organic layers were washed with brine (50 mL×3), dried over Na₂SO₄, filtered and concentrated under reduced pressure. The residue was purified by column chromatography (SiO₂, Petroleum ether:Ethyl acetate=1:0 to 0:1) to give 13g (2.7 g, 3.47 mmol, 88.62% yield) as a white solid. ¹H NMR (CDCl₃, 400 MHz) δ 8.32-8.22 (m, 3H), 7.78 (d, J=7.6 Hz, 2H), 7.67 (br d, J=8.4 Hz, 2H), 7.60 (br d, J=7.6 Hz, 2H), 7.46-7.29 (m, 8H), 7.18 (br d, J=8.4 Hz, 1H), 5.60 (br d, J=7.6 Hz, 1H), 5.25 (s, 2H), 4.70-4.66 (m, 1H), 4.62-4.52 (m, 1H), 4.46-4.28 (m, 3H), 4.28-4.19 (m, 1H), 3.81-3.68 (m, 1H), 3.62-3.58 (m, 1H), 2.48-2.29 (m, 2H), 2.19-1.98 (m, 3H), 1.41 (d, J=7.2 Hz, 3H), 1.10-0.94 (m, 6H). LC/MS [M+H] 778.3 (calculated); LC/MS [M+H] 778.2 (observed).

### Preparation of tert-butyl 3-[2-[2-[2-[2-[2-[2-[2-[2-[2-[2-[[2-(2,5-dioxopyrrol-1-yl) acetyl]amino]ethoxy]ethoxy]ethoxy]ethoxy]ethoxy]ethoxy]ethoxy]ethoxy]ethoxy]ethoxy]propanoate, 13i

To a solution of tert-butyl 3-[2-[2-[2-[2-[2-[2-[2-[2-[2-(2-aminoethoxy)ethoxy]ethoxy]ethoxy]ethoxy]ethoxy]ethoxy]ethoxy]ethoxy]ethoxy]propanoate, 13h (11.3 g, 19.3 mmol, 1 eq), 2-(2,5-dioxopyrrol-1-yl)acetic acid (3 g, 19.3 mmol, 1 eq) and DIPEA (10.0 g, 77.4 mmol, 13.5 mL, 4 eq) in DCM (100 mL) was added HATU (8.09 g, 21.3 mmol, 1.1 eq) at 0° C., and then the mixture was stirred at 0° C. for 30 min. The reaction mixture was concentrated under reduced pressure. The residue was purified by prep-HPLC (TFA condition; column: Phenomenex luna c18 250 mm*100 mm*10 um; mobile phase: [water (0.1% TFA)-ACN]; B %: 25%-55%, 25 min) to give 13i (4.5 g, 6.23 mmol, 32.2% yield) as a yellow oil. ¹H NMR (CDCl₃, 400 MHz) δ6.88-6.80 (m, 1H), 6.78 (s, 2H), 4.22 (s, 2H), 3.77-3.54 (m, 40H), 3.47 (q, J=5.2 Hz, 2H), 2.51 (t, J=6.4 Hz, 2H), 1.46 (s, 9H).

### Preparation of 3-[2-[2-[2-[2-[2-[2-[2-[2-[2-[2-[[2-(2,5-dioxopyrrol-1-yl) acetyl]amino]ethoxy]ethoxy]ethoxy]ethoxy]ethoxy]ethoxy]ethoxy]ethoxy]ethoxy]ethoxy]propanoic Acid, 13j

To a solution of 13i (4.5 g, 6.23 mmol, 1 eq) in CH₃CN (25 mL) and H₂O (25 mL) was added TFA (5.68 g, 49.8 mmol, 3.69 mL, 8 eq) and then stirred at 80° C. for 1 h. The reaction mixture was concentrated under reduced pressure to remove CH₃CN. The residue was extracted with MTBE (10 mL×3) and discarded. The water phase was concentrated under reduced pressure to give a residue. The residue was purified by prep-HPLC (TFA condition; column: Phenomenex Luna c18 250 mm×100 mm×10 um; mobile phase: [water (0.1% TFA)-ACN]; B %: 0%-25%, 24 min) to give 13j (1.6 g, 2.40 mmol, 38.6% yield) as a light yellow oil. ¹H NMR (CDCl₃, 400 MHz) δ6.95 (br s, 1H), 6.78 (s, 2H), 4.22 (s, 2H), 3.78 (t, J=6.4 Hz, 2H), 3.70-3.63 (m, 36H), 3.60-3.54 (m, 2H), 3.46 (q, J=5.2 Hz, 2H), 2.61 (t, J=6.0 Hz, 2H). LCMS (ESI): mass calcd. for C₄₂H₄₃N₅O₁₀ 667.3, m/z found 667.2 [M+H]⁺.

### Preparation of [4-[[(2S)-2-[[(2S)-1-[(2S)-2-aminopropanoyl]pyrrolidine-2-carbonyl]amino]-3-methyl-butanoyl]amino]phenyl]methyl 4-[3-[[(2E)-6-carbamoyl-3-[(E)-4-[(2E)-5-carbamoyl-2-(2-ethyl-5-methyl-pyrazole-3-carbonyl)imino-7-methoxy-3H-benzimidazol-1-yl]but-2-enyl]-2-(2-ethyl-5-methyl-pyrazole-3-carbonyl)imino-1H-benzimidazol-4-yl]oxy]propyl]piperazine-1-carboxylate, 13l

To a solution of (2E)-1-[(E)-4-[(2E)-5-carbamoyl-2-(2-ethyl-5-methyl-pyrazole-3-carbonyl)imino-7-(3-piperazin-1-ylpropoxy)-3H-benzimidazol-1-yl]but-2-enyl]-2-(2-ethyl-5-methyl-pyrazole-3-carbonyl)imino-7-methoxy-3H-benzimidazole-5-carboxamide, 13k (150 mg, 151 umol, 1 eq, 4HCl) and 13g (129 mg, 166 umol, 1.1 eq) in DMF (3.0 mL) was added DIPEA (97.0 mg, 754 umol, 131 uL, 5 eq) at 20° C., the mixture was stirred at this temperature for 2 h, and then piperidine (39.0 mg, 452.35 umol, 45.0 uL, 3 eq) was added. The mixture was stirred at 20° C. for another 2 h. The mixture was filtered and the residue was purified by prep-HPLC (column: Phenomenex Luna 80*30 mm*3 um; mobile phase: [water (0.1% TFA)-ACN]; B %: 20%-40%, 8 min) to give 13l (90 mg, 60.26 umol, 39.97% yield, 2TFA) as light yellow solid. ¹H NMR (MeOD, 400 MHz) δ7.67-7.53 (m, 4H), 7.37 (d, J=8.4 Hz, 2H), 7.25 (dd, J=1.2, 14.0 Hz, 2H), 6.60 (d, J=7.6 Hz, 2H), 5.85-5.68 (m, 2H), 5.14 (s, 2H), 5.00 (br s, 5H), 4.64-4.54 (m, 4H), 4.33-4.18 (m, 2H), 3.95 (br t, J=6.0 Hz, 2H), 3.74-3.58 (m, 5H), 3.25-3.13 (m, 4H), 2.21 (s, 3H), 2.19 (s, 3H), 2.16-1.92 (m, 6H), 1.52 (d, J=7.2 Hz, 3H), 1.40-1.29 (m, 6H), 1.08-0.98 (m, 6H).

### Preparation of BBI-L-13

To a solution 13l (50 mg, 33.5 umol, 1 eq, 2TFA) and 7j (22.0 mg, 33.5 umol, 1 eq) in DMF (1.00 mL) was added Et₃N (7.00 mg, 66.9 umol, 9.00 uL, 2 eq) and 1-Propanephosphonic anhydride, T₃P, CAS Reg. No. 68957-94-8 (32.0 mg, 50.2 umol, 30.0 uL, 50% purity, 1.5 eq), and then stirred at 20° C. for 2 h. The mixture was filtered and the residue was purified by prep-HPLC (column: Phenomenex Luna 80*30 mm*3 um; mobile phase: [water (0.1% TFA)-ACN]; B %: 15%-40%, 8 min) to give BBI-L-13 (16 mg, 7.47 umol, 22.31% yield, 2TFA) as white solid. ¹H NMR (MeOD, 400 MHz) δ7.68-7.53 (m, 4H), 7.37 (d, J=8.4 Hz, 2H), 7.30-7.20 (m, 2H), 6.88 (s, 2H), 6.60 (d, J=12 Hz, 2H), 5.78 (br s, 2H), 5.14 (s, 2H), 4.99 (br s, 4H), 4.68-4.47 (m, 8H), 4.29-4.25 (m, 1H), 4.16 (s, 3H), 3.96 (br t, J=5.6 Hz, 2H), 3.80 (td, J=6.8, 9.2 Hz, 2H), 3.74-3.71 (m, 2H), 3.70 (s, 4H), 3.67-3.56 (m, 36H), 3.53 (br t, J=5.6 Hz, 2H), 3.48 (td, J=1.6, 3.2 Hz, 1H), 3.36 (br t, J=5.2 Hz, 2H), 3.23-3.13 (m, 4H), 2.54-2.43 (m, 2H), 2.20 (d, J=10.4 Hz, 6H), 2.18-2.10 (m, 2H), 2.06-1.94 (m, 5H), 1.40-1.28 (m, 9H), 1.02 (dd, J=5.2, 6.4 Hz, 6H). LCMS (ESI): mass calcd. for C₄₂H₄₃N₅O₁₀ 1913.9, m/z found 1914.0 [M+H]⁺.

### Example L-18 Synthesis of 1-[(E)-4-[5-carbamoyl-7-[3-[4-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl]piperazin-1-yl]propoxy]-2-[(2-ethyl-5-methyl-pyrazole-3-carbonyl)amino]-3a,7a-dihydrobenzimidazol-1-yl]but-2-enyl]-2-[(2-ethyl-5-methyl-pyrazole-3-carbonyl)amino]-7-methoxy-benzimidazole-5-carboxamide, BBI-L-18

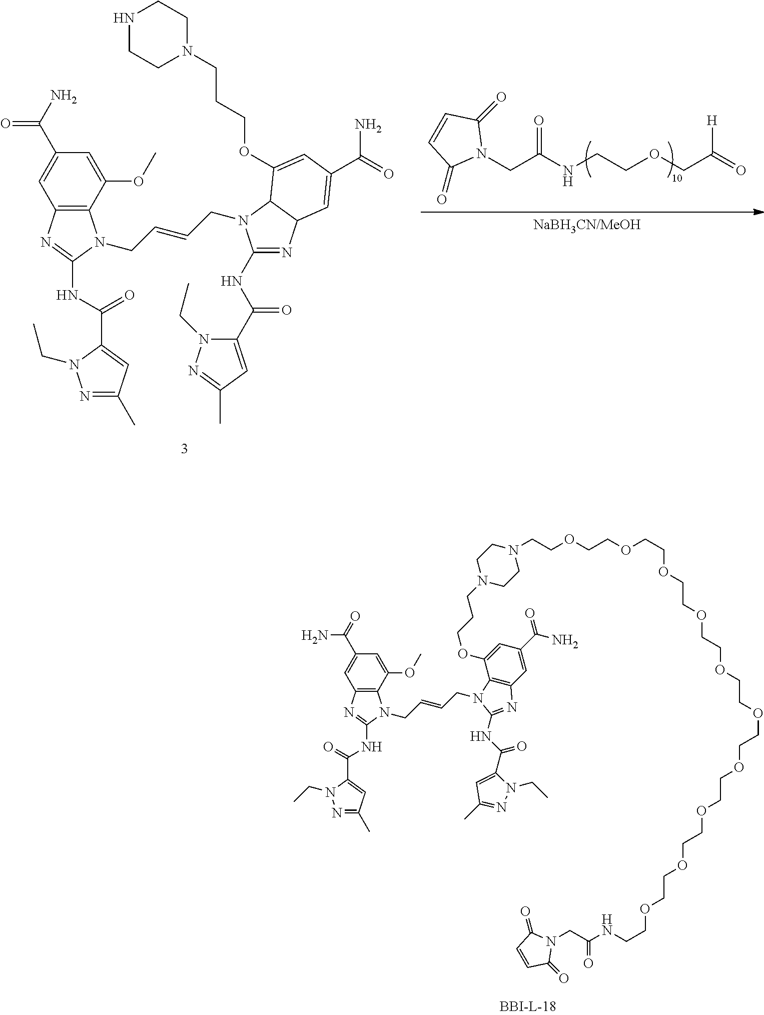

To a mixture of 2-(2,5-dioxopyrrol-1-yl)-N-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]acetamide (68.9 mg, 108 umol, 2 eq) and 1-[(E)-4-[5-carbamoyl-2-[(2-ethyl-5-methyl-pyrazole-3-carbonyl)amino]-7-(3-piperazin-1-ylpropoxy)-3a,7a-dihydrobenzimidazol-1-yl]but-2-enyl-2-[(2-ethyl-5-methyl-pyrazole-3-carbonyl)amino]-7-methoxy-benzimidazole-5-carboxamide, 3 (50 mg, 54.1 umol, 1 eq, 2HCl) in MeOH (3 mL) was added sodium cyanoborohydride, NaBH₃CN (6.8 mg, 108 umol, 2 eq) at 25° C. under N₂ and then stirred at 25° C. for 13 hours. The mixture was concentrated in vacuum to give a residue. The residue was purified by prep-HPLC column: Phenomenex Luna 80*30 mm*3 um; mobile phase: [water (0.1% TFA)-ACN]; B %: 15%-40%, 8 min to give BBI-L-18 (15 mg, 9.46 umol, 17.48% yield, TFA) as light yellow oil. ¹H NMR (400 MHz, MeOD) δ 7.61 (d, J=1.2 Hz, 1H), 7.57 (d, J=1.2 Hz, 1H), 7.31 (d, J=1.2 Hz, 1H), 7.28 (d, J=1.2 Hz, 1H), 6.87 (s, 2H), 6.63 (s, 1H), 6.57 (s, 1H), 5.88-5.83 (m, 2H), 5.08-5.01 (m, 4H), 4.54-4.69 (m, 4H), 4.16 (s, 2H), 4,01-3.95 (m, 2H), 3.80-3.88 (m, 2H), 3.55-3.72 (m, 38H), 3.52 (t, J=5.5 Hz, 2H), 3.38-3.30 (m, 6H), 2.69-2.93 (m, 4H), 2.60 (br t, J=7.2 Hz, 2H), 2.21 (d, J=8.8 Hz, 6H), 1.71-1.82 (m, 2H), 1.40-1.32 (m, 6H). HPLC: 99.67% (220 nm). LC/MS [M+H] 1469.7 (calculated); LC/MS [M+H] 1469.8 (observed).

### Example L-24 Synthesis of 1-[4-[5-carbamoyl-7-[3-[4-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl]piperazin-1-yl]propoxy]-2-[(2-ethyl-5-methyl-pyrazole-3-carbonyl)amino]benzimidazol-1-yl]butyl]-2-[(2-ethyl-5-methyl-pyrazole-3-carbonyl)amino]-7-methoxy-benzimidazole-5-carboxamide, BBI-L-24

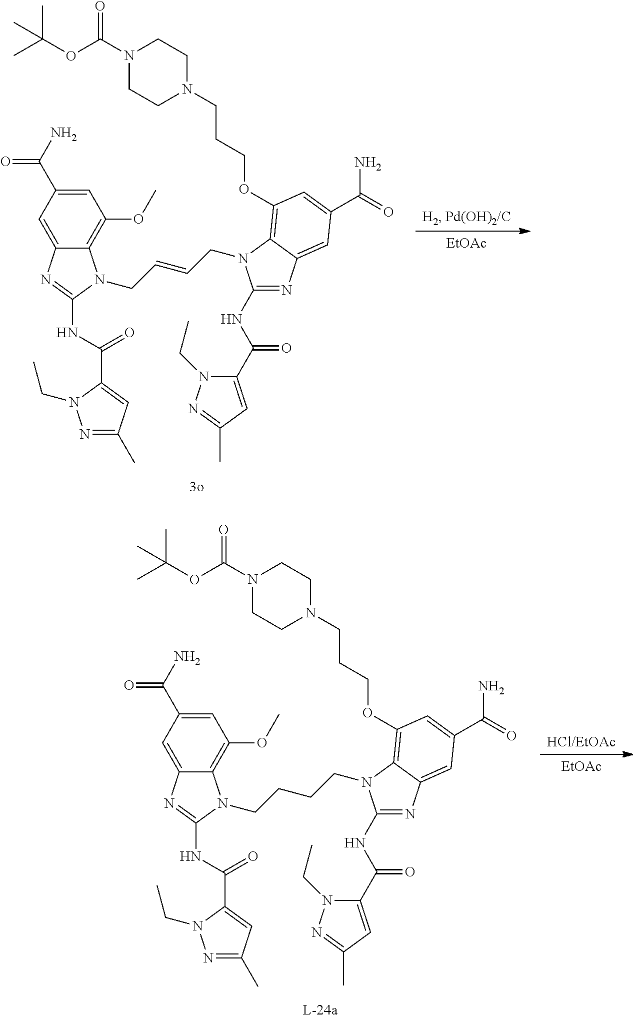

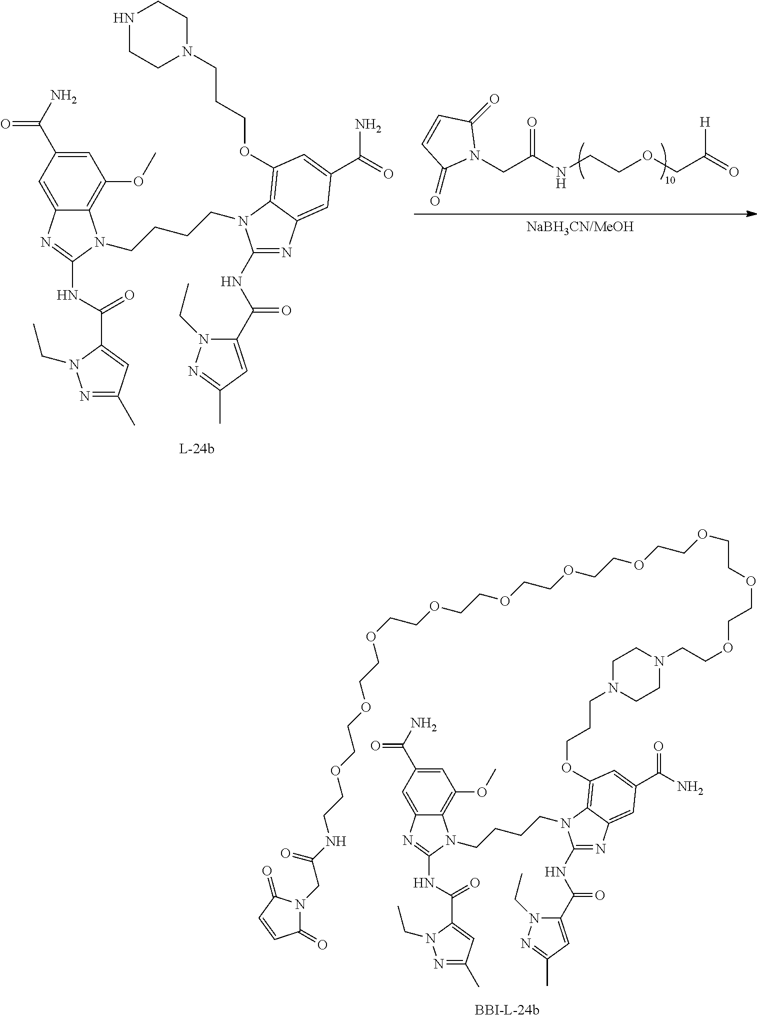

### Preparation of tert-butyl 4-[3-[6-carbamoyl-3-[4-[5-carbamoyl-2-[(2-ethyl-5-methyl-pyrazole-3-carbonyl)amino]-7-methoxy-benzimidazol-1-yl]butyl]-2-[(2-ethyl-5-methyl-pyrazole-3-carbonyl)amino]benzimidazol-4-yl]oxypropyl]piperazine-1-carboxylate, L-24a

To a solution of tert-butyl 4-[3-[6-carbamoyl-3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]benzimidazol-4-yl]oxypropyl]piperazine-1-carboxylate, 3o (0.3 g, 316.10 umol, 1 eq) in EtOAc (20 mL) was added Pd(OH)₂/C (10%, 0.1 g) under N₂. The suspension was degassed under vacuum and purged with H₂ several times, and then stirred under H₂ (50 psi) at 20° C. for 12 hours. The reaction mixture was filtered and concentrated under reduced pressure to give L-24a (0.2 g, 210.29 umol, 66.53% yield) as a black solid. LC/MS [M+H] 951.5 (calculated); LC/MS [M+H] 951.4 (observed).

### Preparation of 1-[4-[5-carbamoyl-2-[(2-ethyl-5-methyl-pyrazole-3-carbonyl) amino]-7-(3-piperazin-1-ylpropoxy)benzimidazol-1-yl]butyl]-2-[(2-ethyl-5-methyl-pyrazole-3-carbonyl)amino]-7-methoxy-benzimidazole-5-carboxamide, L-24b

To a solution of L-24a (0.2 g, 210.29 umol, 1 eq) in EtOAc (2 mL) was added HCl/EtOAc (4 M, 10.0 mL, 190 eq). The mixture was stirred at 20° C. for 2 hr. The reaction mixture was concentrated under reduced pressure to give a residue. The residue was purified by prep-HPLC (column: Phenomenex Luna 80*30 mm*3 um; mobile phase: [water (0.1% TFA)-ACN]; B %: 10%-40%, 8 min) to give L-24b (0.1 g, 117.51 umol, 55.88% yield) as a white solid. ¹H NMR (400 MHZ, DMSO-d₆) δ 7.32-7.27 (m, 2H), 7.22 (d, J=8.0 Hz, 2H), 6.68 (s, 2H), 4.73-4.63 (m, 4H), 4.43-4.34 (m, 4H), 4.13-4.04 (m, 2H), 3.83 (s, 3H), 3.26-3.18 (m, 4H), 2.77-2.63 (m, 4H), 2.60-2.51 (m, 2H), 2.24 (d, J=2.4 Hz, 6H), 2.07-1.96 (m, 4H), 1.94-1.76 (m, 2H), 1.46-1.35 (m, 6H). HPLC: 96.010% (220 nm), 96.753% (254 nm). LC/MS [M+H] 851.4 (calculated); LC/MS [M+H] 851.4 (observed).

### Preparation of BBI-L-24

To a solution of L-24b (80.0 mg, 74.1 umol, 1 eq, 2TFA) in MeOH (5 mL) was added 2-(2,5-dioxopyrrol-1-yl)-N-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]acetamide (142 mg, 222 umol, 3 eq) at 15° C. After addition, the mixture was stirred at this temperature for min, and then NaBH₃CN (14.0 mg, 222 umol, 3 eq) was added. The resulting mixture was stirred at 15° C. for 12 hr. The reaction mixture was concentrated under reduced pressure to give a residue. The residue was purified by prep-HPLC (TFA condition; column: Phenomenex Luna 80*30 mm*3 um; mobile phase: [water (TFA)-ACN]; B %: 5%-35%, 8 min) to give BBI-L-24 (40.1 mg, 23.6 umol, 31.8% yield, 2TFA) as a white solid. ¹H NMR (400 MHz, MeOD) δ7.36-7.29 (m, 2H), 7.23 (d, J=5.2 Hz, 2H), 6.88 (s, 2H), 6.68 (s, 2H), 4.67 (q, J=6.0 Hz, 4H), 4.45-4.31 (m, 4H), 4.16 (s, 2H), 4.10 (br t, J=6.0 Hz, 2H), 3.89-3.77 (m, 5H), 3.72-3.66 (m, 4H), 3.66-3.55 (m, 32H), 3.52 (t, J=5.6 Hz, 2H), 3.47-3.33 (m, 8H), 3.03-2.90 (m, 4H), 2.84-2.72 (m, 2H), 2.23 (d, J=2.0 Hz, 6H), 2.08-1.87 (m, 6H), 1.49-1.34 (m, 6H). HPLC: 97.38% (220 nm). LC/MS [M+H] 1471.7 (calculated); LC/MS [M+H] 1471.7 (observed).

### Example L-26 Synthesis of 7-[3-[4-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl]piperazin-1-yl]propoxy]-2-[(2-ethyl-5-methyl-pyrazole-3-carbonyl)amino-1-[(E)-4-[2-[(2-ethyl-5-methyl-pyrazole-3-carbonyl)amino]benzimidazol-1-yl]but-2-enyl]benzimidazole-5-carboxamide, BBI-L-26

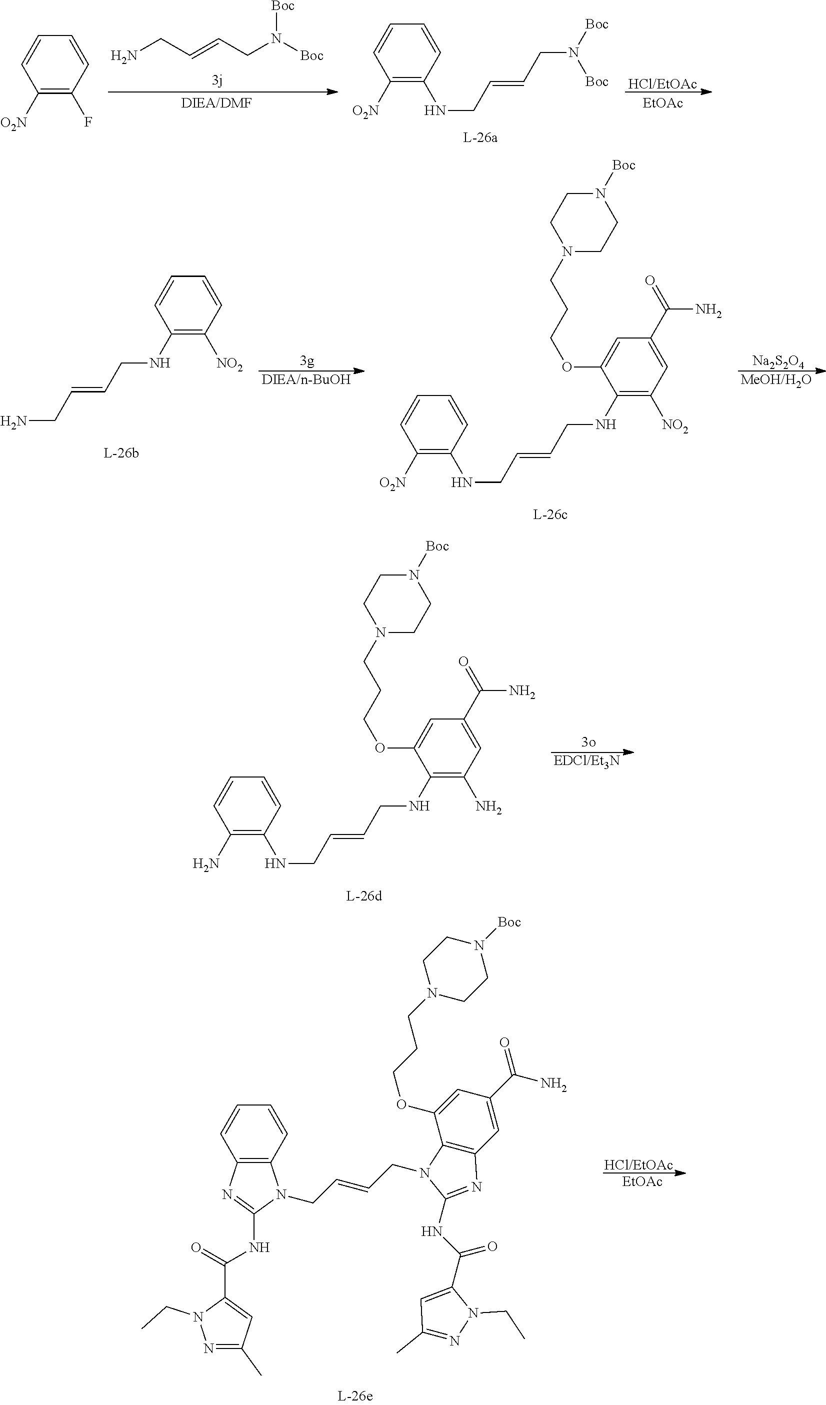

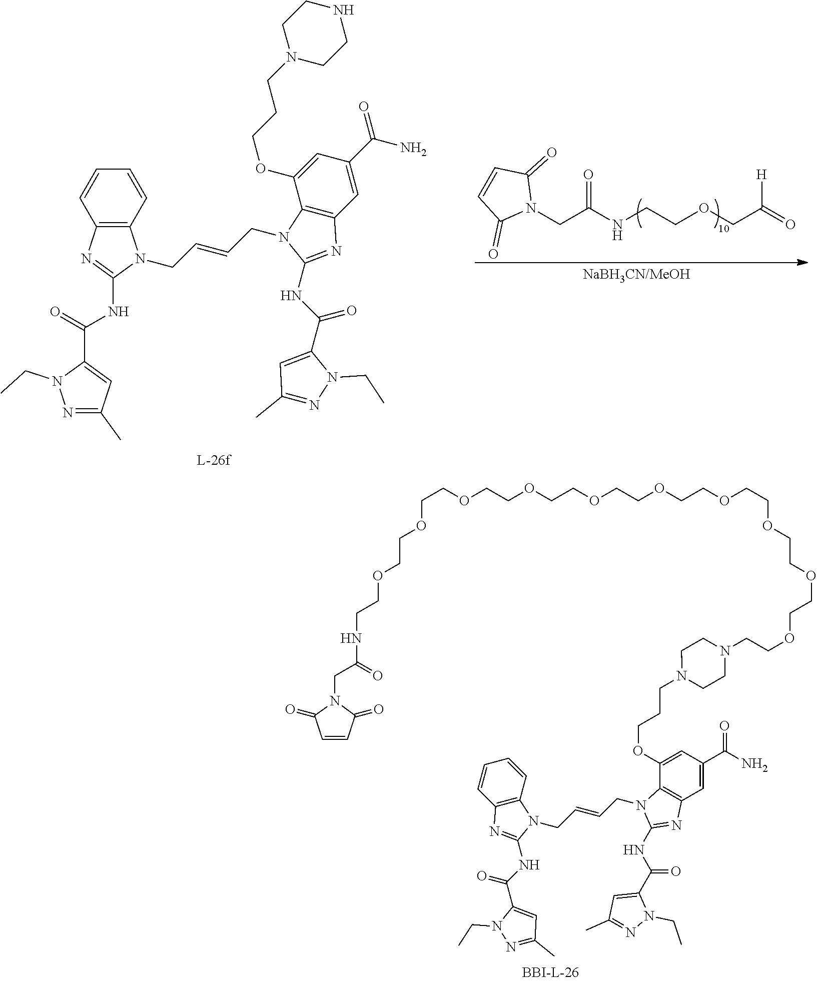

### Preparation of tert-butyl N-tert-butoxycarbonyl-N-[(E)-4-(2-nitroanilino)but-2-enyl]carbamate, L-26a

To a solution of 1-fluoro-2-nitro-benzene (1 g, 7.09 mmol, 746 uL, 1 eq) in DMF (10 mL) was added DIEA (2.75 g, 21.26 mmol, 3.7 mL, 3 eq) and tert-butyl N-[(E)-4-aminobut-2-enyl]-N-tert-butoxycarbonyl-carbamate, 3j (2.23 g, 7.80 mmol, 1.1 eq), and then stirred at 50° C. for 16 hr. The reaction mixture was quenched by addition H₂O (100 mL) at 20° C., and then extracted with EtOAc (50 mL×3). The combined organic layers were washed with H₂O (50 mL) and brine (20 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜5% Ethyl acetate/Petroleum ether gradient @ 100 mL/min) to give L-26a (2 g, 4.91 mmol, 69.26% yield) as brownness solid. ¹H NMR (400 MHz, MeOD) δ8.12 (dd, J=1.6, 8.8 Hz, 1H), 7.46 (td, J=1.6, 7.2, 8.8 Hz, 1H), 6.96 (d, J=8.8 Hz, 1H), 6.67 (d, J=1.6, 7.2, 8.8 Hz, 1H), 5.80-5.72 (m, 2H), 4.17 (d, J=4.0 Hz, 2H), 4.03 (d, J=4.0 Hz, 2H), 1.45 (s, 18H). LC/MS [M+H] 408.2 (calculated); LC/MS [M+H] 408.2 (observed).

### Preparation of (E)-N′-(2-nitrophenyl)but-2-ene-1,4-diamine, L-26b

To a solution of L-26a (500 mg, 1.23 mmol, 1 eq) in EtOAc (1 mL) was added HCl/EtOAc (4 M, 15.34 mL, 50 eq). The mixture was stirred at 20° C. for 2 hr. The reaction mixture was concentrated under reduced pressure to give L-26b (500 mg, crude) as a white solid. ¹H NMR (400 MHz, MeOD) δ8.14 (dd, J=1.6, 8.4 Hz, 1H), 7.49 (td, J=1.6, 7.2, 8.4 Hz, 1H), 6.97 (d, J=8.0 Hz, 1H), 6.70 (td, J=1.6, 7.2, 8.4 Hz, 1H), 6.14-6.02 (m, 1H), 5.86-5.70 (m, 1H), 4.11 (dd, J=1.2, 4.4 Hz, 2H), 3.57 (d, J=6.4 Hz, 2H). LC/MS [M+H] 208.1 (calculated); LCMS [M+H] 208.1 (observed).

### Preparation of tert-butyl 4-[3-[5-carbamoyl-3-nitro-2-[[(E)-4-(2-nitroanilino)but-2-enyl]amino]phenoxy]propyl]piperazine-1-carboxylate, L-26c

A solution of L-26b (0.5 g, 1.78 mmol, 1 eq, 2HCl), DIEA (1.15 g, 8.92 mmol, 1.55 mL, 5 eq) and NaHCO₃ (375 mg, 4.46 mmol, 174 uL, 2.5 eq) in n-BuOH (10 mL) was stirred at 15° C. for 20 min. Then tert-butyl 4-[3-(5-carbamoyl-2-chloro-3-nitro-phenoxy)propyl]piperazine-1-carboxylate, 3g (632 mg, 1.43 mmol, 0.8 eq) was added. The mixture was stirred at 120° C. for 16 hr. The reaction mixture was concentrated under reduced pressure to remove n-BuOH, the residue was quenched by addition H₂O (50 mL) at 20° C., and then extracted with EtOAc (10 mL×2). The combined organic layers were washed with brine (10 mL), dried over Na₂SO₄, filtered and concentrated under reduced pressure to give L-26c (550 mg, 896.26 umol, 50.22% yield) as yellow solid. LC/MS [M+H] 614.3 (calculated); LC/MS [M+H] 614.2 (observed).

### Preparation of tert-butyl 4-[3-[3-amino-2-[[(E)-4-(2-aminoanilino)but-2-enyl]amino]-5-carbamoyl-phenoxy]propyl]piperazine-1-carboxylate, L-26d

To a solution of L-26c (550 mg, 896 umol, 1 eq) in MeOH (1 mL) and THF (1 mL) and H₂O (0.5 mL) was added Na₂CO₃ (570 mg, 5.38 mmol, 6 eq) and disodium dithionite (1.56 g, 8.96 mmol, 1.95 mL, 10 eq), and then stirred at 20° C. for 2 hr. The reaction mixture was filtered and the filtrate was concentrated to remove THF and MeOH. The residue was diluted with H₂O (10 mL) and the precipitate was filtered to give L-26d (5 50 mg, crude) as yellow solid. LC/MS [M+H] 554.3 (calculated); LC/MS [M+H] 554.3 (observed).

### Preparation of tert-butyl 4-[3-[6-carbamoyl-2-[(2-ethyl-5-methyl-pyrazole-3-carbonyl)amino]-3-[(E)-4-[2-[(2-ethyl-5-methyl-pyrazole-3-carbonyl)amino]benzimidazol-1-yl]but-2-enyl]benzimidazol-4-yl]oxypropyl]piperazine-1-carboxylate, L-26e

A solution of L-26d (550 mg, 993 umol, 1 eq) and 2-ethyl-5-methyl-pyrazole-3-carbonyl isothiocyanate, 3o (213 mg, 1.09 mmol, 1.1 eq) in DMF (5 mL) was stirred for 10 min at 0° C., then Et₃N (301 mg, 2.98 mmol, 414 uL, 3 eq) and EDCI (571 mg, 2.98 mmol, 3 eq). The result mixture was stirred at 20° C. for 16 hr. The reaction mixture was quenched by adding saturated sodium bicarbonate (10 mL) and H₂O (10 mL), and the precipitate was filtered to give L-26e (560 mg, 639 umol, 64.36% yield) as white solid. LC/MS [M+H] 876.5 (calculated); LC/MS [M+H] 876.4 (observed).

### Preparation of 2-[(2-ethyl-5-methyl-pyrazole-3-carbonyl)amino]-1-[(E)-4-[2-[(2-ethyl-5-methyl-pyrazole-3-carbonyl)amino]benzimidazol-1-yl]but-2-enyl]-7-(3-piperazin-1-ylpropoxy)benzimidazole-5-carboxamide, L-26f

To a solution of L-26e (560 mg, 639 umol, 1 eq) in EtOAc (5 mL) was added HCl/EtOAc (4 M, 7.99 mL, 50 eq), and then stirred at 20° C. for 2 hr. The reaction mixture was concentrated and the residue was purified by prep-HPLC (column: Phenomenex Luna 80×30 mm×3 um; mobile phase: [water (TFA)-ACN]; B %: 5%-40%, 8 min) to afford L-26e (96.7 mg, 124.63 umol, 19.50% yield) as white solid. ¹H NMR (400 MHz, MeOD) δ7.57 (d, J=1.2 Hz, 1H), 7.48 (d, J=7.6 Hz, 1H), 7.35-7.24 (m, 3H), 7.22-7.16 (m, 1H), 6.63 (s, 1H), 6.57 (s, 1H), 6.00-5.90 (m, 1H), 5.84-5.74 (m, 1H), 5.06 (d, J=5.2 Hz, 2H), 4.83-4.79 (m, 2H), 4.69-4.54 (m, 4H), 4.04 (t, J=6.4 Hz, 2H), 3.33 (s, 2H), 3.27-3.23 (m, 4H), 2.86-2.69 (m, 4H), 2.61 (t, J=7.2 Hz, 2H), 2.23 (s, 3H), 2.21 (s, 3H), 1.88-1.77 (m, 2H), 1.38 (t, J=7.2 Hz, 3H), 1.33 (t, J=7.2 Hz, 3H). LC/MS [M+H] 776.4 (calculated); LC/MS [M+H] 776.4 (observed).

### Preparation of BBI-L-26

A solution of L-26e (30 mg, 29.9 umol, 1 eq, 2TFA) and 2-(2,5-dioxopyrrol-1-yl)-N-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]acetamide (47.6 mg, 74.7 umol, 2.5 eq) in MeOH (3 mL) was stirred for 10 min at 20° C., then NaBH₃CN (5.63 mg, 89.6 umol, 3 eq) was added. The mixture was stirred at 20° C. for another 0.5 hr. The reaction mixture was filtered and purified by prep-HPLC (column: Phenomenex Luna 80×30 mm×3 um; mobile phase: [water (TFA)-ACN]; B %: 10%-40%, 8 min) to afford BBI-L-26 (14.9 mg, 10.67 umol, 35.70% yield) as white solid. ¹H NMR (400 MHz, MeOD) δ7.57 (d, J=1.2 Hz, 1H), 7.48 (d, J=8.0 Hz, 1H), 7.35-7.30 (m, 2H), 7.27 (t, J=7.6 Hz, 1H), 7.22-7.16 (m, 1H), 6.86 (s, 2H), 6.62 (s, 1H), 6.56 (s, 1H), 6.05-5.87 (m, 1H), 5.86-5.70 (m, 1H), 5.06 (d, J=4.8 Hz, 2H), 4.65-4.54 (m, 4H), 4.15 (s, 2H), 4.05 (t, J=6.0 Hz, 2H), 3.86-3.78 (m, 2H), 3.67 (d, J=2.8 Hz, 4H), 3.64-3.54 (m, 34H), 3.51 (t, J=5.6 Hz, 4H), 3.37-3.33 (m, 4H), 3.21-2.75 (m, 6H), 2.66 (t, J=6.8 Hz, 2H), 2.21 (d, J=8.0 Hz, 6H), 1.88-1.78 (m, 2H), 1.37 (t, J=7.2 Hz, 3H), 1.32 (t, J=7.2 Hz, 3H). LC/MS [M+H] 1396.7 (calculated); LC/MS [M+H] 1396.7 (observed).

### Example L-28 Synthesis of 1-[(E)-4-[5-carbamoyl-7-[3-[4-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl]piperazin-1-yl]propoxy]-2-[[2-(2-hydroxyethyl)-5-methyl-pyrazole-3-carbonyl]amino]benzimidazol-1-yl]but-2-enyl]-2-[[2-(2-hydroxyethyl)-5-methyl-pyrazole-3-carbonyl]amino]-7-methoxy-benzimidazole-5-carboxamide, BBI-L-28

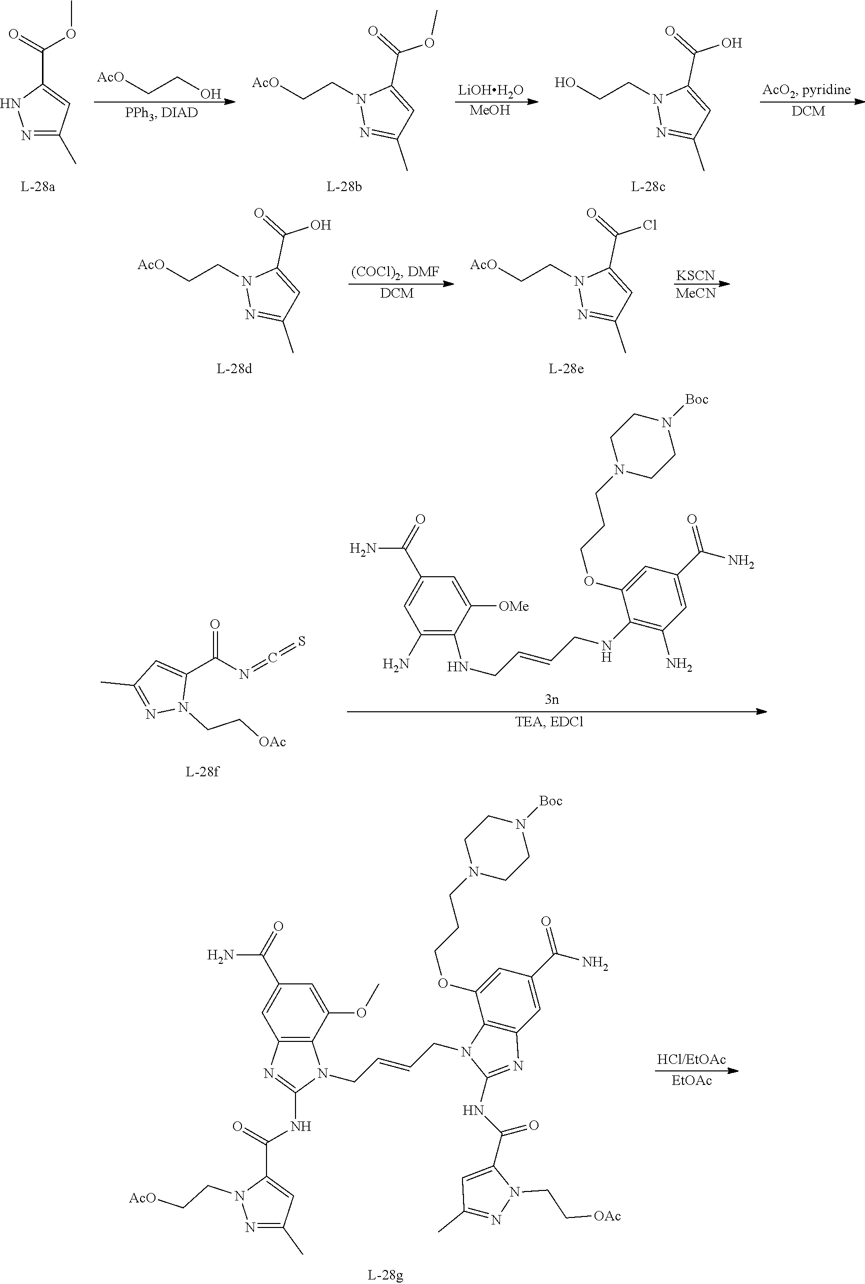

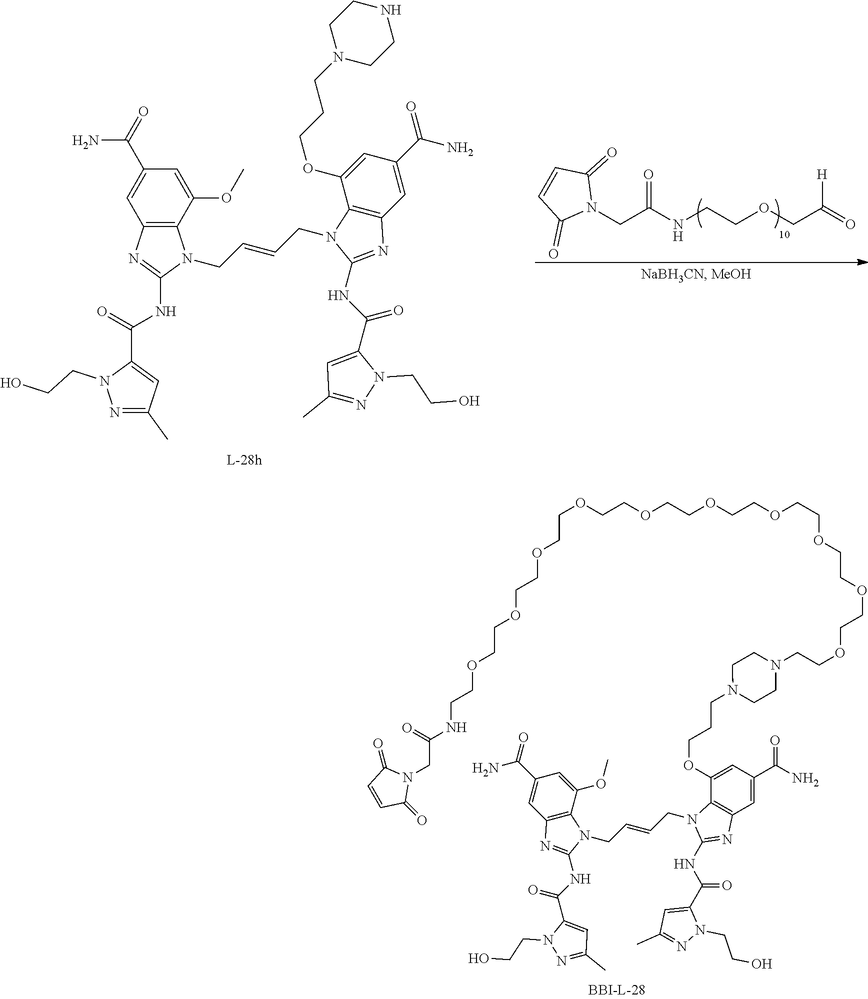

### Preparation of ethyl 2-(2-acetoxyethyl)-5-methyl-pyrazole-3-carboxylate, L-28b

To a solution of ethyl 3-methyl-1H-pyrazole-5-carboxylate, L-28a (3 g, 19.5 mmol, 1 eq) and 2-hydroxyethyl acetate (2.03 g, 19.5 mmol, 1.83 mL, 1 eq) in THF (30 mL) was added triphenylphosphine, PPh₃ (15.3 g, 58.4 mmol, 3 eq) and diisopropyl azo-dicarboxylate, DIAD (11.80 g, 58.4 mmol, 11.4 mL, 3 eq). The mixture was stirred at 0° C. for 2 hr. The reaction mixture was quenched by addition H₂O 50 mL, and extracted with EtOAc (50 mL×3). The combined organic layers were washed with brine (100 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 60 mL/min) to give L-28b (5 g, crude) as a colorless oil. ¹H NMR (400 MHz, CDCl3) δ 6.63 (s, 1H), 4.75 (t, J=5.4 Hz, 2H), 4.41 (t, J=5.4 Hz, 2H), 4.32 (q, J=7.2 Hz, 2H), 2.27 (s, 3H), 1.99 (s, 3H), 1.36 (t, J=7.2 Hz, 3H)

### Preparation of 2-(2-hydroxyethyl)-5-methyl-pyrazole-3-carboxylic Acid, L-28c

To a solution of L-28b (5 g, 20.8 mmol, 1 eq) in MeOH (30 mL) and H₂O (5 mL) was added LiOH·H₂O (2.62 g, 62.4 mmol, 3 eq), and then stirred at 20° C. for 2 h. The pH of the reaction mixture was adjusted to about 1 at 0° C. with HCl (4M), desired solid precipitated from the mixture, and filtered to give L-28c (3 g, 17.6 mmol, 84.71% yield) as a white solid. ¹H NMR (400 MHz, DMSO-d₆) δ 8.87 (s, 1H), 6.57 (s, 1H), 4.46 (t, J=6.4 Hz, 2H), 3.66 (t, J=6.4 Hz, 2H), 2.16 (s, 3H)

### Preparation of 2-(2-acetoxyethyl)-5-methyl-pyrazole-3-carboxylic Acid, L-28d

To a solution of L-28c (3 g, 17.6 mmol, 1 eq) in DCM (50 mL) was added acetic anhydride (3.60 g, 35.3 mmol, 3.30 mL, 2 eq) and pyridine (2.79 g, 35.3 mmol, 2.85 mL, 2 eq). The mixture was stirred at 20° C. for 1 hr. The pH of the reaction mixture was adjusted to about 1 at 0° C. with HCl (4M), desired solid precipitated from the mixture, and filtered to give L-28d (3 g, 14.14 mmol, 80.19% yield) as a white solid. ¹H NMR (400 MHz, DMSO-d₆) δ 8.88 (s, 1H), 6.60 (s, 1H), 4.64 (t, J=5.2 Hz, 2H), 4.30 (t, J=5.2 Hz, 2H), 2.17 (s, 3H), 1.92 (s, 3H)

### Preparation of 2-(5-chlorocarbonyl-3-methyl-pyrazol-1-yl)ethyl acetate, L-28e

To a solution of L-28d (1 g, 4.71 mmol, 1 eq) in DCM (10 mL) was added DMF (344 mg, 4.71 mmol, 363 uL, 1 eq) and oxalyl dichloride, (COCl)₂ (1.20 g, 9.42 mmol, 825 uL, 2 eq), and then stirred at 20° C. for 1 hr. The reaction mixture was concentrated under reduced pressure to give L-28e as a yellow oil.

### Preparation of 2-(5-carbonisothiocyanatidoyl-3-methyl-pyrazol-1-yl)ethyl acetate, L-28f

A solution of L-28e (1 g, 4.34 mmol, 1 eq) and potassium thiocyanate, KSCN (843 mg, 8.67 mmol, 843 uL, 2 eq) in MeCN (5 mL) was stirred at 20° C. for 1 hr. The reaction mixture was filtered and concentrated under reduced pressure to give a residue. The residue was purified by flash silica gel chromatography (ISCO®; 40 g SepaFlash® Silica Flash Column, Eluent of 0˜30% Ethyl acetate/Petroleum ether gradient @ 45 mL/min) to give L-28f (0.4 g, 1.58 mmol, 36.43% yield) as a yellow oil

### Preparation of tert-butyl 4-[3-[2-[[2-(2-acetoxyethyl)-5-methyl-pyrazole-3-carbonyl]amino]-3-[(E)-4-[2-[[2-(2-acetoxyethyl)-5-methyl-pyrazole-3-carbonyl]amino]-5-carbamoyl-7-methoxy-benzimidazol-1-yl]but-2-enyl]-6-carbamoyl-benzimidazol-4-yl]oxypropyl]piperazine-1-carboxylate, L-28g

A solution of tert-butyl 4-[3-[3-amino-2-[[(E)-4-(2-amino-4-carbamoyl-6-methoxy-anilino)but-2-enyl]amino]-5-carbamoyl-phenoxy]propyl]piperazine-1-carboxylate, 3n (0.3 g, 479 umol, 1 eq) and L-28f (242 mg, 957 umol, 2 eq) in DMF (5 mL) was stirred at 0° C. for 0.5 h. Then EDCI (459 mg, 2.39 mmol, 5 eq) and Et₃N (242 mg, 2.39 mmol, 333 uL, 5 eq was added. The result mixture was stirred at 25° C. for another 12 hr. The reaction mixture was quenched by addition aq NaHCO₃ 10 mL at 0° C., desired solid product precipitated from the mixture, filtered to L-28g (0.4 g, 375 umol, 78.46% yield) as a yellow solid.

### Preparation of 1-[(E)-4-[5-carbamoyl-2-[[2-(2-hydroxyethyl)-5-methyl-pyrazole-3-carbonyl]amino]-7-(3-piperazin-1-ylpropoxy)benzimidazol-1-yl]but-2-enyl]-2-[[2-(2-hydroxyethyl)-5-methyl-pyrazole-3-carbonyl]amino]-7-methoxy-benzimidazole-5-carboxamide, L-28h

To a solution of L-28g (0.4 g, 375 umol, 1 eq) in EtOAc (10 mL) was added HCl/EtOAc (4 M, 5 mL, 53.3 eq), and then stirred at 20° C. for 2 hr. The reaction mixture was concentrated under reduced pressure to give a residue. The residue was purified by prep-HPLC (column: Phenomenex Luna 80*30 mm*3 um; mobile phase: [water (TFA)-ACN]; B %: 5%-35%, 8 min) to give L-28h (0.05 g, 56.8 umol, 15.11% yield) as a white solid. ¹H NMR (400 MHz, DMSO-d₆) δ13.06-12.67 (m, 2H), 8.95-8.44 (m, 2H), 7.96 (d, J=9.6 Hz, 3H), 7.65 (s, 3H), 7.36 (s, 2H), 7.28 (d, J=11.6 Hz, 2H), 6.55 (d, J=7.2 Hz, 2H), 5.85-5.80 (m, 2H), 4.91 (dd, J=3.6, 9.6 Hz, 4H), 4.61 (d, J=2.8 Hz, 4H), 3.95 (d, J=5.2 Hz, 2H), 3.72 (t, J=6.4 Hz, 4H), 3.67 (s, 3H), 3.15-3.10 (m, 4H), 2.58-2.50 (m, 6H), 2.12 (d, J=5.2 Hz, 6H), 1.75-1.70 (m, 2H). HPLC: 98.124% (220 nm), 98.808% (254 nm). LC/MS [M+H] 881.4 (calculated); LC/MS [M+H] 881.4 (observed).

### Preparation of BBI-L-28

To a solution of L-28h (35 mg, 31.6 umol, 1 eq, 2TFA) and 2-(2,5-dioxopyrrol-1-yl)-N-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]acetamide (40.2 mg, 63.1 umol, 2 eq) in MeOH (1 mL) was added sodium cyanoborohydride, NaBH₃CN (5.95 mg, 94.7 umol, 3 eq), and then stirred at 20° C. for 12 hr. The reaction mixture was filtered. The residue was purified by prep-HPLC (column: Phenomenex Luna 80*30 mm*3 um; mobile phase: [water (TFA)-ACN]; B %: 5%-35%, 8 min) to give BBI-L-28 as a white solid. ¹H NMR (400 MHz, DMSO-d₆) δ13.01-12.71 (m, 1H), 8.21 (t, J=4.6 Hz, 1H), 7.96 (d, J=11.8 Hz, 2H), 7.65 (s, 2H), 7.37 (s, 2H), 7.29 (s, 1H), 7.25 (s, 1H), 7.08 (s, 2H), 6.55 (d, J=7.6 Hz, 2H), 5.98-5.68 (m, 2H), 4.93-4.90 (4, 3H), 4.63-4.59 (m, 4H), 4.01 (s, 2H), 3.95-3.90 (m, 2H), 3.72 (t, J=6.4 Hz, 4H), 3.66 (s, 3H), 3.55-3.45 (m, 38H), 3.44-3.40 (m, 6H), 3.26-3.14 (m, 6H), 2.12 (d, J=6.8 Hz, 6H), 1.80-1.60 (m, 2H). HPLC: 96.878% (220 nm), 99.512% (254 nm). LC/MS [M+H] 1501.7 (calculated); LC/MS [M+H] 1501.8 (observed).

### Example L-31 Synthesis of 1-[(E)-4-[5-carbamoyl-7-[3-[4-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxy]ethoxy]ethoxy]ethoxy]ethoxy]ethoxy]ethyl]piperazin-1-yl]propoxy]-2-[(2-ethyl-5-methyl-pyrazole-3-carbonyl)amino]benzimidazol-1-yl]but-2-enyl]-2-[[2-(2,3-dihydroxypropyl)-5-methyl-pyrazole-3-carbonyl]amino]-7-methoxy-benzimidazole-5-carboxamide, BBI-L-31

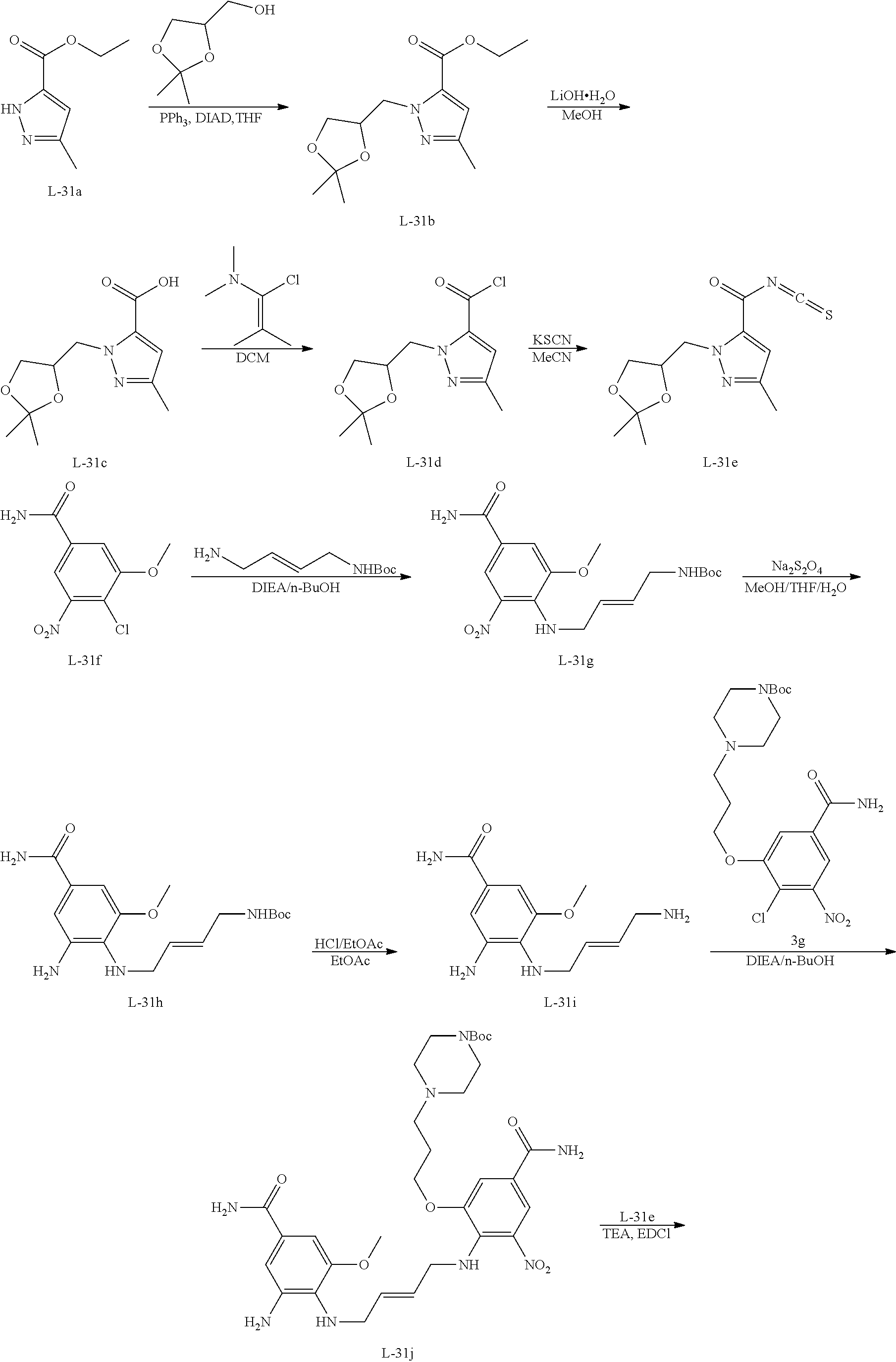

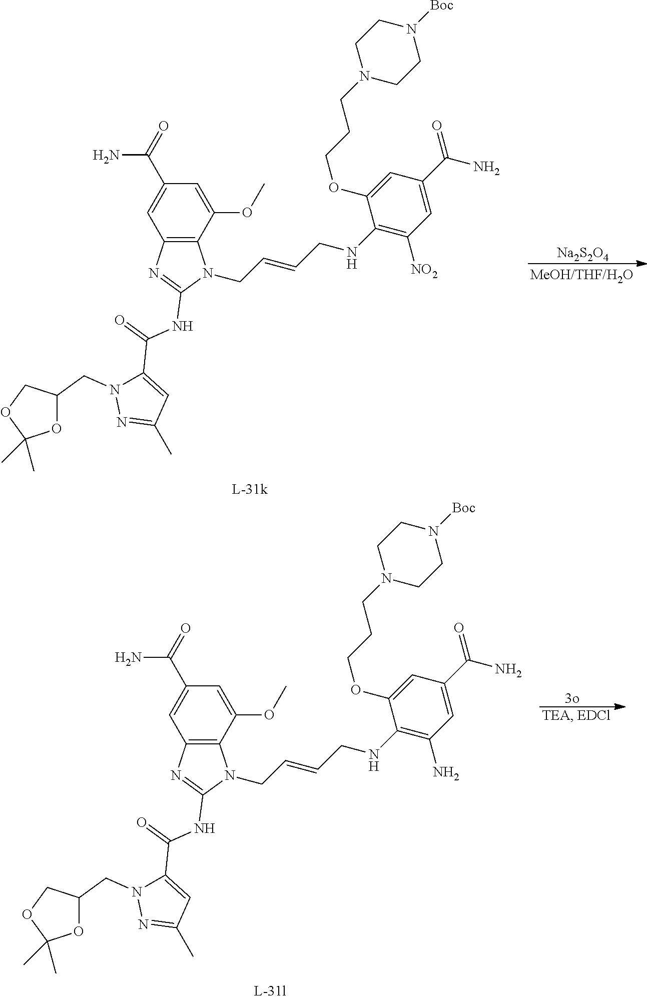 [SMILES: COc1cc(C(N)=O)cc2nc(NC(=O)c3cc(C)nn3CC3COC(C)(C)O3)n(C/C=C/CNc3c(OCCCN4CCN(C(=O)OC(C)(C)C)CC4)cc(C(N)=O)cc3[N+](=O)[O-])c12 ; "Na2S2O4" ; COc1cc(C(N)=O)cc2nc(NC(=O)c3cc(C)nn3CC3COC(C)(C)O3)n(C/C=C/CNc3c(N)cc(C(N)=O)cc3OCCCN3CCN(C(=O)OC(C)(C)C)CC3)c12 ; "TEA, EDCl" ; "3o"]

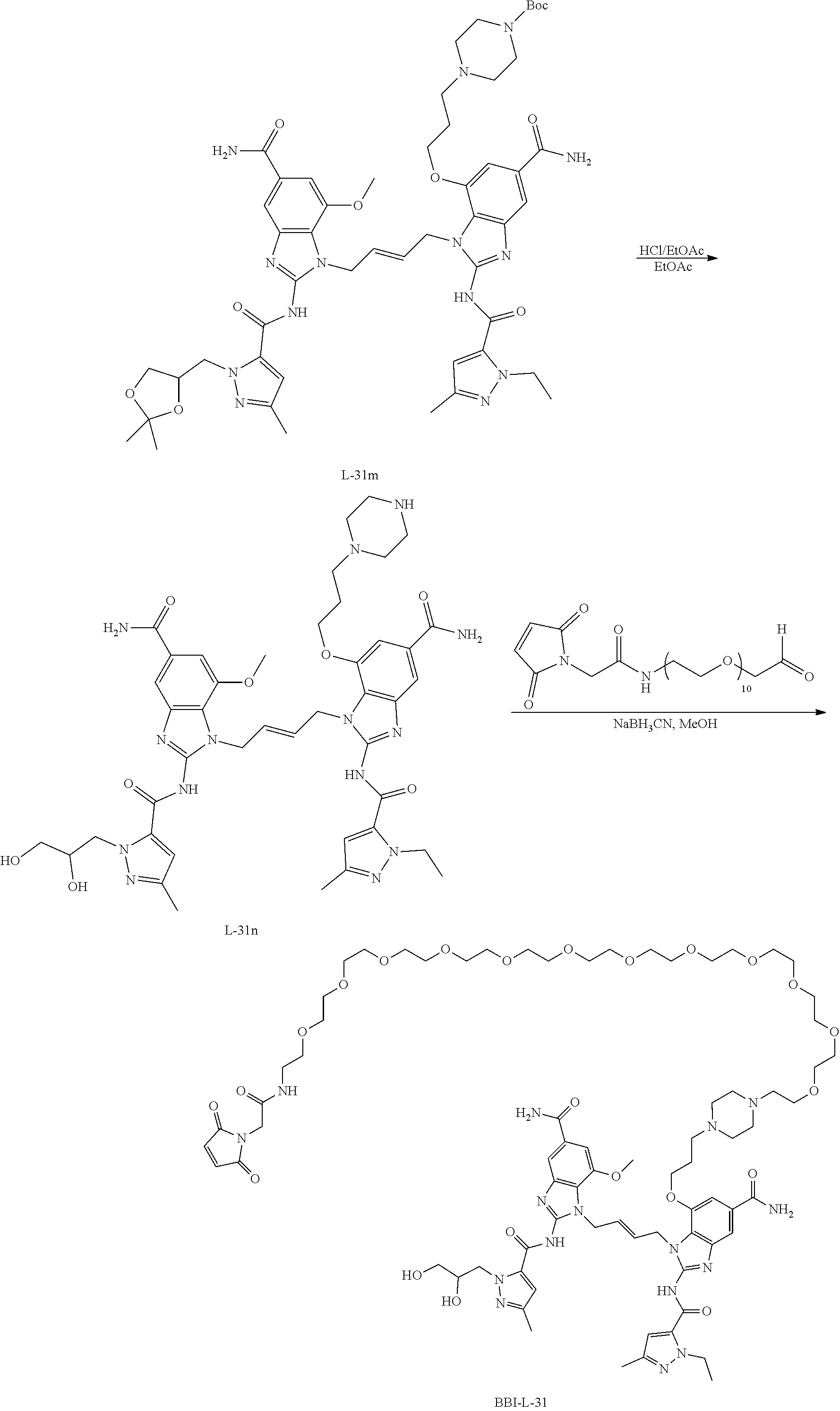

### Preparation of ethyl 2-[(2,2-dimethyl-1,3-dioxolan-4-yl)methy]-5-methyl-pyrazole-3-carboxylate, L-31b

To a solution of ethyl 3-methyl-1H-pyrazole-5-carboxylate, L-31a (5 g, 32.43 mmol, 1 eq) in THF (100) mL) was added PPh₃ (25.52 g, 97.30 mmol, 3 eq) and (2,2-dimethyl-1,3-dioxolan-4-yl)methanol (5.14 g, 38.92 mmol, 4.8 mL, 1.2 eq) followed by DEAD (11.30 g, 64.87 mmol, 11.8 mL, 2 eq). The mixture was stirred at 15° C. for 16 hr. The mixture was quenched with H₂O (300 mL) and extracted with EtOAc (50 mL×3). The combined organic layers were washed with brine (100 mL), dried over Na₂SO₄, filtered and concentrated under reduced pressure to give a residue. The residue was purified by flash silica gel chromatography (ISCO®; 120 g SepaFlash® Silica Flash Column, Eluent of 0˜8% Ethyl acetate/Petroleum ether gradient @ 100 mL/min) to give L-31b (5 g, 18.64 mmol, 57.46% yield) as colorless oil. ¹H NMR (400 MHz, CDCl₃) δ 6.60 (s, 1H), 4.75 (dd, J=5.6, 13.2 Hz, 1H), 4.59-4.51 (m, 1H), 4.51-4.42 (m, 1H), 4.32 (q, J=7.2 Hz, 2H), 4.01 (dd, J=6.0, 8.4 Hz, 1H), 3.83 (dd, J=6.0, 8.4 Hz, 1H), 2.26 (s, 3H), 1.42-1.28 (m, 9H). LC/MS [M+H] 269.1 (calculated); LC/MS [M+H] 269.0 (observed).

### Preparation of 2-[(2,2-dimethyl-1,3-dioxolan-4-yl)methyl]-5-methyl-pyrazole-3-carboxylic Acid, L-31c

To a solution of L-31b (2.5 g, 9.32 mmol, 1 eq) in THF (20 mL) and H₂O (5 mL) was added LiOH·H₂O (1.96 g, 46.59 mmol, 5 eq), and then stirred at 15° C. for 1 hr. The mixture was concentrated to remove THF, then the pH of aqueous phase was adjusted pH=˜5 with TFA and extracted with EtOAc (50 mL×2). The combined organic layers were dried over Na₂SO₄, filtered and concentrated under reduced pressure to give the crude product L-31c (2.05 g, 8.53 mmol, 91.57% yield) as white solid was used into the next step without further purification. ¹H NMR (400 MHz, MeOD) δ6.64 (s, 1H), 4.72 (dd, J=6.0, 13.6 Hz, 1H), 4.59-4.49 (m, 1H), 4.42 (q, J=6.0 Hz, 1H), 4.01 (dd, J=6.0, 8.4 Hz, 1H), 3.80 (dd, J=6.0, 8.4 Hz, 1H), 2.24 (s, 3H), 1.33 (s, 3H), 1.28 (s, 3H). LC/MS [M+H] 241.1 (calculated); LC/MS [M+H] 241.1 (observed).

### Preparation of 2-[(2,2-dimethyl-1,3-dioxolan-4-yl)methyl]-5-methyl-pyrazole-3-carbonyl Chloride, L-31d

To a solution of L-31c (1 g, 4.16 mmol, 1 eq) in DCM (15 mL) was added 1-chloro-N,N,2-trimethyl-prop-1-en-1-amine (1.11 g, 8.32 mmol, 1.1 mL, 2 eq) at 0° C., and then stirred at 15° C. for 5 hr. The mixture was concentrated to give L-31d (0.6 g, 2.32 mmol, 55.72% yield) as light yellow oil which was used into the next step without further purification.

### Preparation of 2-[(2,2-dimethyl-1,3-dioxolan-4-yl)methyl]-5-methyl-pyrazole-3-carbonyl isothiocyanate, L-31e

To a solution of L-31d (0.6 g, 2.32 mmol, 1 eq) in MeCN (2 mL) was added potassium thiocyanate, KSCN (676.2 mg, 6.96 mmol, 3 eq), and then stirred at 15° C. for 1 hr. The mixture was filtered, the filtrate was concentrated. The residue was purified by flash silica gel chromatography (ISCO®; 40 g SepaFlash® Silica Flash Column, Eluent of 0˜40% Ethyl acetate/Petroleum ether gradient @ 75 mL/min) to afford L-31e (0.5 g, 1.78 mmol, 76.63% yield) as yellow oil as light yellow oil. ¹H NMR (400 MHz, CDCl₃) δ6.76 (s, 1H), 4.74 (dd, J=6.0, 13.2 Hz, 1H), 4.59-4.43 (m, 2H), 4.06 (dd, J=6.0, 8.8 Hz, 1H), 3.83 (dd, J=5.6, 8.8 Hz, 1H), 2.30 (s, 3H), 1.39 (s, 3H), 1.33 (s, 3H).

### Preparation of tert-butyl N-[(E)-4-(4-carbamoyl-2-methoxy-6-nitro-anilino)but-2-enyl]carbamate, L-31g

To a solution of 4-chloro-3-methoxy-5-nitro-benzamide, L-31f (4 g, 17.35 mmol, 1 eq) and tert-butyl N-[(E)-4-aminobut-2-enyl]carbamate (4.52 g, 24.28 mmol, 1.4 eq) in n-BuOH (70 mL) was added diisopropylethylamine, DIEA (11.21 g, 86.73 mmol, 15.11 mL, 5 eq), and then stirred at 120° C. for 18 h under N₂. The reaction mixture was concentrated to remove n-BuOH, and the residue was diluted with H₂O 100 mL at 15° C. and stirred for 30 min, filtered to give the crude product. The crude product was triturated with EtOAc (50 mL) at 15° C. for 30 min to afford L-31g (6 g, 15.77 mmol, 90.93% yield) as red solid. ¹H NMR (400 MHz, DMSO-d₆, δ8.18 (d, J=2.0 Hz, 1H), 8.01 (s, 1H), 7.74 (t, J=6.0 Hz, 1H), 7.55 (d, J=1.6 Hz, 1H), 7.31 (s, 1H), 6.92 (s, 1H), 5.53 (s, 2H), 4.09 (d, J=4.0 Hz, 2H), 3.87 (s, 3H), 3.48 (s, 2H), 1.44-1.30 (m, 9H). LC/MS [M+H] 381.2 (calculated); LC/MS [M−55] 325.0 (observed).

### Preparation of tert-butyl N-[(E)-4-(2-amino-4-carbamoyl-6-methoxy-anilino)but-2-enyl]carbamate, L-31h

To a solution of L-31g (6 g, 15.77 mmol, 1 eq) in MeOH (30 mL), THF (30 mL) and H₂O (15 mL) was added Na₂CO₃ (6.69 g, 63.09 mmol, 4 eq) and disodium dithionite (19.22 g, 110.41 mmol, 7 eq). The mixture was stirred at 15° C. for 1 hr. The mixture was concentrated to remove THF and MeOH, then H₂O (50 mL) was added and it was stirred for 10 min and filtered to give crude product L-31h (4 g, 11.42 mmol, 72.37% yield) as off-white solid which was used into the next step without further purification. ¹H NMR (400 MHz, MeOD) δ6.93 (d, J=2.0 Hz, 1H), 6.90 (d, J=2.0 Hz, 1H), 5.74-5.57 (m, 2H), 3.85 (s, 3H), 3.60-3.57 (m, 4H), 1.43 (s, 9H). LC/MS [M+H] 351.2 (calculated); LC/MS [M+H] 351.1 (observed).

### Preparation of 3-amino-4-[[(E)-4-aminobut-2-enyl]amino]-5-methoxy-benzamide, L-31i

To a solution of L-31h (1.2 g, 3.42 mmol, 1 eq) in EtOAc (15 mL) was added HCl/EtOAc (4 M, 42.8 mL, 50 eq), and then stirred at 15° C. for 0.5 hr, the mixture was concentrated to give L-31i (1.1 g, 3.40 mmol, 99.38% yield, 2HCl) as white solid which was used into the next step without further purification. LC/MS [M+H] 251.2 (calculated); LC/MS [M+H] 251.1 (observed).

### Preparation of tert-butyl 4-[3-[2-[[(E)-4-(2-amino-4-carbamoyl-6-methoxy-anilino)but-2-enyl]amino]-5-carbamoyl-3-nitro-phenoxy]propyl]piperazine-1-carboxylate, L-31j

A solution of L-31i (1.1 g, 3.40 mmol, 1 eq, 2HCl) and DIEA (3.52 g, 27.2 mmol, 4.7 mL, 8 eq) in n-BuOH (30 mL) was stirred at 15° C. for 0.5 h., then tert-butyl 4-[3-(5-carbamoyl-2-chloro-3-nitro-phenoxy)propyl]piperazine-1-carboxylate (829 mg, 1.87 mmol, 0.55 eq) was added, and the mixture was stirred at 120° C. for 12 h. The mixture was concentrated in vacuum to removed n-BuOH, then the residue was diluted with water 20 mL and extracted with DCM/i-PrOH=3/1 (30 mL×3). The combined organic layers were washed with brine 20 mL, dried over Na₂SO₄, filtered and concentrated under reduced pressure to give a residue. The crude product was triturated with MTBE/EtOAc=5/1 (30 ml) at 15° C. for 10 min to give L-31j (1 g, 1.52 mmol, 44.74% yield) as red solid was used into the next step without further purification. ¹H NMR (400 MHz, MeOD) δ8.32 (d, J=2.0 Hz, 1H), 7.50 (d, J=2.0 Hz, 1H), 6.92 (d, J=2.0 Hz, 1H), 6.85 (d, J=2.0 Hz, 1H), 5.77-5.61 (m, 2H), 4.23 (d, J=3.6 Hz, 2H), 4.04 (t, J=6.0 Hz, 2H), 3.77 (s, 3H), 3.71-3.57 (m, 2H), 3.48-3.44 (m, 4H), 2.54 (t, J=7.2 Hz, 2H), 2.46 (t, J=5.2 Hz, 4H), 2.06-1.98 (m, 2H), 1.47 (s, 9H). LC/MS [M+H] 657.3 (calculated); LC/MS [M+H] 657.3 (observed).

### Preparation of tert-butyl 4-[3-[5-carbamoyl-2-[[(E)-4-[5-carbamoyl-2-[[2-[(2,2-dimethyl-1,3-dioxolan-4-yl)methyl]-5-methyl-pyrazole-3-carbonyl]amino]-7-methoxy-benzimidazol-1-yl]but-2-enyl]amino]-3-nitro-phenoxy]propyl]piperazine-1-carboxylate, L-31k

To a solution of L-31e (111 mg, 396 umol, 1.3 eq) in DMF (4 mL) was added L-31j (200 mg, 305 umol, 1 eq) and stirred for 30 min at 0° C., then Et₃N (92.5 mg, 914 umol, 127 uL, 3 eq) and 1-ethyl-3-(3-dimethylaminopropyl)carbodiimide, EDCI, EDAC, EDC, CAS Reg. No. 25952-53-8 (175 mg, 914 umol, 3 eq) was added. The mixture was stirred at 15° C. for 16 hr. The reaction was quenched with NaHCO₃·aq (5 mL) and water (5 mL), the desired solid precipitated from the mixture, filtered to give L-31k (0.2 g, 221 umol, 72.65% yield) as light yellow solid. LC/MS [M+H] 904.4 (calculated); LC/MS [M+H] 904.3 (observed).

### Preparation of tert-butyl 4-[3-[3-amino-5-carbamoyl-2-[[(E)-4-[5-carbamoyl-2-[[2-[(2,2-dimethyl-1,3-dioxolan-4-yl)methyl]-5-methyl-pyrazole-3-carbonyl]amino]-7-methoxy-benzimidazol-1-yl]but-2-enyl]amino]phenoxy]propyl]piperazine-1-carboxylate-L-31l

To a solution of L-31k (0.2 g, 221 umol, 1 eq) in THF (1 mL) MeOH (1 mL) and H₂O (1 mL) was added Na₂CO₃ (93.8 mg, 885 umol, 4 eq) and disodium; dithionite (270 mg, 1.55 mmol, 7 eq), and then stirred at 15° C. for 0.5 hr. The mixture was concentrated to remove THF and MeOH. The desired solid precipitated from the aqueous phase, filtered and the cake was washed with H₂O (5 mL) to give L-31l (170 mg, 194.51 umol, 87.92% yield) as yellow solid. LC/MS [M+H] 874.4 (calculated); LC/MS [M+H] 874.4 (observed).

### Preparation of tert-butyl 4-[3-[6-carbamoyl-3-[(E)-4-[5-carbamoyl-2-[[2-[(2,2-dimethyl-1,3-dioxolan-4-yl)methyl]-5-methyl-pyrazole-3-carbonyl]amino]-7-methoxy-benzimidazol-1-yl]but-2-enyl]-2-[(2-ethyl-5-methyl-pyrazole-3-carbonyl)amino]benzimidazol-4-yl]oxypropyl]piperazine-1-carboxylate, L-31m

A solution of L-31l (170 mg, 195 umol, 1 eq) and 2-ethyl-5-methyl-pyrazole-3-carbonyl isothiocyanate, 3o (49.4 mg, 253 umol, 1.3 eq) in DMF (2 mL) was stirred for 0.5 h at 0° C., then Et₃N (59.1 mg, 584 umol, 81.2 uL, 3 eq) and 1-ethyl-3-(3-dimethylaminopropyl)carbodiimide, EDCI, EDAC, EDC, CAS Reg. No. 25952-53-8 (112 mg, 584 umol, 3 eq) was added. The result mixture was stirred at 15° C. for 16 hr. The reaction was quenched with NaHCO₃·aq (5 mL) and water (5 mL). The desired solid precipitated from the mixture, filtered to give L-31m (0.15 g, 144.91 umol, 74.50% yield) as yellow solid. LC/MS [M+H] 1035.5 (calculated); LC/MS [M+H] 1035.5 (observed).

### Preparation of 1-[(E)-4-[5-carbamoyl-2-[(2-ethyl-5-methyl-pyrazole-3-carbonyl)amino]-7-(3-piperazin-1-ylpropoxy)benzimidazol-1-yl]but-2-enyl]-2-[[2-(2,3-dihydroxypropyl)-5-methyl-pyrazole-3-carbonyl]amino]-7-methoxy-benzimidazole-5-carboxamide, L-31n

To a solution of L-31m (0.15 g, 145 umol, 1 eq) in EtOAc (2 mL) was added HCl/EtOAc (4 M, 7.25 mL, 200 eq), and then stirred at 15° C. for 0.5 hr. The mixture was concentrated and the residue was purified by prep-HPLC (column: Phenomenex Luna 80*30 mm*3 um; mobile phase: [water (TFA)-ACN]; B %: 5%-30%, 8 min) to afford L-31n (75 mg, 66.78 umol, 46.09% yield, 2TFA) as a white solid. ¹H NMR (400 MHz, MeOD) δ7.60 (d, J=1.2 Hz, 1H), 7.56 (d, J=1.2 Hz, 1H), 7.26 (d, J=1.2 Hz, 1H), 7.22 (d, J=1.2 Hz, 1H), 6.65 (s, 1H), 6.63 (s, 1H), 5.92-5.82 (m, 2H), 5.05-5.01 (m, 4H), 4.79-4.72 (m, 1H), 4.69-4.59 (m, 3H), 4.09-4.01 (m, 1H), 3.90 (t, J=5.6 Hz, 2H), 3.69 (s, 3H), 3.54 (t, J=4.8 Hz, 2H), 3.20 (t, J=5.2 Hz, 4H), 2.70-2.64 (m, 4H), 2.51 (t, J=7.2 Hz, 2H), 2.22 (s, 3H), 2.20 (s, 3H), 1.78-1.69 (m, 2H), 1.39 (t, J=7.2 Hz, 3H). LC/MS [M+H] 895.4 (calculated); LC/MS [M+H] 895.4 (observed).

### Preparation of BBI-L-31

To a solution of 2-(2,5-dioxopyrrol-1-yl)-N-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]acetamide (42.5 mg, 66.8 umol, 2.5 eq) in MeOH (0.3 mL) was added L-31n (30 mg, 26.7 umol, 1 eq, 2TFA) and NaBH₃CN (5.04 mg, 80.1 umol, 3 eq), and then stirred at 15° C. for 12 hrs. The mixture was filtered and purified by prep-HPLC (column: Phenomenex Luna 80*30 mm*3 um; mobile phase: [water (TFA)-ACN]; B %: 10%-40%, 8 min) to give BBI-L-31 (15.6 mg, 10.29 umol, 38.53% yield) as light yellow oil. ¹H NMR (400 MHz, MeOD) δ7.63 (s, 1H), 7.59 (s, 1H), 7.31 (s, 1H), 7.27 (s, 1H), 6.89 (s, 2H), 6.66 (s, 1H), 6.61 (s, 1H), 5.96-5.83 (m, 2H), 5.10-5.02 (m, 4H), 4.79-4.61 (m, 4H), 4.21-4.16 (m, 2H), 4.11-4.02 (m, 1H), 3.96 (t, J=6.0 Hz, 2H), 3.86-3.81 (m, 2H), 3.75 (s, 3H), 3.72-3.69 (m, 4H), 3.67-3.59 (m, 38H), 3.56-3.52 (m, 4H), 3.39-3.36 (m, 3H), 2.99-2.72 (m, 4H), 2.62 (t, J=6.8 Hz, 2H), 2.24 (s, 3H), 2.22 (s, 3H), 1.79 (td, J=6.8, 13.6 Hz, 2H), 1.41 (t, J=7.2 Hz, 3H). LC/MS [M+H] 1515.7 (calculated); LC/MS [M+H] 1515.7 (observed).

### Example L-34 Synthesis of 1-[(E)-4-[5-carbamoyl-2-[(2-ethyl-5-methyl-pyrazole-3-carbonyl)amino]benzimidazol-1-yl]but-2-enyl-7-[3-[4-[2-[2-[2-[2-[2-[2-[2-[2-[2-[2-[2-[[2-(2,5-dioxopyrrol-1-yl)acetyl]amino]ethoxy]ethoxy]ethoxy]ethoxy]ethoxyl]ethoxy]ethoxy]ethoxy]ethoxy]ethoxy]ethyl]piperazin-1-yl]propoxy]-2-[(2-ethyl-5-methyl-pyrazole-3-carbonyl)amino]benzimidazole-5-carboxamide, BBI-L-34

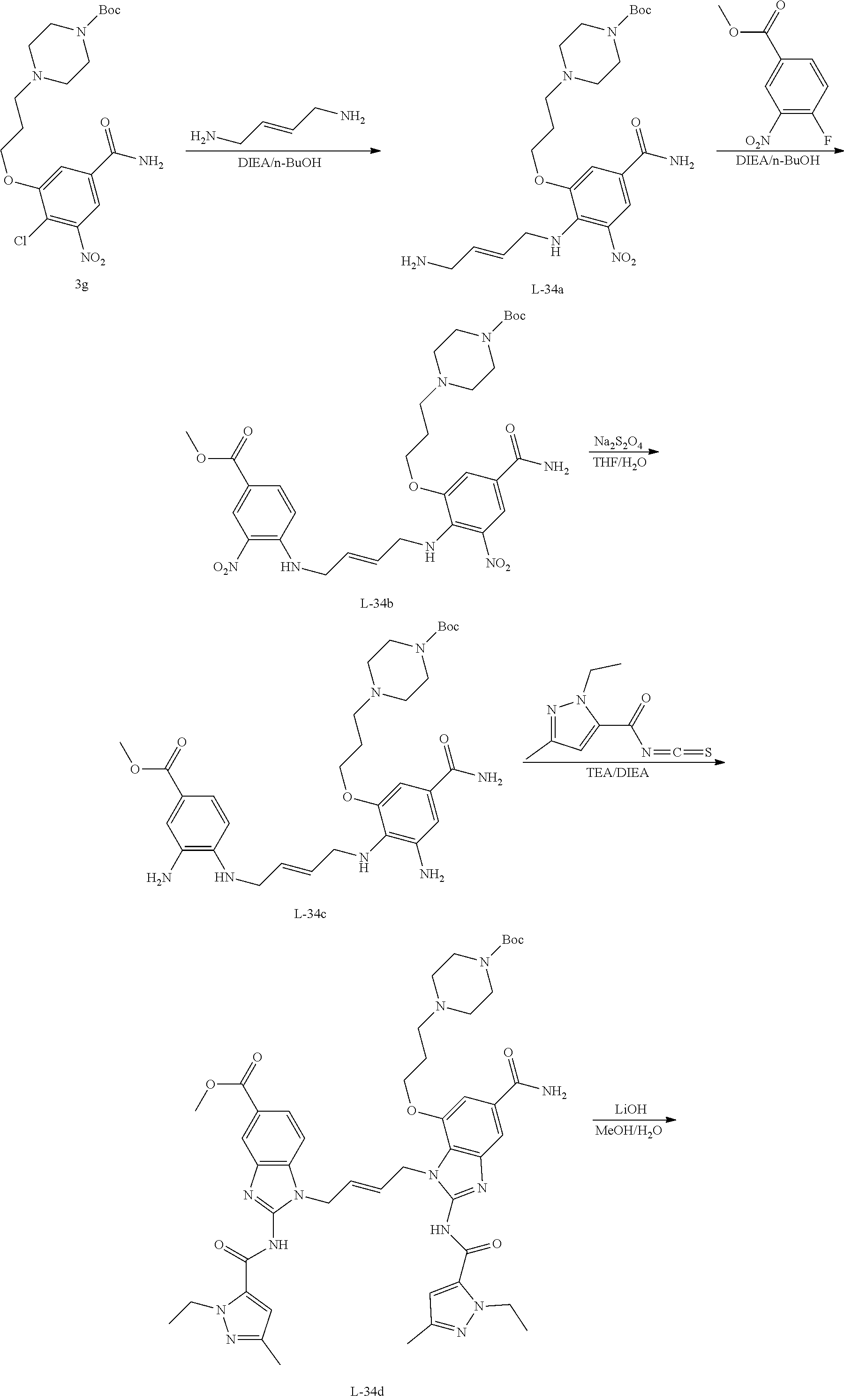

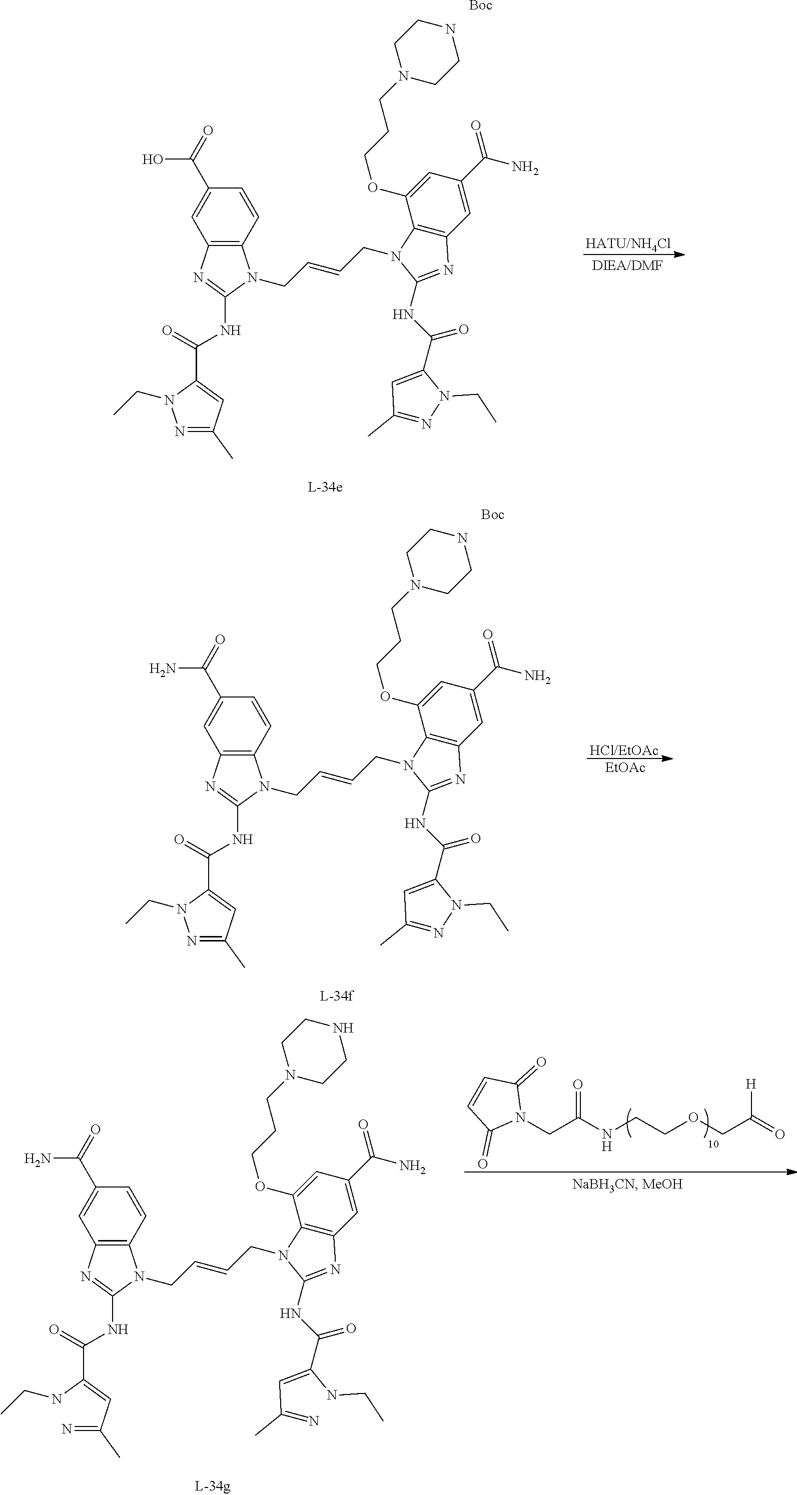

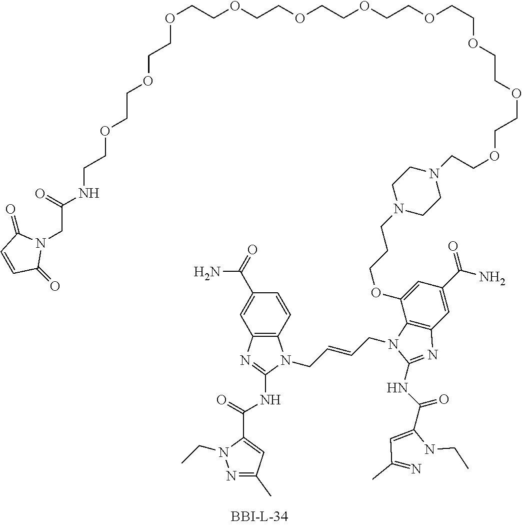 [SMILES: CCN/C(=C\C(C)=N)C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2CC)nc2cc(C(N)=O)ccc21]

### Preparation of tert-butyl 4-[3-[2-[[(E)-4-aminobut-2-enyl]amino]-5-carbamoyl-3-nitro-phenoxy]propyl]piperazine-1-carboxylate, L-34a

To a solution of (E)-but-2-ene-1,4-diamine (8.98 g, 56.45 mmol, 5 eq, 2HCl) in n-BuOH (150 mL) was added DIEA (29.18 g, 225.79 mmol, 39.3 mL, 20 eq) and tert-butyl 4-[3-(5-carbamoyl-2-chloro-3-nitro-phenoxy)propyl]piperazine-1-carboxylate, 3g (5 g, 11.29 mmol, 1 eq). The mixture was stirred at 120° C. for 12 hr under N. The mixture was filtered to remove the insoluble substance. The filtrate was concentrated to give a residue. The residue was purified by prep-HPLC (column: Xtimate C18 10 u 250 mm*80 mm; mobile phase: [water (NH4HCO3)-ACN]; B %: 10%-50%, 27 min) to give L-34a (4 g, 8.12 mmol, 35.97% yield) as red solid. LC/MS [M+H] 493.3 (calculated); LC/MS [M+H] 493.2 (observed).

### Preparation of tert-butyl 4-[3-[5-carbamoyl-2-[[(E)-4-(4-methoxycarbonyl-2-nitro-anilino)but-2-enyl]amino]-3-nitro-phenoxy]propyl]piperazine-1-carboxylate, L-34b

To a solution of L-34a (400 mg, 812 umol, 1 eq) in n-BuOH (1.20 g, 16.2 mmol, 1.49 mL, 20 eq) was added DIEA (524 mg, 4.06 mmol, 707 uL, 5 eq) and methyl 4-fluoro-3-nitro-benzoate (242 mg, 1.22 mmol, 1.5 eq). The mixture was stirred at 120° C. for 12 hr under N₂. The reaction mixture was concentrated to remove the n-BuOH. The residue was diluted with H₂O (10 mL), and then extracted with EtOAc (10 mL×3). The combined organic layers were dried over Na₂SO₄, filtered and concentrated under reduced pressure to give L-34b (600 mg, crude) as a yellow solid. LC/MS [M+H] 672.3 (calculated); LC/MS [M+H] 672.3 (observed).

### Preparation of tert-butyl 4-[3-[3-amino-2-[[(E)-4-(2-amino-4-methoxycarbonyl-anilino)but-2-enyl]amino]-5-carbamoyl-phenoxy]propyl]piperazine-1-carboxylate, L-34c

To a solution of L-34b (600 mg, 893 umol, 1 eq) in MeOH (3 mL), THF (3 mL) and H₂O (1.5 mL) was added Na₂CO₃ (473 mg, 4.47 mmol, 5 eq) and disodium; dithionite (1.09 g, 6.25 mmol, 7 eq), and then stirred at 15° C. for 1 hr. The reaction mixture was concentrated under reduced pressure to give a residue. The residue was diluted with EtOAc (10 mL), filtered to remove the insoluble substance, and the filtrate was concentrated under reduced pressure to give L-34c (570 mg, crude) as a white solid. LC/MS [M+H] 612.3 (calculated); LC/MS [M+H] 612.3 (observed).

### Preparation of methyl 1-[(E)-4-[7-[3-(4-tert-butoxycarbonylpiperazin-1-yl)propoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazole-5-carboxylate, L-34d

To a solution of L-34c (570 mg, 931 umol, 1 eq) in DMF (5 mL) was added 2-ethyl-5-methyl-pyrazole-3-carbonyl isothiocyanate (400 mg, 2.05 mmol, 2.2 eq). After addition the mixture was stirred 30 min at 0° C., then Et₃N (565 mg, 5.59 mmol, 778 uL, 6 eq), and EDCI (1.07 g, 5.59 mmol, 6 eq) were added at 0° C. The resultant mixture was stirred at 15° C. for another 12 hrs. The mixture was quenched with sat. NaHCO₃ (20 mL), the aqueous phase was extracted with EtOAc (50 mL×3), the combined organic phase was washed with brine (30 mL), dried over Na₂SO₄, filtered and concentrated to give L-34d (800 mg, crude) as a yellow solid. LC/MS [M+H] 934.5 (calculated); LC/MS [M+H] 934.5 (observed).

### Preparation of 1-[(E)-4-[7-[3-(4-tert-butoxycarbonylpiperazin-1-yl)propoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazole-5-carboxylic Acid, L-34e

To a solution of L-34d (700 mg, 749 umol, 1 eq) in H₂O (2 mL) and THF (10 mL) was added lithium hydroxide hydrate, LiOH·H₂O (125 mg, 3.00 mmol, 4 eq), and then stirred at 50° C. for 4 hr. The reaction mixture was quenched with HCl (2M) until pH was about 5, the mixture was concentrated under reduced pressure to give a residue, the residue was triturated with H₂O (10 mL), filtered to give L-34e (800 mg, crude) as a white solid. LC/MS [M+H] 920.5 (calculated); LC/MS [M+H] 920.5 (observed).

### Preparation of tert-butyl 4-[3-[6-carbamoyl-3-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]benzimidazol-4-yl]oxypropyl]piperazine-1-carboxylate, L-34f

To a solution of L-34e (250 mg, 272 umol, 1 eq) in DMF (0.5 mL) was added NH₄Cl (145 mg, 2.72 mmol, 10 eq), DIEA (105 mg, 815 umol, 142 uL, 3 eq) and 1-bis(dimethylamino)methylene-1H-1,2,3-triazolo[4,5-b]pyridinium 3-oxide hexafluorophosphate, Hexafluorophosphate Azabenzotriazole Tetramethyl Uronium, HATU, CAS Reg. No. 148893-10-1 (155 mg, 408 umol, 1.5 eq). The mixture was stirred at 15° C. for 1 hr. the mixture was poured into water (15 mL), the aqueous phase was extracted with EtOAc (20 mL×3), the combined organic phase was washed with brine (15 mL), dried over Na₂SO₄, filtered and concentrated to give L-34f (250 mg, crude) as a white solid. LC/MS [M+H] 919.4 (calculated); LC/MS [M+H] 919.4 (observed).

### Preparation of 1-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]-7-(3-piperazin-1-ylpropoxy)benzimidazole-5-carboxamide, L-34g

To a solution of L-34f (250 mg, 272 umol, 1 eq) in EtOAc (1 mL) was added HCl/EtOAc (4 M, 4.17 mL, 61.3 eq). The mixture was stirred at 25° C. for 0.5 hr. The mixture was concentrated in vacuum to give a residue. The residue was purified by prep-HPLC (column: Phenomenex C18 80*30 mm*3 um; mobile phase: [water (TFA)-ACN]; B %: 1%-25%, 8 min) to give L-34g (21 mg, 25.64 umol, 9.43% yield) as a white solid. ¹H NMR (400 MHz, MeOD) δ 7.94 (d, J=1.2 Hz, 1H), 7.72 (dd, J=1.6, 8.4 Hz, 1H), 7.53 (d, J=1.2 Hz, 1H), 7.35 (d, J=8.4 Hz, 1H), 7.26 (s, 1H), 6.58 (s, 1H), 6.53 (s, 1H), 6.00-5.86 (m, 1H), 5.76-5.73 (m, 1H), 5.02 (br d, J=4.0 Hz, 2H), 4.92-4.84 (m, 2H), 4.65-4.48 (m, 4H), 4.01 (br t, J=6.0 Hz, 2H), 3.36 (br t, J=4.8 Hz, 4H), 3.02-2.98 (m, 4H), 2.80 (br t, J=7.2 Hz, 2H), 2.18 (d, J=9.5 Hz, 6H), 1.94-1.79 (m, 2H), 1.43-1.25 (m, 6H). HPLC: 96.66% (220 nm). LC/MS [M+H] 819.4 (calculated); LC/MS [M+H] 819.4 (observed).

### Preparation of BBI-L-34

To a solution of L-34g (50 mg, 61.06 umol, 1 eq) in MeOH (1 mL) was added 2-(2,5-dioxopyrrol-1-yl)-N-[2-[2-[2-[2-[2-[2-[2-[2-[2-[2-(2-oxoethoxy)ethoxy]ethoxy]ethoxy]ethoxy]ethoxy]ethoxy]ethoxy]ethoxy]ethoxy]ethyl]acetamide (38.87 mg, 61.06 umol, 1 eq) at 0° C., the mixture was stirred at 0° C. for 15 min. Then NaBH3CN (11.51 mg, 183.17 umol, 3 eq) was added, and the result mixture was stirred at 15° C. for 16 hrs. The mixture was filtered and purified by prep-HPLC (column: Phenomenex Luna 80*30 mm*3 um; mobile phase: [water (TFA)-ACN]; B %: 10%-40%, 8 min) to give BBI-L-34 (60 mg, 35.98 umol, 58.93% yield, 2TFA) as a white solid. ¹H NMR (400 MHz, MeOD) δ 7.97 (d, J=1.6 Hz, 1H), 7.75 (dd, J=1.6, 8.4 Hz, 1H), 7.56 (d, J=1.2 Hz, 1H), 7.39 (d, J=8.4 Hz, 1H), 7.31 (s, 1H), 6.87 (s, 2H), 6.62 (s, 1H), 6.56 (s, 1H), 6.02-5.89 (m, 1H), 5.86-5.73 (m, 1H), 5.09-5.03 (m, 2H), 4.90-4.80 (m, 2H), 4.67-4.53 (m, 4H), 4.16 (s, 2H), 4.05 (br t, J=6.0 Hz, 2H), 3.89-3.80 (m, 2H), 3.74-3.46 (m, 42H), 3.37-3.33 (m, 4H), 2.98-2.75 (m, 4H), 2.66 (br t, J=7.2 Hz, 2H), 2.20 (d, J=9.6 Hz, 6H), 1.87-1.76 (m, 2H), 1.38 (t, J=7.2 Hz, 3H), 1.32 (t, J=7.2 Hz, 3H). HPLC: 97.16% (220 nm). LC/MS [M+H] 1439.7 (calculated), LC/MS [M+H] 1439.9 (observed).

### Example 201 Preparation of Immunoconjugates (IC)

In an exemplary procedure, for preparation for Lysine-based conjugation, an antibody is buffer exchanged into a conjugation buffer containing 100 mM Borate, 50 mM sodium chloride, 1 mM ethylenediaminetetraacetic acid at pH 8.3 using Zebat Spin Desalting Columns (Thermo Fisher Scientific). The concentration of the buffer-exchanged antibody was adjusted to approximately 5-25 mg/ml using the conjugation buffer and sterile-filtered. The bis-benzimidazole-linker (BBI-L) intermediate compound of Formula II is either dissolved in dimethylsulfoxide (DMSO) or dimethylacetamide (DMA) to a concentration of 5-20 mM. For conjugation, the antibody is mixed with 4-20 molar equivalents of BBI-L. In some instances, additional DMA or DMSO up to 20% (v/v), was added to improve the solubility of BBI-L in the conjugation buffer. The reaction is allowed to proceed for approximately 30 min to 4 hours at 20° C. or 30° C. or 37° C. The resulting conjugate is purified away from the unreacted BBI-L using two successive Zeba™ Spin Desalting Columns. The columns are pre-equilibrated with phosphate-buffered saline (PBS), pH 7.2. Adjuvant to antibody ratio (DAR) is estimated by liquid chromatography mass spectrometry analysis using a C4 reverse phase column on an ACQUITY™ UPLC H-class (Waters Corporation, Milford, MA) connected to a XEVO™ G2-XS TOF mass spectrometer (Waters Corporation).

In an exemplary procedure, for preparation for Cysteine-based conjugation, an antibody is buffer exchanged into a conjugation buffer containing PBS, pH 7.2 with 2 mM EDTA using Zeba™ Spin Desalting Columns (Thermo Fisher Scientific). The interchain disulfides are reduced using 2-4 molar excess of Tris (2-carboxyethyl) phosphine (TCEP) or dithiothreitol (DTT) at 37° C. for 30 min-2 hours. Excess TCEP or DTT was removed using a Zeba™ Spin Desalting column pre-equilibrated with the conjugation buffer. The concentration of the buffer-exchanged antibody was adjusted to approximately 5-20 mg/ml using the conjugation buffer and sterile-filtered. The BBI-L is either dissolved in dimethylsulfoxide (DMSO) or dimethylacetamide (DMA) to a concentration of 5-20 mM. For conjugation, the antibody is mixed with 10-20 molar equivalents of BBI-L. In some instances, additional DMA or DMSO up to 20% (v/v), was added to improve the solubility of the BBI-L in the conjugation buffer. The reaction is allowed to proceed for approximately 30 min to 4 hours at 20° C. The resulting conjugate is purified away from the unreacted BBI-L using two successive Zeba™ Spin Desalting Columns. The columns are pre-equilibrated with phosphate-buffered saline (PBS), pH 7.2. Adjuvant to antibody ratio (DAR) is estimated by liquid chromatography mass spectrometry analysis using a C4 reverse phase column on an ACQUITY™ UPLC H-class (Waters Corporation, Milford, MA) connected to a XEVO™ G2-XS TOF mass spectrometer (Waters Corporation).

Following conjugation, to potentially remove unreacted BBI-L and/or higher-molecular weight aggregate, the conjugates may be purified further using size exclusion chromatography, hydrophobic interaction chromatography, ion exchange chromatography, chromatofocusing, ultrafiltration, centrifugal ultrafiltration, tangential flow filtration, and combinations thereof.

In another exemplary procedure, an antibody is buffer exchanged into a conjugation buffer containing 100 mM boric acid, 50 mM sodium chloride, 1 mM ethylenediaminetetraacetic acid at pH 8.3, using G-25 SEPHADEX™ desalting columns (Sigma-Aldrich, St. Louis, MO). The eluates are then each adjusted to a concentration of about 1-10 mg/ml using the buffer and then sterile filtered. The antibody is pre-warmed to 20-30° C. and rapidly mixed with 2-20 (e.g., 7-10) molar equivalents of bis-benzimidazole-linker (BBI-L) intermediate compound of Formula II. The reaction is allowed to proceed for about 16 hours at 30° C. and the immunoconjugate (IC) is separated from reactants by running over two successive G-25 desalting columns equilibrated in phosphate buffered saline (PBS) at pH 7.2 to provide the Immunoconjugate (IC) of Table 2. Adjuvant-antibody ratio (DAR) is determined by liquid chromatography mass spectrometry analysis using a C4 reverse phase column on an ACQUITY™ UPLC H-class (Waters Corporation, Milford, MA) connected to a XEVO™ G2-XS TOF mass spectrometer (Waters Corporation).

For conjugation, the antibody may be dissolved in a aqueous buffer system known in the art that will not adversely impact the stability or antigen-binding specificity of the antibody. Phosphate buffered saline may be used. The BBI-L is dissolved in a solvent system comprising at least one polar aprotic solvent as described elsewhere herein. In some such aspects, the BBI-L is dissolved to a concentration of about 5 mM, about 10 mM, about 20 mM, about 30 mM, about 40 mM or about 50 mM, and ranges thereof such as from about 5 mM to about 50 mM or from about 10 mM to about 30 mM in pH 8 Tris buffer (e.g., 50 mM Tris). In some aspects, the BBI-L is dissolved in DMSO (dimethylsulfoxide), DMA (dimethylacetamide) or acetonitrile, or another suitable dipolar aprotic solvent.

Alternatively in the conjugation reaction, an equivalent excess of BBI-L solution may be diluted and combined with antibody solution. The BBI-L solution may suitably be diluted with at least one polar aprotic solvent and at least one polar protic solvent, examples of which include water, methanol, ethanol, n-propanol, and acetic acid. The molar equivalents of thienoazepine-linker intermediate to antibody may be about 1.5:1, about 3:1, about 5:1, about 10:1, about 15:1, or about 20:1, and ranges thereof, such as from about 1.5:1 to about 20:1 from about 1.5:1 to about 15:1, from about 1.5:1 to about 10:1, from about 3:1 to about 15:1, from about 3:1 to about 10:1, from about 5:1 to about 15:1 or from about 5:1 to about 10:1. The reaction may suitably be monitored for completion by methods known in the art, such as LC-MS. The conjugation reaction is typically complete in a range from about 1 hour to about 16 hours. After the reaction is complete, a reagent may be added to the reaction mixture to quench the reaction. If antibody thiol groups are reacting with a thiol-reactive group such as maleimide of the BBI-L, unreacted antibody thiol groups may be reacted with a capping reagent. An example of a suitable capping reagent is ethylmaleimide.

Following conjugation, the immunoconjugates may be purified and separated from unconjugated reactants and/or conjugate aggregates by purification methods known in the art such as, for example and not limited to, size exclusion chromatography, hydrophobic interaction chromatography, ion exchange chromatography, chromatofocusing, ultrafiltration, centrifugal ultrafiltration, tangential flow filtration, and combinations thereof. For instance, purification may be preceded by diluting the immunoconjugate, such in 20 mM sodium succinate, pH 5. The diluted solution is applied to a cation exchange column followed by washing with, e.g., at least 10 column volumes of 20 mM sodium succinate, pH 5. The conjugate may be suitably eluted with a buffer such as PBS.

### Example 202 Functional Assessment of Immunoconjugates

The immunoconjugates of the invention can be assessed in a co-culture assay using primary human peripheral blood mononuclear cells (PBMC) co-cultured with target antigen-expressing tumor cells. Briefly, PBMCs are freshly isolated from healthy human donor blood (Stanford Blood Center) by density centrifugation. PBMCs are then co-cultured with antigen-expressing tumor cells at a 10:1 effector to target ratio in complete medium (RPMI supplemented with 10% FBS) and incubated overnight with a range of concentrations of the indicated test articles. Activation is measured by secretion of pro-inflammatory cytokines, such as IFNλ1 and TNFα, by BioLegend LEGENDPLEX™ cytokine bead array.

All references, including publications, patent applications, and patents, cited herein are hereby incorporated by reference to the same extent as if each reference were individually and specifically indicated to be incorporated by reference and were set forth in its entirety herein.

1. An immunoconjugate comprising an antibody covalently attached to one or more STING agonist moieties by a linker, and having Formula I:
Ab-[L-D]ₚ I
or a pharmaceutically acceptable salt thereof,
wherein:
Ab is the antibody;
p is an integer from 1 to 8;
D is the STING agonist moiety having the formula: 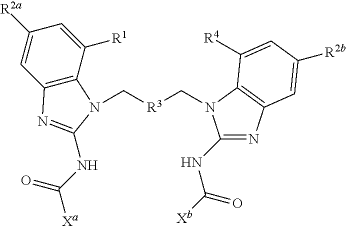
X^{a} and X^{b} are independently selected from a five-membered heteroaryl, optionally substituted with R⁵;
R¹ and R⁴ are independently selected from the group consisting of H, F, Cl, Br, I, —CN, —OH, —O—(C₁-C₆ alkyl), and R⁵;
R^{2a} and R^{2b} are independently selected from H, —C(═O)N(R⁶)₂, and R⁵;
where one of X^{a}, X^{b}, R¹, R⁴, R^{2a} and R^{2b} is substituted with R⁵;
R³ is selected from C₁-C₆ alkyldiyl, —(C₁-C₃ alkyldiyl)-O—(C₁-C₃ alkyldiyl)-, C₂-C₆ alkenyldiyl and C₂-C₆ alkynyldiyl, optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂;
R⁵ is selected from the group consisting of:
(C₁-C₁₂ alkyldiyl)-*;
(C₁-C₁₂ alkyldiyl)-N(R⁶)—*;
(C₁-C₁₂ alkyldiyl)-O—*;
(C₁-C₁₂ alkyldiyl)-(C₂-C₂₀ heterocyclyldiyl)-*;
O—(C₁-C₁₂ alkyldiyl)-*;
O—(C₁-C₁₂ alkyldiyl)-N(R⁶)—*;
O—(C₁-C₁₂ alkyldiyl)-O—*;
O—(C₁-C₁₂ alkyldiyl)-(C₂-C₂₀ heterocyclyldiyl)-*;
(O)—(C₁-C₁₂ alkyldiyl)-C₂-C₂₀ heterocyclyldiyl)-N(R⁶)—*;
OC(═O)N(R⁶)—*;
OC(═O)N(R⁶)—(C₁-C₁₂ alkyldiyl)-N(R⁶)—*;
N(R⁶)—*;
N(R⁶)—(C₁-C₁₂ alkyldiyl)-*;
N(R⁶)—(C₁-C₁₂ alkyldiyl)-N(R⁶)—*;
N(R⁶)—(C₁-C₁₂ alkyldiyl)-O—*;
N(R⁶)—(C₁-C₁₂ alkyldiyl)-(C₂-C₂ heterocyclyldiyl)-*;
C(═O)N(R⁶)—*;
C(═O)N(R⁶)—(C₁-C₁₂ alkyldiyl)-*;
C(═O)N(R⁶)—(C₁-C₁₂ alkyldiyl)-N(R⁶)—*;
C(═O)N(R⁶)—(C₁-C₁₂ alkyldiyl)-O—*;
(C₂-C₂₀ heterocyclyldiyl)-*;
S(═O)₂—(C₂-C₂₀ heterocyclyldiyl)-*; and
S(═O)₂—(C₂-C₂₀ heterocyclyldiyl)-(C₁-C₁₂ alkyldiyl)-N(R⁶)—*;
where the asterisk * indicates the attachment site of L;
R⁶ is independently H or C₁-C₆ alkyl;
L is the linker selected from the group consisting of:
C(═O)-PEG-;
C(═O)-PEG-C(═O)N(R⁶)—(C₁-C₁₂ alkyldiyl)-C(═O)-Gluc-;
C(═O)-PEG-O—;
C(═O)-PEG-O—C(═O)—;
C(═O)-PEG-C(═O)—;
C(═O)-PEG-C(═O)-PEP-;
C(═O)-PEG-N(R⁶)—;
C(═O)-PEG-N(R⁶)—C(═O)—;
C(═O)-PEG-N(R⁶)-PEG-C(═O)-PEP-;
C(═O)-PEG-N⁺(R⁶)₂-PEG-C(═O)-PEP-;
C(═O)-PEG-C(═O)-PEP-N(R⁶)—(C₁-C₁₂ alkyldiyl)-;
C(═O)-PEG-C(═O)-PEP-N(R⁶)—(C₁-C₁₂ alkyldiyl)N(R⁶)C(═O)—(C₂-C₅ monoheterocyclyldiyl)-;
C(═O)-PEG-C(═O)N(R⁶)—(C₁-C₁₂ alkyldiyl)-C(═O)-PEP-;
C(═O)-PEG-SS-(C₁-C₁₂ alkyldiyl)-OC(═O)—;
C(═O)-PEG-SS-(C₁-C₁₂ alkyldiyl)-C(═O)—;
C(═O)—(C₁-C₁₂ alkyldiyl)-C(═O)-PEP-;
C(═O)—(C₁-C₁₂ alkyldiyl)-C(═O)-PEP-N(R⁶)—(C₁-C₁₂ alkyldiyl)-;
C(═O)—(C₁-C₁₂ alkyldiyl)-C(═O)-PEP-N(R⁶)—(C₁-C₁₂ alkyldiyl)-N(R⁵)—C(═O);
C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
succinimidyl-(CH₂)ₘ—C(═O)N(R⁶)-PEG-;
succinimidyl-(CH₂)ₘ—C(═O)N(R⁶)-PEG-C(═O)N(R⁶)—(C₁-C₁₂ alkyldiyl)-C(═O)-Gluc-;
succinimidyl-(CH₂)ₘ—C(═O)N(R⁶)-PEG-O—;
succinimidyl-(CH₂)ₘ—C(═O)N(R⁶)-PEG-O—C(═O)—;
succinimidyl-(CH₂)ₘ—C(═O)N(R⁶)-PEG-C(═O)—;
succinimidyl-(CH₂)ₘ—C(═O)N(R⁶)-PEG-N(R⁵)—;
succinimidyl-(CH₂)ₘ—C(═O)N(R⁶)-PEG-N(R⁵)—C(═O)—;
succinimidyl-(CH₂)ₘ—C(═O)N(R⁶)-PEG-C(═O)-PEP-;
succinimidyl-(CH₂)ₘ—C(═O)N(R⁶)-PEG-SS-(C₁-C₁₂ alkyldiyl)-OC(═O)—;
succinimidyl-(CH₂)ₘ—C(═O)-PEP-N(R⁶)—(C₁-C₁₂ alkyldiyl)-;
succinimidyl-(CH₂)ₘ—C(═O)N(R⁶)-PEG-C(═O)N(R⁶)—(C₁-C₁₂ alkyldiyl)-C(═O)—PEP-;
succinimidyl-(CH₂)ₘ—C(═O)-PEP-N(R⁶)—(C₁-C₁₂ alkyldiyl)N(R⁶)C(═O)—; and
succinimidyl-(CH₂)ₘ—C(═O)-PEP-N(R⁶)—(C₁-C₁₂ alkyldiyl)N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
Gluc has the formula: 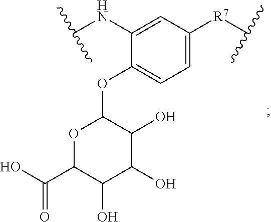
PEP has the formula: 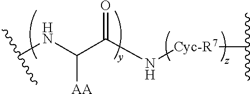
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 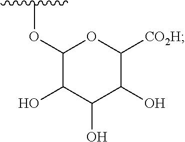 [SMILES: *OC1OC(C(=O)O)C(O)C(O)C1O]
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

2. The immunoconjugate of claim 1 wherein the antibody is an antibody construct that has an antigen binding domain that binds a target selected from PD-L1, HER2, CEA, and Trop2.

3. The immunoconjugate of claim 2 wherein the antibody is selected from the group consisting of atezolizumab, durvalumab, avelumab, trastuzumab, pertuzumab, labetuzumab, and sacituzumab.

4-9. (canceled)

10. The immunoconjugate of claim 1 wherein X^{a} and X^{b} are independently selected from the group consisting of imidazolyl, pyrazolyl, triazolyl, tetrazolyl, furyl, thienyl, isoxazolyl, thiazolyl, oxadiazolyl, oxazolyl, isothiazolyl, pyrrolyl, oxadiazolyl, and thiadiazolyl.

11. The immunoconjugate of claim 10 wherein X^{a} and X^{b} are each pyrazolyl, substituted with one or more groups selected from —CH₃, —CH₂CH₃, —CH═CH₂, —C≡CH, —C≡CCH₃, —CH₂CH₂CH₃, —CH(CH₃)₂, and —CH₂CH(CH₃)₂.

12. The immunoconjugate of claim 1 wherein one of X^{a} and X^{b} is substituted with R⁵.

13. The immunoconjugate of claim 1 wherein R¹ is selected from the group consisting of —OCH₃, —OCH₂CH₃, —OCH₂CH₂OCH₃, —OCH₂CH₂OH, and —OCH₂CH₂N(CH₃)₂.

14. The immunoconjugate of claim 1 wherein R¹ is —OCH₃ or F.

15. (canceled)

16. The immunoconjugate of claim 1 wherein R^{2a} and R^{2b} are each —C(═O)NH₂.

17. The immunoconjugate of claim 1 wherein one of R^{2a} and R^{2b} is substituted with R⁵.

18. The immunoconjugate of claim 1 wherein R³ is selected from —CH₂CH₂—, —CH═CH—, and —C≡C—.

19. The immunoconjugate of claim 1 wherein R³ is C₂-C₄ alkenyldiyl, substituted with one or more groups selected from F, —OH, and —OCH₃.

20. The immunoconjugate of claim 1 wherein R⁴ is —O—(C₁-C₁₂ alkyldiyl)-(C₂-C₂₀ heterocyclyldiyl)-*.

21. The immunoconjugate of claim 20 wherein C₁-C₁₂ alkyldiyl is propyldiyl and C₂-C₂₀ heterocyclyldiyl is piperidiyl.

22. The immunoconjugate of claim 1 wherein one of R¹ and R⁴ is substituted with R⁵.

23. The immunoconjugate of claim 1 wherein L is —C(═O)-PEG- or —C(═O)-PEG-C(═O)—.

24. The immunoconjugate of claim 1 wherein L is attached to a cysteine thiol of the antibody.

25. The immunoconjugate of claim 1 wherein for the PEG, m is 1 or 2, and n is an integer from 2 to 10.

26. The immunoconjugate of claim 25 wherein n is 10.

27. The immunoconjugate of claim 1 wherein L comprises PEP and PEP is a dipeptide and has the formula:

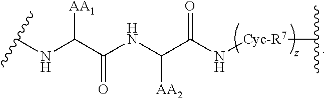

28. The immunoconjugate of claim 1 wherein L comprises PEP and PEP is a tripeptide and has the formula:

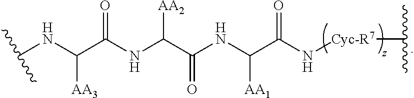

29. The immunoconjugate of claim 1 wherein L comprises PEP and PEP is a tetrapeptide and has the formula:

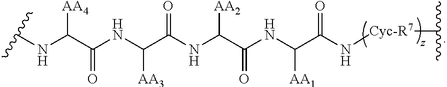

30. The immunoconjugate of claim 1 wherein L is selected from the structures: 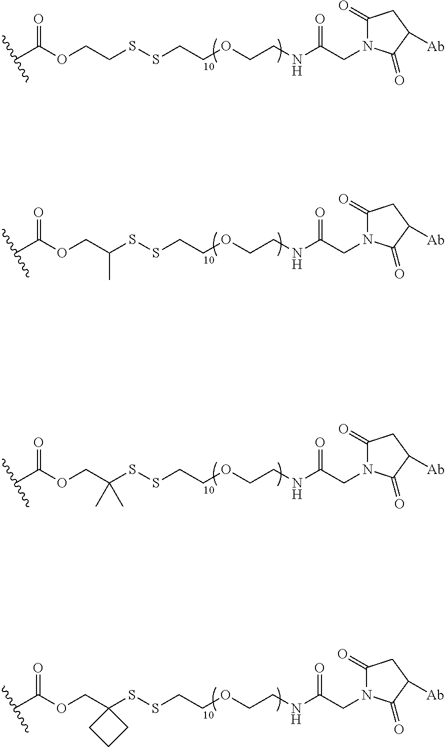
where the wavy line indicates the attachment to R⁵.

31. A STING agonist-linker intermediate compound having Formula II: 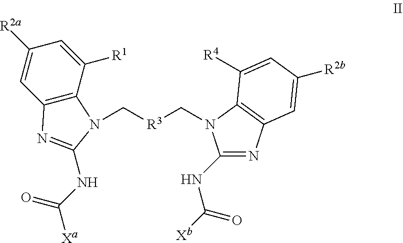
wherein
X^{a} and X^{b} are independently selected from a five-membered heteroaryl, optionally substituted with R⁵;
R¹ and R⁴ are independently selected from the group consisting of F, Cl, Br, I, —CN, —OH, —O—(C₁-C₆ alkyl), and R⁵;
R^{2a} and R^{2b} are independently selected from —C(═O)N(R⁶)₂ and R⁵;
where one of X^{a}, X^{b}, R¹, R⁴, R^{2a} and R^{2b} is substituted with R⁵;
R³ is selected from C₁-C₆ alkyldiyl, —(C₁-C₃ alkyldiyl)-O—(C₁-C₃ alkyldiyl)-, C₂-C₆ alkenyldiyl and C₂-C₆ alkynyldiyl, optionally substituted with one or more groups selected from F, Cl, —OH, —OCH₃, —OCH₂CH₃, —OCH₂CH₂OCH₃, —OCH₂CH₂OH, —OCH₂CH₂N(CH₃)₂;
R⁵ is selected from the group consisting of:
(C₁-C₁₂ alkyldiyl)-L;
(C₁-C₁₂ alkyldiyl)-N(R⁶)-L;
(C₁-C₁₂ alkyldiyl)-O-L;
(C₁-C₁₂ alkyldiyl)-(C₂-C₂₀ heterocyclyldiyl)-L;
O—(C₁-C₁₂ alkyldiyl)-L;
O—(C₁-C₁₂ alkyldiyl)-N(R⁶)-L;
O—(C₁-C₁₂ alkyldiyl)-O-L;
O—(C₁-C₁₂ alkyldiyl)-(C₂-C₂₀ heterocyclyldiyl)-L;
O—(C₁-C₁₂ alkyldiyl)-(C₂-C₂₀ heterocyclyldiyl)-N(R⁶)-L;
OC(═O)N(R⁶)-L;
OC(═O)N(R⁶)—(C₁-C₁₂ alkyldiyl)-N(R⁶)-L;
N(R⁶)-L;
N(R⁶)—(C₁-C₁₂ alkyldiyl)-L;
N(R⁶)—C₁-C₁₂ alkyldiyl)-N(R⁶)-L;
N(R⁶)—(C₁-C₁₂ alkyldiyl)-O-L;
N(R⁶)—(C₁-C₁₂ alkyldiyl)-(C₂-C₂₀ heterocyclyldiyl)-L;
C(═O)N(R⁶)-L;
C(═O)N(R⁶)—(C₁-C₁₂ alkyldiyl)-L;
C(═O)N(R⁶)—(C₁-C₁₂ alkyldiyl)-N(R⁶)-L;
C(═O)N(R⁶)—(C₁-C₁₂ alkyldiyl)-O-L;
(C₂-C₂₀ heterocyclyldiyl)-L;
S(═O)₂—(C₂-C₂₀ heterocyclyldiyl)-L; and
S(═O)₂—(C₂-C₂₀ heterocyclyldiyl)-(C₁-C₁₂ alkyldiyl)-N(R⁶)-L;
R⁶ is independently H or C₁-C₆ alkyl;
L is a linker selected from the group consisting of:
Q-C(═O)-PEG-;
Q-C(═O)-PEG-C(═O)N(R⁶)—(C₁-C₁₂ alkyldiyl)-C(═O)-Gluc-;
Q-C(═O)-PEG-O—;
Q-C(═O)-PEG-O—C(═O)—;
Q-C(═O)-PEG-C(═O)—;
Q-C(═O)-PEG-C(═O)-PEP-;
Q-C(═O)-PEG-N(R⁶)—;
Q-C(═O)-PEG-N(R⁶)—C(═O)—;
Q-C(═O)-PEG-N(R⁶)-PEG-C(═O)-PEP-;
Q-C(═O)-PEG-N⁺(R⁶)₂-PEG-C(═O)-PEP-;
Q-C(═O)-PEG-C(═O)-PEP-N(R⁶)—(C₁-C₁₂ alkyldiyl)-;
Q-C(═O)-PEG-C(═O)-PEP-N(R⁶)—(C₁-C₁₂ alkyldiyl)N(R⁶)C(═O)—(C₂-C₅ monoheterocyclyldiyl)-;
Q-C(═O)-PEG-C(═O)N(R⁶)—(C₁-C₁₂ alkyldiyl)-C(═O)-PEP-;
Q-C(═O)-PEG-SS-(C₁-C₁₂ alkyldiyl)-OC(═O)—;
Q-C(═O)-PEG-SS-(C₁-C₁₂ alkyldiyl)-C(═O)—;
Q-C(═O)—(C₁-C₁₂ alkyldiyl)-C(═O)-PEP-;
Q-C(═O)—(C₁-C₁₂ alkyldiyl)-C(═O)-PEP-N(R⁶)—(C₁-C₁₂ alkyldiyl)-;
Q-C(═O)—(C₁-C₁₂ alkyldiyl)-C(═O)-PEP-N(R⁶)—(C₁-C₁₂ alkyldiyl)-N(R⁵)—C(═O);
Q-C(═O)—(C₁-C₁₂ alkyldiyl)-C(═O)-PEP-N(R⁶)—(C₁-C₁₂ alkyldiyl)-N(R⁶)C(═O)—(C₂-C₅ monoheterocyclyldiyl)-;
Q-(CH₂)ₘ—C(═O)N(R⁶)-PEG-;
Q-(CH₂)ₘ—C(O)N(R⁶)-PEG-C(═O)N(R⁶)—(C₁-C₁₂ alkyldiyl)-C(═O)-Gluc-;
Q-(CH₂)ₘ—C(═O)N(R⁶)-PEG-O—;
Q-(CH₂)ₘ—C(═O)N(R⁶)-PEG-O—C(═O)—;
Q-(CH₂)ₘ—C(═O)N(R⁶)-PEG-C(═O)—;
Q-(CH₂)ₘ—C(═O)N(R⁶)-PEG-N(R⁶)—;
Q-(CH₂)ₘ—C(═O)N(R⁶)-PEG-N(R⁶)—C(═O)—;
Q-(CH₂)ₘ—C(═O)N(R⁶)-PEG-C(═O)-PEP-;
Q-(CH₂)ₘ—C(═O)N(R⁶)-PEG-SS-(C₁-C₁₂ alkyldiyl)-OC(═O)—;
Q-(CH₂)ₘ—C(═O)-PEP-N(R⁶)—(C₁-C₁₂ alkyldiyl)-;
Q-(CH₂)ₘ—C(═O)N(R⁶)-PEG-C(═O)N(R⁶)—(C₁-C₁₂ alkyldiyl)-C(═O)-PEP-;
Q-(CH₂)ₘ—C(═O)-PEP-N(R⁶)—(C₁-C₁₂ alkyldiyl)N(R⁶)C(═O)—; and
Q-(CH₂)ₘ—C(═O)-PEP-N(R⁶)—(C₁-C₁₂ alkyldiyl)N(R⁶)C(═O)—(C₂-C₅ monoheterocyclyldiyl)-;
PEG has the formula: —(CH₂CH₂O)ₙ—(CH₂)ₘ—; m is an integer from 1 to 5, and n is an integer from 2 to 50;
Gluc has the formula: 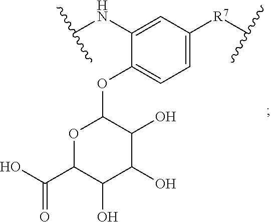
PEP has the formula: 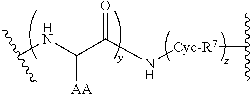
where AA is independently selected from a natural or unnatural amino acid side chain, or one or more of AA, and an adjacent nitrogen atom form a 5-membered ring proline amino acid, and the wavy line indicates a point of attachment;
Cyc is selected from C₆-C₂₀ aryldiyl and C₁-C₂₀ heteroaryldiyl, optionally substituted with one or more groups selected from F, Cl, NO₂, —OH, —OCH₃, and a glucuronic acid having the structure: 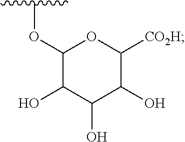 [SMILES: *OC1OC(C(=O)O)C(O)C(O)C1O]
R⁷ is selected from the group consisting of —CH(R⁸)O—, —CH₂—, —CH₂N(R⁸)—, and —CH(R⁸)O—C(═O)—, where R⁸ is selected from H, C₁-C₆ alkyl, C(═O)—C₁-C₆ alkyl, and —C(═O)N(R⁹)₂, where R⁹ is independently selected from the group consisting of H, C₁-C₁₂ alkyl, and —(CH₂CH₂O)ₙ—(CH₂)ₘ—OH, where m is an integer from 1 to 5, and n is an integer from 2 to 50, or two R⁹ groups together form a 5- or 6-membered heterocyclyl ring;
y is an integer from 2 to 12;
z is 0 or 1;
Q is selected from the group consisting of N-hydroxysuccinimidyl, N-hydroxysulfosuccinimidyl, maleimide, and phenoxy substituted with one or more groups independently selected from F, Cl, NO₂, and SO₃⁻; and
alkyl, alkyldiyl, alkenyl, alkenyldiyl, alkynyl, alkynyldiyl, aryl, aryldiyl, carbocyclyl, carbocyclyldiyl, heterocyclyl, heterocyclyldiyl, heteroaryl, and heteroaryldiyl are independently and optionally substituted with one or more groups independently selected from F, Cl, Br, I, —CN, —CH₃, —CH₂CH₃, —CH═CH₂, —C≡CH, —C≡CCH₃, —CH₂CH₂CH₃, —CH(CH₃)₂, —CH₂CH(CH₃)₂, —CH₂OH, —CH₂OCH₃, —CH₂CH₂OH, —C(CH₃)₂OH, —CH(OH)CH(CH₃)₂, —C(CH₃)₂CH₂OH, —CH₂CH₂SO₂CH₃, —CH₂OP(O)(OH)₂, —CH₂F, —CHF₂, —CF₃, —CH₂CF₃, —CH₂CHF₂, —CH(CH₃)CN, —C(CH₃)₂CN, —CH₂CN, —CH₂NH₂, —CH₂NHSO₂CH₃, —CH₂NHCH₃, —CH₂N(CH₃)₂, —CO₂H, —COCH₃, —CO₂CH₃, —CO₂C(CH₃)₃, —COCH(OH)CH₃, —CONH₂, —CONHCH₃, —CON(CH₃)₂, —C(CH₃)₂CONH₂, —NH₂, —NHCH₃, —N(CH₃)₂, —NHCOCH₃, —N(CH₃)COCH₃, —NHS(O)₂CH₃, —N(CH₃)C(CH₃)₂CONH₂, —N(CH₃)CH₂CH₂S(O)₂CH₃, —NHC(═NH)H, —NHC(═NH)CH₃, —NHC(═NH)NH₂, —NHC(═O)NH₂, —NO₂, ═O, —OH, —OCH₃, —OCH₂CH₃, —OCH₂CH₂OCH₃, —OCH₂CH₂OH, —OCH₂CH₂N(CH₃)₂, —O(CH₂CH₂O)ₙ—(CH₂)ₘCO₂H, —O(CH₂CH₂O)ₙH, —OCH₂F, —OCHF₂, —OCF₃, —OP(O)(OH)₂, —S(O)₂N(CH₃)₂, —SCH₃, —S(O)₂CH₃, and —S(O)₃H.

32. The STING agonist-linker intermediate compound of claim 31 wherein Q is selected from:

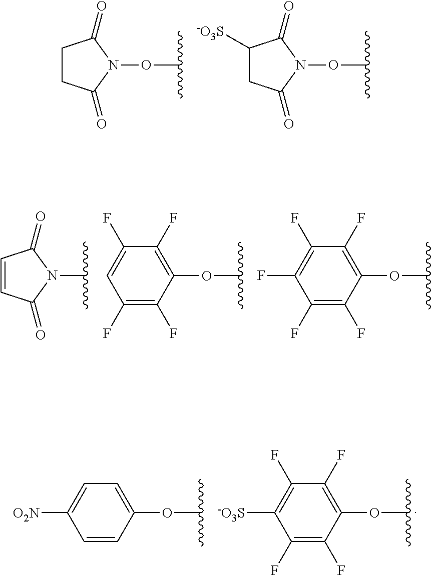

33. The STING agonist-linker intermediate compound of claim 31 wherein Q is phenoxy substituted with one or more groups independently selected from F, Cl, NO₂, and SO₃⁻.

34. The STING agonist-linker intermediate compound of claim 31 wherein Q is 2,3,5,6-tetrafluorophenoxy or 2,3,5,6-tetrafluoro-4-sulfonato-phenoxy.

35. (canceled)

36. The STING agonist-linker intermediate compound of claim 31 wherein Q is maleimide.

37. The STING agonist-linker intermediate compound of claim 31 wherein L is selected from the structures: 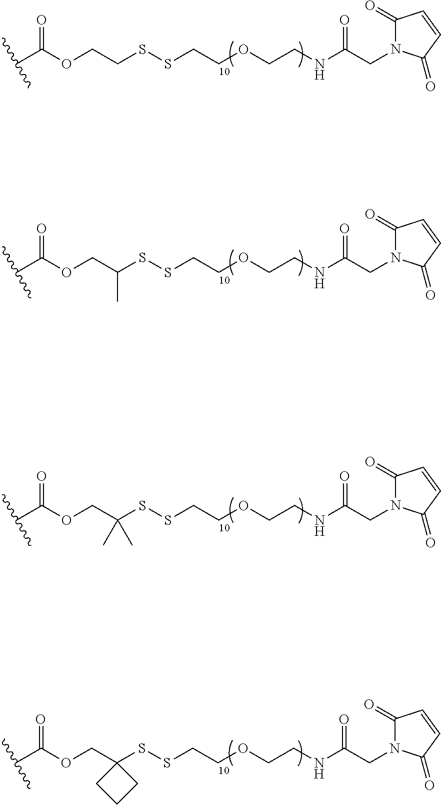
where the wavy line indicates the attachment to R⁵.

38. The STING agonist-linker intermediate compound of claim 31 selected from the group consisting of.

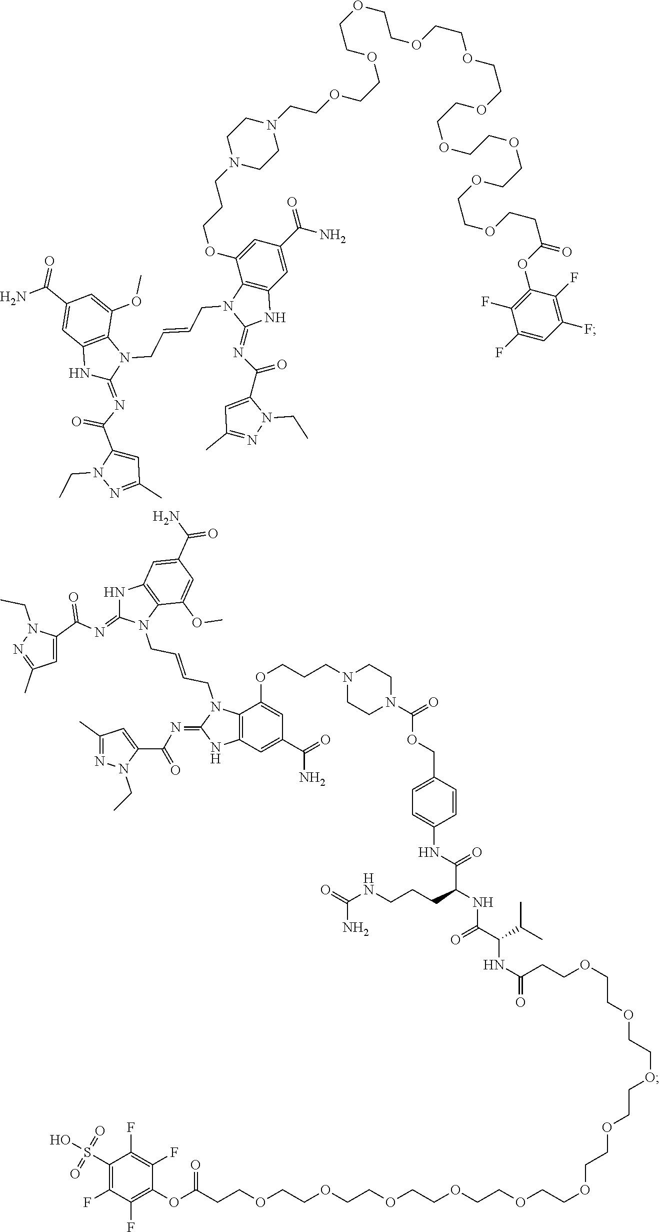

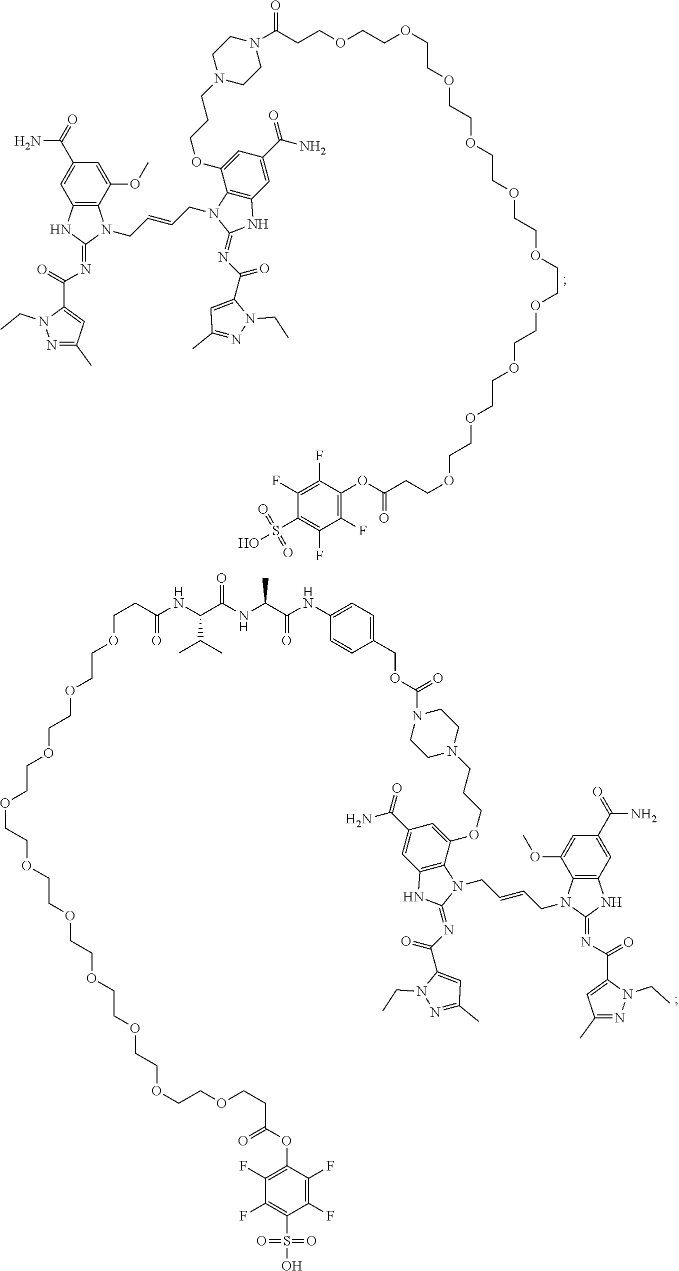

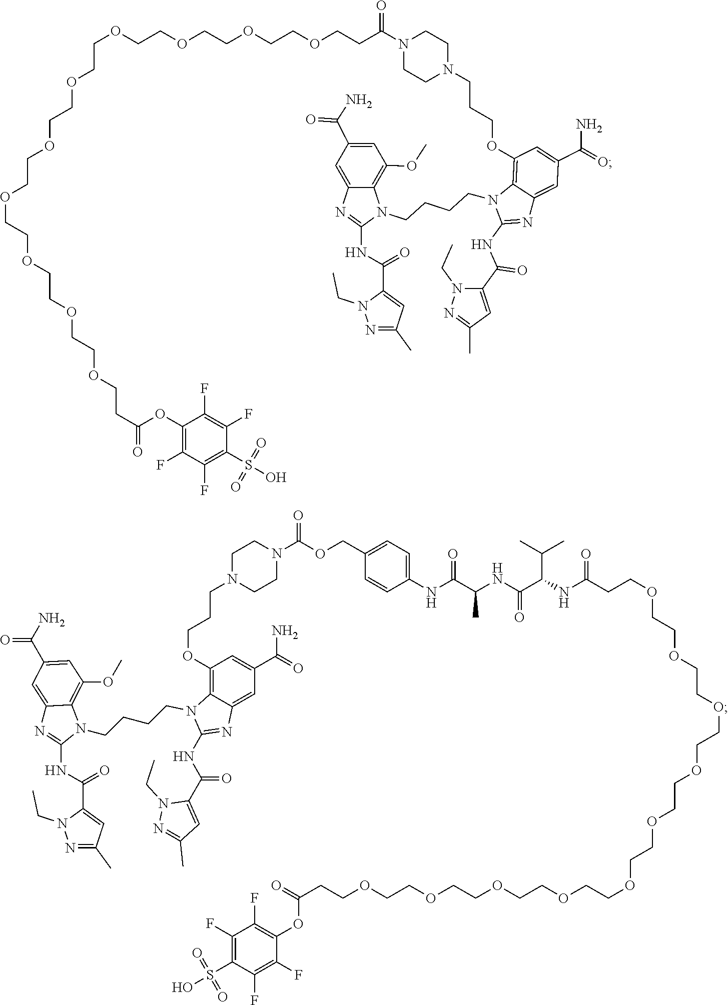 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(C(=O)CCOCCOCCOCCOCCOCCOCCOCCOCCOCCOCCC(=O)Oc4c(F)c(F)c(S(=O)(=O)O)c(F)c4F)CC3)c21 ; CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21]

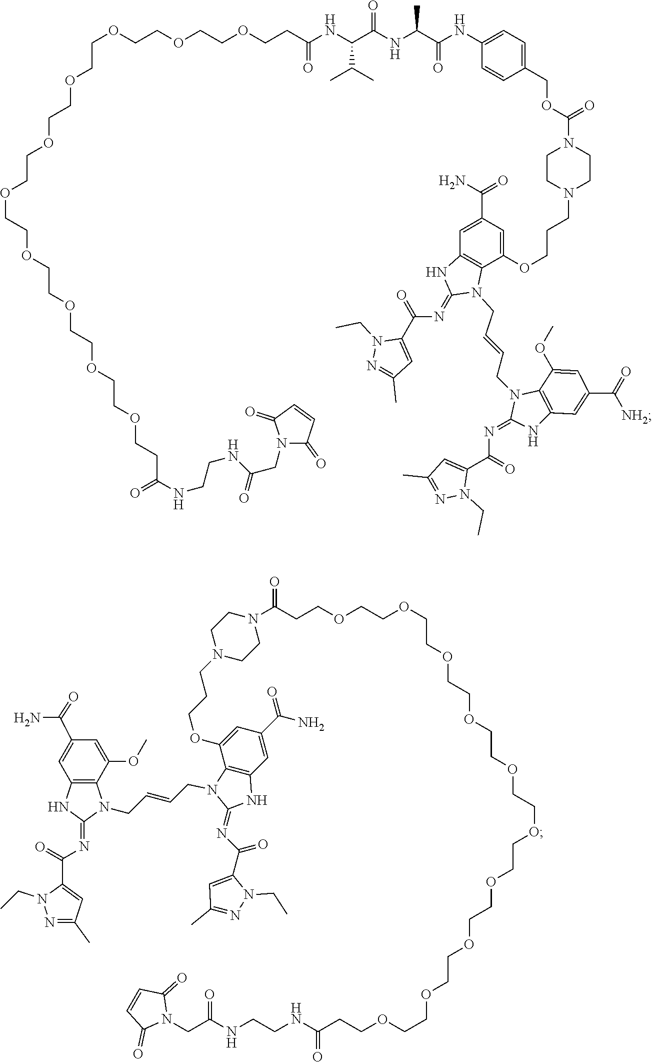 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCC(=O)NCCNC(=O)CN5C(=O)C=CC5=O)C(C)C)cc4)CC3)c21 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)CCOCCOCCOCCOCCOCCOCCOCCOCCOCCOCCC(=O)NCCNC(=O)CN4C(=O)C=CC4=O)CC3)c21]

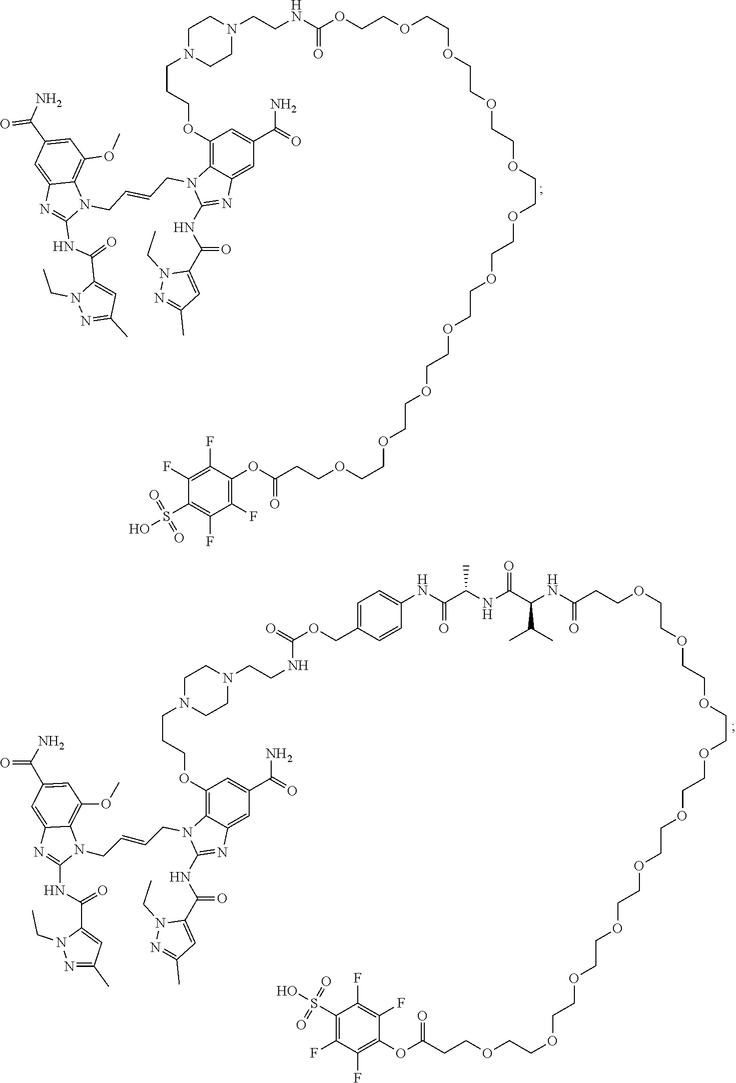 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCNC(=O)OCCOCCOCCOCCOCCOCCOCCOCCOCCOCCOCCC(=O)Oc4c(F)c(F)c(S(=O)(=O)O)c(F)c4F)CC3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCNC(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21]

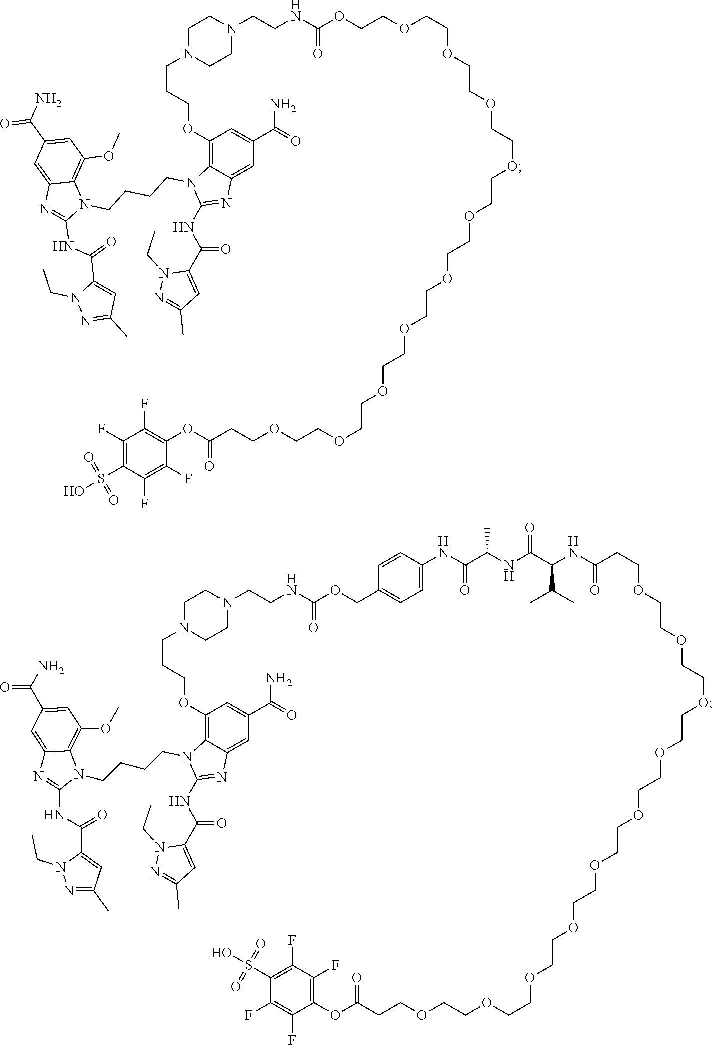 [SMILES: CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(CCNC(=O)OCCOCCOCCOCCOCCOCCOCCOCCOCCOCCOCCC(=O)Oc4c(F)c(F)c(S(=O)(=O)O)c(F)c4F)CC3)c21 ; CCn1nc(C)cc1C(=O)Nc1nc2cc(C(N)=O)cc(OC)c2n1CCCCn1c(NC(=O)c2cc(C)nn2CC)nc2cc(C(N)=O)cc(OCCCN3CCN(CCNC(=O)OCc4ccc(NC(=O)[C@H](C)NC(=O)[C@@H](NC(=O)CCOCCOCCOCCOCCOCCOCCOCCOCCOCCOCCC(=O)Oc5c(F)c(F)c(S(=O)(=O)O)c(F)c5F)C(C)C)cc4)CC3)c21]

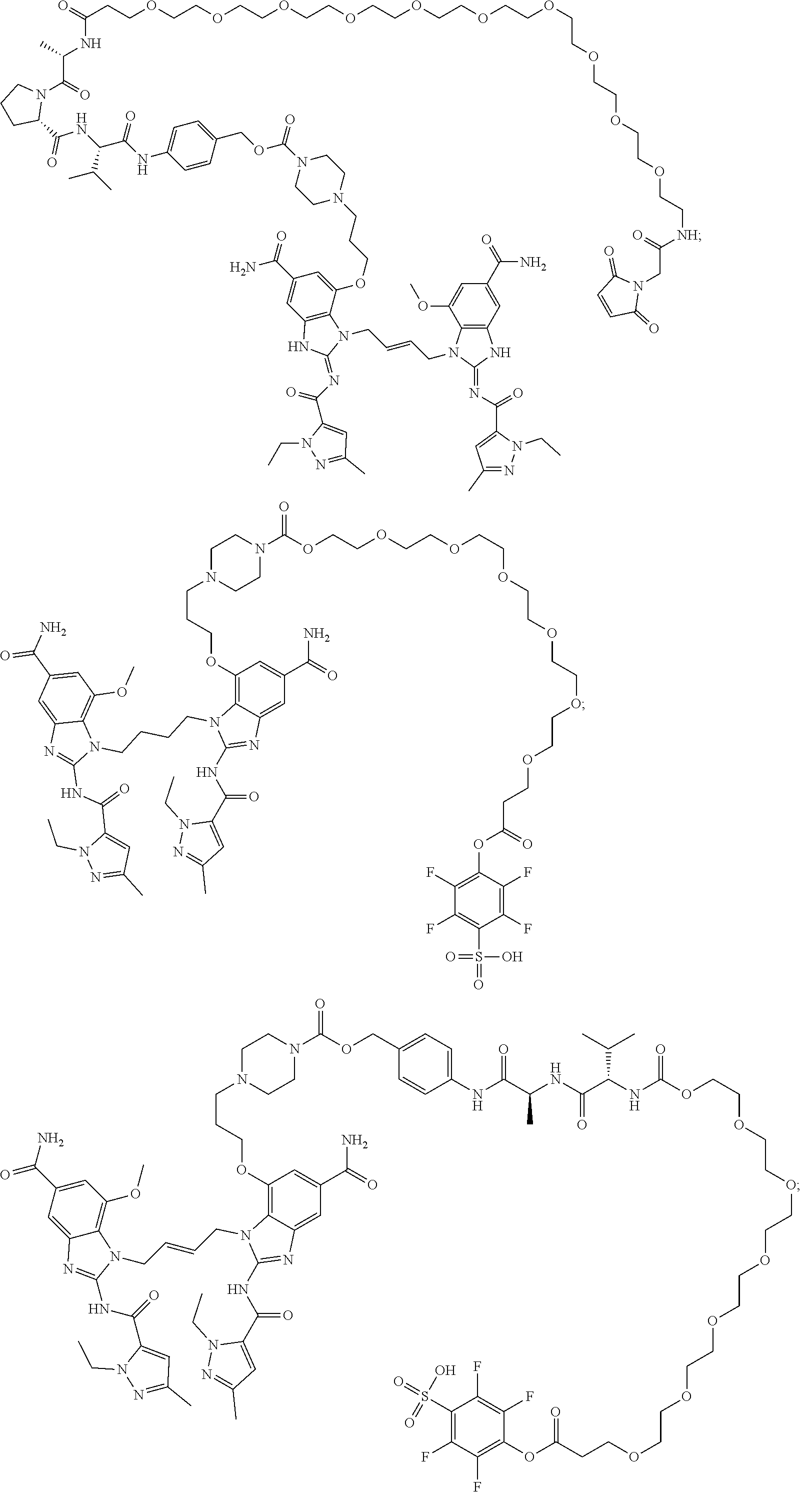

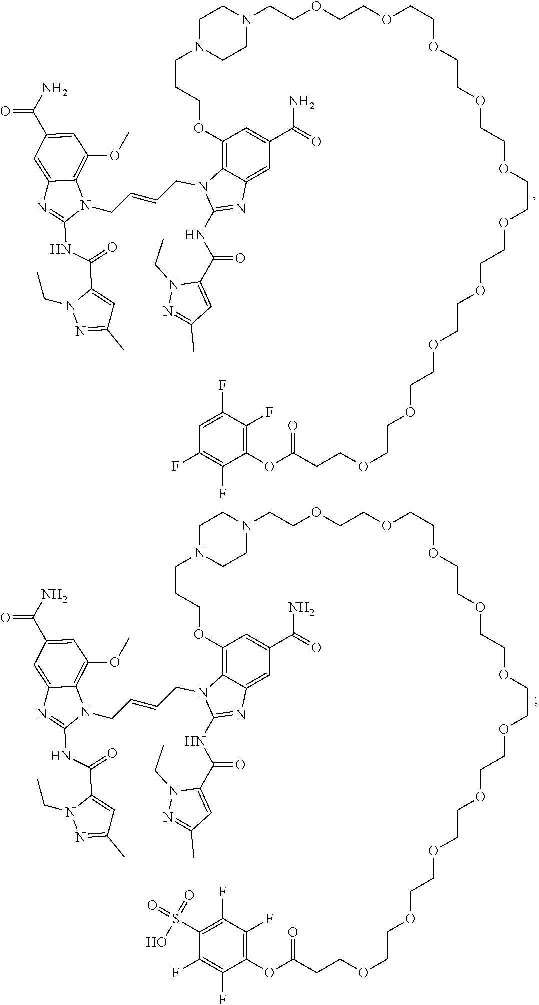

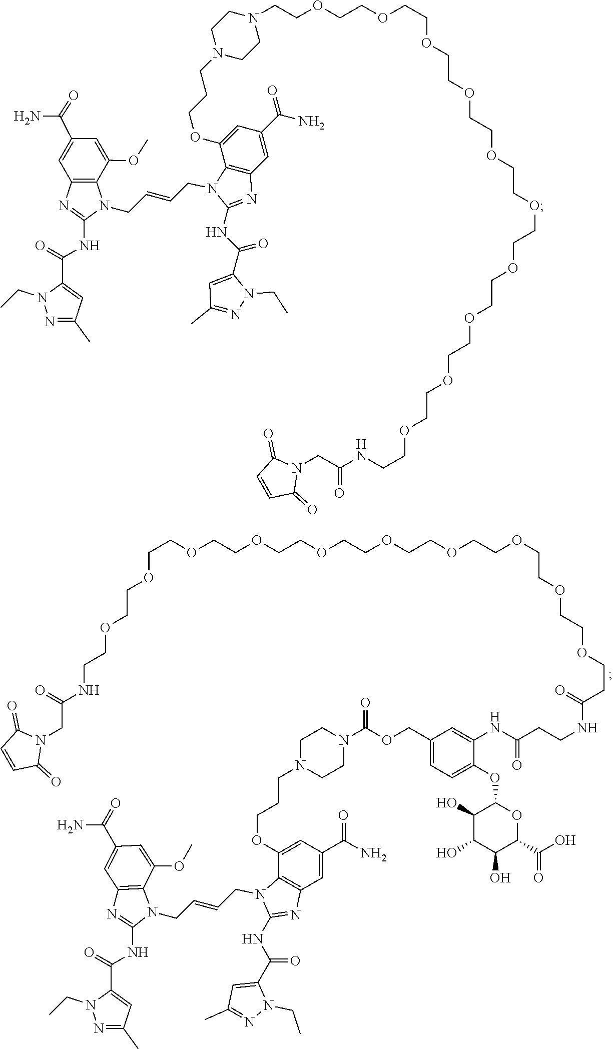

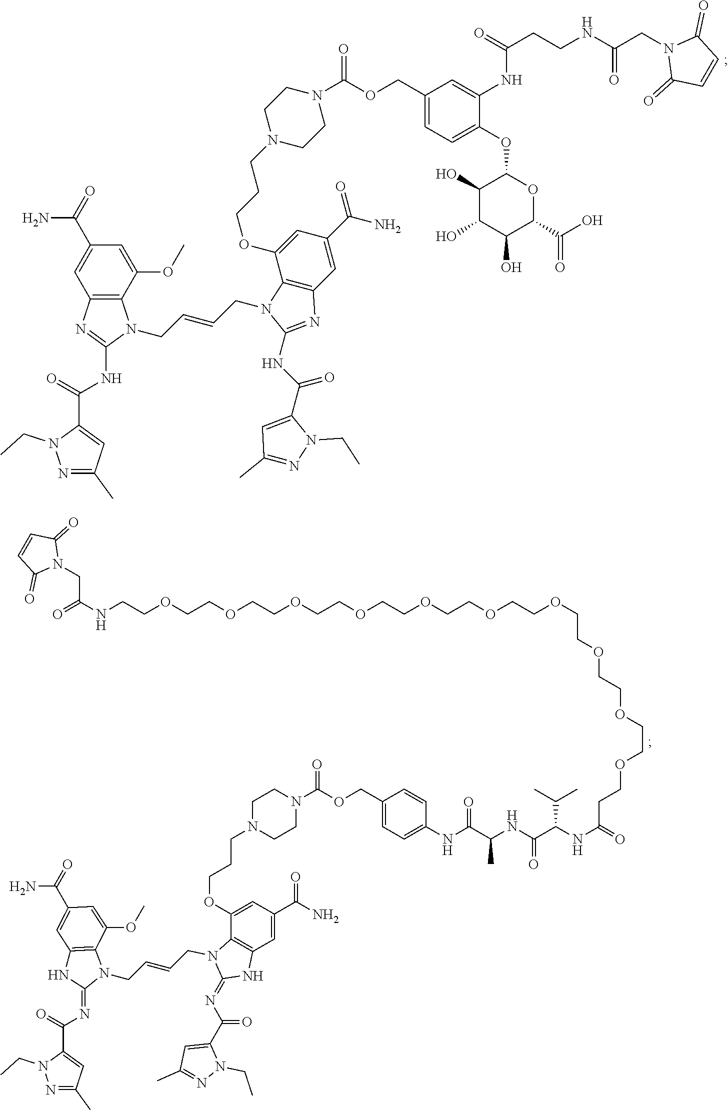 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(C(=O)OCc4ccc(O[C@@H]5O[C@H](C(=O)O)[C@@H](O)[C@H](O)[C@H]5O)c(NC(=O)CCNC(=O)CN5C(=O)C=CC5=O)c4)CC3)c21 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](C)NC(=O)[C@@H](NC(=O)CCOCCOCCOCCOCCOCCOCCOCCOCCOCCOCCNC(=O)CN5C(=O)C=CC5=O)C(C)C)cc4)CC3)c21]

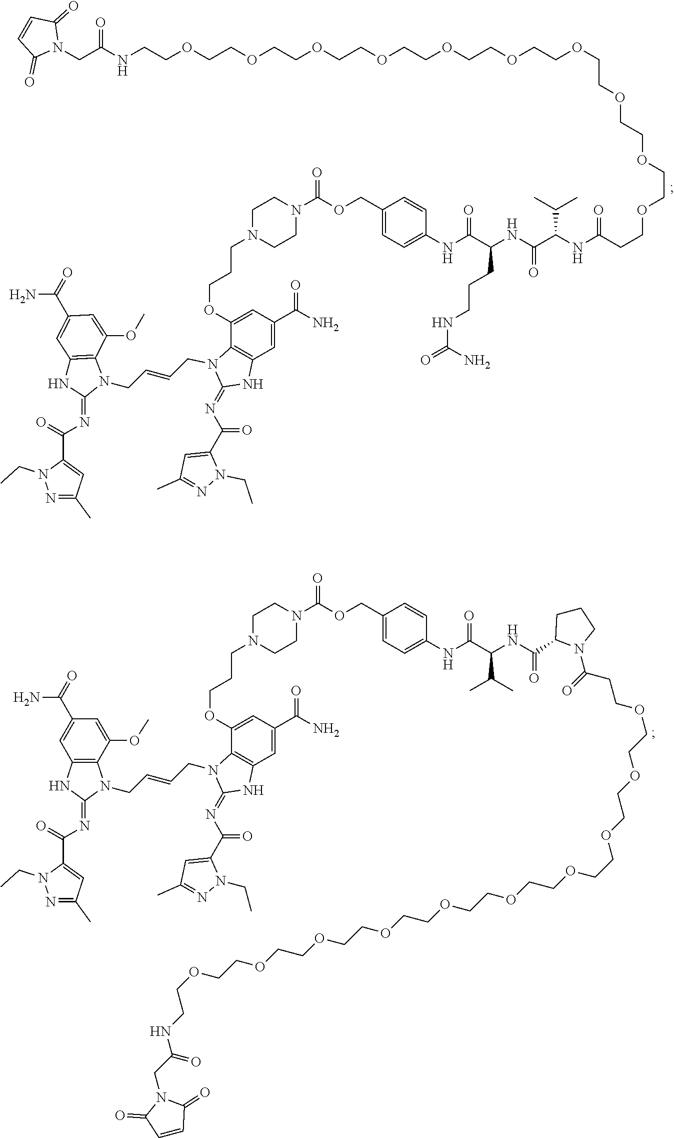 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@H](CCCNC(N)=O)NC(=O)[C@@H](NC(=O)CCOCCOCCOCCOCCOCCOCCOCCOCCOCCOCCNC(=O)CN5C(=O)C=CC5=O)C(C)C)cc4)CC3)c21 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCN3CCN(C(=O)OCc4ccc(NC(=O)[C@@H](NC(=O)[C@@H]5CCCN5C(=O)CCOCCOCCOCCOCCOCCOCCOCCOCCOCCOCCNC(=O)CN5C(=O)C=CC5=O)C(C)C)cc4)CC3)c21]

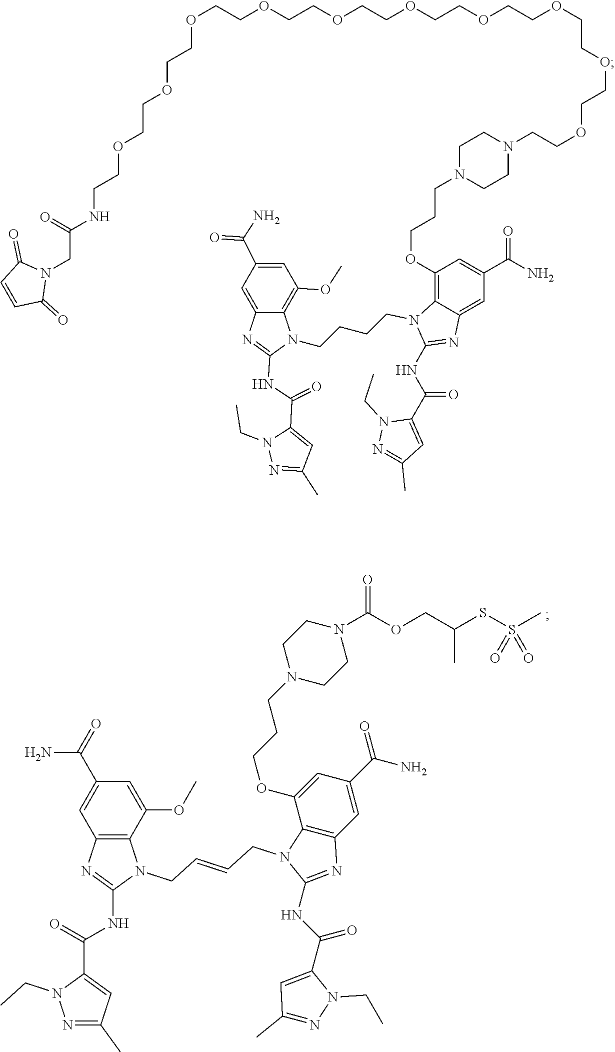

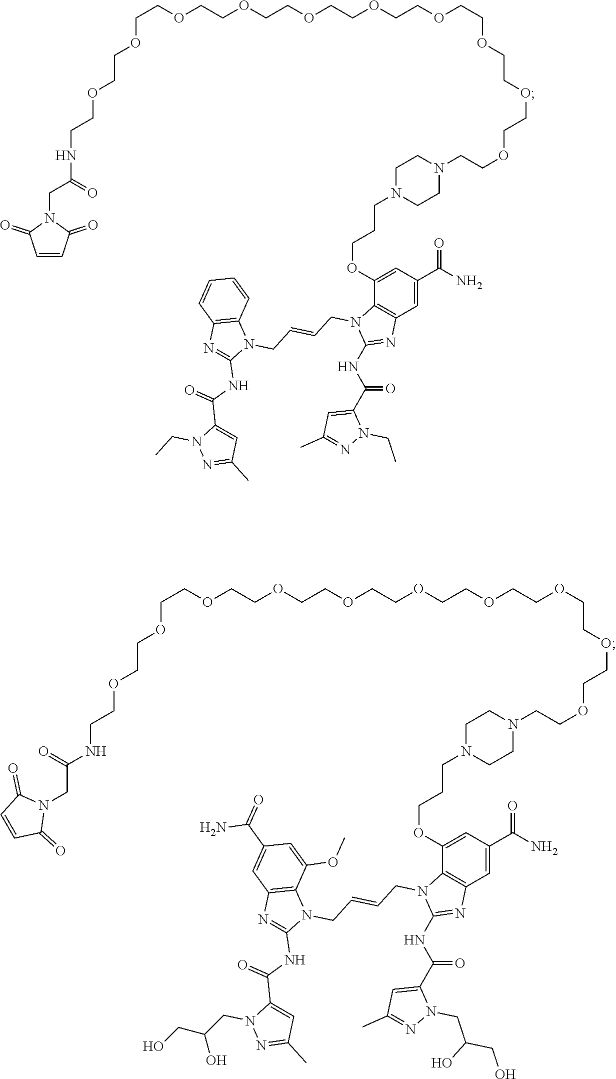 [SMILES: CCn1nc(C)cc1C(=O)Nc1nc2ccccc2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21 ; COc1cc(C(N)=O)cc2nc(NC(=O)c3cc(C)nn3CC(O)CO)n(C/C=C/Cn3c(NC(=O)c4cc(C)nn4CC(O)CO)nc4cc(C(N)=O)cc(OCCCN5CCN(CCOCCOCCOCCOCCOCCOCCOCCOCCOCCOCCNC(=O)CN6C(=O)C=CC6=O)CC5)c43)c12]

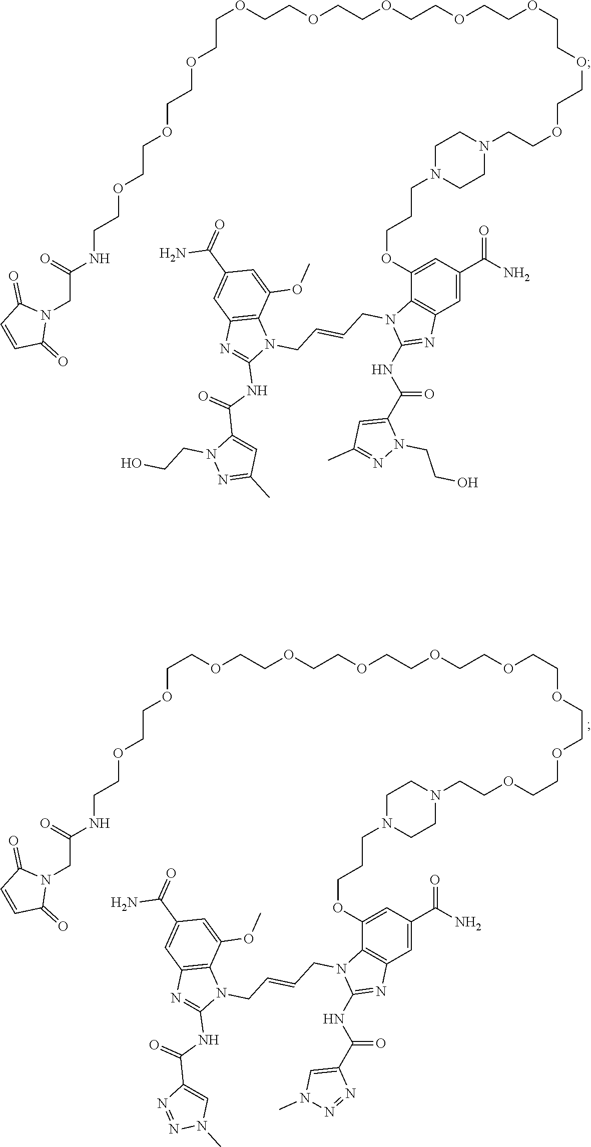 [SMILES: COc1cc(C(N)=O)cc2nc(NC(=O)c3cc(C)nn3CCO)n(C/C=C/Cn3c(NC(=O)c4cc(C)nn4CCO)nc4cc(C(N)=O)cc(OCCCN5CCN(CCOCCOCCOCCOCCOCCOCCOCCOCCOCCOCCNC(=O)CN6C(=O)C=CC6=O)CC5)c43)c12 ; COc1cc(C(N)=O)cc2nc(NC(=O)c3cn(C)nn3)n(C/C=C/Cn3c(NC(=O)c4cn(C)nn4)nc4cc(C(N)=O)cc(OCCCN5CCN(CCOCCOCCOCCOCCOCCOCCOCCOCCOCCOCCNC(=O)CN6C(=O)C=CC6=O)CC5)c43)c12]

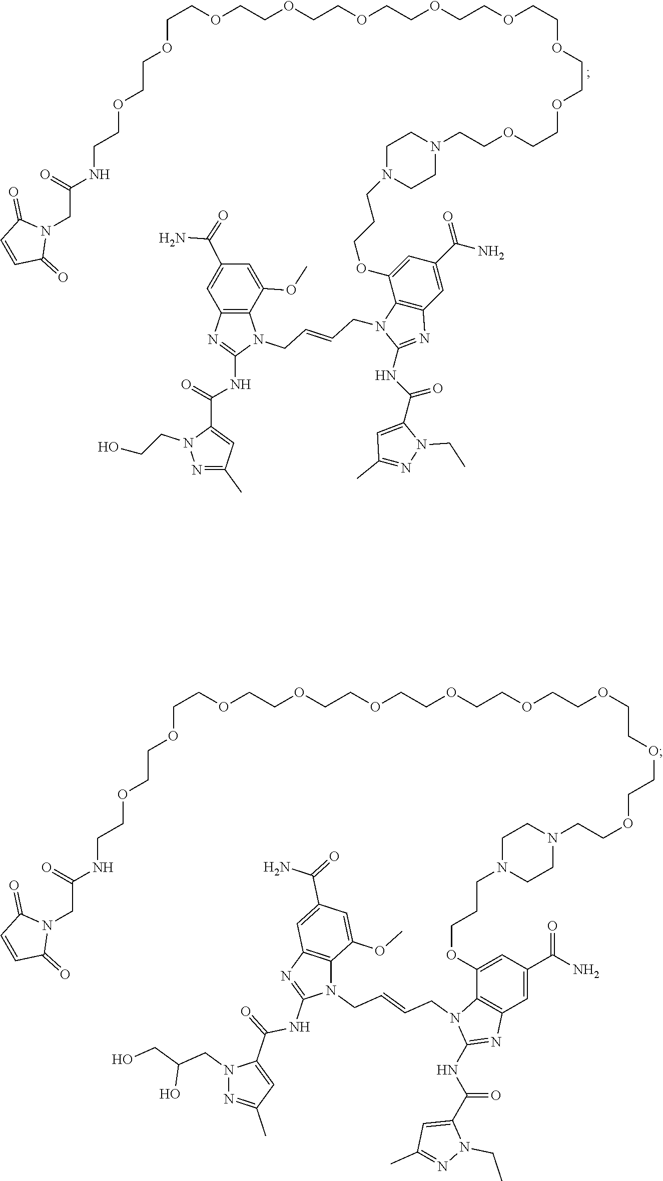 [SMILES: CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2CCO)nc2cc(C(N)=O)cc(OC)c21 ; CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2CC(O)CO)nc2cc(C(N)=O)cc(OC)c21]

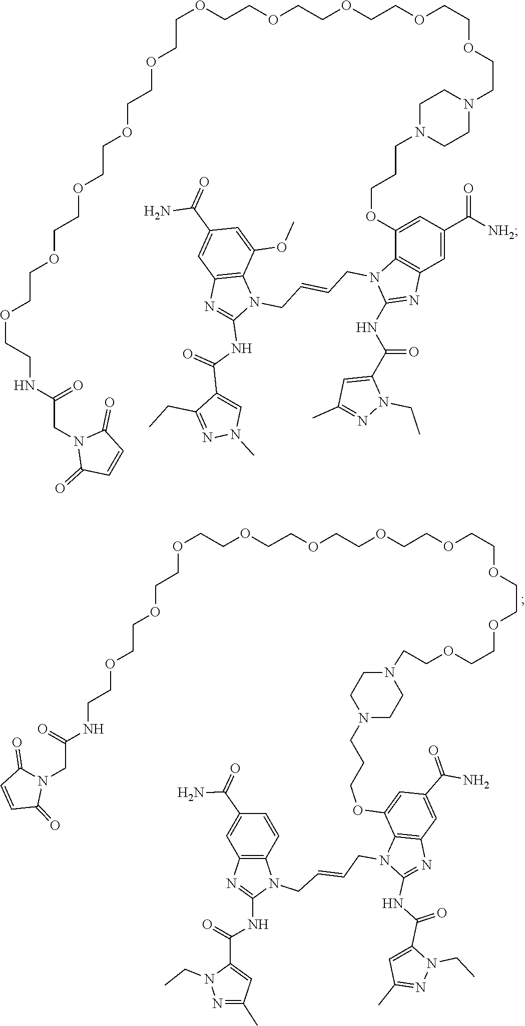 [SMILES: CCc1nn(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21 ; CCn1nc(C)cc1C(=O)Nc1nc2cc(C(N)=O)ccc2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21]

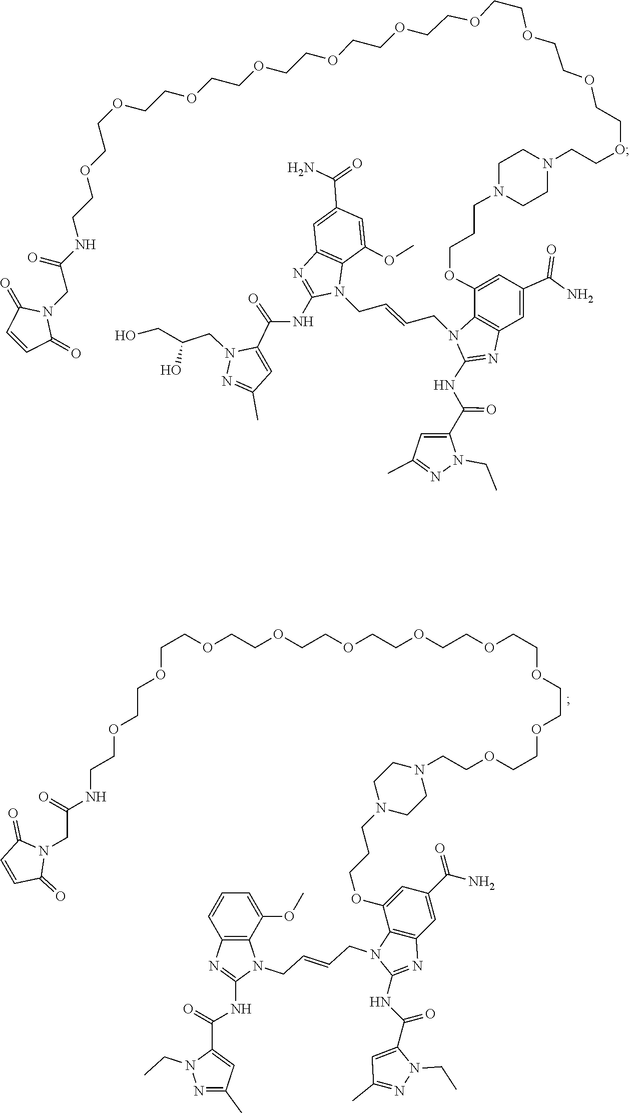 [SMILES: CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2C[C@H](O)CO)nc2cc(C(N)=O)cc(OC)c21 ; CCn1nc(C)cc1C(=O)Nc1nc2cccc(OC)c2n1C/C=C/Cn1c(NC(=O)c2cc(C)nn2CC)nc2cc(C(N)=O)cc(OCCCN3CCN(CCOCCOCCOCCOCCOCCOCCOCCOCCOCCOCCNC(=O)CN4C(=O)C=CC4=O)CC3)c21]

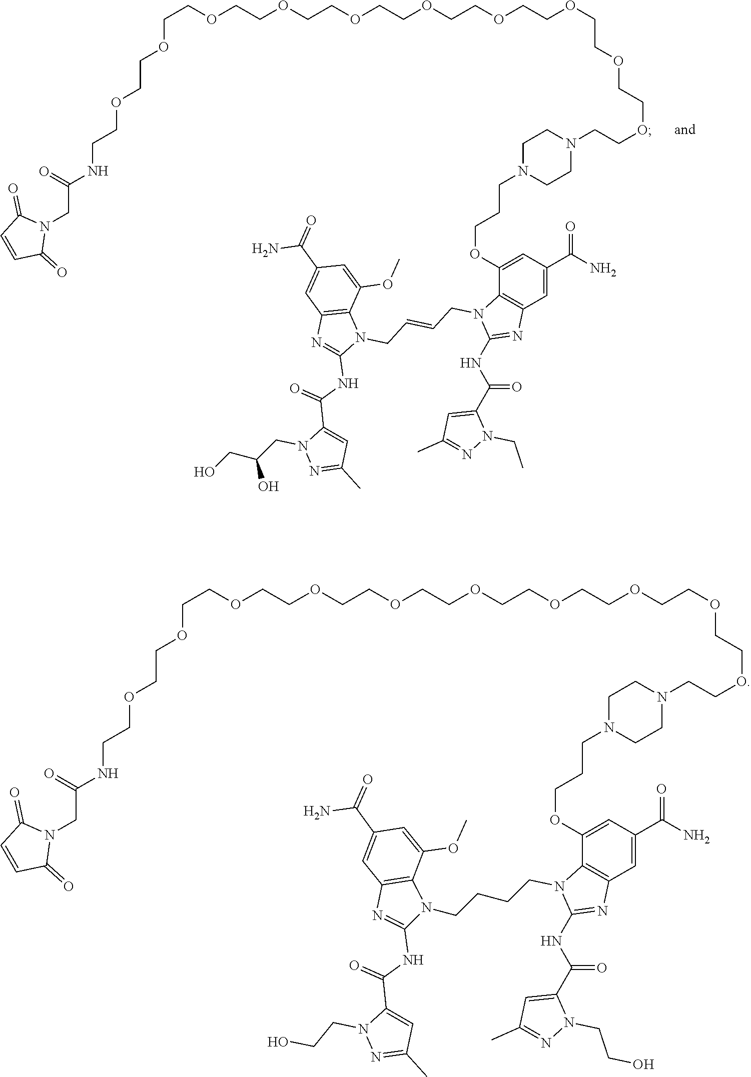 [SMILES: CCn1nc(C)cc1C(=O)Nc1nc2cc(C(N)=O)cc(OCCCN3CCN(CCOCCOCCOCCOCCOCCOCCOCCOCCOCCOCCNC(=O)CN4C(=O)C=CC4=O)CC3)c2n1C/C=C/Cn1c(NC(=O)c2cc(C)nn2C[C@@H](O)CO)nc2cc(C(N)=O)cc(OC)c21 ; COc1cc(C(N)=O)cc2nc(NC(=O)c3cc(C)nn3CCO)n(CCCCn3c(NC(=O)c4cc(C)nn4CCO)nc4cc(C(N)=O)cc(OCCCN5CCN(CCOCCOCCOCCOCCOCCOCCOCCOCCOCCOCCNC(=O)CN6C(=O)C=CC6=O)CC5)c43)c12]

39. An immunoconjugate prepared by conjugation of an antibody with a STING agonist-linker intermediate compound of claim 31.

40. A pharmaceutical composition comprising a therapeutically effective amount of an immunoconjugate of claim 1, and one or more pharmaceutically acceptable diluent, vehicle, carrier or excipient.

41. A method for treating cancer comprising administering a therapeutically effective amount of an immunoconjugate according to claim 1, to a patient in need thereof, wherein the cancer is selected from bladder cancer, salivary gland cancer, endometrial cancer, urinary tract cancer, urothelial carcinoma, lung cancer, non-small cell lung cancer, Merkel cell carcinoma, colon cancer, colorectal cancer, gastric cancer, and breast cancer.

42. The method of claim 41, wherein the cancer is susceptible to a pro-inflammatory response induced by STING agonism.

43. (canceled)

44. (canceled)

45. A method of preparing an immunoconjugate of Formula I of claim 1 wherein a STING agonist-linker intermediate compound selected from Table 1 is conjugated with the antibody.
